(19) United States
(12) Patent Application Publication
(10) Pub. No.: US 2012/0157471 A1
(43) Pub. Date: Jun. 21, 2012

(54) BENZIMIDAZOLE DERIVATIVES

(75) Inventors: Sajiv Krishnan Nair, Vista, CA (US); Simon Paul Planken, San Diego, CA (US); Michael Bruno Plewe, San Diego, CA (US); William Francois Vernier, San Diego, CA (US); Yi Yang, San Diego, CA (US); Huichun Zhu, San Diego, CA (US)

(73) Assignee: PFIZER INC., New York, NY (US)

(21) Appl. No.: 13/393,121

(22) PCT Filed: Aug. 11, 2010
(86) PCT No.: PCT/IB2010/053634

§ 371 Date: Feb. 28, 2012

Related U.S. Application Data
(60) Provisional application No. 61/238,953, filed on Sep. 1, 2009.

(51) Int. Cl. A61K 31/497; A61K 31/437; A61P 35/00; A61K 31/438; C07D 401/14; A61K 31/4545; C07D 471/04; C07D 498/10

(52) U.S. Cl. 514/253.09; 546/118; 514/303; 546/19; 514/278; 546/193; 514/318; 544/364

(57) ABSTRACT

The present invention relates to a compound of formula (I) or a pharmaceutically acceptable salt thereof, wherein R^{1A}, R^{1B}, R^{1C}, R², R³, R⁴, R⁵, R_{A}, R_{B}, R_{C} and X are as defined herein. These novel benzimidazole derivatives are useful in therapy, in particular for treating diseases or conditions mediated by SMO, including the treatment of abnormal cell growth, such as cancer, in mammals. This invention also relates to a method of using such compounds in the treatment of abnormal cell growth in mammals, especially humans, and to pharmaceutical compositions containing such compounds.

This application claims priority to U.S. Provisional application No. 61/238,953 filed on Sep. 1, 2009, which is incorporated herein by reference in its entirety.

### FIELD OF INVENTION

This invention relates to novel benzimidazole derivatives that are useful in therapy, in particular for treating diseases or conditions mediated by SMO, including the treatment of abnormal cell growth, such as cancer, in mammals. This invention also relates to a method of using such compounds in the treatment of abnormal cell growth in mammals, especially humans, and to pharmaceutical compositions containing such compounds.

### BACKGROUND OF THE INVENTION

Hedgehog (Hh) proteins are secreted morphogens that are involved in many biological processes during embryonic development. Postnatally, Hh has important roles in tissue homeostasis and aberrant Hh signaling is associated with developmental disorders and several types of cancer. At the cell surface, the Hh signal is thought to be relayed by the 12 transmembrane domain protein Patched (Ptc) (Hooper and Scott, Cell 59: 75 1-65 (1989); Nakano et al., Nature 341: 508-13 (1989)) and the G-protein-coupled-like receptor Smoothened (Smo) (Alcedo et al., Cell 86: 221-232 (1996); van den Heuvel and Tngham, Nature 382: 547-551 (1996)). Both genetic and biochemical evidence support a receptor model where Ptc and Smo are part of a multi-component receptor complex (Chen and Struhl, Cell 87: 553-63 (1996); Mango et al., Nature 384: 176-9 (1996); Stone et al., Nature 384:129-34 (1996)). Upon binding of Hh to Ptc, the normal inhibitory effect of Ptc on Smo is relieved, allowing Smo to transduce the Hh signal across the plasma membrane. However, the exact mechanism by which Ptc controls Smo activity still has yet to be clarified.

The signaling cascade initiated by Smo results in activation of Gli transcription factors that translocate into the nucleus where they control transcription of target genes. Gli has been shown to influence transcription of Hh pathway inhibitors such as Ptc and Hip I in a negative feedback loop indicating that tight control of the Hh pathway activity is required for proper cellular differentiation and organ formation. Uncontrolled activation of Hh signaling pathway is associated with malignancies in particular those of the brain, skin and muscle as well as angiogenesis. An explanation for this is that the Hh pathway has been shown to regulate cell proliferation in adults by activation of genes involved in cell cycle progression such as cyclin D which is involved in G1-S transition. Also, Sonic Hedgehog (SHh), an ortholog of Hh, blocks cell-cycle arrest mediated by p21, an inhibitor of cyclin dependent kinases. Hh signaling is further implicated in cancer by inducing components in the EGFR pathway (EGF, Her2) involved in proliferation as well as components in the PDGF (PDGFa) and VEGF pathways involved in angiogenesis. Loss of function mutations in the Ptc gene have been identified in patients with the basal cell nevus syndrome (BCNS), a hereditary disease characterized by multiple basal cell carcinomas (BCCs). Dysfunctional Ptc gene mutations have also been associated with a large percentage of sporadic basal cell carcinoma tumors (Chidambaram et al., Cancer Research 56: 4599-601 (1996); Gailani et al., Nature Genet. 14: 78-81 (1996); Hahn et al., Cell 85: 841-51 (1996); Johnson et al., Science 272: 1668-71 (1996); Unden et al., Cancer Res. 56: 4562-5; Wicking et al., Am. J. Hum. Genet. 60: 21-6 (1997)). Loss of Ptc function is thought to cause an uncontrolled Smo signaling in basal cell carcinoma. Similarly, activating Smo mutations have been identified in sporadic BCC tumors (Xie et al., Nature 391: 90-2 (1998)), emphasizing the role of Smo as the signaling subunit in the receptor complex for SHh. Various inhibitors of hedgehog signaling have been investigated such as Cyclopamine, a natural alkaloid that has been shown to arrest cell cycle at G0-GI and to induce apoptosis in SCLC. Cyclopamine is believed to inhibit Smo by binding to its heptahelical bundle. Forskolin has been shown to inhibit the Hh pathway downstream from Smo by activating protein kinase A (PKA) which maintains Gli transcription factors inactive. Despite advances with these and other compounds, there remains a need for potent inhibitors of the hedgehog signaling pathway.

### SUMMARY OF THE INVENTION

The present invention relates to a compound of Formula I:

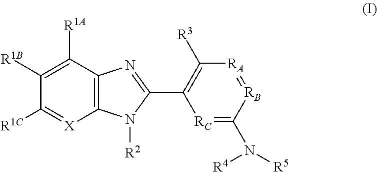

wherein X is selected from N and CR⁶; R_{A}, R_{B}, and R_{C} are each independently selected from CH and N, provided that at least one of R_{A}, R_{B}, and R_{C} is N; R^{1A}, R^{1B}, R^{1C} and R² are each independently selected from H, halo, —CN, C₁₋₁₀ alkyl, C₂₋₆ alkenyl, C₂₋₆ alkynyl, —NR⁶R⁷, —OR⁶, —C(O)R⁶, —C(O)OR⁶, —C(O)NR⁶R⁷, C₃₋₁₀ cycloalkyl, 3-12 membered heterocyclyl, C₆₋₁₀ aryl and 5-12 membered heteroaryl; R³ is selected from H, halo, —CN, C₁₋₁₀ alkyl, C₂₋₆ alkenyl, C₂₋₆ alkynyl, —NR⁶R⁷, —OR⁶, —C(O)R⁶, —C(O)OR⁶, C₃₋₁₀ cycloalkyl, 3-12 membered heterocyclyl, C₆₋₁₀ aryl and 5-12 membered heteroaryl, wherein each of said C₃₋₁₀ cycloalkyl, 3-12 membered heterocyclyl, C₆₋₁₀ aryl and 5-12 membered heteroaryl of said R³ moiety is optionally substituted with at least one R⁶ group; R⁴ and R⁵ are each independently selected from H, —(CR¹³R¹⁴)ₘCN, —(CR¹³R¹⁴)ₘ C₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘS(O)₂(R⁷), —(CR¹³R¹⁴)ₘNR⁶R⁷, —(CR¹³R¹⁴)ₘNR⁶OR⁷, —(CR¹³R¹⁴)ₘNR⁶C(O)R⁷, —(CR¹³R¹⁴)ₘNR⁶C(O)OR⁷, —(CR¹³R¹⁴)ₘNR⁶S(O)₂R⁷, —NR⁶(CR¹³R¹⁴)ₘS(O)₂NR⁶R⁷, —(CR¹³R¹⁴)ₘNR¹³(CR¹³R¹⁴)ₘOR⁷, —(CR¹³R¹⁴)ₘS(O)₂NR⁶R⁷, —(CR¹³R¹⁴)ₘOR⁶, —(CR¹³R¹⁴)ₘC(O)R⁶, —(CR¹³R¹⁴)ₘC(O)OR⁶, —(CR¹³R¹⁴)ₘC(O)NR⁶R⁷, —(CR¹³R¹⁴)ₘ(O)C(O)NR⁶R⁷, —(CR¹³R¹⁴)ₘ(O)(CR¹³R¹⁴)ₘOR⁶, —(CR¹³R¹⁴)ₘ(O)(CR¹³R¹⁴)ₘNR⁶R⁷, —(CR¹³R¹⁴)ₘC₃₋₁₀ cycloalkyl, —(CR¹³R¹⁴)ₘ(3-12 membered heterocyclyl), —(CR¹³R¹⁴)ₘ(C₆₋₁₀ aryl) and —(CR¹³R¹⁴)ₘ(5-12 membered heteroaryl), wherein each of said R⁴ and R⁵ moieties is optionally substituted with at least one R¹⁰ group; or R⁴ and R⁵, together with the nitrogen atom to which they are attached, form a 3-12 membered heterocyclyl optionally substituted with at least one R⁶ group; each R⁶ and R⁷ is independently selected from H, —(CR¹³R¹⁴)ₘhalo, —(CR¹³R¹⁴)ₘOH, —(CR¹³R¹⁴)ₘCN, —(CR¹³R¹⁴)ₘC₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘNR⁸R⁹, —(CR¹³R¹⁴)ₘNR⁸C(O)R⁹, —(CR¹³R¹⁴)ₘNR⁸C(O)OR⁹, —(CR¹³R¹⁴)ₘN(R⁸)S(O)₂R⁹, —(CR¹³R¹⁴)ₘN(R⁸)(CR¹³R¹⁴)ₘNR⁸R⁹, —(CR¹³R¹⁴)ₘN(R⁸)(CR¹³R¹⁴)ₘN(R⁸)S(O)₂R⁹, —(CR¹³R¹⁴)ₘN(R⁸)(CR¹³R¹⁴)ₘS(O)₂NR⁸R⁹, —(CR¹³R¹⁴)ₘ(O)(CR¹³R¹⁴)ₘNR⁸R⁹, —(CR¹³R¹⁴)ₘ(O)(CR¹³R¹⁴)ₘC(O)NR⁸R⁹, —(CR¹³R¹⁴)ₘS(O)₂R⁸, —(CR¹³R¹⁴)ₘS(O)₂NR⁸R⁹, —(CR¹³R¹⁴)ₘC(O)R⁸, —(CR¹³R¹⁴)ₘC(O)OR⁸, —(CR¹³R¹⁴)ₘC(O)NR⁸R⁹, —(CR¹³R¹⁴)ₘ(O)C(O)R⁸, —(CR¹³R¹⁴)ₘOC(O)NR⁸R⁹, —(CR¹³R¹⁴)ₘOR⁸, —(CR¹³R¹⁴)ₘ(O)(CR¹³R¹⁴)ₘOR⁸, —(CR¹³R¹⁴)ₘ(C₃₋₁₀ cycloalkyl), —(CR¹³R¹⁴)ₘ(3-12 membered heterocyclyl), —(CR¹³R¹⁴)ₘC₆₋₁₀ aryl and —(CR¹³R¹⁴)ₘ(5-12 membered heteroaryl), wherein each of said R⁶ and R⁷ moieties is optionally substituted with at least one R¹⁰ group; each R⁸, R⁹ and R¹⁰ is independently selected from H, —(CR¹³R¹⁴)ₘhalo, —(CR¹³R¹⁴)ₘCN, —(CR¹³R¹⁴)ₘC₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘC₃₋₁₀ cycloalkyl, —(CR¹³R¹⁴)ₘC(O)R¹¹, —(CR¹³R¹⁴)ₘC(O)OR¹¹, —(CR¹³R¹⁴)ₘC(O)NR¹¹R¹², —(CR¹³R¹⁴)ₘ NR¹¹R¹², —(CR¹³R¹⁴)ₘS(O)₂R¹¹, —(CR¹³R¹⁴)ₘ N(R¹¹)C(O)R¹², —(CR¹³R¹⁴)ₘ(O)(CR¹³R¹⁴)ₘC(O)NR¹¹R¹², —(CR¹³R¹⁴)ₘOR¹¹, —(CR¹³R¹⁴)ₘ(3-12 membered heterocyclyl), —(CR¹³R¹⁴)ₘ(C₆₋₁₀ aryl) and —(CR¹³R¹⁴)ₘ(5-12 membered heteroaryl); each R¹¹ and R¹² is independently selected from H, halo, —(CR¹³R¹⁴)ₘOH, —(CR¹³R¹⁴)ₘCN, —(CR¹³R¹⁴)ₘ(C₁₋₁₀ alkyl), —(CR¹³R¹⁴)ₘ(C₂₋₆ alkenyl), —(CR¹³R¹⁴)ₘ(C₂₋₆ alkynyl), —(CR¹³R¹⁴)ₘ(C₃₋₁₀ cycloalkyl), —(CR¹³R¹⁴)ₘ(3-12 membered heterocyclyl), —(CR¹³R¹⁴)ₘ(C₆₋₁₀ aryl) and —(CR¹³R¹⁴)ₘ(5-12 membered heteroaryl); each R¹³ and R¹⁴ is independently selected from H, C₁₋₁₀ alkyl, —OH and halo; and each m is independently selected from 0, 1, 2, 3, 4, 5 and 6; or a pharmaceutically acceptable salt thereof.

In another embodiment, the invention provides a compound of formula (I) as described herein, or a pharmaceutically acceptable salt thereof, wherein R² is selected from H, halo, —CN, C₁₋₁₀ alkyl, C₂₋₆ alkenyl, C₂₋₆ alkynyl, —NR⁶R⁷, —OR⁶, —C(O)R⁶, —C(O)OR⁶ and —C(O)NR⁶R⁷.

In a further embodiment, the invention provides a compound of formula (I) as described herein, or a pharmaceutically acceptable salt thereof, wherein: X is CH; R^{1A}, R^{1B} and R^{1C} are H; R² is H, halo, C₁₋₁₀ alkyl or C₃₋₁₀ cycloalkyl; and R³ is halo or C₁₋₁₀ alkyl.

In yet a further embodiment, the invention provides a compound of formula (I) as described herein, or a pharmaceutically acceptable salt thereof, wherein: X is N; R^{1A}, R^{1B} and R^{1C} are H; R² is H, halo, C₁₋₁₀ alkyl or C₃₋₁₀ cycloalkyl; and R³ is halo or C₁₋₁₀ alkyl.

In still another embodiment, the invention provides a compound of formula (I) as described herein, or a pharmaceutically acceptable salt thereof, wherein: X is CH; R_{B} is N; R^{1A}, R^{1B} and R^{1C} are H; R² is H, halo, C₁₋₁₀ alkyl or C₃₋₁₀ cycloalkyl; and R³ is halo or C₁₋₁₀ alkyl.

In another embodiment, the invention provides a compound of formula (I) as described herein, or a pharmaceutically acceptable salt thereof, wherein: X is N; R_{B} is N; R^{1A}; R^{1B} and R^{1C} are H; R² is H, halo, C₁₋₁₀ alkyl or C₃₋₁₀ cycloalkyl; and R³ is halo or C₁₋₁₀ alkyl.

In a further embodiment, the invention provides a compound of formula (I) according to claim 1, or a pharmaceutically acceptable salt thereof, wherein: X is CH; R_{C} is N; R^{1A}, R^{1B} and R^{1C} are H; R² is H, halo, C₁₋₁₀ alkyl or C₃₋₁₀ cycloalkyl; and R³ is halo or C₁₋₁₀ alkyl.

In yet a further embodiment, the invention provides a compound of formula (I) as described herein, or a pharmaceutically acceptable salt thereof, wherein: X is N; R_{C} is N; R^{1A}, R^{1B} and R^{1C} are H; R² is H, halo, C₁₋₁₀ alkyl or C₃₋₁₀ cycloalkyl; and R³ is halo or C₁₋₁₀ alkyl.

In still another embodiment, the invention provides a compound of formula (I) as described herein, or a pharmaceutically acceptable salt thereof, wherein: X is CH; R_{B} and R_{C} are N; R^{1A}, R^{1B} and R^{1C} are H; R² is H, halo, C₁₋₁₀ alkyl or C₃₋₁₀ cycloalkyl; and R³ is halo or C₁₋₁₀ alkyl.

In another embodiment, the invention provides a compound of formula (I) as described herein, or a pharmaceutically acceptable salt thereof, wherein: X is N; R_{B} and R_{C} are N; R^{1A}, R^{1B} and R^{1C} are H; R² is H, halo, C₁₋₁₀ alkyl or C₃₋₁₀ cycloalkyl; and R³ is halo or C₁₋₁₀ alkyl.

In a further embodiment, the invention provides a compound of formula (I) as described herein, or a pharmaceutically acceptable salt thereof, wherein R³ is selected from H, halo, —CN, C₁₋₁₀ alkyl, C₂₋₆ alkenyl, C₂₋₆ alkynyl, —NR⁶R⁷, —OR⁶, —C(O)R⁶, —C(O)OR⁶, C₃₋₁₀ cycloalkyl, 3-12 membered heterocyclyl, C₆₋₁₀ aryl and 5-12 membered heteroaryl.

In yet a further embodiment, the invention provides a compound of formula (I) as described herein, or a pharmaceutically acceptable salt thereof, wherein R³ is selected from H, halo, —CN, C₁₋₁₀ alkyl, C₂₋₆ alkenyl, C₂₋₆ alkynyl, —NR⁶R⁷, —OR⁶, —C(O)R⁶ and —C(O)OR⁶.

In still another embodiment, the invention provides a compound of formula (I) as described herein, or a pharmaceutically acceptable salt thereof, wherein: X is CH; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵ are independently selected from H, —(CR¹³R¹⁴)ₘCN, —(CR¹³R¹⁴)ₘC₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘS(O)₂(R⁷), —(CR¹³R¹⁴)ₘNR⁶R⁷, —(CR¹³R¹⁴)ₘNR⁶OR⁷, —(CR¹³R¹⁴)ₘNR⁶C(O)R⁷, —(CR¹³R¹⁴)ₘNR⁶C(O)OR⁷, —(CR¹³R¹⁴)ₘNR⁶S(O)₂R⁷, —NR⁶(CR¹³R¹⁴)ₘS(O)₂NR⁶R⁷, —(CR¹³R¹⁴)ₘNR¹³(CR₁₃R¹⁴)ₘOR⁷; —(CR¹³R¹⁴)ₘS(O)₂NR⁶R⁷, —(CR¹³R¹⁴)ₘOR⁶, —(CR¹³R¹⁴)ₘC(O)R⁶, —(CR¹³R¹⁴)ₘC(O)OR⁶, —(CR¹³R¹⁴)ₘC(O)NR⁶R⁷, —(CR¹³R¹⁴)ₘC(O)NR⁶R⁷, —(CR¹³R¹⁴)ₘ(O)(CR¹³R¹⁴)ₘOR⁶; and —(CR¹³R¹⁴)ₘ(O)(CR¹³R¹⁴)ₘNR⁶R⁷, wherein each of said R⁴ and R⁵ moieties is optionally substituted with at least one R¹⁰ group.

In another embodiment, the invention provides a compound of formula (I) as described herein, or a pharmaceutically acceptable salt thereof, wherein: X is CH; R_{B} is N; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵ are independently selected from H, —(CR¹³R¹⁴)ₘCN, —(CR¹³R¹⁴)ₘC₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘS(O)₂(R⁷), —(CR¹³R¹⁴)ₘNR⁶R⁷, —(CR¹³R¹⁴)ₘNR⁶OR⁷, —(CR¹³R¹⁴)ₘNR⁶C(O)R⁷, —(CR¹³R¹⁴)ₘNR⁶C(O)OR⁷, —(CR¹³R¹⁴)ₘNR⁶S(O)₂R⁷, —NR⁶(CR¹³R¹⁴)ₘS(O)₂NR⁶R⁷, —(CR¹³R¹⁴)ₘNR¹³(CR¹³R¹⁴)ₘOR⁷, —(CR¹³R¹⁴)ₘS(O)₂NR⁶R⁷, —(CR¹³R¹⁴)ₘOR⁶, —(CR¹³R¹⁴)ₘC(O)R⁶, —(CR¹³R¹⁴)ₘC(O)OR⁶, —(CR¹³R¹⁴)ₘC(O)NR⁶R⁷, —(CR¹³R¹⁴)ₘ(O)C(O)NR⁶R⁷, —(CR¹³R¹⁴)ₘ(O)(CR¹³R¹⁴)ₘOR⁶, and —(CR¹³R¹⁴)ₘ(O)(CR¹³R¹⁴)ₘNR⁶R⁷, wherein each of said R⁴ and R⁵ moieties is optionally substituted with at least one R¹⁰ group.

In a further embodiment, the invention provides a compound of formula (I) according to claim 1, or a pharmaceutically acceptable salt thereof, wherein: X is CH; R_{C} is N; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵ are independently selected from H, —(CR¹³R¹⁴)ₘCN, —(CR¹³R¹⁴)ₘC₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘS(O)₂(R⁷), —(CR¹³R¹⁴)ₘNR⁶R⁷, —(CR¹³R¹⁴)ₘNR⁶OR⁷, —(CR¹³R¹⁴)ₘNR⁶C(O)R⁷, —(CR¹³R¹⁴)ₘNR⁶C(O)OR⁷, —(CR¹³R¹⁴)ₘNR⁶S(O)₂R⁷, —NR⁶(CR¹³R¹⁴)ₘS(O)₂NR⁶R⁷, —(CR¹³R¹⁴)ₘNR¹³(CR¹³R¹⁴)ₘOR⁷, —(CR¹³R¹⁴)ₘS(O)₂NR⁶R⁷, —(CR¹³R¹⁴)ₘOR⁶, —(CR¹³R¹⁴)ₘC(O)R⁶, —(CR¹³R¹⁴)ₘC(O)OR⁶, —(CR¹³R¹⁴)ₘC(O)NR⁶R⁷, —(CR¹³R¹⁴)ₘ(O)C(O)NR⁶R⁷, —(CR¹³R¹⁴)ₘ(O)(CR¹³R¹⁴)ₘOR⁶, and —(CR¹³R¹⁴)ₘ(O)(CR¹³R¹⁴)ₘNR⁶R⁷, wherein each of said R⁴ and R⁵ moieties is optionally substituted with at least one R¹⁰ group.

In a further embodiment, the invention provides a compound of formula (I) as described herein, or a pharmaceutically acceptable salt thereof, wherein: X is CH; R_{B} and R_{C} are N; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵ are independently selected from H, —(CR¹³R¹⁴)ₘCN, —(CR¹³R¹⁴)ₘC₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘS(O)₂(R⁷), —(CR¹³R¹⁴)ₘNR⁶R⁷, —(CR¹³R¹⁴)ₘNR⁶OR⁷, —(CR¹³R¹⁴)ₘNR⁶C(O)R⁷, —(CR¹³R¹⁴)ₘNR⁶C(O)OR⁷, —(CR¹³R¹⁴)ₘNR⁶S(O)₂R⁷, —NR⁶(CR¹³R¹⁴)ₘS(O)₂NR⁶R⁷, —(CR¹³R¹⁴)ₘNR¹³(CR¹³R¹⁴)ₘOR⁷, —(CR¹³R¹⁴)ₘS(O)₂NR⁶R⁷, —(CR¹³R¹⁴)ₘOR⁶, —(CR¹³R¹⁴)ₘC(O)R⁶, —(CR¹³R¹⁴)ₘC(O)OR⁶, —(CR¹³R¹⁴)ₘC(O)NR⁶R⁷, —(CR¹³R¹⁴)ₘ(O)C(O)NR⁶R⁷, —(CR¹³R¹⁴)ₘ(O)(CR¹³R¹⁴)ₘOR⁶, and —(CR¹³R¹⁴)ₘ(O)(CR¹³R¹⁴)ₘNR⁶R⁷ wherein each of said R⁴ and R⁵ moieties is optionally substituted with at least one R¹⁰ group.

In yet a further embodiment, the invention provides a compound of formula (I) as described herein, or a pharmaceutically acceptable salt thereof, wherein: X is CH; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵, together with the nitrogen atom to which they are attached, form a 3-12 membered heterocyclyl optionally substituted with at least one R¹⁰ group.

In still another embodiment, the invention provides a compound of formula (I) as described herein, or a pharmaceutically acceptable salt thereof, wherein: X is CH; R_{B} is N; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵, together with the nitrogen atom to which they are attached, form a 3-12 membered heterocyclyl optionally substituted with at least one R¹⁰ group.

In another embodiment, the invention provides a compound of formula (I) as described herein, or a pharmaceutically acceptable salt thereof, wherein: X is CH; R_{C} is N; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵, together with the nitrogen atom to which they are attached, form a 3-12 membered heterocyclyl optionally substituted with at least one R¹⁰ group.

In a further embodiment, the invention provides a compound of formula (I) as described herein, or a pharmaceutically acceptable salt thereof, wherein: X is CH; R_{B} and R_{C} are N; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵, together with the nitrogen atom to which they are attached, form a 3-12 membered heterocyclyl optionally substituted with at least one R¹⁰ group.

In yet a further embodiment, the invention provides a compound of formula (I) as described herein, or a pharmaceutically acceptable salt thereof, wherein: X is N; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵, together with the nitrogen atom to which they are attached, form a 3-12 membered heterocyclyl optionally substituted with at least one R¹⁰ group.

In still another embodiment, the invention provides a compound of formula (I) as described herein, or a pharmaceutically acceptable salt thereof, wherein: X is N; R_{B} is N; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵, together with the nitrogen atom to which they are attached, form a 3-12 membered heterocyclyl optionally substituted with at least one R¹⁰ group.

In another embodiment, the invention provides a compound of formula (I) as described herein, or a pharmaceutically acceptable salt thereof, wherein: X is N; R_{C} is N; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵, together with the nitrogen atom to which they are attached, form a 3-12 membered heterocyclyl optionally substituted with at least one R¹⁰ group.

In a further embodiment, the invention provides a compound of formula (I) as described herein, or a pharmaceutically acceptable salt thereof, wherein: X is N; R_{B} and R_{C} are N; R^{1A}, R^{1B} and R^{1C} are H; R² is H, halo, C₁₋₁₀ alkyl or C₃₋₁₀ cycloalkyl; R³ is halo or C₁₋₁₀ alkyl; and R⁴ and R⁵, together with the nitrogen atom to which they are attached, form a 3-12 membered heterocyclyl optionally substituted with at least one R¹⁰ group.

In yet a further embodiment, the invention provides a method for the treatment of abnormal cell growth in a mammal comprising administering to said mammal an amount of a compound of formula (I) as described herein, or a pharmaceutically acceptable salt thereof, that is effective in treating abnormal cell growth.

In still another embodiment, the invention provides a method for the treatment of abnormal cell growth in a mammal as described herein wherein said abnormal cell growth is cancer.

In another embodiment, the invention provides a method for the treatment of abnormal cell growth in a mammal as described herein wherein said cancer is selected from the group consisting of basal cell cancer, medulloblastoma cancer, liver cancer, rhabdomyosarcoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma and pituitary adenoma, or a combination of one or more of the foregoing cancers.

In a further embodiment, the invention provides a pharmaceutical composition comprising a compound of formula (I) as described herein, or a pharmaceutically acceptable salt thereof, and a pharmaceutically acceptable carrier or diluent.

In yet a further embodiment, the invention provides a kit comprising: (i) a pharmaceutical composition comprising a compound of formula (I) as described herein, or a pharmaceutically acceptable salt thereof; and (ii) instructions for use of said pharmaceutical composition.

In still another embodiment, the invention provides a pharmaceutical composition comprising: (i) a compound of formula (I) as described herein, or a pharmaceutically acceptable salt thereof; (ii) at least one substance selected from an anti-angiogenesis agent, a signal transduction inhibitor, and an antiproliferative agent; and (iii) a pharmaceutically acceptable carrier or diluent.

In another embodiment, the invention provides a kit comprising: (i) a pharmaceutical composition comprising: (a) a compound of formula (I) as described herein, or a pharmaceutically acceptable salt thereof; and (b) at least one substance selected from an anti-angiogenesis agent, a signal transduction inhibitor, and an antiproliferative agent; and (ii) instructions for use of said pharmaceutical composition.

In a further embodiment, the invention provides the use of a compound of formula (I) as described herein, or a pharmaceutically acceptable salt thereof, for the manufacture of a medicament for treating abnormal cell growth in a mammal.

In another embodiment, the invention provides a compound of formula (I) as described herein, or a pharmaceutically acceptable salt thereof, for use as a medicament.

In still another embodiment, the invention provides a compound of formula (I) as described herein, or a pharmaceutically acceptable salt thereof, for use in the treatment of abnormal cell growth.

In a further embodiment, the invention provides the use of a compound of formula (I) as described herein, or a pharmaceutically acceptable salt thereof, for the preparation of a medicament for use in the treatment of abnormal cell growth.

In yet a further embodiment, the invention provides a compound selected from:

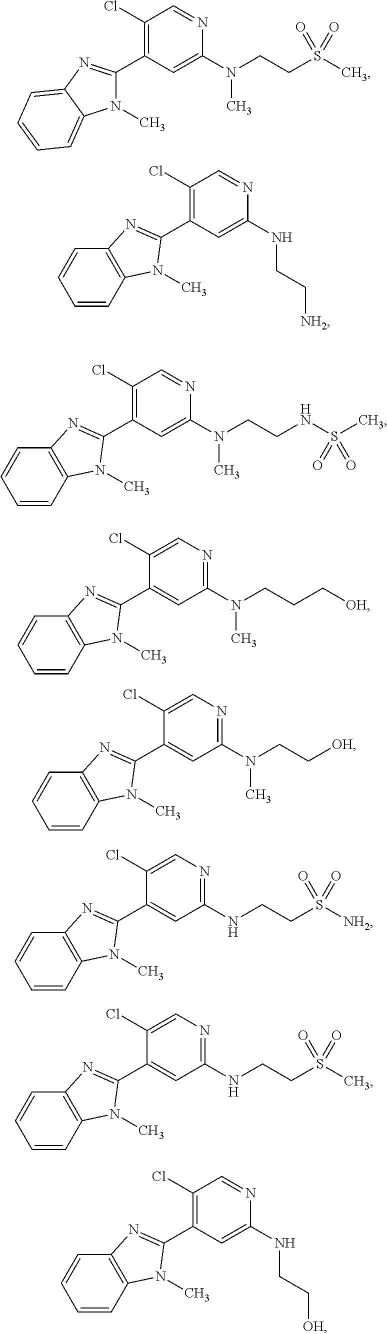

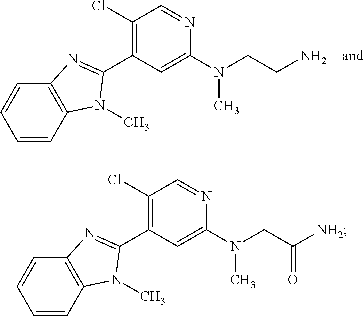

or a pharmaceutically acceptable salt thereof.

In still another embodiment, the invention provides a compound selected from:

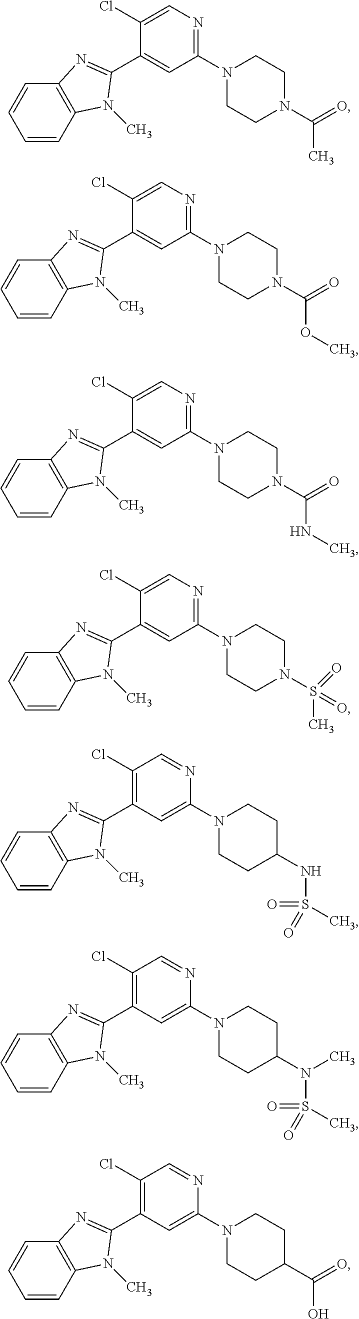

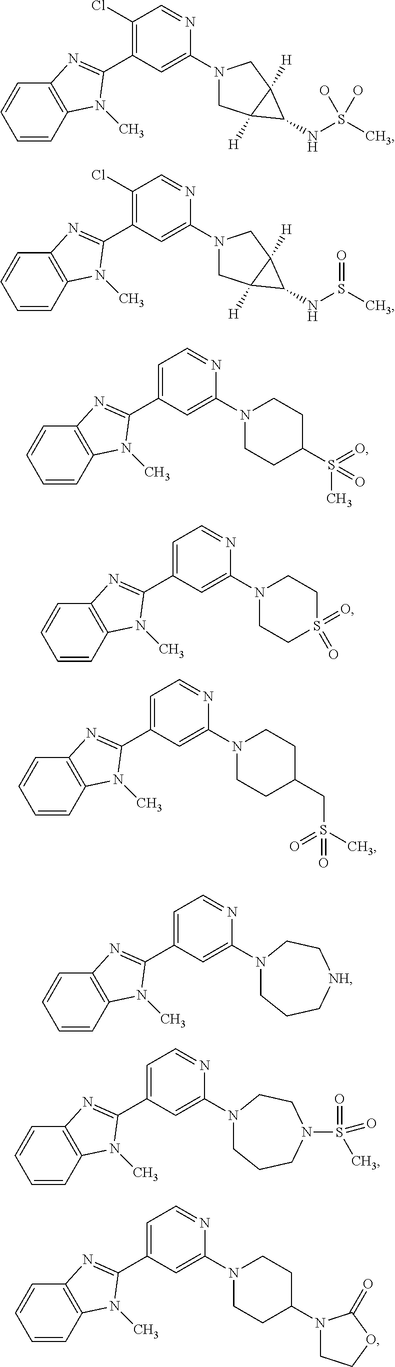

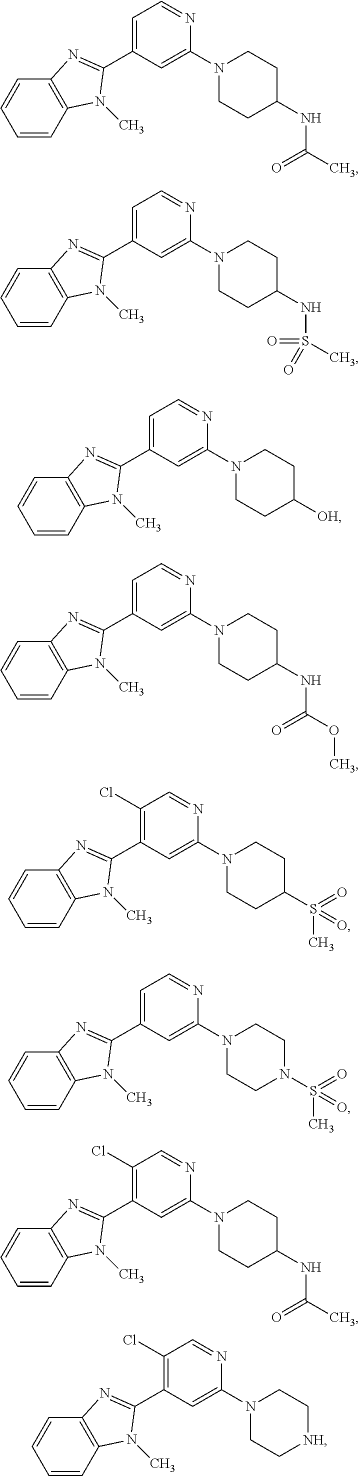

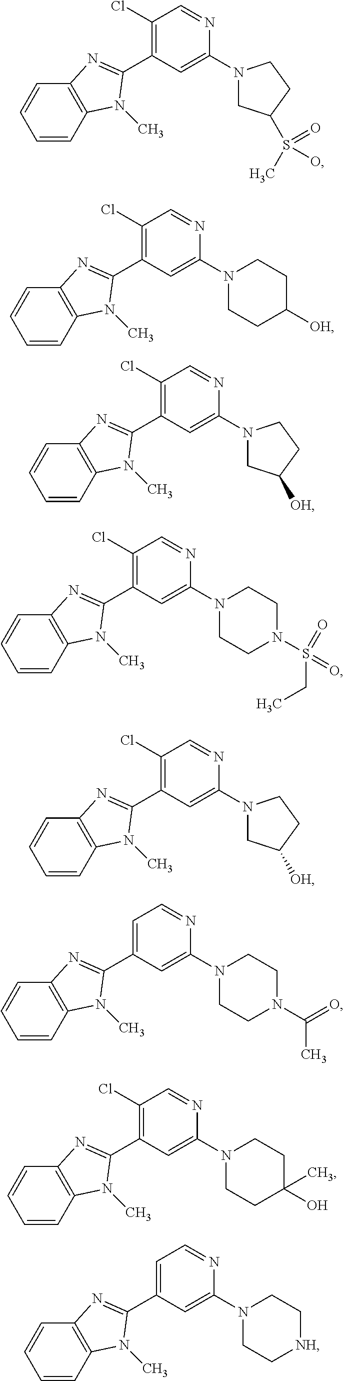

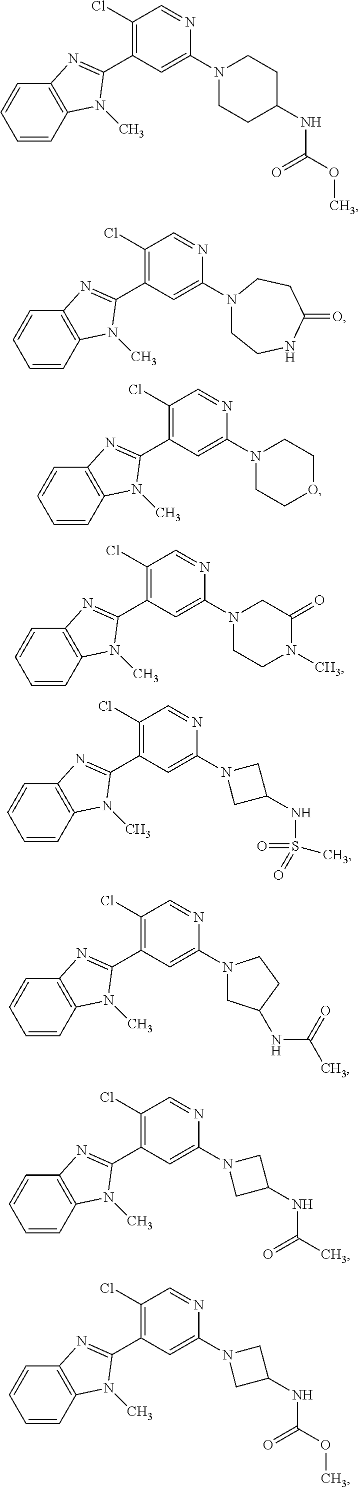

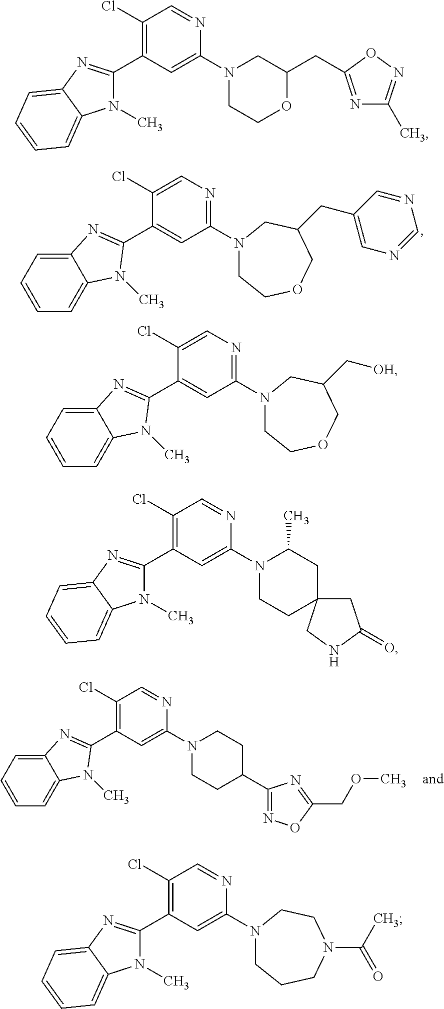

or a pharmaceutically acceptable salt thereof.

In another embodiment, the invention provides a compound selected from:

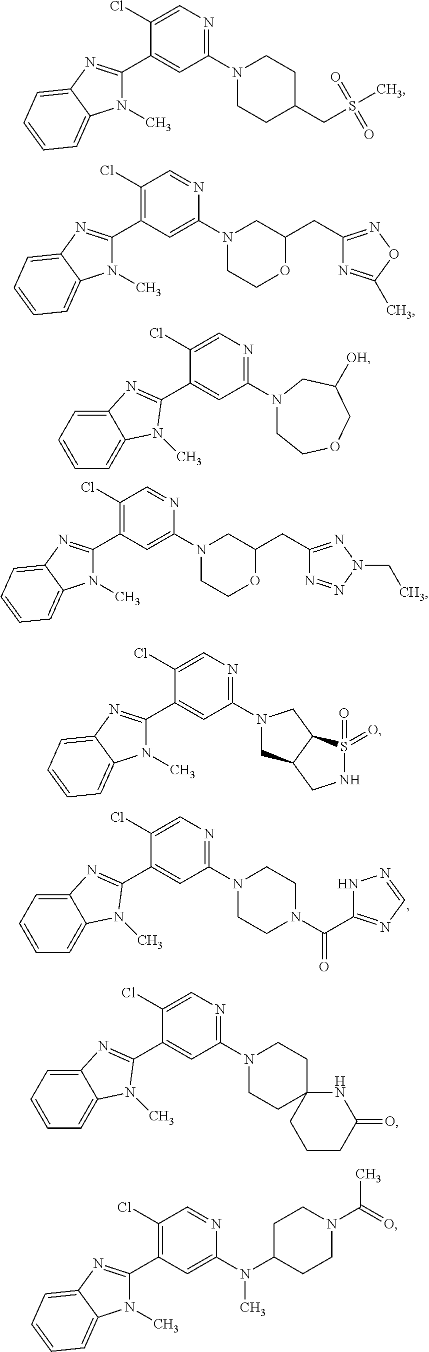

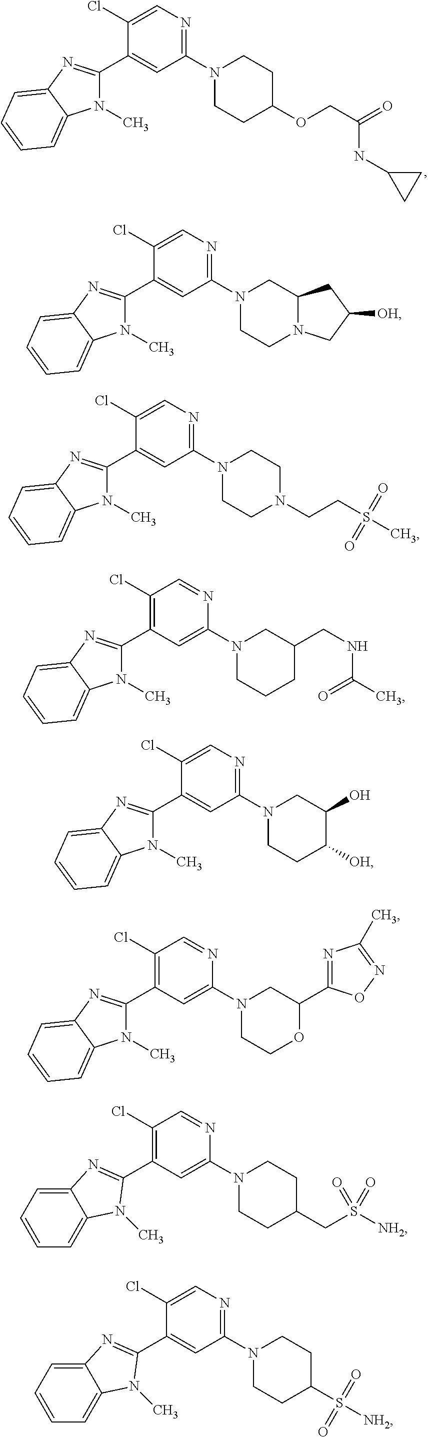

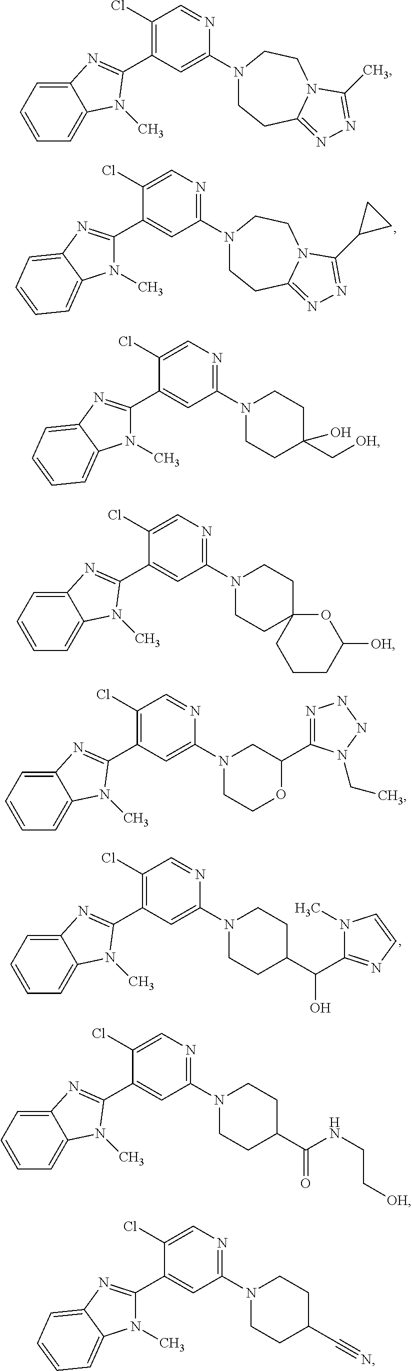

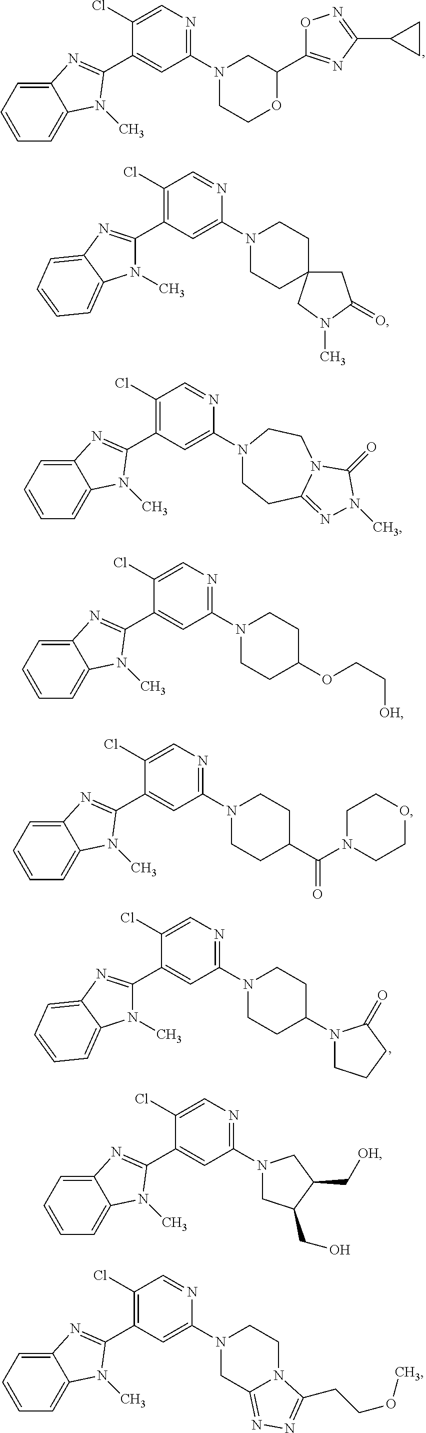

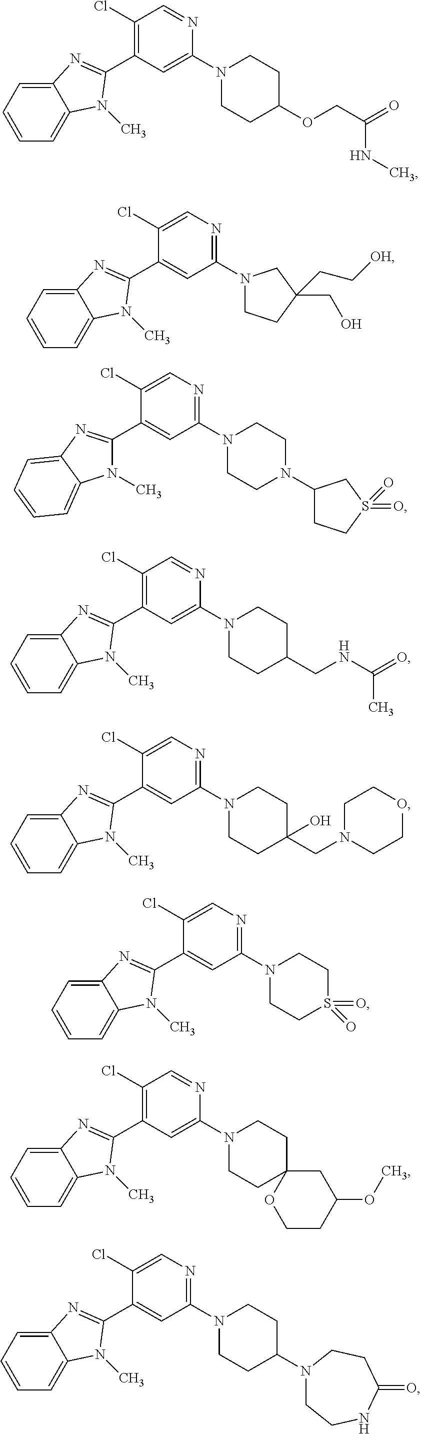

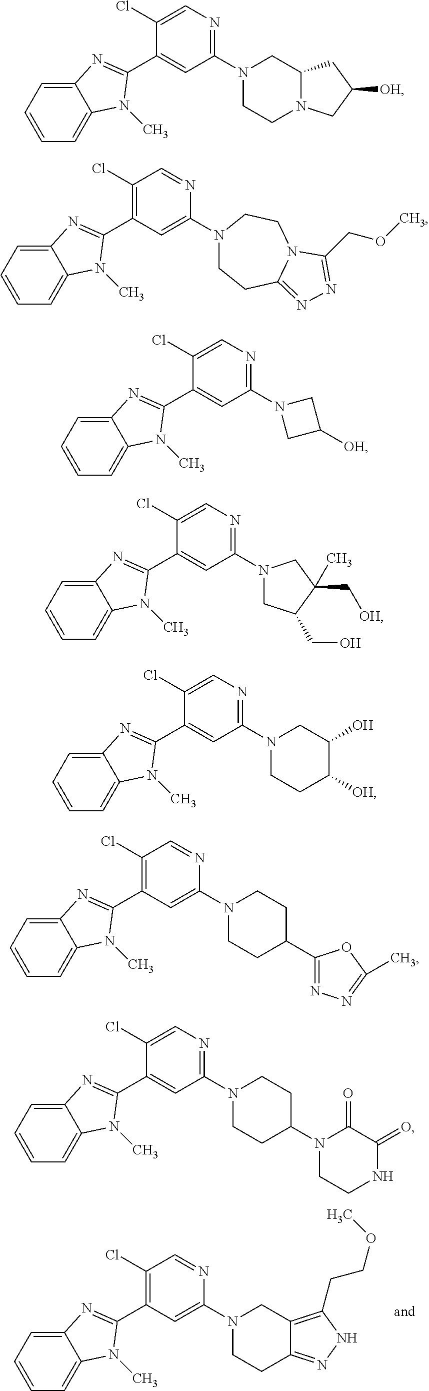

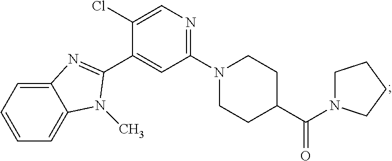

or a pharmaceutically acceptable salt thereof.

In a further embodiment, the invention provides a compound selected from:

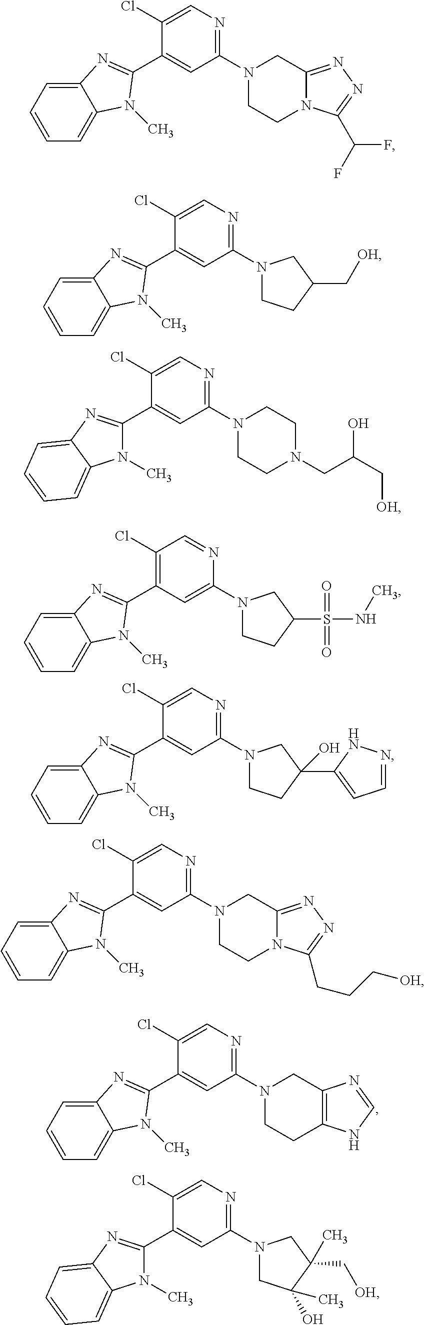

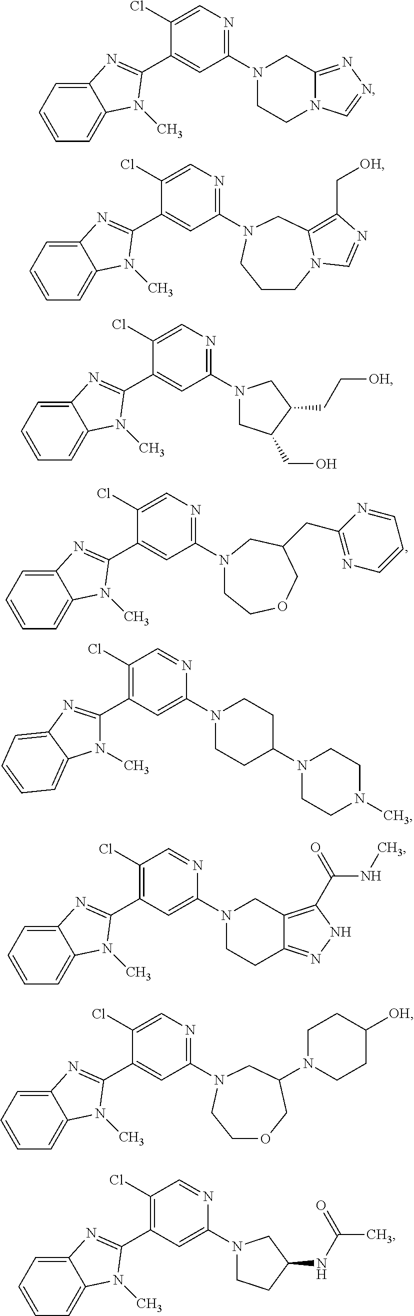

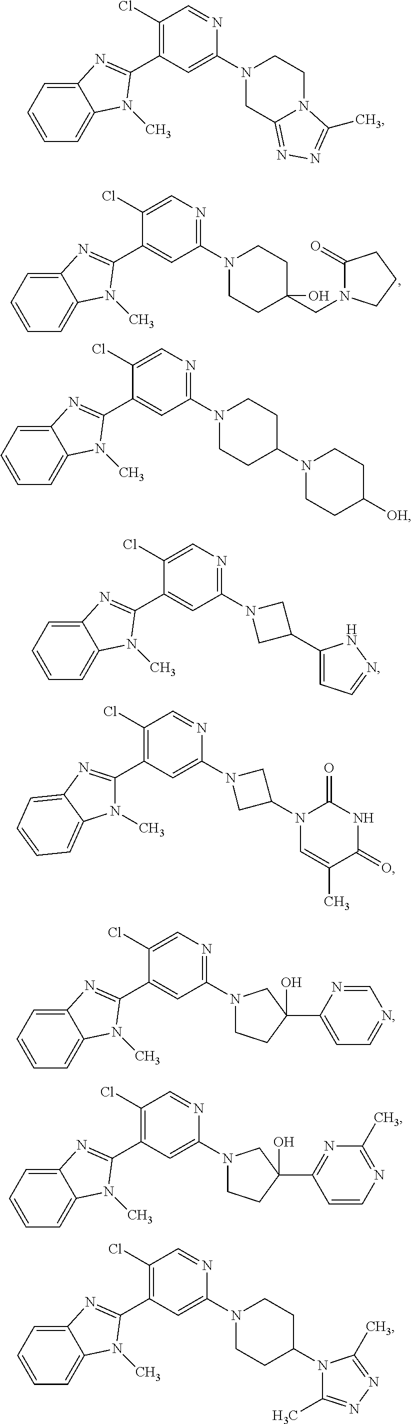

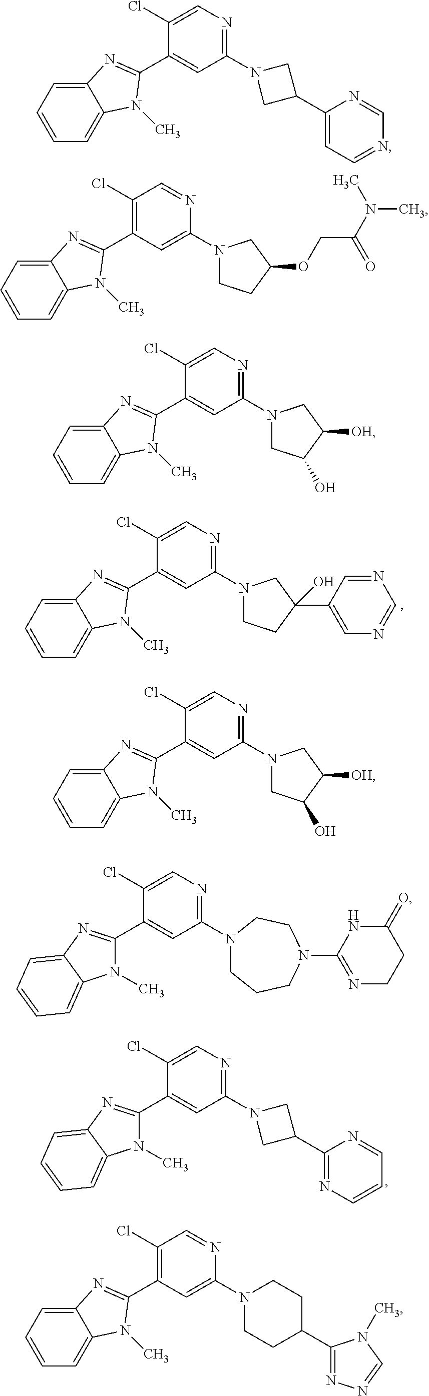

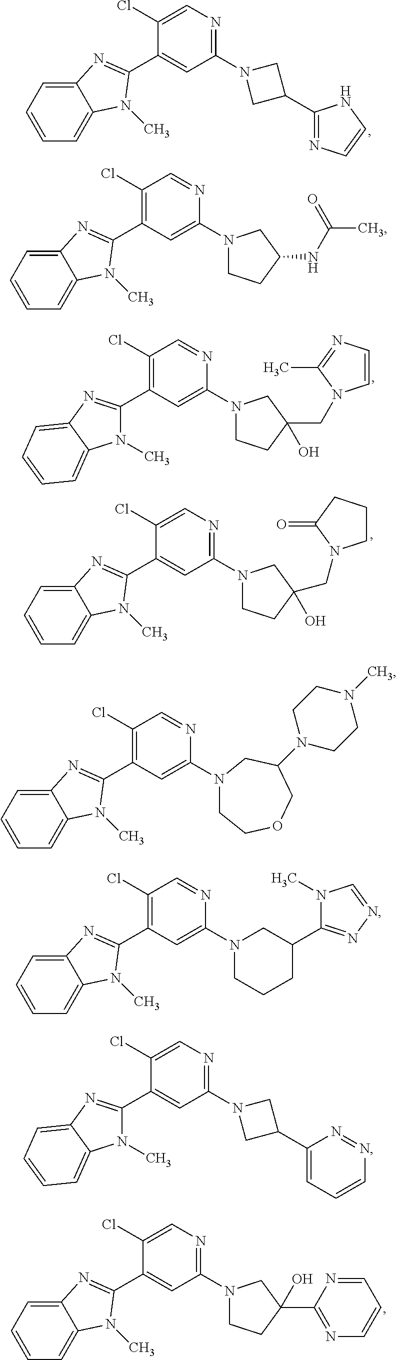

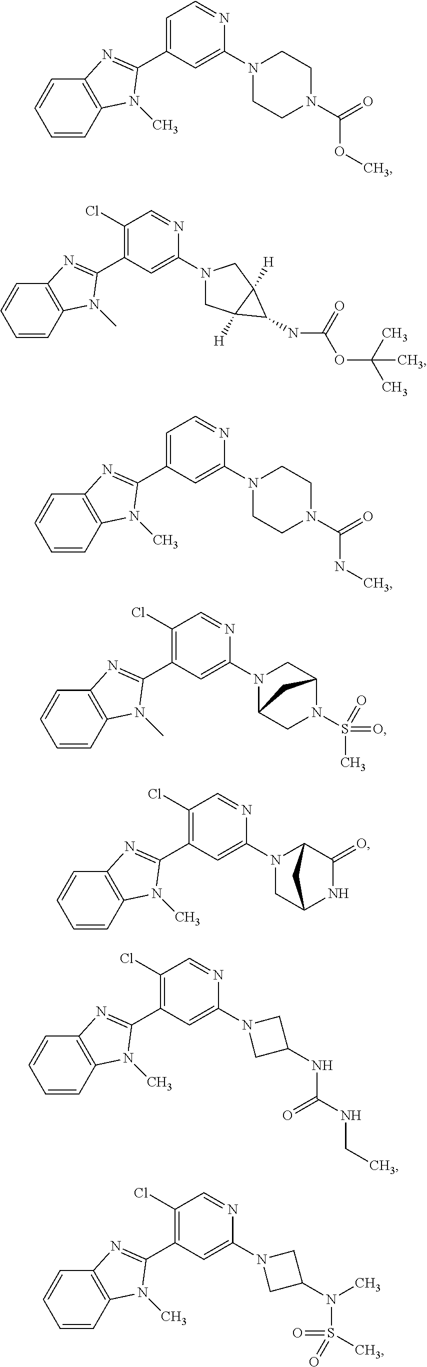

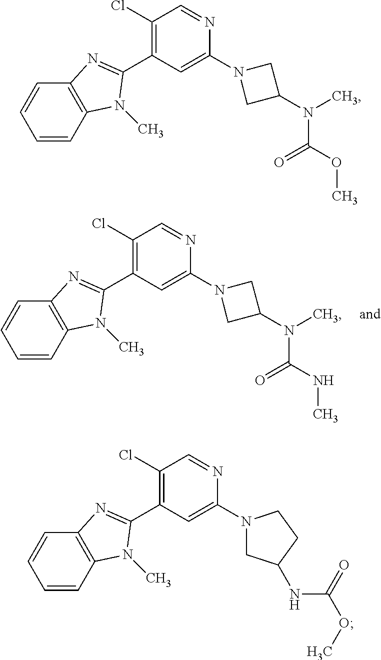

or a pharmaceutically acceptable salt thereof.

In yet a further embodiment, the invention provides a compound selected from:

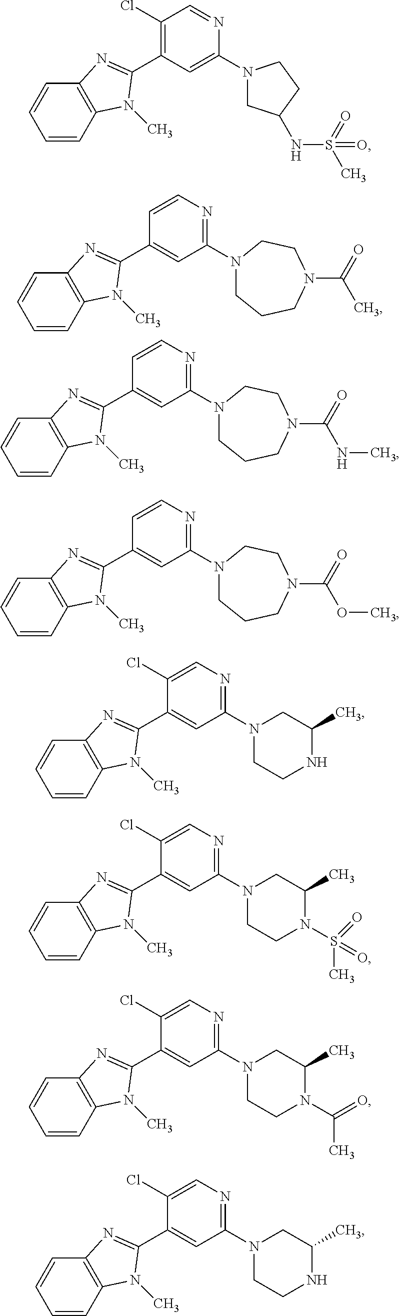

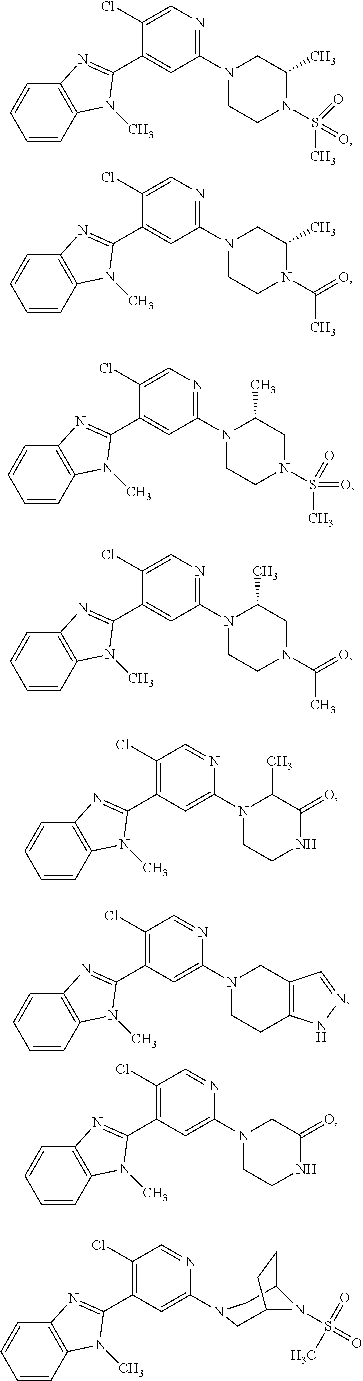

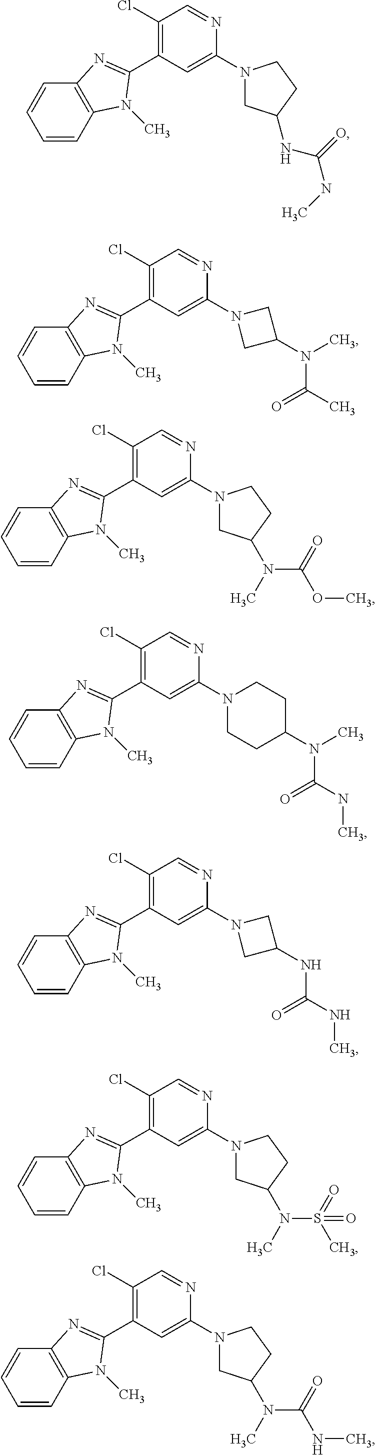

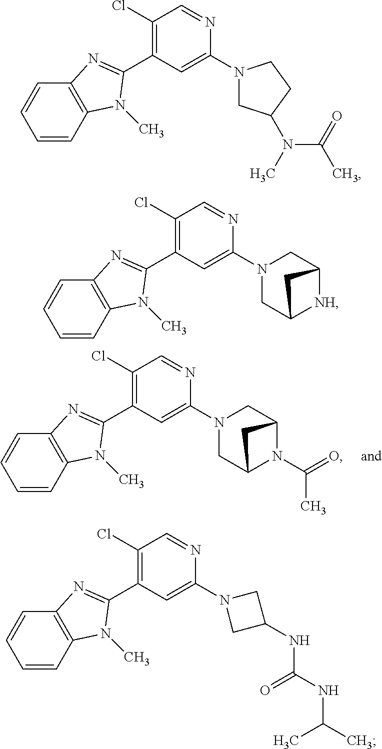

or a pharmaceutically acceptable salt thereof.

In still another embodiment, the invention provides a compound selected from:

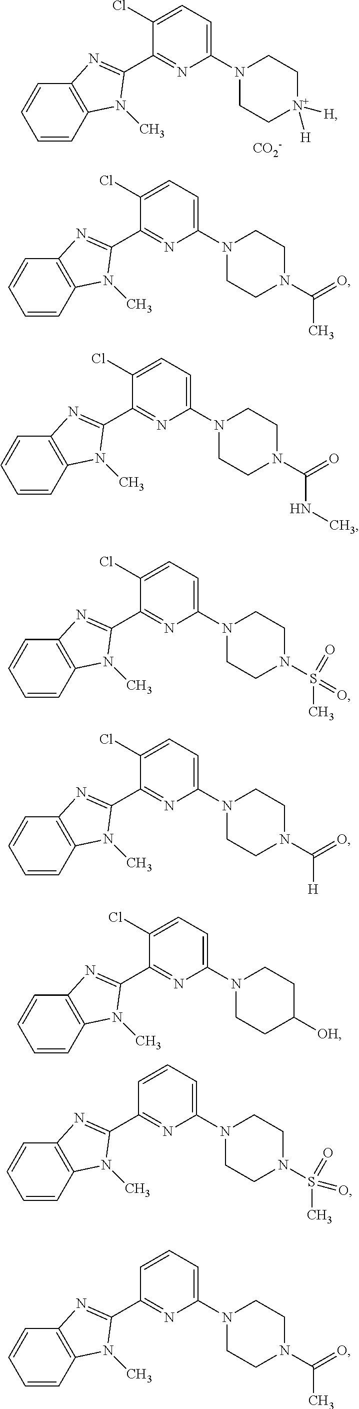

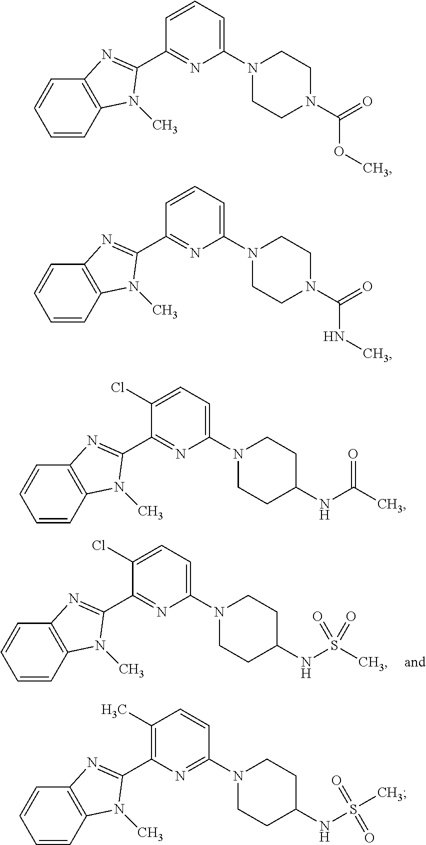

or a pharmaceutically acceptable salt thereof.

In another embodiment, the invention provides a compound selected from:

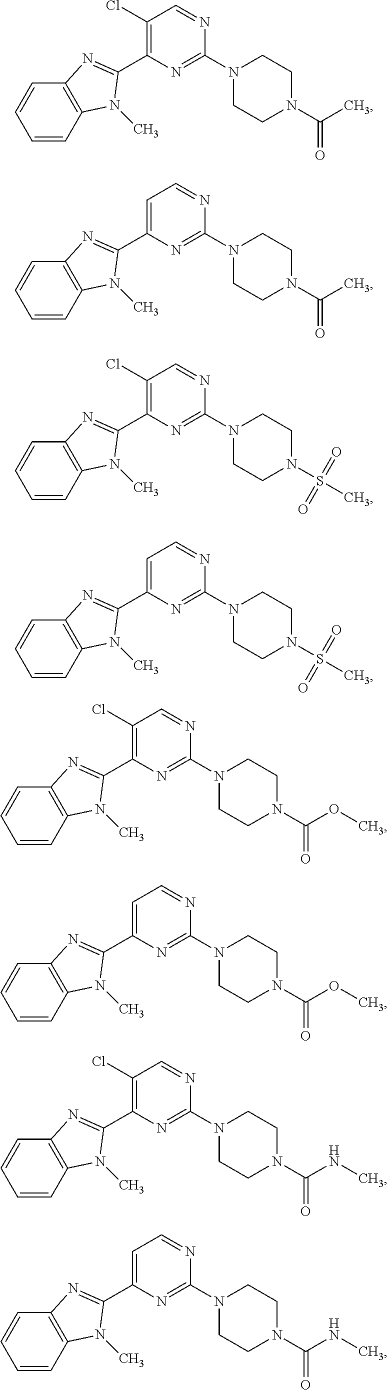

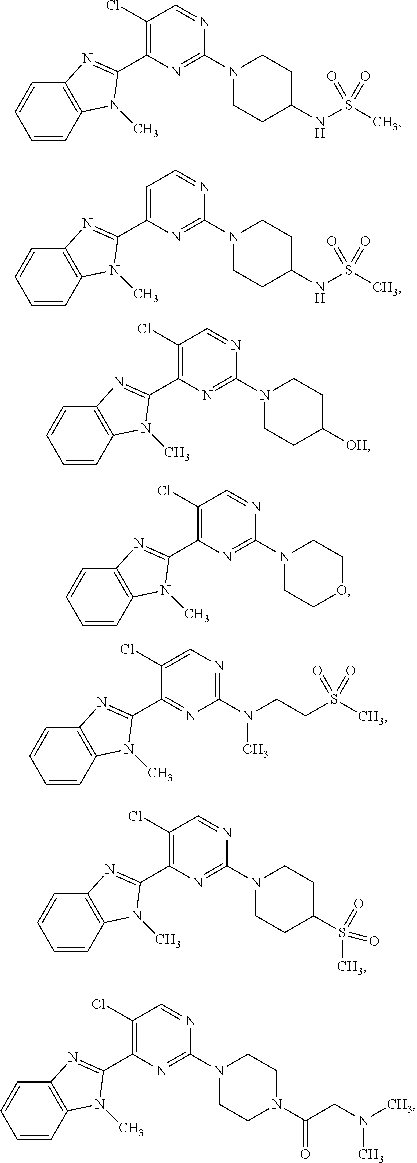

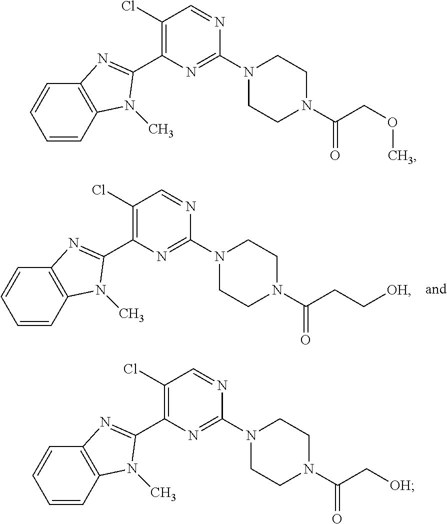

or a pharmaceutically acceptable salt thereof.

In a further embodiment, the invention provides a compound selected from:

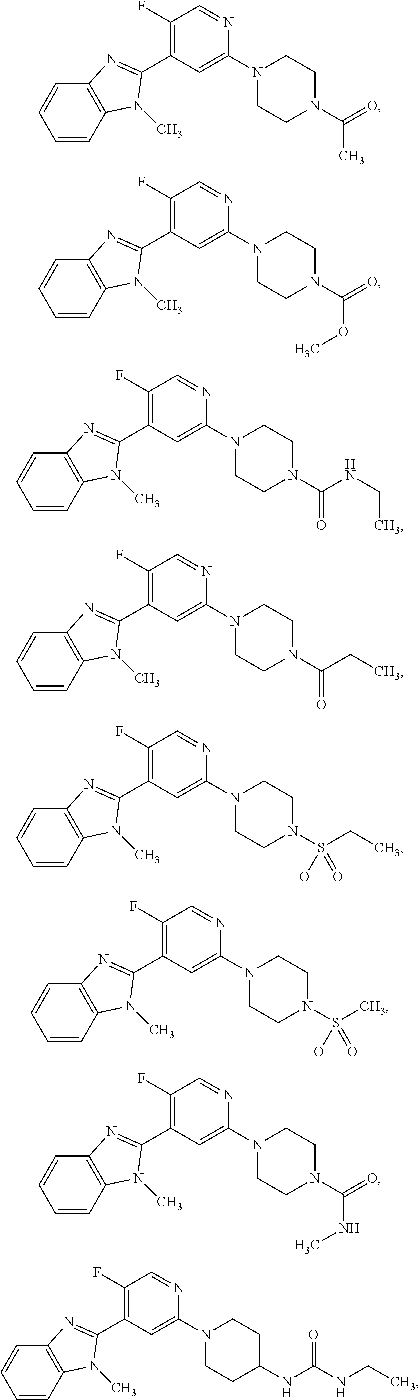

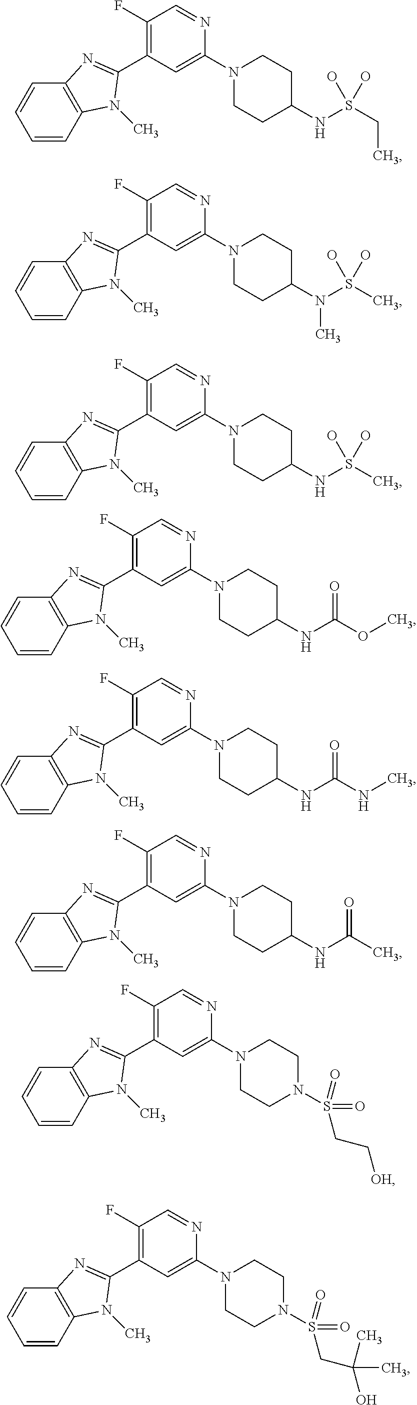

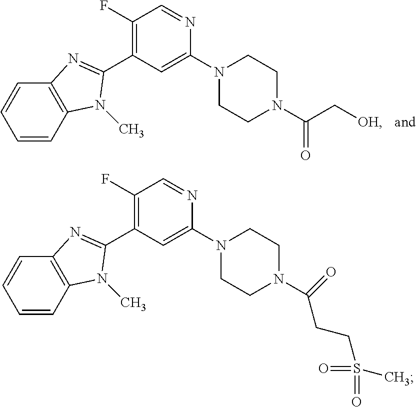

or a pharmaceutically acceptable salt thereof.

In yet a further embodiment, the invention provides a compound selected from:

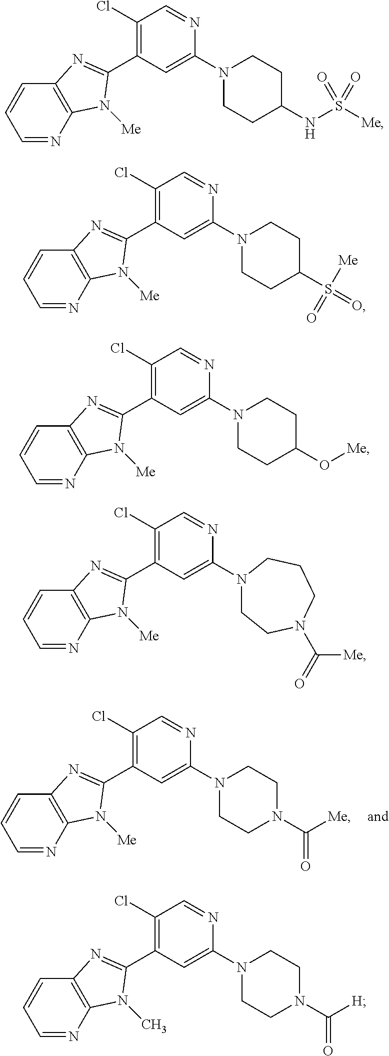

or a pharmaceutically acceptable salt thereof.

In a further embodiment, the invention provides a compound selected from:

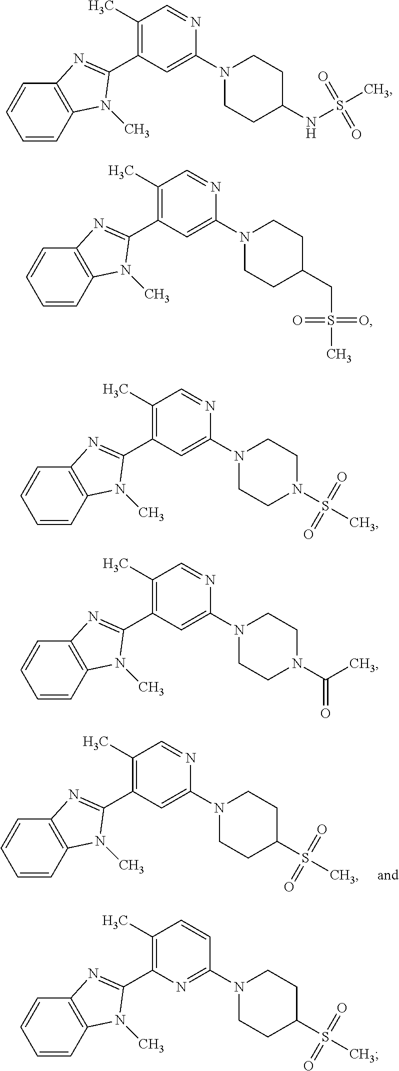

or a pharmaceutically acceptable salt thereof.

In yet a further embodiment, the invention provides a compound selected from:

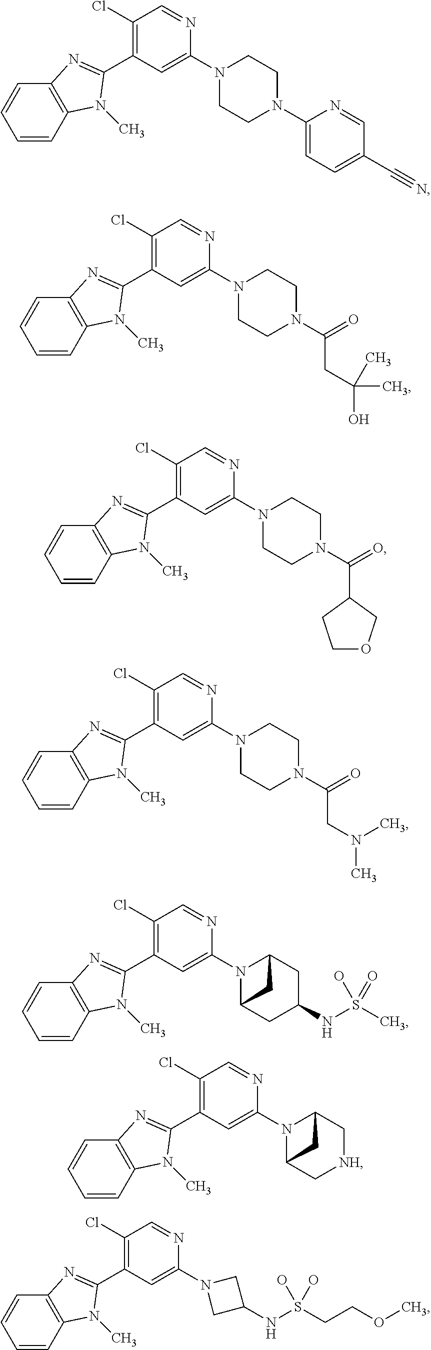

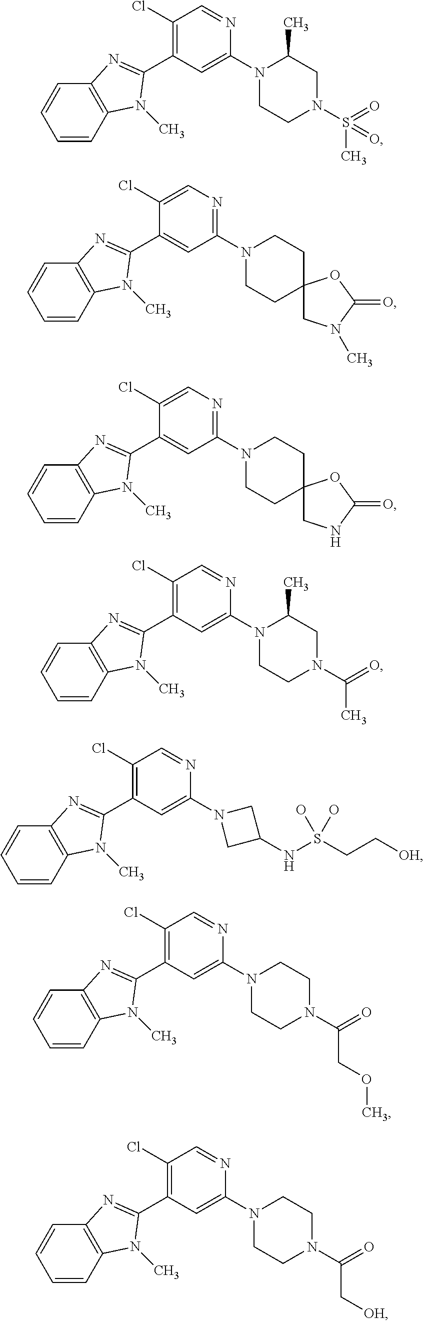

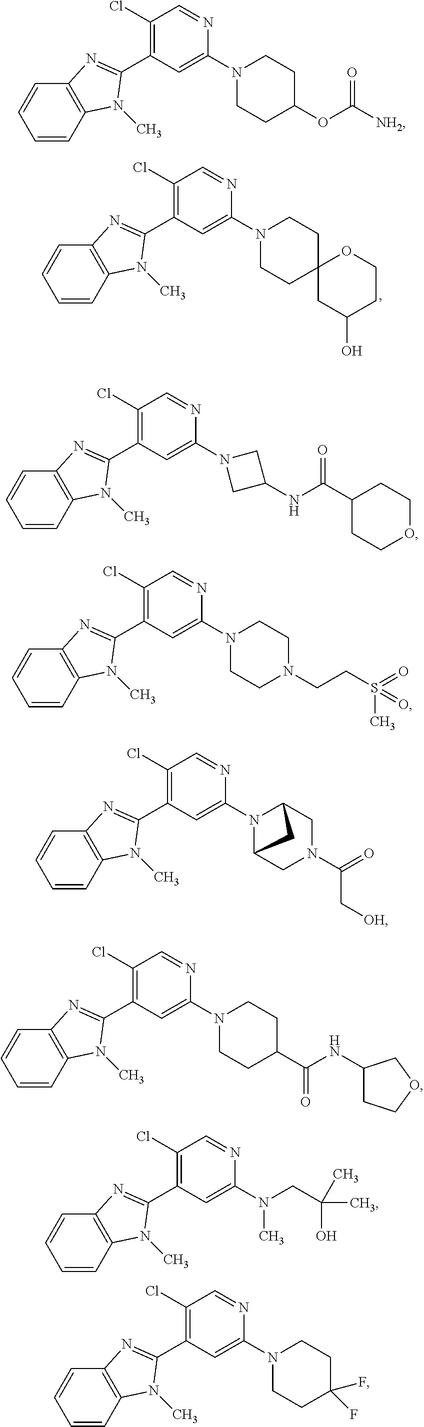

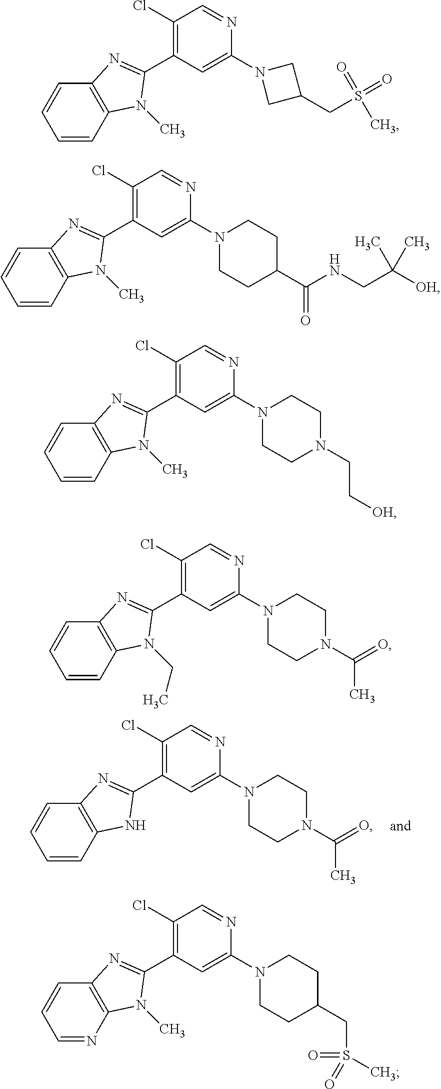

or a pharmaceutically acceptable salt thereof.

### Definitions

The term “alkyl”, as used herein means one to ten, preferably one to six, saturated monovalent hydrocarbon radicals having straight or branched moieties.

The terms “carbocycle”, “carbocyclyl”, “carbocyclo”, or “carbocyclic” as used herein means an aliphatic ring system having three to twelve members. The terms “carbocycle”, “carbocyclyl”, “carbocyclo”, or “carbocyclic”, whether saturated or partially unsaturated, also refers to rings that are optionally substituted. The terms “carbocycle”, “carbocyclyl”, “carbocyclo”, or “carbocyclic” also include aliphatic rings that are fused to one or more aromatic or non-aromatic rings, such as in a decahydronaphthyl or tetrahydronaphthyl, where the radical or point-of attachment is on the aliphatic ring.

As used herein, the term “cycloalkyl” refers to a mono, fused or bridged bicyclic or tricyclic carbocyclic rings, (e.g., cyclopropyl, cyclobutyl, cyclopentyl, cyclohexyl, cycloheptyl, cyclopentenyl, cyclohexenyl, bicyclo[2.2.1]heptanyl, bicyclo[3.2.1]octanyl and bicyclo[5.2.0]nonanyl, norbornyl, adamantanyl, etc.); said rings may optionally contain 1 or 2 double bonds. The term “cycloalkyl” also includes spiro cycloalkyl groups, including multi-ring systems joined by a single atom.

The term “alkoxy”, as used herein means O-alkyl groups wherein alkyl is as defined above.

The terms “hydroxyalkyl”, “alkoxyalkyl”, and alkoxycarbonyl”, used alone or as part of a larger moiety, includes both straight and branched chains containing one to six carbon atoms.

The term “alkenyl” used alone or as part of a larger moiety shall include both straight and branched chains containing two to ten carbon atoms having at least one carbon-carbon double bond. The term “alkynyl” used alone or as part of a larger moiety shall include both straight and branched chains containing two to ten carbon atoms having at least one carbon-carbon triple bond.

The terms “haloalkyl”, ‘haloalkenyl” and haloalkoxy” means alkyl, alkenyl or alkoxy, as the case may be, substituted with one or more halogen atoms. The term “halo” is used herein interchangeably with the term “halogen”, which denotes F, Cl, Br, or I. Preferred halo groups are F, Cl, and Br.

The term “heteroatom”, means nitrogen, oxygen, or sulfur and includes any oxidized form of nitrogen and sulfur, and the quaternized form of any basic nitrogen. Also, the term “nitrogen” includes a substitutable nitrogen of a heterocyclic ring. As an example, in a saturated or partially unsaturated ring having 0 to 3 heteroatoms selected from oxygen, sulfur or nitrogen, the nitrogen may be N (as in 3,4-dihydro-2H-pyrrolyl), NH (as in pyrrolidinyl) or NOR (as in N-substituted pyrrolidinyl).

The term “C₆₋₁₀ aryl”, as used herein, means a group derived from an aromatic hydrocarbon containing from 6 to 10 carbon atoms. Examples of such groups include, but are not limited to, phenyl or naphthyl. The terms “Ph” and “phenyl,” as used herein, mean a —C₆H₅ group. The term “benzyl,” as used herein, means a —CH₂C₆H₅ group. “Aryl” also includes fused polycyclic aromatic ring systems in which an aromatic ring is fused to one or more rings. Examples include 1-naphthyl, 2-naphthyl, 1-anthracyl and 2-anthracyl. Also included within the scope of the term “aryl”, as it is used herein, is a group in which an aromatic ring is fused to one or more non-aromatic rings, such as in an indanyl, phenanthridinyl, or tetrahydronaphthyl, where the radical or point of attachment is on the aromatic ring. The term “aryl” also refers to rings that are optionally substituted.

The term “heteroaryl”, used alone or as part of a larger moiety as in “heteroaralkyl” or “heteroarylalkoxy”, refers to an aromatic heterocyclic group having a total of from 5 to 12 atoms in its ring, and containing from 2 to 9 carbon atoms and from one to four heteroatoms each independently selected from O, S and N, with the proviso that the ring of said group does not contain two adjacent O atoms or two adjacent S atoms. The heterocyclic groups include benzo-fused ring systems. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

Examples of typical monocyclic heteroaryl groups include, but are not limited to:

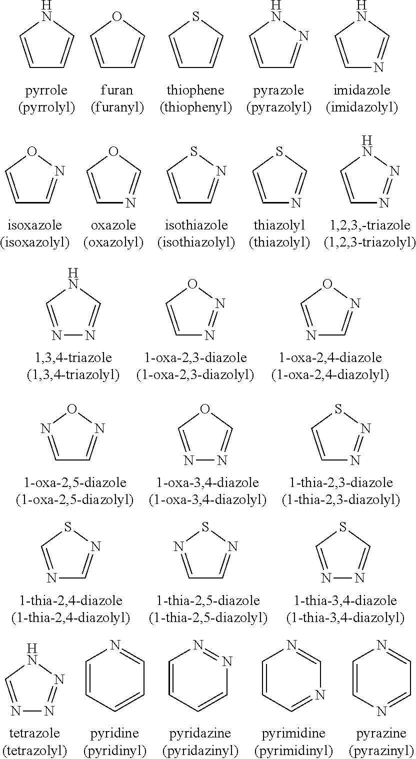

Examples of suitable fused ring heteroaryl groups include, but are not limited to:

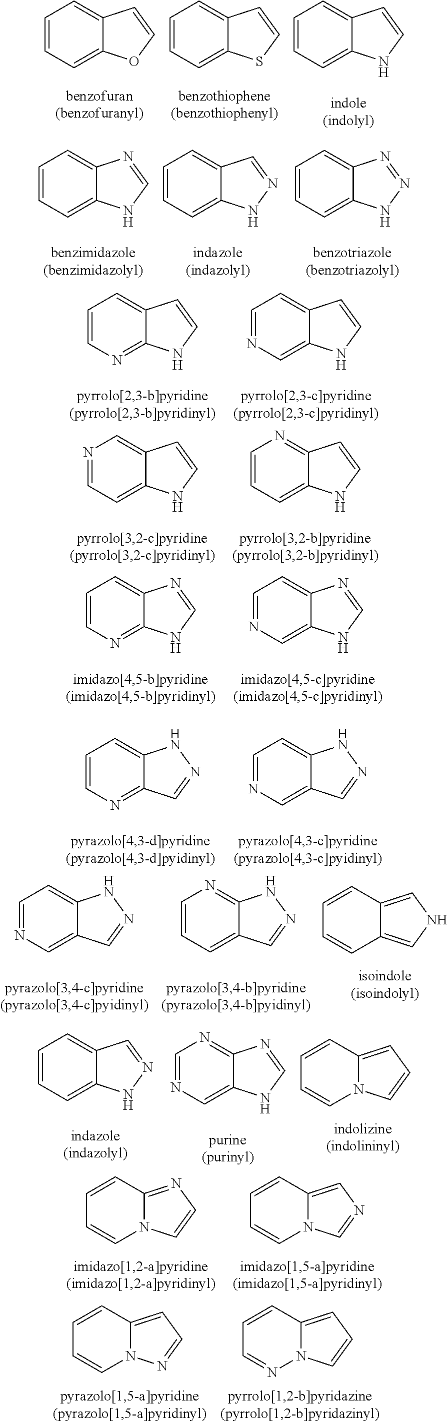

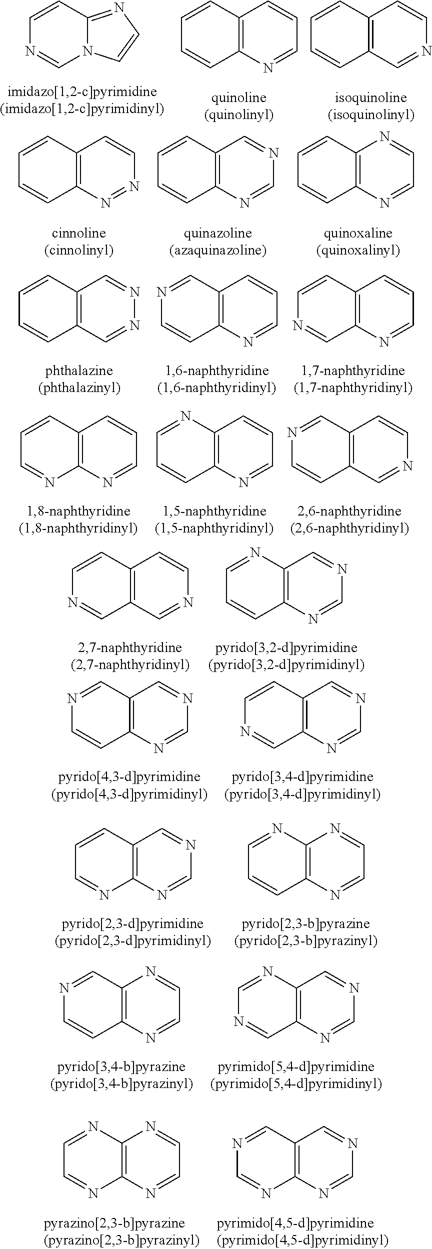

Also included within the scope of the term “heteroaryl”, as it is used herein, is a group in which a heteroatomic ring is fused to one or more aromatic or nonaromatic rings where the radical or point of attachment is on the heteroaromatic ring. Examples include tetrahydroquinolinyl, tetrahydroisoquinolinyl, and pyrido[3,4-d]pyrimidinyl.

“Heterocyclyl” (also known as heterocycle, or heteroalicyclic) refers to a non-aromatic, monocyclic, bicyclic, tricyclic or spirocyclic ring group having a total of 3 to 12 ring atoms, in which 1 to 4 ring atoms are heteroatoms selected from N, O, and S, and wherein the S atom may be optionally oxidized with one or two oxygen atoms, the remaining ring atoms being C, with the proviso that such ring systems may not contain two adjacent O atoms or two adjacent S atoms. The heterocyclic ring may also be substituted by an oxo (═O) group at any available C atom. The rings may also have one or more double bonds. Furthermore, such groups may be bonded to the remainder of the compounds of the present invention through either a carbon atom or a heteroatom, if possible. Examples of suitable saturated heterocyclyl groups include, but are not limited to:

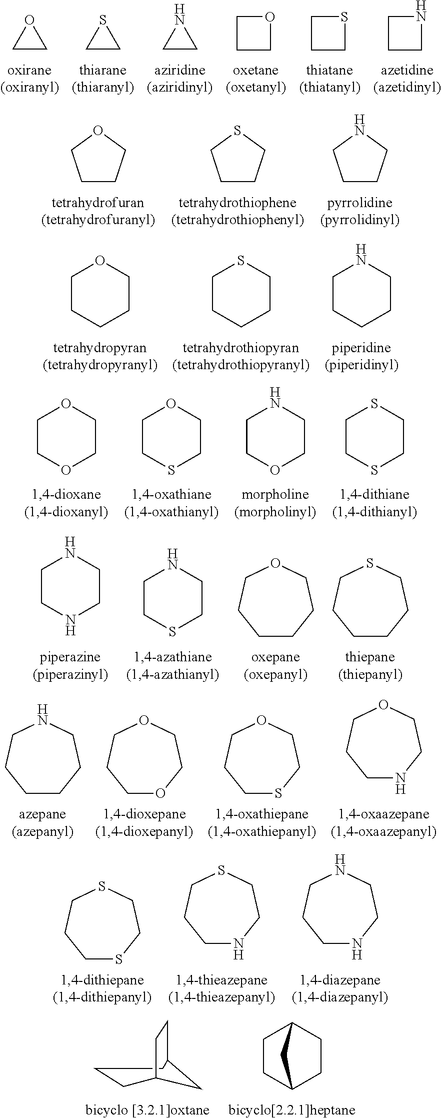

Examples of suitable partially unsaturated heterocyclyl groups include, but are not limited to:

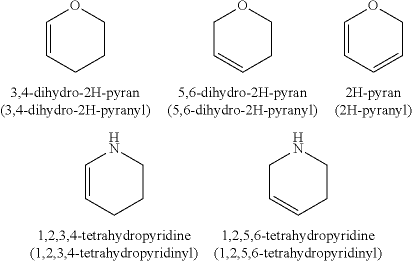

The term “heterocyclyl” or “heterocycle”, as previously noted, also includes spirocyclic moieties containing at least one heteroatom in one or more of the spirocyclic rings (also known as “heterospirocyclic” or “heterospirocyclic ring”). Such heterospirocyclic moieties may be optionally substituted at any ring position, including substitution on the heteratom(s) within the spirocyclic ring(s). Examples of spirocyclic moieties include, but are not limited to:

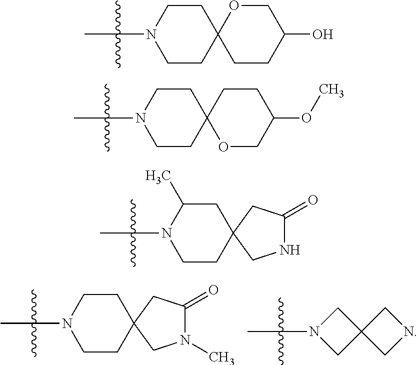

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

As used herein, an “effective” amount refers to an amount of a substance, agent, compound, or composition that is of sufficient quantity to result in a decrease in severity of disease symptoms, an increase in frequency and duration of disease symptom-free periods, or a prevention of impairment or disability due to the disease affliction—either as a single dose or according to a multiple dose regimen, alone or in combination with other agents or substances. One of ordinary skill in the art would be able to determine such amounts based on such factors as the subject's size, the severity of the subject's symptoms, and the particular composition or route of administration selected. The subject may be a human or non-human mammal (e.g., rabbit, rat, mouse, monkey or other lower-order primate).

The present invention includes isotopically-labeled compounds, which are identical to those recited in formula I, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as, but not limited to, ²H, ³H, ¹³C, ¹⁴C, ¹⁵N, ¹⁸O, 17O, ³¹P, ³²P, ³⁵S_{,} ¹⁸F, and ³⁶Cl, respectively. Compounds of the present invention and pharmaceutically acceptable salts of said compounds which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds of this invention can generally be prepared by carrying out the procedures disclosed in the Schemes and/or in the Examples and Preparations below, by substituting a readily available isotopically-labeled reagent for a non-isotopically-labeled reagent.

The present invention also relates to the pharmaceutically acceptable acid addition salts of the compounds of the invention. The acids which are used to prepare the pharmaceutically acceptable acid addition salts of the aforementioned base compounds of this invention are those which form nontoxic acid addition salts, i.e., salts containing pharmacologically acceptable anions, such as, but not limited to, the chloride, bromide, iodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p toluenesulfonate and pamoate [i.e., 1,1′ methylene bis(2 hydroxy 3 naphthoate)] salts.

The invention also relates to base addition salts of the compounds of the invention. The chemical bases that may be used as reagents to prepare pharmaceutically acceptable base salts of those compounds of the compounds of the invention that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of the invention are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above.

The compounds of this invention include all stereoisomers (e.g., cis and trans isomers) and all optical isomers of compounds of the invention (e.g., R and S enantiomers), as well as racemic, diastereomeric and other mixtures of such isomers. While all stereoisomers are encompassed within the scope of our claims, one skilled in the art will recognize that particular stereoisomers may be preferred.

The compounds of the present invention can exist in several tautomeric forms, including the enol and imine form, and the keto and enamine form and geometric isomers and mixtures thereof. All such tautomeric forms are included within the scope of the present invention. Tautomers exist as mixtures of a tautomeric set in solution. In solid form, usually one tautomer predominates. Even though one tautomer may be described, the present invention includes all tautomers of the present compounds.

The present invention also includes atropisomers of the present invention. Atropisomers refer to compounds of the invention that can be separated into rotationally restricted isomers.

The invention also relates to methods for making intermediate compounds that are useful for making the compounds of the invention.

As noted above, this invention also relates to the pharmaceutically acceptable salts of the compounds of the invention. Pharmaceutically acceptable salts of the compounds of the invention include the acid addition and base salts thereof. Suitable acid addition salts are formed from acids which form non-toxic salts. Non-limiting examples of suitable acid addition salts include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Non-limiting examples of suitable base salts include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds of the invention are known to one of skill in the art.

The compounds of the invention may also exist in unsolvated and solvated forms. Accordingly, the invention also relates to the hydrates and solvates of the compounds of the invention.

The term ‘solvate” is used herein to describe a molecular complex comprising the compound of the invention and one or more pharmaceutically acceptable solvent molecules, for example, ethanol.

Compounds of the invention containing one or more asymmetric carbon atoms can exist as two or more stereoisomers. Where a compound of the invention contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety. A single compound may exhibit more than one type of isomerism.

Included within the scope of the present invention are all stereoisomers, geometric isomers and tautomeric forms of the compounds of the invention, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

The invention also relates to methods for the treatment of abnormal cell growth in a mammal. In one embodiment, the invention relates to a method for the treatment of abnormal cell growth in a mammal comprising administering to said mammal an amount of a compound of the invention that is effective in treating abnormal cell growth.

In another embodiment the abnormal cell growth is cancer.

In another embodiment the cancer is selected from the group consisting of lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

The invention also relates to methods for the treatment of cancer solid tumors in a mammal. In one embodiment, the invention relates to the treatment of cancer solid tumor in a mammal comprising administering to said mammal an amount of a compound of the invention that is effective in treating said cancer solid tumor.

In another embodiment, the cancer solid tumor is breast, lung, colon, brain, prostate, stomach, pancreatic, ovarian, skin (melanoma), endocrine, uterine, testicular, or bladder.

In another embodiment, the invention relates to a method for the treatment of abnormal cell growth in a mammal which comprises administering to said mammal an amount of a compound of the invention that is effective in treating abnormal cell growth in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

In still another embodiment the invention relates to a pharmaceutical composition for the treatment of abnormal cell growth in a mammal comprising an amount of a compound of the invention that is effective in treating abnormal cell growth, and a pharmaceutically acceptable carrier.

This invention also relates to a method for the treatment of abnormal cell growth in a mammal, including a human, comprising administering to said mammal an amount of a compound of the invention, as defined above, or a pharmaceutically acceptable salt, solvate, hydrate or prodrug thereof, that is effective in treating abnormal cell growth. In one embodiment of this method, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In one embodiment the method comprises comprising administering to a mammal an amount of a compound of the invention that is effective in treating said cancer solid tumor. In one preferred embodiment the solid tumor is breast, lung, colon, brain, prostate, stomach, pancreatic, ovarian, skin (melanoma), endocrine, uterine, testicular, and bladder cancer.

In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of a compound of the invention, or a pharmaceutically acceptable salt, solvate, hydrate or prodrug thereof, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a pharmaceutical composition for the treatment of abnormal cell growth in a mammal, including a human, comprising an amount of a compound of the invention, as defined above, or a pharmaceutically acceptable salt, solvate, hydrate or prodrug thereof, that is effective in treating abnormal cell growth, and a pharmaceutically acceptable carrier. In one embodiment of said composition, said abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of a compound of the invention, or a pharmaceutically acceptable salt, solvate, or hydrate thereof, that is effective in treating abnormal cell growth in combination with another anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens. The invention also contemplates a pharmaceutical composition for treating abnormal cell growth wherein the composition includes a compound of the invention, as defined above, or a pharmaceutically acceptable salt, solvate, or hydrate thereof, that is effective in treating abnormal cell growth, and another anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a method for the treatment of a disorder associated with angiogenesis in a mammal, including a human, comprising administering to said mammal an amount of a compound of the invention, as defined above, or a pharmaceutically acceptable salt, solvate, hydrate or prodrug thereof, that is effective in treating said disorder in combination with one or more anti-tumor agents listed above. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

This invention also relates to a method of (and to a pharmaceutical composition for) treating abnormal cell growth in a mammal which comprise an amount of a compound of the invention, or a pharmaceutically acceptable salt, solvate, or hydrate thereof, in combination with an amount of one or more substances selected from anti-angiogenesis agents, signal transduction inhibitors inhibitor (e.g., inhibiting the means by which regulatory molecules that govern the fundamental processes of cell growth, differentiation, and survival communicated within the cell), and antiproliferative agents, which amounts are together effective in treating said abnormal cell growth.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound of the invention in the methods and pharmaceutical compositions described herein. Examples of useful COX-II inhibitors include CELEBREX™ (celecoxib), Bextra (valdecoxib), paracoxib, Vioxx (rofecoxib), and Arcoxia (etoricoxib). Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340, RO 32-3555, RS 13-0830, and the following compounds:
3-[[4-(4-fluoro-phenoxy)-benzenesulfonyl]-(1-hydroxycarbamoyl-cyclopentyl)-amino]-propionic acid;
3-exo-3-[4-(4-fluoro-phenoxy)-benzenesulfonylamino]-8-oxa-bicyclo[3.2.1]octane-3-carboxylic acid hydroxyamide;
(2R,3R) 1-[4-(2-chloro-4-fluoro-benzyloxy)-benzenesulfonyl]-3-hydroxy-3-methyl-piperidine-2-carboxylic acid hydroxyamide;
4-[4-(4-fluoro-phenoxy)-benzenesulfonylamino]-tetrahydro-pyran-4-carboxylic acid hydroxyamide;
3-[[4-(4-fluoro-phenoxy)-benzenesulfonyl]-(1-hydroxycarbamoyl-cyclobutyl)-amino]-propionic acid;
4-[4-(4-chloro-phenoxy)-benzenesulfonylamino]-tetrahydro-pyran-4-carboxylic acid hydroxyamide;
3-[4-(4-chloro-phenoxy)-benzenesulfonylamino]-tetrahydro-pyran-3-carboxylic acid hydroxyamide;
(2R,3R) 1-[4-(4-fluoro-2-methyl-benzyloxy)-benzenesulfonyl]-3-hydroxy-3-methyl-piperidine-2-carboxylic acid hydroxyamide;
3-[[4-(4-fluoro-phenoxy)-benzenesulfonyl]-(1-hydroxycarbamoyl-1-methyl-ethyl)-amino]-propionic acid;
3-[[4-(4-fluoro-phenoxy)-benzenesulfonyl]-(4-hydroxycarbamoyl-tetrahydro-pyran-4-yl)-amino]-propionic acid;
3-exo-3-[4-(4-chloro-phenoxy)-benzenesulfonylamino]-8-oxa-bicyclo[3.2.1]octane-3-carboxylic acid hydroxyamide;
3-endo-3-[4-(4-fluoro-phenoxy)-benzenesulfonylamino]-8-oxa-bicyclo[3.2.1]octane-3-carboxylic acid hydroxyamide; and
3-[4-(4-fluoro-phenoxy)-benzenesulfonylamino]-tetrahydro-furan-3-carboxylic acid hydroxyamide;

and pharmaceutically acceptable salts and solvates of said compounds.

VEGF inhibitors, for example, SU-11248, SU-5416 and SU-6668 (Sugen Inc. of South San Francisco, Calif., USA), can also be combined with a compound of the invention. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); Avastin, an anti-VEGF monoclonal antibody of Genentech, Inc. of South San Francisco, Calif.; and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with a compound of the invention. Such erbB2 inhibitors include Herceptin, 2C4, and pertuzumab. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety. Other erbb2 receptor inhibitors include TAK-165 (Takeda) and GW-572016 (Glaxo-Wellcome).

Various other compounds, such as styrene derivatives, have also been shown to possess tyrosine kinase inhibitory properties, and some of tyrosine kinase inhibitors have been identified as erbB2 receptor inhibitors. More recently, five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are World Patent Application WO00/44728 (published Aug. 3, 2000), EP 1029853A1 (published Aug. 23, 2000), and WO01/98277 (published Dec. 12, 2001) all of which are incorporated herein by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following United States patent applications: 09/221,946 (filed Dec. 28, 1998); 09/454,058 (filed Dec. 2, 1999); 09/501,163 (filed Feb. 9, 2000); 09/539,930 (filed Mar. 31, 2000); 09/202,796 (filed May 22, 1997); 09/384,339 (filed Aug. 26, 1999); and 09/383,755 (filed Aug. 26, 1999); and the compounds disclosed and claimed in the following United States provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

A compound of the invention may also be used with other agents useful in treating abnormal cell growth or cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase inhibitors described in the references cited in the “Background” section, supra. Specific CTLA4 antibodies that can be used in the present invention include those described in U.S. Provisional Application 60/113,647 (filed Dec. 23, 1998) which is herein incorporated by reference in its entirety.

A compound of the invention may be applied as a sole therapy or may involve one or more other anti-tumor substances, for example those selected from, for example, mitotic inhibitors, for example vinblastine; alkylating agents, for example cis-platin, oxaliplatin, carboplatin and cyclophosphamide; anti-metabolites, for example 5-fluorouracil, capecitabine, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex (tamoxifen) or, for example anti-androgens such as Casodex (4′-cyano-3-(4-fluorophenylsulphonyl)-2-hydroxy-2-methyl-3′-(trifluoromethyl)propionanilide).

The compounds of the present invention may be used alone or in combination with one or more of a variety of anti-cancer agents or supportive care agents. For example, the compounds of the present invention may be used with cytotoxic agents, e.g., one or more selected from the group consisting of a camptothecin, irinotecan HCl (Camptosar), edotecarin, SU-11248, epirubicin (Ellence), docetaxel (Taxotere), paclitaxel, rituximab (Rituxan) bevacizumab (Avastin), imatinib mesylate (Gleevac), Erbitux, gefitinib (Iressa), and combinations thereof. The invention also contemplates the use of the compounds of the present invention together with hormonal therapy, e.g., exemestane (Aromasin), Lupron, anastrozole (Arimidex), tamoxifen citrate (Nolvadex), Trelstar, and combinations thereof. Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof. Such conjoint treatment may be achieved by way of the simultaneous, sequential or separate dosing of the individual components of the treatment.

The compounds of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers. In this regard, the following is a non-limiting list of examples of secondary agents that may be used with the compounds of the invention.

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, carboplatin, eptaplatin, lobaplatin, nedaplatin, oxaliplatin or satrplatin.

Antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, gemcitabine, fludarabin, 5-azacitidine, capecitabine, cladribine, clofarabine, decitabine, eflornithine, ethynylcytidine, cytosine arabinoside, hydroxyurea, TS-1, melphalan, nelarabine, nolatrexed, ocfosfate, disodium premetrexed, pentostatin, pelitrexol, raltitrexed, triapine, trimetrexate, vidarabine, vincristine, vinorelbine; or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid.

Antibiotics include but are not limited to: aclarubicin, actinomycin D, amrubicin, annamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin or zinostatin.

Hormonal therapy agents, e.g., exemestane (Aromasin), Lupron, anastrozole (Arimidex), doxercalciferol, fadrozole, formestane, anti-estrogens such as tamoxifen citrate (Nolvadex) and fulvestrant, Trelstar, toremifene, raloxifene, lasofoxifene, letrozole (Femara), or anti-androgens such as bicalutamide, flutamide, mifepristone, nilutamide, Casodex® (4′-cyano-3-(4-fluorophenylsulphonyl)-2-hydroxy-2-methyl-3′-(trifluoromethyl)propionanilide) and combinations thereof.

Plant derived anti-tumor substances include for example those selected from mitotic inhibitors, for example vinblastine, docetaxel (Taxotere) and paclitaxel.

Cytotoxic topoisomerase inhibiting agents include one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, and topotecan, and combinations thereof.

Immunologicals include interferons and numerous other immune enhancing agents. Interferons include interferon alpha, interferon alpha-2a, interferon, alpha-2b, interferon beta, interferon gamma-1a or interferon gamma-n1. Other agents include PF3512676, filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab, Provenge.

Biological response modifiers are agents that modify defense mechanisms of living organisms or biological responses, such as survival, growth, or differentiation of tissue cells to direct them to have anti-tumor activity. Such agents include krestin, lentinan, sizofuran, picibanil, or ubenimex.

Other anticancer agents include alitretinoin, ampligen, atrasentan bexarotene, bortezomib. Bosentan, calcitriol, exisulind, finasteride, fotemustine, ibandronic acid, miltefosine, mitoxantrone, l-asparaginase, procarbazine, dacarbazine, hydroxycarbamide, pegaspargase, pentostatin, tazarotne, TLK-286, Velcade, Tarceva, or tretinoin.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain and Vitaxin.

Platinum-coordinated compounds include but are not limited to, cisplatin, carboplatin, nedaplatin, or oxaliplatin.

Camptothecin derivatives include but are not limited to camptothecin, 10-hydroxycamptothecin, 9-aminocamptothecin, irinotecan, SN-38, edotecarin, and topotecan.

Tyrosine kinase inhibitors are Iressa or SU5416.

Antibodies include Herceptin, Erbitux, Avastin, or Rituximab.

Interferons include interferon alpha, interferon alpha-2a, interferon, alpha-2b, interferon beta, interferon gamma-1a or interferon gamma-n1.

Biological response modifiers are agents that modify defense mechanisms of living organisms or biological responses, such as survival, growth, or differentiation of tissue cells to direct them to have anti-tumor activity. Such agents include krestin, lentinan, sizofuran, picibanil, or ubenimex.

Other antitumor agents include mitoxantrone, 1-asparaginase, procarbazine, dacarbazine, hydroxycarbamide, pentostatin, or tretinoin.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; and (5) benign and malignant cells of other proliferative diseases in which aberrant serine/threonine kinase activation occurs.

The compounds of the present invention are potent inhibitors of SMO, and thus are all adapted to therapeutic use as antiproliferative agents (e.g., anticancer), antitumor (e.g., effective against solid tumors), antiangiogenesis (e.g., stop or prevent proliferation of blood vessels) in mammals, particularly in humans. In particular, the compounds of the present invention are useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.g., psoriasis) and benign hyperplasia of the prostate (e.g., BPH). It is, in addition, expected that a compound of the present invention may possess activity against a range of leukemias and lymphoid malignancies.

In one embodiment of the present invention cancer is selected from lung cancer, bone cancer, pancreatic cancer, gastric, skin cancer, cancer of the head or neck, cutaneous or intraocular melanoma, uterine cancer, ovarian cancer, gynecological, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, squamous cell, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain, pituitary adenoma, or a combination of one or more of the foregoing cancers.

In another embodiment cancer is selected a solid tumor, such as, but not limited to, breast, lung, colon, brain (e.g., glioblastoma), prostate, stomach, pancreatic, ovarian, skin (melanoma), endocrine, uterine, testicular, and bladder.

The methods of the present invention include the use of small molecules which inhibit Smo, in the regulation of repair and/or functional performance of a wide range of cells, tissues and organs, including normal cells, tissues, and organs, as well as those having the phenotype of ptc loss-of-function, hedgehog gain-of-function, or smoothened gain-of-function. For instance, the subject method has therapeutic and cosmetic applications ranging from regulation of neural tissues, bone and cartilage formation and repair, regulation of spermatogenesis, regulation of smooth muscle, regulation of lung, liver and other organs arising from the primative gut, regulation of hematopoietic function, regulation of skin and hair growth, etc. Moreover, the subject methods can be performed on cells that are provided in culture (in vitro), or on cells in a whole animal (in vivo). See, for example, PCT publications WO 95/18856 and WO 96/17924.

The present invention also provides a pharmaceutical composition comprising a compound of formula I, or a pharmaceutically acceptable salt or solvate thereof, as hereinbefore defined in association with a pharmaceutically acceptable adjuvant, diluent or carrier.

The invention further relates to a pharmaceutical composition of the invention which comprises mixing a compound of formula I, or a pharmaceutically acceptable salt or solvate thereof, as hereinbefore defined with a pharmaceutically acceptable adjuvant, diluent or carrier.

For the above-mentioned therapeutic uses the dosage administered will, of course, vary with the compound employed, the mode of administration, the treatment desired and the disorder indicated. The daily dosage of the compound of formula I or pharmaceutically acceptable salt may be in the range from 1 mg to 1 gram, preferably 1 mg to 250 mg, more preferably 10 mg to 100 mg.

The present invention also encompasses sustained release compositions.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The active compound may be applied as a sole therapy or may involve one or more other anti-tumor substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex® (tamoxifen) or, for example anti-androgens such as Casodex® (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

### DETAILED DESCRIPTION OF THE INVENTION

The examples and preparations provided below further illustrate and exemplify the compounds of the present invention and methods of preparing such compounds. The scope of the present invention is not limited in any way by the following examples and preparations. In the following examples, molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

In general, the compounds of the invention may be prepared by processes known in the chemical arts, particularly in light of the description contained herein. Certain processes for the manufacture of the compounds of the invention are provided as further features of the invention and are illustrated in the reaction schemes provided below and in the experimental section.

The following abbreviations may be used herein: Et₂O (diethyl ether); DMF (N,N-dimethylformamide); THF (tetrahydrofuran); DCM (dichloromethane); DMA (dimethyl acetal); DBU (1,8-diazabicyclo[5.4.0]undec-7-ene); HATU (2-(1H-7-azabenzotriazol-1-yl)-1,1,3,3-tetramethyl uronium hexafluorophosphate methanaminium); LDA (lithium diisopropylamide); DMSO (dimethylsulfoxide); DIPEA (N,N-Diisopropylethylamine); mCPBA (meta-Chloroperoxybenzoic acid); TFA (Trifluoroacetic acid); N-BOC(N-tert-Butoxycarbonyl); MeOH (methanol); EtOH (ethanol); EtOAc (ethyl acetate); Ac (acetyl); Me (methyl); Et (ethyl); MEM (minimal essential medium); PBS (phosphate-buffered saline); FBS (fetal bovine serum); R.T. (room temperature); mins (minutes) conc. (concentrated); CV (column volume); and ND (not determined).

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The compounds of the invention can be prepared by the following general methods and by methods described in detail as follows.

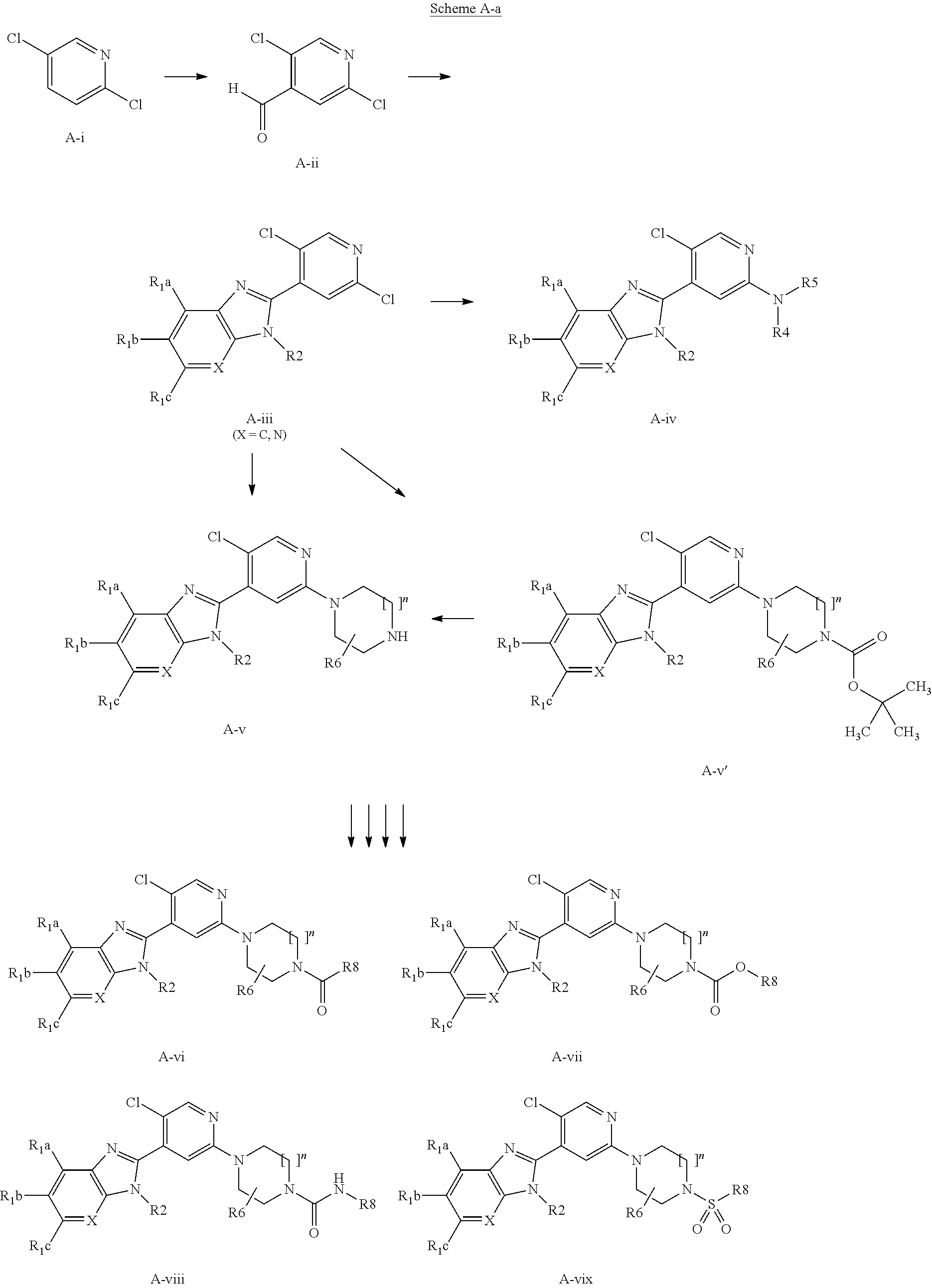

As illustrated in Scheme A-a, 2,5-dichloropyridine was treated with LDA followed by quenching with DMF to afford the aldehyde A-ii. This was treated with an N-alkyl-o-phenylenediamine to afford the benzimidazole A-iii, which was reacted with various amines in the presence of cesium fluoride in a suitable solvent (such as DMSO) to yield products A-iv. Where applicable, the product amines A-iv (illustrated by A-v) were reacted with acylating agents (under standard conditions known in the art) such as acyl chlorides to yield amides (A-vi), carbamoyl chlorides to yield carbamates (A-vii), isocyanates to yield ureas (A-viii), and sulfonyl chlorides to yield sulfonamides (A-ix). Alternatively, an N-BOC protected amine was used in the conversion of A-iii to A-v′, which was deprotected under standard conditions known in the art to afford A-v for subsequent functionalization. Each “n” depicted in the schemes herein is independently selected from 0, 1, 2, 3, 4, 5, or 6.

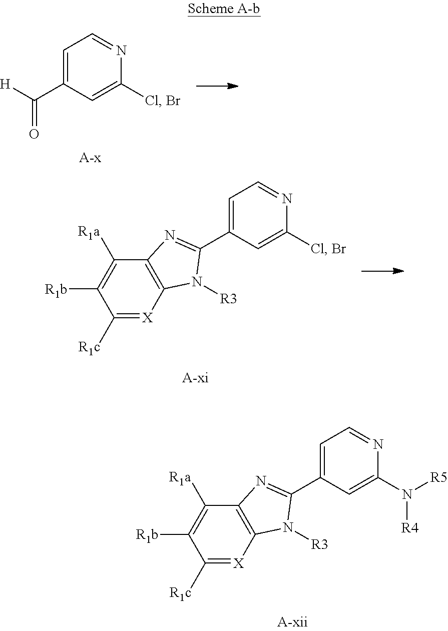

In the case of the des-halo products, a 2-halo-pyridine-4-carboxaldehyde, A-x was used as a starting point as illustrated in Scheme A-b. Coupling with an N-alkyl-o-phenylenediamine as in scheme A-a to afford the benzimidazole A-xi which was treated with amines to yield products A-xii, which were acylated where appropriate as in previous Scheme A-a.

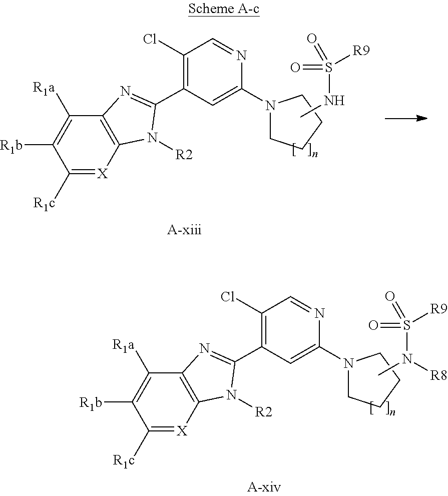

As illustrated in Scheme A-c, certain N-alkylated amides, carbamates and sulfonamides were simply accessed via alkylation of the products A-iv from Scheme A-a by standard conditions known in the art (eg: methyl iodide with sodium hydride in THF), as depicted by the conversion of sulfonamide A-xiii to the alkylated product A-

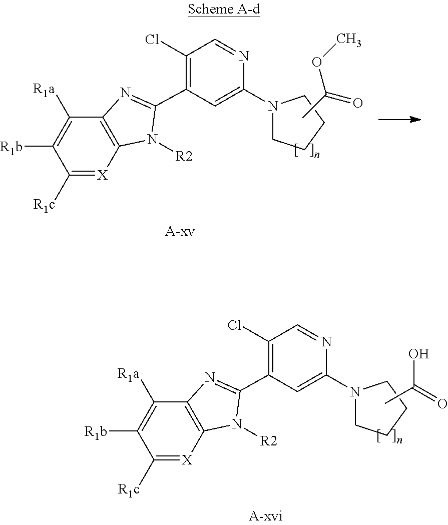

In the case of carboxylic acid products, these were accessed via hydrolysis of the ester bearing products A-iv from Scheme A-a by standard conditions known in the art (eg: sodium hydroxide solution in THF/methanol), illustrated in Scheme A-d by the conversion of the ester A-xv to the carboxylic acid product A-xvi.

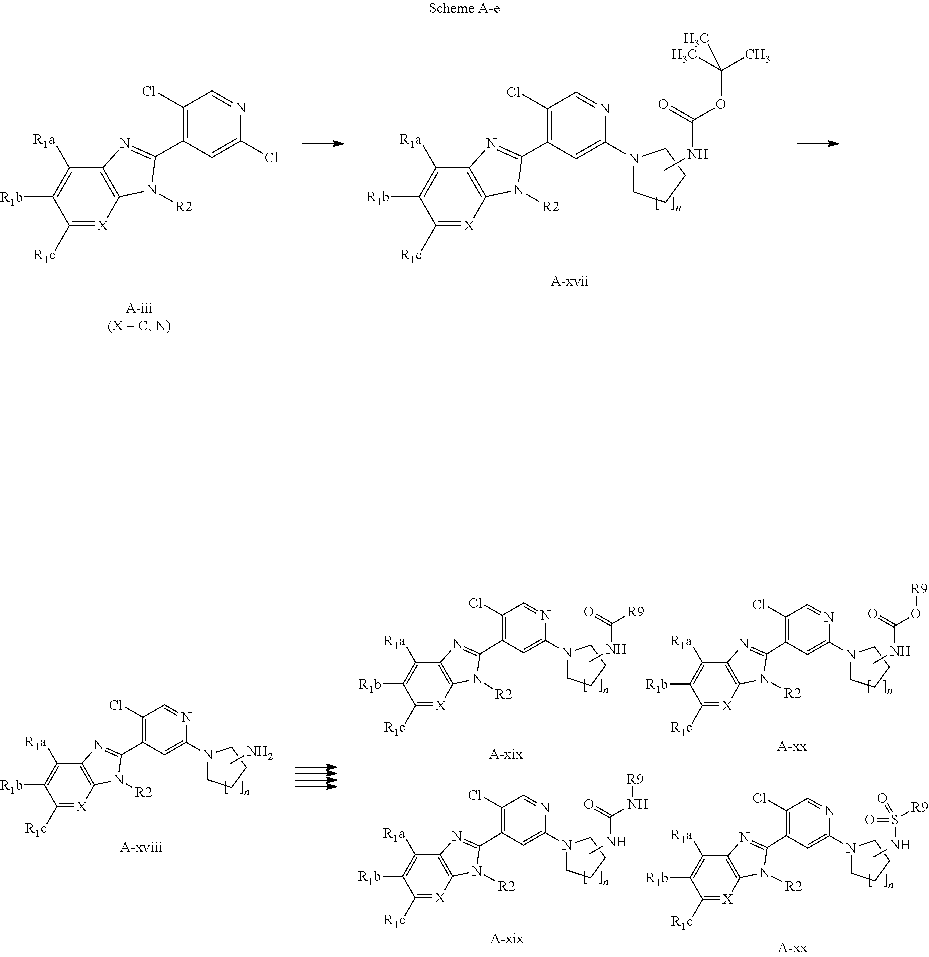

In cases where the chloro displacement was to be done with an amine that could give rise to selectivity issues, a suitably protected derivative was used, as illustrated in Scheme A-e. Dichloropyridine A-iii (from Scheme A-a) was reacted with a protected amine to yield the aminopyridine A-xvii. Deprotection of the protected intermediate A-xvii was achieved under standard conditions known in the art (in the N-BOC case with HCl or TFA), then the resulting amines A-xviii were reacted with acylating agents (under standard conditions known in the art) such as acyl chlorides to yield amides (A-xix), carbamoyl chlorides to yield carbamates (A-xx), isocyanates to yield ureas (A-xxi), and sulfonyl chlorides to yield sulfonamides (A-xxii).

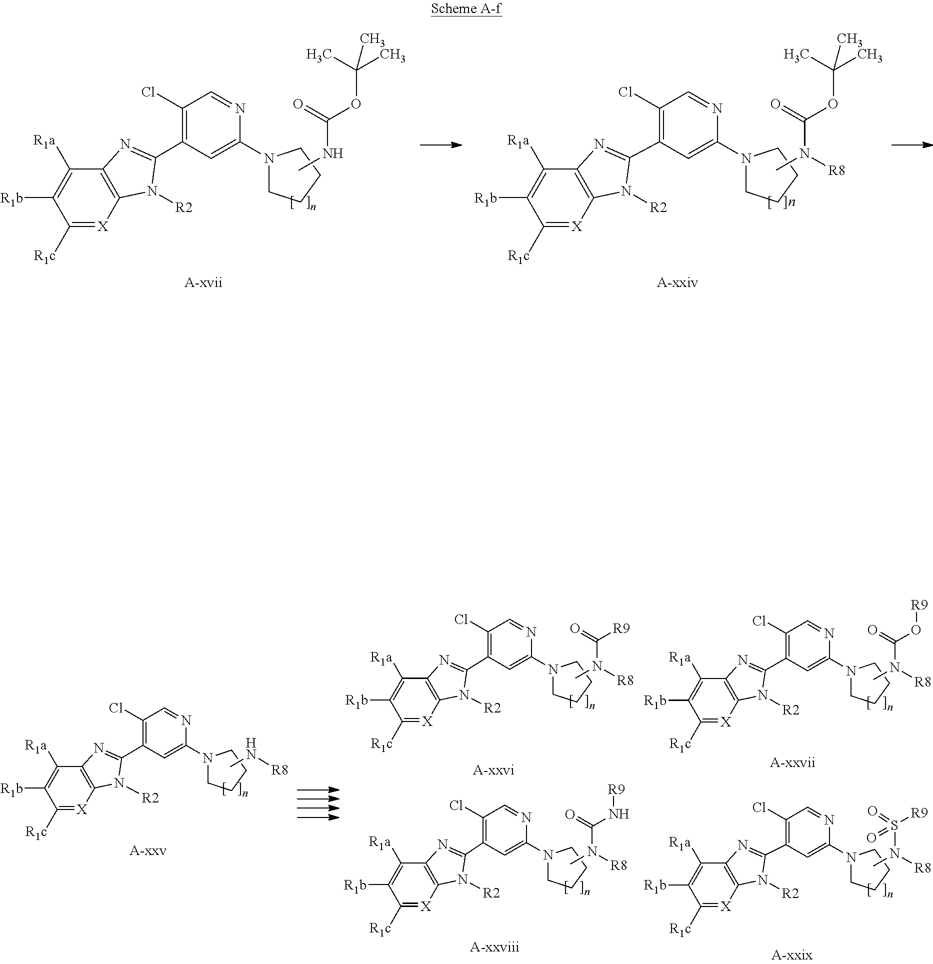

As illustrated in Scheme A-f, certain examples of N-alkylated amides, carbamates, ureas and sulfonamides were accessed via alkylation of the protected amine intermediates (exemplified by A-xvii from Scheme A-e) under standard conditions known in the art (eg: methyl iodide with sodium hydride in THF) to protected tertiary carbamates A-xxiv which were subsequently deprotected and functionalized with acyl chlorides to yield product amides (A-xxvi), carbamoyl chlorides to yield carbamates (A-xxvii), isocyanates to yield ureas (A-xxviii) and sulfonyl chlorides to yield sulfonamides (A-xxix).

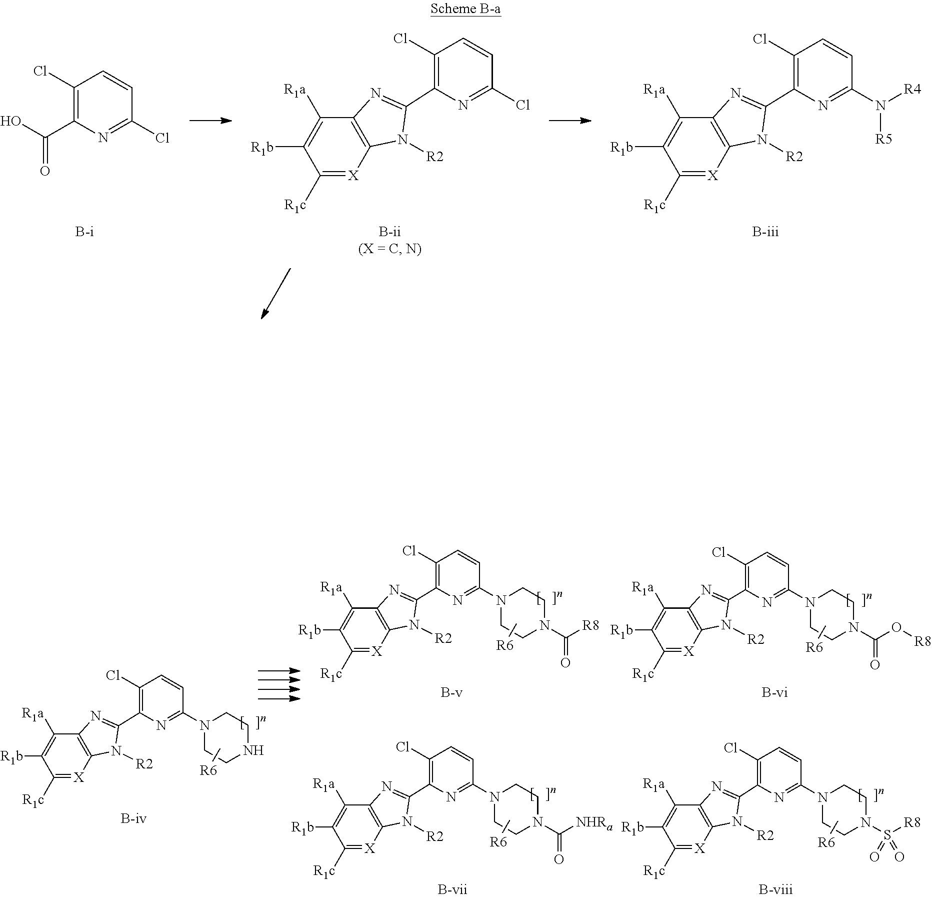

As exemplified in Scheme B-a, 2,5-dichloropyridine-6-carboxylic acid B-i was treated with an N-alkyl-o-phenylenediamine in the presence of triphenyl phosphite in pyridine solvent under microwave conditions (see Tett. Lett., 47, 2006, 2883-2886) to afford the benzimidazole B-ii. Intermediate B-ii was subjected to analogous chemistry as intermediate A-iii (from Scheme A-a), making non critical method changes and appropriate substitutions to yield products B-iii-B-viii.

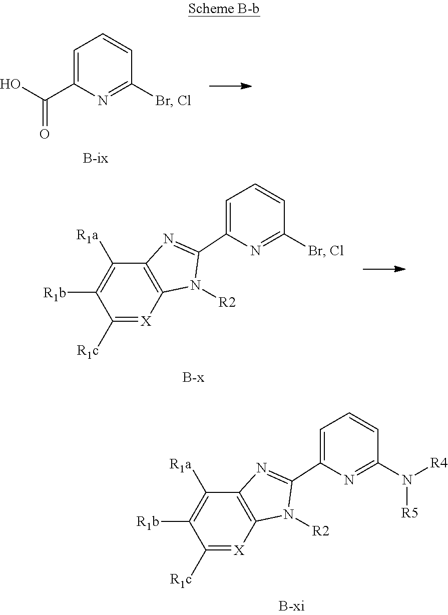

In the case of the des-halo products, a 2-halo-pyridine-6-carboxaldehyde, B-ix was used as a starting point as illustrated in scheme B-b, coupling with an N-alkyl-o-phenylenediamine (as per scheme A-b) to afford the benzimidazole B-x which was treated with amines to yield products B-xi, which were acylated where appropriate in an analogous fashion as shown in previous scheme B-a where halopyridines B-ii and B-x are interchangeable.

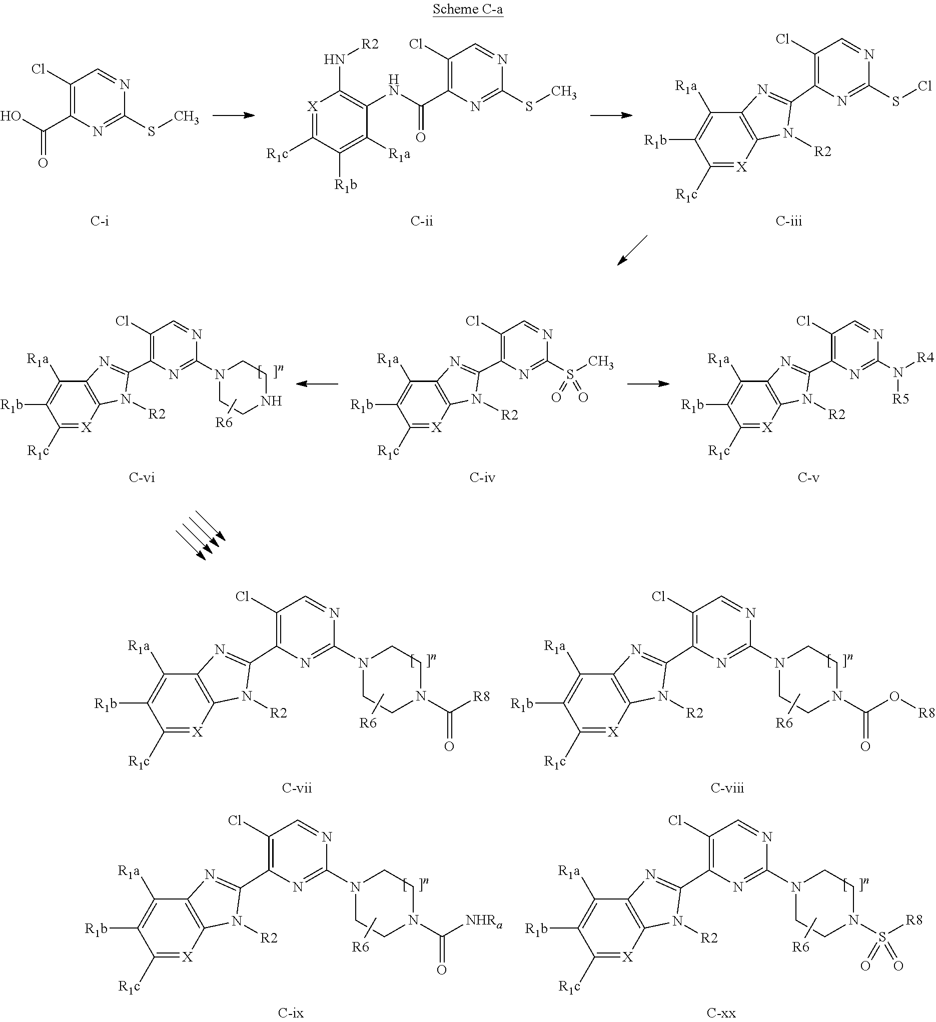

As exemplified in Scheme C-a, the pyrimidine carboxylic acid derivative C-i was converted to its corresponding aryl amide C-ii under standard conditions known in the art (such as HATU and DIPEA in DMF with an N-alkyl-o-phenylenediamine derivative), followed by conversion to the benzimidazole C-iii by acid mediated cyclisation. C-iii was then oxidized to the methyl sulfone C-iv using standard conditions known in the art (such as potassium peroxymonosulfate), which was subsequently reacted with amines in a suitable solvent (such as THF) to yield the aminopyrimidines products C-v and C-vi. Where applicable, the amines C-vi were reacted with acylating agents such as acyl chlorides to yield amides (C-vii), carbamoyl chlorides to yield carbamates (C-viii), isocyanates to yield ureas (C-ix), or sulfonyl chlorides to yield sulfonamides (C-x).

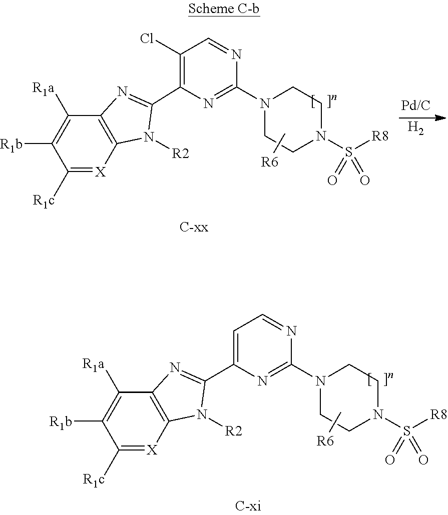

In the case for the des chloro products, the products C-vii-C-x were treated with hydrogen over a suitable catalyst (such as palladium on carbon) to yield the corresponding 5-H products, as exemplified by C-xi in scheme C-b.

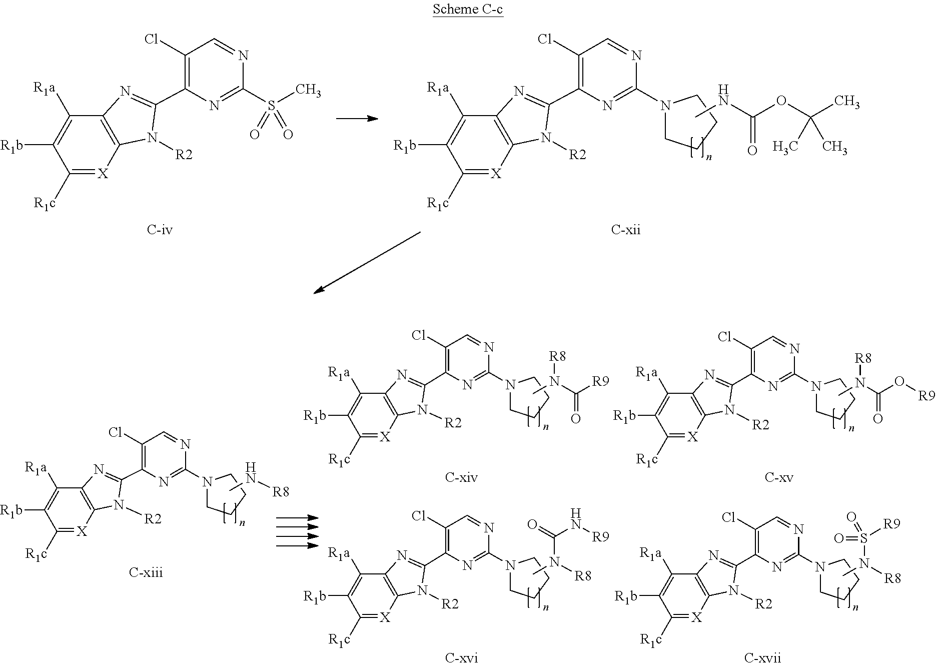

In cases where the sulfone displacement was to be done with an amine that could give rise to selectivity issues, a suitably protected derivative was used, as illustrated in Scheme C-c. Deprotection of the coupled products C-xii was achieved under standard conditions known in the art (in the N-BOC case with HCl or TFA), then the product amines C-xiii were reacted with acylating agents under standard conditions known in the art, such as acyl chlorides to yield amides (C-xiv), carbamoyl chlorides to yield carbamates (C-xv), isocyanates to yield ureas (C-xvi), or sulfonyl chlorides to yield sulfonamides (C-xvii).

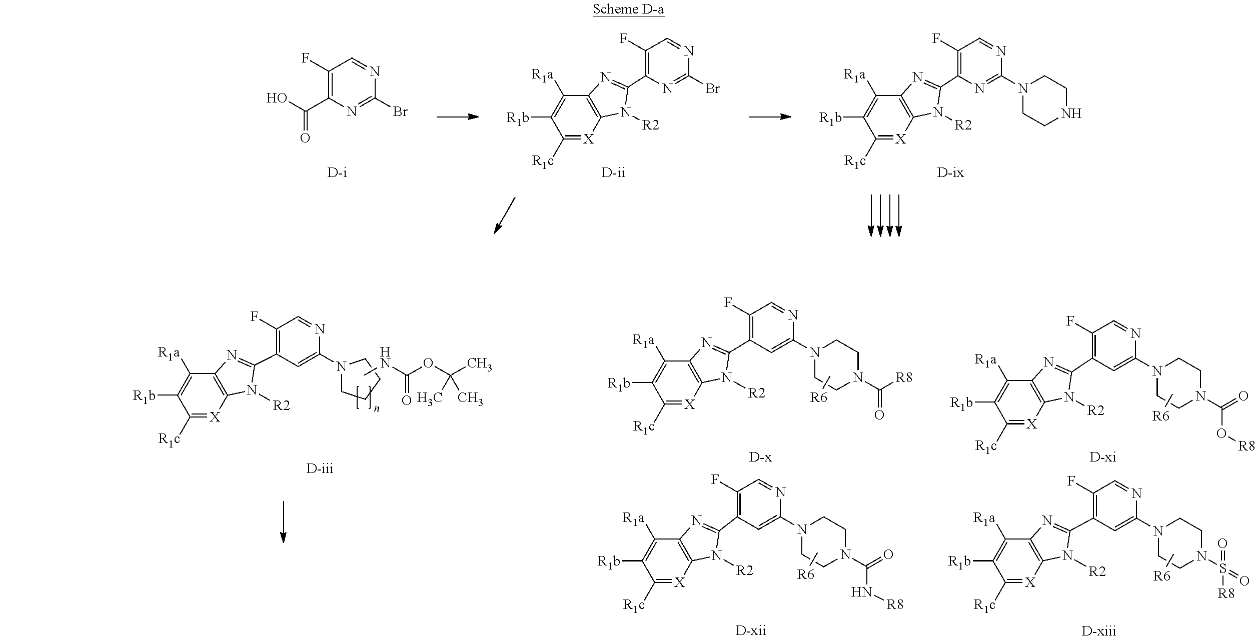

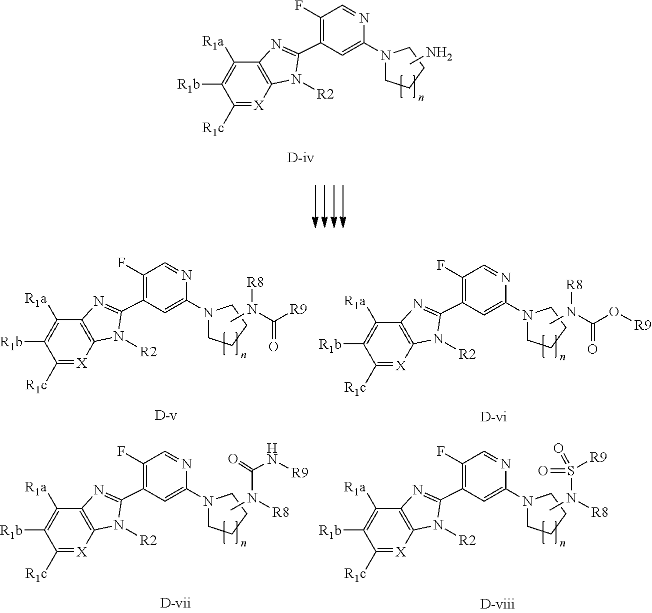

As shown in scheme D-a, the fluorinated intermediate D-ii was obtained by condensation of the acid D-i with a suitable phenyl-1,2-diamine, followed by acid mediated cyclisation to afford the benzimidazole. Substitution of the bromine was achieved using standard palladium mediated Buchwald type amination conditions to afford the amine products D-iii and D-ix. These were subjected to similar chemistry as illustrated in schemes A and B, to afford the products.

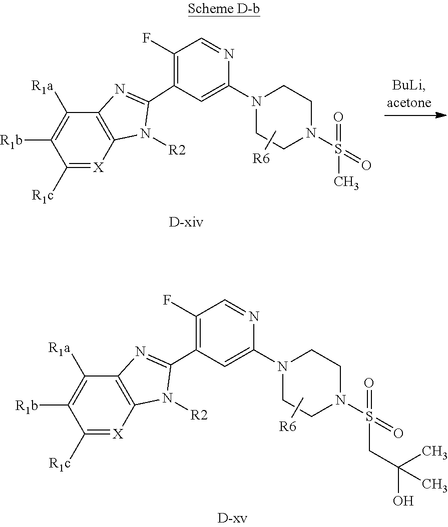

As shown in scheme D-b, the methylsulphonamide intermediate D-xiv was treated with butyl lithium and subsequently quenched with dry acetone to furnish the gem-dimethyl compound D-xv.

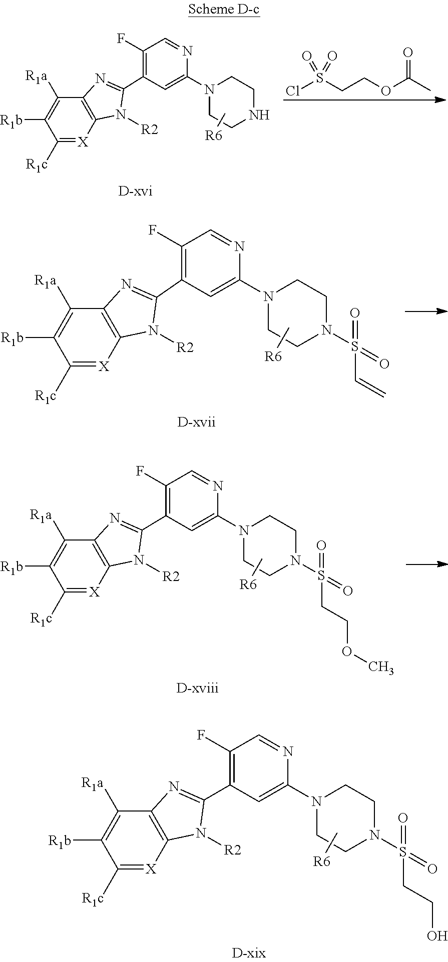

As shown in scheme D-c, the piperazine intermediate D-xvi was sulphonylated with simultaneous elimination to afford the sulphonamide D-xvii. This was treated with sodium hydroxide in methanol to furnish the methoxy derivative D-xviii that was demethylated with BBr₃ to afford the hydroxyethyl product, D-xix.

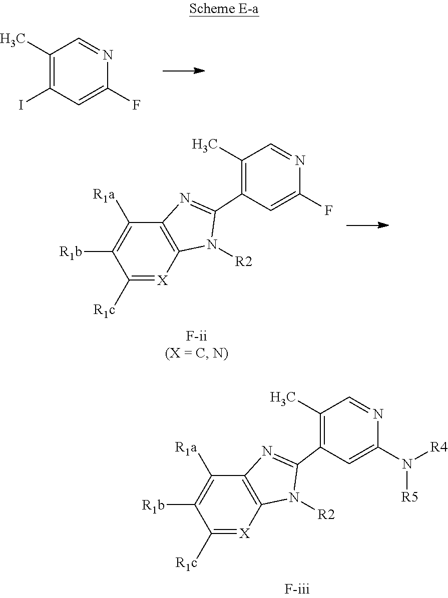

As shown in scheme E-a, the iododpyridine E-i was treated with N-methylbenzimidazole or derivative thereof with copper iodide, triphenyl phosphine and sodium carbonate to afford the direct coupled product E-ii which was subsequently reacted with various amines in the presence of cesium fluoride in a suitable solvent (such as DMSO) to yield products E-iii.

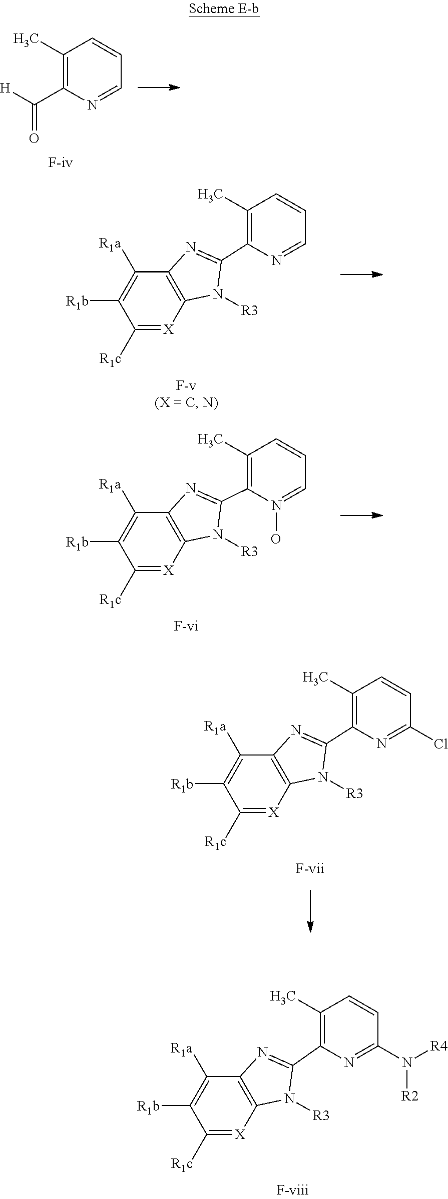

As illustrated in scheme E-b, the pyridine aldehyde E-iv was treated with an N-alkyl-o-phenylenediamine to afford the benzimidazole E-v. This was subsequently oxidized to the N-oxide with a suitable reagent (such as mCPBA) followed by treatment with phosphorusoxychloride to afford the chlorinated pyridine derivative E-vii after isomeric separation where necessary. The chloropyridine E-vii was reacted with various amines in the presence of cesium fluoride in a suitable solvent (such as DMSO) to yield products E-viii.

### Experimentals

### Preparation of intermediate 1: 2,5-dichloropyridine-4-carbaldehyde

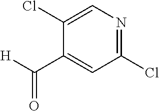

A solution of 2,5-dichloropyridine (27.0 g, 180 mmol) in THF (65 mL) was added via cannula to a cooled solution of LDA (100 mL of a 1.8 M solution, 180 mmol) in THF (80 mL) at −78° C. The mixture was stirred at −78° C. for 30 mins, then a solution of DMF (21.1 mL, 271 mmol) in THF (25 mL) was added slowly via syringe. The reaction was stirred at −78° C. for 3 hours and was then warmed to R.T. gradually. The solution was poured into a mixture of ice (800 mL) and conc. HCl (150 mL) and stirred for 20 mins before being basified with NaOH (3.0 M) to pH 9-10, and extracted with Et₂O (2×500 mL). The combined organic layers were dried over MgSO₄ and concentrated to give the crude product as pale yellow solid. This solid was suspended in n-hexane with trace EtOAc and boiled for 5 mins. The liquors were decanted and stripped to yield a yellow solid which was purified by Biotage flash chromatography (65i, loaded in DCM/EtOAc, eluted with heptane—20% EtOAc/heptane over 8 CV, then holding for 5 CV) to afford the title compound (17.9 g, 56%) as a pale yellow solid, ¹H NMR (400 MHz, DMSO-d6) δ 7.85 (s, 1H) 8.76 (s, 1H) 10.22 (s, 1H).

### Preparation of intermediate 2: 2-(2,5-dichloropyridin-4-yl)-1-methyl-1H-benzimidazole

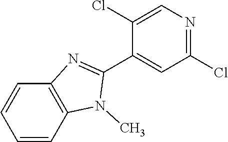

To a solution of 2,5-dichloropyridine-4-carbaldehyde (2.75 g, 15.62 mmol) in DMSO (63 mL) was added N-methyl-O-phenylenediamine (1.91 g, 15.62 mmol) and the mixture stirred at ambient temperature for 5 mins. Sulfur (500 mg, 15.62 mmol) was added and the mixture warmed to 60° C. and allowed to stir for 2.5 hrs. The reaction was then cooled to R.T. and added to a bi-phasic stirred solution of DCM and water (200 mL ea). The resulting emulsion was extracted with DCM (3×100 mL) and the combined organics were washed with water (3×100 mL), dried over MgSO₄, filtered and stripped to a crude red gum which was purified by Biotage flash chromatography (45 M loaded with DCM, eluting with EtOAc/heptane 5-30% over 10 CV, then holding for 5 CV) to afford the title compound (3.22 g, 74%) as a pale orange solid. ¹H NMR (400 MHz, DMSO-d6) δ ppm 3.72 (s, 3H) 7.26-7.35 (m, 1H) 7.35-7.44 (m, 1H) 7.69 (d, J=8.1 Hz, 1H) 7.74 (d, J=8.1 Hz, 1H) 7.95 (s, 1H) 8.78 (s, 1H). m/z (APCI+) for C₁₃H₉N₃Cl₂ 278.05/280.00 (M+H)⁺.

### Preparation of intermediate 3: 2-(5-chloro-2-piperazin-1-ylpyridin-4-yl)-1-methyl-1H-benzimidazole

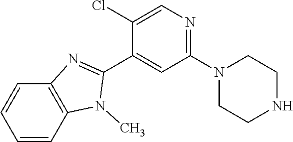

To a solution of 2-(2,5-dichloropyridin-4-yl)-1-methyl-1H-benzimidazole (538 mg, 1.93 mmol) in DMSO (5 mL) was added piperazine (1330 mg, 15.5 mmol) and cesium fluoride (588 mg, 3.87 mmol) and the mixture heated to 120° C. overnight. The reaction was cooled to R.T., diluted with water (30 mL) and extracted with DCM (3×60 mL). The combined organics were washed with water (2×30 mL), dried over MgSO₄, filtered and stripped to yield a clear, colorless gum which was purified by Biotage flash chromatography (25 S eluting with DCM/MeOH/NH₃ gradient; 98/2/0.2-95/5/0.5 over 10 CV, then holding for 3 CV, then increasing to 93/7/0.7 over 5 CV) to afford the title compound (643 mg, 100%) as an off-white solid. ¹H NMR (400 MHz, DMSO-d6) δ ppm 2.72-2.82 (m, 4H) 3.42-3.50 (m, 4H) 3.68 (s, 3H) 7.03 (s, 1H) 7.24-7.31 (m, 1H) 7.31-7.40 (m, 1H) 7.64 (d, J=7.8 Hz, 1H) 7.70 (d, J=7.8 Hz, 1H) 8.31 (s, 1H). m/z (APCI+) for C₁₇H₁₈N₅Cl 328.15/330.10 (M+H)⁺.

### Preparation of example A1: 2-[2-(4-acetylpiperazin-1-yl)-5-chloropyridin-4-yl]-1-methyl-1H-benzimidazole

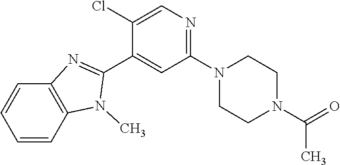

To a solution of 2-(5-chloro-2-piperazin-1-ylpyridin-4-yl)-1-methyl-1H-benzimidazole (100 mg, 0.31 mmol) in DCM (5 mL) was added Hunig's base (62 μL, 0.35 mmol) followed by acetyl chloride (23 μL, 0.32 mmol) and the solution stirred at RT overnight. The mixture was purified directly by Biotage flash chromatography (25 M column eluted with a DCM/MeOH gradient; 0-4% over 10 CV) to afford the title compound (90 mg, 80%) as a white solid. ¹H NMR (400 MHz, DMSO-d6) δ ppm 2.04 (s, 3H) 3.55 (s, 6H) 3.60-3.66 (m, 2H) 3.68 (s, 3H) 7.12 (s, 1H) 7.25-7.32 (m, 1H) 7.32-7.40 (m, 1H) 7.66 (d, J=8.1 Hz, 1H) 7.71 (d, J=8.1 Hz, 1H) 8.36 (s, 1H). m/z (APCI+) for C₁₉H₂₀N₅OC1370.15/372.10 (M+H)⁺.

### Preparation of example A2: methyl 4-[5-chloro-4-(1-methyl-1H-benzimidazol-2-yl)pyridin-2-yl]piperazine-1-carboxylate

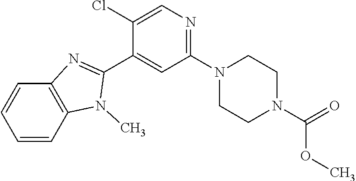

To a solution of 2-(5-chloro-2-piperazin-1-ylpyridin-4-yl)-1-methyl-1H-benzimidazole (70 mg, 0.21 mmol) in DCM (5 mL) was added Hunig's base (43 μL, 0.25 mmol) followed by methyl chloroformate (17 μL, 0.23 mmol) and the solution stirred at RT overnight. The mixture was purified directly by Biotage flash chromatography (25 M column eluted with a DCM/MeOH gradient; 0-4% over 10 CV) to afford the title compound (48 mg, 40%) as a white solid. ¹H NMR (400 MHz, DMSO-d6) δ ppm 3.44-3.51 (m, 4H) 3.55-3.62 (m, 4H) 3.63 (s, 3H) 3.68 (s, 3H) 7.11 (s, 1H) 7.24-7.32 (m, 1H) 7.32-7.42 (m, 1H) 7.65 (d, J=7.8 Hz, 1H) 7.71 (d, J=7.8 Hz, 1H) 8.35 (s, 1H). m/z (APCI+) for C₁₉H₂₀N₅O₂C1386.15/388.10 (M+H)⁺.

### Preparation of example A3: 4-[5-chloro-4-(1-methyl-1H-benzimidazol-2-yflpyridin-2-yl]-N-methylpiperazine-1-carboxamide

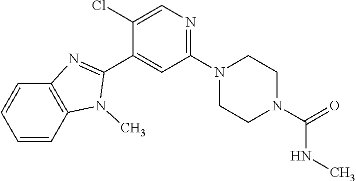

To a solution of 2-(5-chloro-2-piperazin-1-ylpyridin-4-yl)-1-methyl-1H-benzimidazole (100 mg, 0.31 mmol) in DCM (2.5 mL) was added methyl isocyanate (18 μL, 0.31 umol) and the mixture stirred at RT for 17 mins. Ethyl acetate (5 mL) was added and the resulting suspension was sonicated and filtered and the resulting precipitate dried in vacuo to afford the title compound (85 mg, 72%) as a white solid. ¹H NMR (400 MHz, DMSO-d6) δ ppm 2.58 (d, J=4.3 Hz, 3H) 3.35-3.44 (m, 4H) 3.50-3.59 (m, 4H) 3.68 (s, 3H) 6.52 (d, 1H) 7.12 (s, 1H) 7.25-7.32 (m, 1H) 7.32-7.42 (m, 1H) 7.65 (d, J=8.1 Hz, 1H) 7.71 (d, J=7.8 Hz, 1H) 8.34 (s, 1H). m/z (APCI+) for C₁₉H₂₁N₆OCl 385.15/387.10 (M+H)⁺.

### Preparation of example A4: 2-{5-chloro-2-[4-(methylsulfonyl)piperazin-1-yl]pyridin-4-yl}-1-methyl-1H-benzimidazole

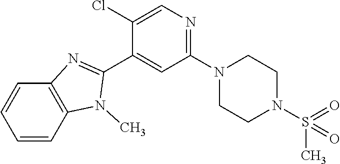

To a solution of 2-(5-chloro-2-piperazin-1-ylpyridin-4-yl)-1-methyl-1H-benzimidazole (100 mg, 0.31 mmol) in DCM (2.5 mL) was added Hunig's base (53 μL, 0.31 mmol) followed by methanesulfonyl chloride (24 μL, 0.31 mmol) and the mixture stirred at R.T. for 2 mins. The mixture was purified directly by Biotage flash chromatography (25 M column eluted with a DCM/MeOH gradient; 0-4% over 10 CV) to afford the title compound (85 mg, 69%) as a white solid. ¹H NMR (400 MHz, DMSO-d6) δ ppm 2.91 (s, 3H) 3.16-3.24 (m, 4H) 3.65-3.76 (m, 7H) 7.18 (s, 1H) 7.26-7.32 (m, 1H) 7.32-7.40 (m, 1H) 7.66 (d, J=7.8 Hz, 1H) 7.71 (d, J=7.8 Hz, 1H) 8.37 (s, 1 H). m/z (APCI+) for C₁₈H₂₀N₅O₂SCl 406.15/408.10 (M+H)⁺.

### Preparation of example A5: N-{1-[5-chloro-4-(1-methyl-1H-benzimidazol-2-yl)pyridin-2-yl]piperidin-4-yl}methanesulfonamide

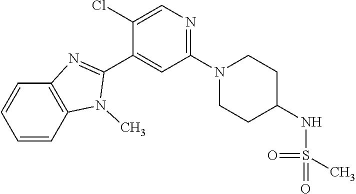

To a solution of 2-(2,5-dichloropyridin-4-yl)-1-methyl-1H-benzimidazole (165 mg, 0.59 mmol) in DMSO (3 mL) was added N-piperidine-4-yl-methylsulfonamide (317 mg, 1.78 mmol) and cesium fluoride (180 mg, 1.19 mmol) and the mixture heated to 107° C. overnight. The reaction was cooled to R.T. and partitioned between DCM (75 mL) and water (25 mL). The phases were separated and the aqueous layer extracted with DCM (20 mL). The combined organics were washed with water (25 mL), dried over MgSO₄, filtered and stripped to a gum which was purified by Biotage flash chromatography (25 M, eluting with DCM/MeOH gradient, 0-3% over 10 CV, then holding for 3 CV) to afford the title compound (238 mg, 96%) as a white foam. ¹H NMR (400 MHz, DMSO-d6) δ ppm 1.31-1.54 (m, 2H) 1.89 (d, J=10.1 Hz, 2H) 2.94 (s, 3H) 3.05 (t, J=11.5 Hz, 2H) 3.38-3.53 (m, 1H) 3.68 (s, 3H) 4.23 (d, 2H) 7.04-7.17 (m, 2H) 7.25-7.31 (m, 1 H) 7.31-7.42 (m, 1H) 7.65 (d, J=7.8 Hz, 1H) 7.71 (d, J=7.8 Hz, 1H) 8.32 (s, 1H). m/z (APCI+) for C₁₉H₂₂N₅ClO₂S 420.05/422.10 (M+H)⁺.

(Note: in the cases where the amine coupling partner was a salt, a stoichiometric amount of either DBU or DIPEA was added to the reaction.)

### Preparation of example A6: N-{1-[5-chloro-4-(1-methyl-1H-benzimidazol-2-yl)pyridin-2-yl]piperidin-4-yl}-N-methylmethanesulfonamide

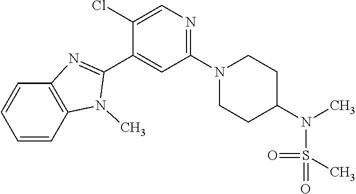

To a suspension of N-{1-[5-chloro-4-(1-methyl-1H-benzimidazol-2-yl)pyridin-2-yl]piperidin-4-yl}methanesulfonamide (158 mg, 0.376 mmol) in dry THF (3 mL) and DMF (1 mL) was added NaH (17 mg, 0.425 mmol). This formed a solution within 1 minute. To this solution was added methyl iodide (26 μL, 0.414 mmol), and the solution stirred at R.T. for 60 mins. The reaction was quenched with water (5 mL) then diluted with EtOAc (75 mL), and washed with water (2×20 mL), brine (15 mL), dried over MgSO₄, filtered and stripped to a clear gum. Purified via Biotage flash chromatography (25 M, eluting with DCM-4% MeOH/DCM over 10 CV) to yield the product as a gum. Recrystallized from ethyl acetate/diethyl ether to afford the title compound (130 mg, 80%) as a white solid. ¹H NMR (400 MHz, DMSO-d6) δ ppm 8.32 (s, 1H) 7.71 (d, J=7.8 Hz, 1H) 7.65 (d, J=7.8 Hz, 1H) 7.32-7.40 (m, 1H) 7.24-7.32 (m, 1H) 7.13 (s, 1H) 4.46 (d, J=13.1 Hz, 2H) 3.80-3.94 (m, 1H) 3.68 (s, 3H) 2.87-3.02 (m, 5H) 2.67 (s, 3 H) 1.60-1.76 (m, 4H). m/z (APCI+) for C₁₉H₂₂N₅ClO₂S 434.05/436.15 (M+H)⁺.

### Preparation of example A7: 1-[5-chloro-4-(1-methyl-1H-benzimidazol-2-yl)pyridin-2-yl]piperidine-4-carboxylic acid

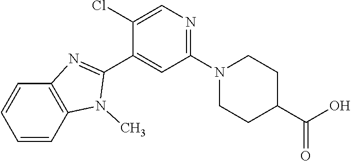

To a solution of methyl 1-[5-chloro-4-(1-methyl-1H-benzimidazol-2-yl)pyridin-2-yl]piperidine-4-carboxylate (190 mg. 0.45 mmol-prepared as in example A5 with appropriate substitutions and non-critical method changes) in THF (5 mL) was added 2 M NaOH (0.741 mL, 1.48 mmol) and MeOH (1 mL) to yield a solution, and the mixture stirred at R.T. for 30 mins. The reaction was stripped to dryness and neutralized with a stoichiometric amount of 1 N HCl (1.48 mL). This caused the product to gum out quickly, but then it was noticed that crystallization started to occur. Diluted with water (10 mL) and stirred vigorously overnight to induce full crystallization. The resulting white precipitate was filtered off and dried with desiccation overnight in vacuo to afford the title compound (122 mg, 67%) as a white solid. ¹H NMR (400 MHz, DMSO-d6) δ ppm 12.26 (br. s., 1H) 8.31 (s, 1H) 7.71 (d, J=7.8 Hz, 1H) 7.65 (d, J=7.8 Hz, 1H) 7.31-7.41 (m, 1H) 7.24-7.31 (m, 1H) 7.09 (s, 1H) 4.22 (d, J=13.4 Hz, 2H) 3.68 (s, 3H) 2.95-3.10 (m, 2H) 2.52-2.60 (m, 1H) 1.87 (dd, J=13.1, 3.0 Hz, 2H) 1.45-1.62 (m, 2 H). m/z (APCI+) for C₁₉H₁₉N₄ClO₂ 371.20/373.15 (M+H)⁺.

### Preparation of intermediate 4: tert-butyl {(1R,5S,6S)-3-[5-chloro-4-(1-methyl-1H-benzimidazol-2-yl)pyridin-2-yl]-3-azabicyclo[3.1.0]hex-6-yl}carbamate

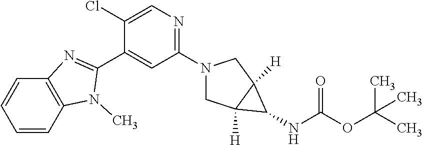

To a solution of 2-(2,5-dichloropyridin-4-yl)-1-methyl-1H-benzimidazole (222 mg, 0.8 mmol) in DMSO (5 mL) was added (1R,5S)-tert-butyl 3,6-diazabicyclo[3.1.0]hexane-6-carboxylate (476 mg, 2.4 mmol) and cesium fluoride (365 mg, 2.4 mmol), and the mixture was heated to 75° C. overnight. The reaction was cooled to room temperature and diluted with water (10 mL), and this was vigorously stirred for 30 minutes. After filtration, the filtrate was washed with water (20 mL) and concentrated to afford a crude product which was purified by Biotage flash chromatography (25 S eluting with EtOAc/Heptane 0-60% over 8 CV) to yield the title compound (210 mg, 60%) as a white solid. ¹H NMR (400 MHz, CHLOROFORM-d) δ ppm 8.23 (s, 1H) 7.84 (d, J=7.07 Hz, 1H) 7.42-7.49 (m, 1H) 7.29-7.42 (m, 2H) 6.56 (s, 1H) 4.77 (br. s., 1H) 3.88 (s, 1H) 3.74 (s, 3H) 3.55-3.64 (m, 1H) 3.26 (d, J=9.60 Hz, 1H) 3.11 (s, 2H) 2.63 (s, 9H) 1.85-1.98 (m, 2H). m/z (APCI+) for C₂₃H₂₆N₅ClO₂ 339.95/440.20 (M+H)⁺.

### Preparation of intermediate 5: (1R,5S,6S)-3-[5-chloro-4-(1-methyl-1H-benzimidazol-2-yl)pyridin-2-yl]-3-azabicyclo[3.1.0]hexan-6-amine

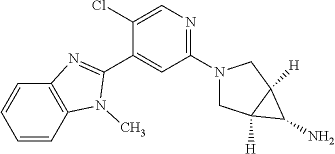

4 N HCl in dioxane (0.164 mL, 0.656 mmol) was added to an ice-cooled solution of the tert-butyl {(1R,5S,6S)-3-[5-chloro-4-(1-methyl-1H-benzimidazol-2-yl)pyridin-2-yl]-3-azabicyclo[3.1.0]hex-6-yl}carbamate (72 mg, 0.16 mmol) in dichloromethane (0.82 mL), and the mixture was allowed to warm to room temperature and stirred for 2 hours. The reaction was then concentrated and the product (56 mg, 90%) was carried to the next step without further purification.

### Preparation of example A8: N-{(1R,5S,6S)-3-[5-chloro-4-(1-methyl-1H-benzimidazol-2-yl)pyridin-2-yl]-3-azabicyclo[3.1.0]hex-6-yl}methanesulfonamide

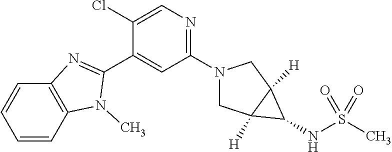

To a solution of (1R,5S,6S)-3-[5-chloro-4-(1-methyl-1H-benzimidazol-2-yl)pyridin-2-yl]-3-azabicyclo[3.1.0]hexan-6-amine (56 mg, 0.15 mmol) in DCM (5 mL) was added Hunig's base (83 μL, 0.60 mmol) followed by methanesulfonyl chloride (17 μL, 0.22 mmol), and the solution was stirred at room temperature overnight. The solution was then quenched with water (4 mL), extracted with DCM (2×10 mL), dried over MgSO₄, and the concentrated crude product was purified by Biotage flash chromatography (12 M column eluted with a DCM/MeOH gradient; 0-4% over 10 CV) to afford the title compound (38 mg, 61%) as a white solid. ¹H NMR (400 MHz, DMSO-d₆) δ ppm 8.29 (s, 1H) 7.70 (d, J=8.08 Hz, 1H) 7.65 (d, J=7.83 Hz, 1H) 7.49 (d, J=2.02 Hz, 1H) 7.25-7.38 (m, 2H) 6.70 (s, 1H) 3.72 (d, J=10.86 Hz, 2H) 3.67 (s, 3H) 3.47 (d, J=10.11 Hz, 2H) 3.38 (q, J=6.99 Hz, 2H) 2.97 (s, 3H) 2.33 (d, J=1.77 Hz, 1H). m/z (APCI+) for C₁₆H₂₀N₆O₂Cl 417.91/418.20 (M+H)⁺.

### Preparation of example A9: N-{(1R,5S,6S)-3-[5-chloro-4-(1-methyl-1H-benzimidazol-2-yl)pyridin-2-yl]-3-azabicyclo[3.1.0]hex-6-yl}acetamide

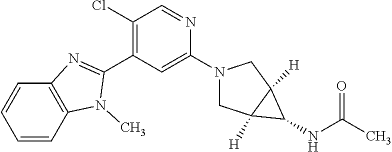

To a solution of (1R,5S,6S)-3-[5-chloro-4-(1-methyl-1H-benzimidazol-2-yl)pyridin-2-yl]-3-azabicyclo[3.1.0]hexan-6-amine (60 mg, 0.16 mmol) in DCM (0.8 mL) was added acetyl chloride (17 μL, 0.24 mmol) and the mixture was stirred at room temperature for 10 minutes. DCM (5 mL) and water (2 mL) was added. The reaction mixture was extracted with DCM (2×5 mL). The combined organic layer was washed with brine, dried over MgSO₄, and concentrated to crude product that was purified by Biotage (12 M column eluted with a DCM/MeOH gradient; 0-4% over 10 CV) to afford the title compound (44 mg, 65%) as a white solid. ¹H NMR (400 MHz, DMSO-d₆) δ ppm 8.29 (s, 1H) 8.03 (d, J=3.79 Hz, 1H) 7.71 (d, J=7.83 Hz, 1H) 7.65 (d, J=7.83 Hz, 1H) 7.31 (dd, J=17.05, 7.20 Hz, 2H) 6.71 (s, 1H) 3.71 (d, J=10.36 Hz, 2H) 3.67 (s, 3H) 3.42-3.49 (m, 2H) 2.39 (d, J=3.03 Hz, 1H) 1.75-1.81 (m, 5H). m/z (APCI+) for C₂₀H₂₀N₅OCl 381.86/382.20 (M+H)⁺.

### Preparation of intermediate 6: 2-(2-bromopyridin-4-yl)-1-methyl-1H-benzimidazole

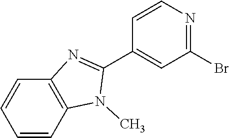

To a solution of 2-bromopyridine-4-carboxaldehyde (128 mg, 0.688 mmol) in DMA (3 mL) was added N-methyl-O-phenylenediamine (84 mg, 0.688 mmol) followed by sulfur (22 mg, 0.688 mmol) and the mixture stirred at 65° C. for 1 hour, followed by heating to 85° C. for 30 mins. The reaction was cooled and quenched with water (10 mL), extracted with EtOAc (25 mL), dried over MgSO₄, filtered and stripped to a dark oil. The crude product was purified by Biotage flash chromatography (25 S, eluting with 20-50% EtOAc/heptane over 10 CV, then holding for 2 CV) to afford the title compound (80 mg, 40%) as a pale brown solid. ¹H NMR (400 MHz, DMSO-d6) d ppm 3.98 (s, 3 H) 7.29-7.35 (m, 1H) 7.35-7.44 (m, 1H) 7.71 (d, J=7.8 Hz, 1H) 7.76 (d, J=7.6 Hz, 1H) 7.97 (d, J=5.1 Hz, 1H) 8.13 (s, 1H) 8.61 (d, J=5.1 Hz, 1H).

### Preparation of example A10:1-methyl-2-{2-[4-(methylsulfonyl)piperidin-1-yl]pyridin-4-yl}-1H-benzimidazole

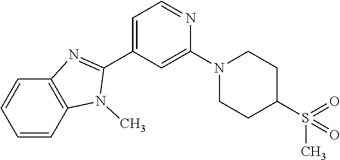

The title compound was prepared in an analogous way to example A-5 making non critical method changes and appropriate substitutions to yield an off-white solid (76 mg, 42%). ¹H NMR (400 MHz, DMSO-d6) δ ppm 1.51-1.73 (m, 2H) 2.10 (d, J=12.9 Hz, 2H) 2.87-3.08 (m, 5H) 3.38-3.52 (m, 1H) 3.93 (s, 3H) 4.58 (d, J=12.9 Hz, 2H) 7.08 (d, J=4.8 Hz, 1H) 7.20-7.32 (m, 2H) 7.31-7.38 (m, 1H) 7.67 (d, J=7.8 Hz, 1H) 7.72 (d, J=7.8 Hz, 1H) 8.31 (d, J=5.1 Hz, 1H). m/z (APCI+) for C₁₉H₂₂N₄O₂S 371.20 (M+H)⁺.

The following examples listed in Table 1 were prepared with appropriate substitutions in analogous ways to examples A1-A10.

TABLE 1

| | | | LRMS | |
|---|---|---|---|---|
| Example | | Compound | m/z | |
| Number | Structure | Name | (M + H) | ¹H NMR |
| A-1 | 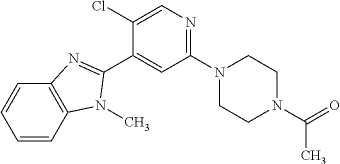 | 2-[2-(4- acetylpiperazin- 1-yl)-5- chloropyridin-4- yl]-1-methyl-1H- benzimidazole | 370.15/ 372.10 | ¹H NMR (400 MHz, DMSO-d6) δ ppm 2.04 (s, 3 H) 3.55 (s, 6 H) 3.60-3.66 (m, 2 H) 3.68 (s, 3 H), 7.12 (s, 1H) 7.25-7.32 (m, 1 H) 7.32-7.40 (m, 1 H) 7.66 (d, J = 8.1 Hz, 1 H) 7.71 (d, J = 8.1 Hz, 1 H) 8.36 (s, 1 H) |
| A-2 | 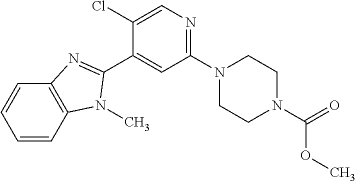 | methyl 4-[5- chloro-4-(1- methyl-1H- benzimidazol- 2-yl)pyridin-2- yl]piperazine-1- carboxylate | 386.15/ 388.10 | ¹H NMR (400 MHz, DMSO-d6) δ ppm 3.44-3.51 (m, 4 H) 3.55-3.62 (m, 4 H) 3.63 (s, 3 H) 3.68 (s, 3 H) 7.11 (s, 1 H) 7.24-7.32 (m, 1 H) 7.32- 7.42 (m, 1 H) 7.65 (d, J = 7.8 Hz, 1 H) 7.71 (d, J = 7.8 Hz, 1 H) 8.35 (s, 1 H) |
| A-3 | 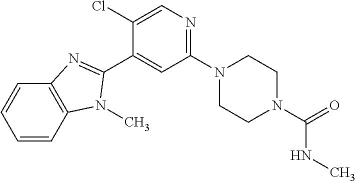 | 4-[5-chloro-4- (1-methyl-1H- benzimidazol- 2-yl)pyridin-2- yl]-N-methyl- piperazine-1- carboxamide | 385.15/ 387.10 | ¹H NMR (400 MHz, DMSO-d6) δ ppm 2.58 (d, J = 4.3 Hz, 3 H) 3.35-3.44 (m, 4 H) 3.50- 3.59 (m, 4 H) 3.68 (s, 3 H) 6.52 (d, 1 H) 7.12 (s, 1 H) 7.25- 7.32 (m, 1 H) 7.32-7.42 (m, 1 H) 7.65 (d, J = 8.1 Hz, 1 H) 7.71 (d, J = 7.8 Hz, 1 H) 8.34 (s, 1 H) |
| A-4 | 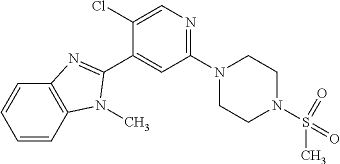 | 2-{5-chloro- 2-[4-(methyl- sulfonyl) piperazin-1- yl]pyridin-4-yl}- 1-methyl-1H- benzimidazole | 406.15/ 408.10 | ¹H NMR (400 MHz, DMSO-d6) δ ppm 2.91 (s, 3 H) 3.16-3.24 (m, 4 H) 3.65-3.76 (m, 7 H) 7.18 (s, 1 H) 7.26-7.32 (m, 1 H) 7.32-7.40 (m, 1 H) 7.66 (d, J = 7.8 Hz, 1 H) 7.71 (d, J = 7.8 Hz, 1 H) 8.37 (s, 1 H) |
| A-5 | 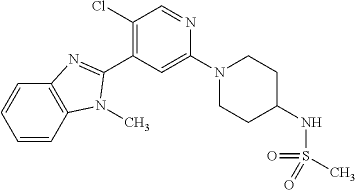 | N-{1-[5-chloro- 4-(1-methyl-1H- benzimidazol- 2-yl)pyridin-2- yl]piperidin-4- yl}methane- sulfonamide | 420.05/ 422.10 | ¹H NMR (400 MHz, DMSO-d6) δ ppm 8.32 (s, 1 H) 7.71 (d, J = 7.6 Hz, 1 H) 7.65 (d, J = 7.8 Hz, 1 H) 7.32-7.41 (m, 1 H) 7.25-7.32 (m, 1 H) 7.06-7.17 (m, 2 H) 4.23 (d, 2 H) 3.68 (s, 3 H) 3.45 (br. s., 1 H) 3.05 (t, J = 11.4 Hz, 2 H) 2.94 (s, 3 H) 1.82-1.94 (m, 2 H) 1.32- 1.53 (m, 2 H) |
| A-6 | 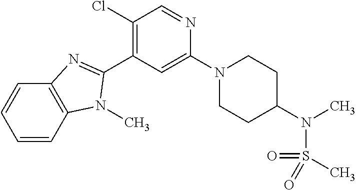 | N-{1-[5-chloro- 4-(1-methyl-1H- benzimidazol- 2-yl)pyridin-2- yl]piperidin-4- yl}-N-methyl- methane sulfonamide | 434.05/ 436.15 | ¹H NMR (400 MHz, DMSO-d6) δ ppm 8.32 (s, 1 H) 7.71 (d, J = 7.8 Hz, 1 H) 7.65 (d, J = 7.8 Hz, 1 H) 7.32-7.40 (m, 1 H) 7.24-7.32 (m, 1 H) 7.13 (s, 1 H) 4.46 (d, J = 13.1 Hz, 2 H) 3.80- 3.94 (m, 1 H) 3.68 (s, 3 H) 2.87-3.02 (m, 5 H) 2.67 (s, 3 H) 1.60-1.76 (m, 4 H) |
| A-7 | 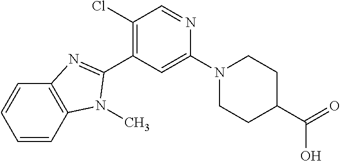 | 1-[5-chloro-4- (1-methyl-1H- benzimidazol- 2-yl)pyridin-2- yl]piperidine-4- carboxylic acid | 371.20/ 373.15 | ¹H NMR (400 MHz, DMSO-d6) δ ppm 12.26 (br. s., 1 H) 8.31 (s, 1 H) 7.71 (d, J = 7.8 Hz, 1 H) 7.65 (d, J = 7.8 Hz, 1 H) 7.31- 7.41 (m, 1 H) 7.24-7.31 (m, 1 H) 7.09 (s, 1 H) 4.22 (d, J = 13.4 Hz, 2 H) 3.68 (s, 3 H) 2.95-3.10 (m, 2 H) 2.52-2.60 (m, 1 H) 1.87 (dd, J = 13.1, 3.0 Hz, 2 H) 1.45-1.62 (m, 2 H) |
| A-8 | 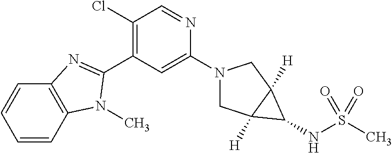 | N-{(1R,5S,6s)- 3-[5-chloro-4- (1-methyl-1H- benzimidazol- 2-yl)pyridin-2- yl]-3-azabicyclo [3.1.0]hex-6-yl} methane- sulfonamide | 417.92/ 418.15 | ¹H NMR (400 MHz, DMSO-d₆) δ ppm 8.29 (s, 1 H) 7.70 (d, J = 8.08 Hz, 1 H) 7.65 (d, J = 7.83 Hz, 1 H) 7.49 (d, J = 2.02 Hz, 1 H) 7.25-7.38 (m, 2 H) 6.70 (s, 1 H) 3.72 (d, J = 10.86 Hz, 2 H) 3.67 (s, 3 H) 3.47 (d, J = 10.11 Hz, 2 H) 3.38 (q, J = 6.99 Hz, 2 H) 2.97 (s, 3 H) 2.33 (d, J = 1.77 Hz, 1 H) |
| A-9 | 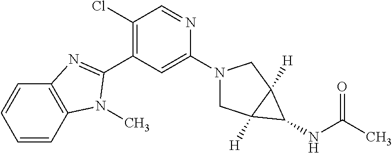 | N-{(1R,5S,6s)- 3-[5-chloro-4- (1-methyl-1H- benzimidazol- 2-yl)pyridin-2- yl]-3-azabicyclo [3.1.0]hex-6-yl} methane- sulfonamide | 381.14/ 382.20 | ¹H NMR (400 MHz, DMSO-d₆) δ ppm 8.29 (s, 1 H) 8.03 (d, J = 3.79 Hz, 1 H) 7.71 (d, J = 7.83 Hz, 1 H) 7.65 (d, J = 7.83 Hz, 1 H) 7.31 (dd, J = 17.05, 7.20 Hz, 2 H) 6.71 (s, 1 H) 3.71 (d, J = 10.36 Hz, 2 H) 3.67 (s, 3 H) 3.42-3.49 (m, 2 H) 2.39 (d, J = 3.03 Hz, 1 H) 1.75-1.81 (m, 5 H) |
| A-10 | 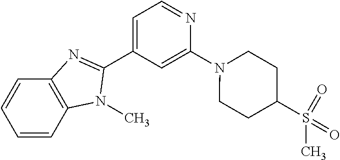 | 1-methyl-2-{2- [4-(methyl- sulfonyl) piperidin-1-yl] pyridin-4-yl}- 1H- benzimidazole | 371.20 | ¹H NMR (400 MHz, DMSO-d₆) δ ppm 1.51-1.73 (m, 2 H) 2.10 (d, J = 12.9 Hz, 2 H) 2.87- 3.08 (m, 5 H) 3.38-3.52 (m, 1 H) 3.93 (s, 3 H) 4.58 (d, J = 12.9 Hz, 2 H) 7.08 (d, J = 4.8 Hz, 1 H) 7.20-7.32 (m, 2 H) 7.31-7.38 (m, 1 H) 7.67 (d, J = 7.8 Hz, 1 H) 7.72 (d, J = 7.8 Hz, 1 H) 8.31 (d, J = 5.1 Hz, 1 H) |
| A-11 | 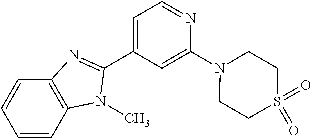 | 2-[2-(1,1- dioxidothio- morpholin-4- yl)pyridin-4-yl]- 1-methyl-1H- benzimidazole | 343.15 | ¹H NMR (400 MHz, DMSO-d6) δ ppm 3.17 (br. s., 4 H) 3.94 (s, 3 H) 4.17 (br. s., 4 H) 7.18 (d, J = 5.1 Hz, 1 H) 7.25-7.32 (m, 1 H) 7.32-7.38 (m, 1 H) 7.40 (s, 1 H) 7.68 (d, J = 7.8 Hz, 1 H) 7.73 (d, J = 7.8 Hz, 1 H) 8.36 (d, J = 5.1 Hz, 1 H) |
| A-12 | 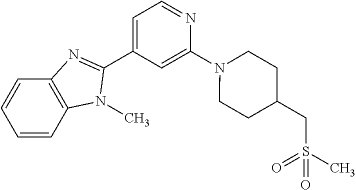 | 1-methyl-2-(2- {4-[(methyl- sulfonyl)methyl] piperidin-1-yl} pyridin-4-yl)-1H- benzimidazole | 385.15 | ¹H NMR (400 MHz, DMSO-d6) δ ppm 1.29-1.43 (m, 2 H) 1.88-2.00 (m, 2 H) 2.18- 2.30 (m, 1 H) 2.91-2.98 (m, 2 H) 3.01 (s, 3 H) 3.16 (d, J = 6.6 Hz, 2 H) 3.92 (s, 3 H) 4.37 (d, J = 13.1 Hz, 2 H) 7.02 (d, J = 5.1 Hz, 1 H) 7.20 (s, 1 H) 7.25-7.31 (m, 1 H) 7.31-7.40 (m, 1 H) 7.66 (d, J = 7.8 Hz, 1 H) 7.72 (d, J = 7.8 Hz, 1 H) 8.29 (d, J = 5.1 Hz, 1 H) |
| A-13 | 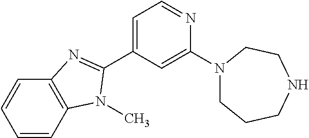 | 2-[2-(1,4- diazepan-1- yl)pyridin-4-yl]- 1-methyl-1H- benzimidazole | 308.20 | ¹H NMR (400 MHz, DMSO-d6) δ ppm 1.80 (dq, J = 6.1, 5.8 Hz, 2 H) 2.70 (t, J = 5.8 Hz, 2 H) 2.81-2.93 (m, 2 H) 3.18 (s, 1 H) 3.64-3.73 (m, 2 H) 3.75 (t, J = 5.9 Hz, 2 H) 3.92 (s, 3 H) 6.93 (d, J = 5.1 Hz, 1 H) 6.97 (s, 1 H) 7.24-7.31 (m, 1 H) 7.31-7.38 (m, 1 H) 7.65 (d, J = 7.8 Hz, 1 H) 7.72 (d, J = 7.8 Hz, 1 H) 8.24 (d, J = 5.1 Hz, 1 H) |
| A-14 | 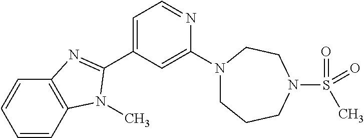 | 1-methyl-2-{2- [4-(methyl- sulfonyl)-1,4- diazepan-1-yl] pyridin-4-yl}-1H- benzimidazole | 386.20 | ¹H NMR (400 MHz, DMSO-d6) δ ppm 1.91 (dq, J = 5.8, 5.6 Hz, 2 H) 2.85 (s, 3 H) 3.27 (t, J = 5.7 Hz, 2 H) 3.47 (t, J = 5.3 Hz, 2 H) 3.81 (t, J = 6.1 Hz, 2 H) 3.89 (t, J = 5.3 Hz, 2 H) 3.92 (s, 3 H) 7.00 (d, J = 5.3 Hz, 1 H) 7.07 (s, 1 H) 7.25- 7.31 (m, 1 H) 7.31-7.40 (m, 1 H) 7.66 (d, J = 8.1 Hz, 1 H) 7.73 (d, J = 7.8 Hz, 1 H) 8.28 (d, J = 5.1 Hz, 1 H) |
| A-15 | 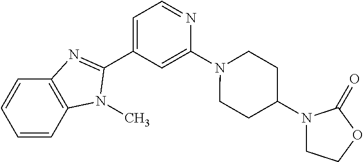 | 3-{1-[4-(1- methyl-1H- benzimidazol-2- yl)pyridin-2- yl]piperidin-4- yl}-1,3-oxa- zolidin-2-one | 378.25 | ND |
| A-16 | 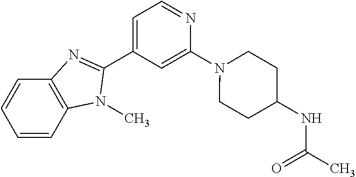 | N-{1-[4-(1- methyl-1H- benzimidazol-2- yl)pyridin-2-yl] piperidin-4-yl} acetamide | 350.20 | ¹H NMR (400 MHz, DMSO-d6) δ ppm 1.20-1.37 (m, 2 H) 1.65-1.78 (m, 5 H) 2.87-3.02 (m, 2 H) 3.69-3.80 (m, 1 H) 3.82 (s, 3 H) 4.21 (d, J = 13.4 Hz, 2 H) 6.94 (dd, J = 5.1, 1.0 Hz, 1 H) 7.12 (s, 1 H) 7.15- 7.22 (m, 1 H) 7.22-7.29 (m, 1 H) 7.56 (d, J = 7.8 Hz, 1 H) 7.62 (d, J = 7.8 Hz, 1 H) 7.73 (d, J = 7.6 Hz, 1 H) 8.20 (d, J = 5.3 Hz, 1 H) |
| A-17 | 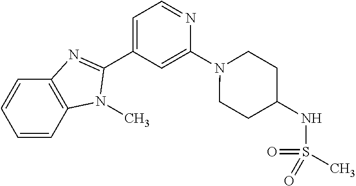 | N-{1-[4-(1- methyl-1H- benzimidazol-2- yl)pyridin-2-yl] piperidin-4-yl} methane- sulfonamide | 386.20 | ¹H NMR (400 MHz, DMSO-d6) δ ppm 1.28-1.45 (m, 2 H) 1.83 (dd, J = 12.5, 2.7 Hz, 2 H) 2.87 (s, 3 H) 2.90-3.02 (m, 2 H) 3.31-3.44 (m, 1 H) 3.82 (s, 3 H) 4.21 (d, J = 13.4 Hz, 2 H) 6.94 (dd, J = 5.1, 1.0 Hz, 1 H) 7.04 (d, J = 7.1 Hz, 1 H) 7.12 (s, 1 H) 7.15-7.22 (m, 1 H) 7.22-7.30 (m, 1 H) 7.57 (d, J = 7.8 Hz, 1 H) 7.62 (d, J = 7.8 Hz, 1 H) 8.19 (d, J = 5.1 Hz, 1 H) |
| A-18 | 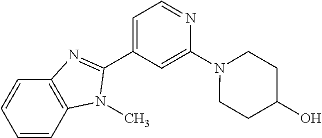 | 1-[4-(1-methyl- 1H-benz- imidazol-2-yl) pyridin-2-yl] piperidin-4-ol | 309.25 | ¹H NMR (400 MHz, DMSO-d6) δ ppm 1.23-1.39 (m, 2 H) 1.65-1.79 (m, 2 H) 3.09 (ddd, J = 13.1, 10.2, 2.8 Hz, 2 H) 3.65 (td, J = 8.6, 4.3 Hz, 1 H) 3.82 (s, 3 H) 3.94-4.11 (m, 2 H) 4.62 (d, J = 4.3 Hz, 1 H) 6.92 (d, J = 5.1 Hz, 1 H) 7.10 (s, 1 H) 7.15-7.21 (m, 1 H) 7.21- 7.31 (m, 1 H) 7.56 (d, J = 7.8 Hz, 1 H) 7.62 (d, J = 7.8 Hz, 1 H) 8.18 (d, J = 5.1 Hz, 1 H) |
| A-19 | 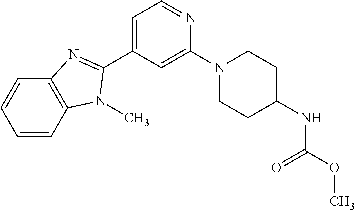 | methyl {1-[4-(1- methyl-1H- benzimidazol- 2-yl)pyridin-2- yl]piperidin-4- yl}carbamate | 366.25 | ¹H NMR (400 MHz, DMSO-d6) δ ppm 1.22-1.39 (m, 2 H) 1.73 (d, J = 10.1 Hz, 2 H) 2.94 (t, J = 11.9 Hz, 2 H) 3.44 (s, 3 H) 3.51 (d, J = 7.3 Hz, 1 H) 3.82 (s, 3 H) 4.22 (d, J = 13.1 Hz, 2 H) 6.93 (d, J = 5.1 Hz, 1 H) 7.08 (d, J = 7.6 Hz, 1 H) 7.11 (s, 1 H) 7.15-7.21 (m, 1 H) 7.21-7.32 (m, 1 H) 7.56 (d, J = 8.1 Hz, 1 H) 7.62 (d, J = 7.8 Hz, 1 H) 8.19 (d, J = 5.1 Hz, 1 H) |
| A-20 | 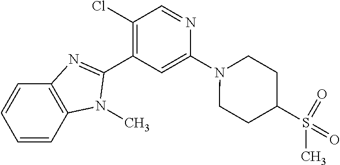 | 2-{5-chloro-2-[4- (methylsulfonyl) piperidin-1-yl] pyridin-4-yl}- 1-methyl-1H- benzimidazole | 405.10/ 407.05 | ¹H NMR (400 MHz, DMSO-d6) δ ppm 1.59 (qd, J = 12.4, 4.0 Hz, 2 H) 2.07 (d, J = 10.9 Hz, 2 H) 2.88-3.03 (m, 5 H) 3.41 (tt, J = 12.0, 3.6 Hz, 1 H) 3.69 (s, 3 H) 4.50 (d, 2 H) 7.17 (s, 1 H) 7.25-7.32 (m, 1 H) 7.32- 7.39 (m, 1H) 7.65 (d, J = 7.8 Hz, 1 H) 7.71 (d, J = 7.8 Hz, 1 H) 8.34 (s, 1 H) |
| A-21 | 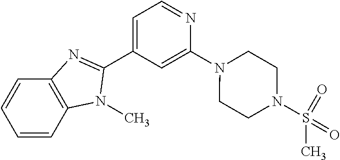 | 1-methyl-2-{2- [4-(methyl- sulfonyl) piperazin-1- yl]pyridin-4- yl}-1H- benzimidazole | 372.20 | ¹H NMR (400 MHz, DMSO-d6) δ ppm 2.93 (s, 3 H) 3.25 (d, J = 4.5 Hz, 4 H) 3.69-3.79 (m, 4 H) 3.93 (s, 3 H) 7.13 (d, J = 5.1 Hz, 1 H) 7.25-7.31 (m, 2 H) 7.32-7.39 (m, 1 H) 7.67 (d, J = 7.8 Hz, 1 H) 7.72 (d, J = 7.8 Hz, 1 H) 8.34 (d, J = 5.1 Hz, 1 H) |
| A-22 | 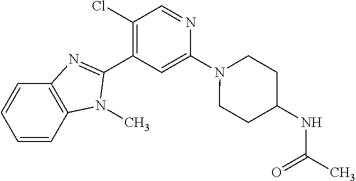 | N-{1-[5-chloro- 4-(1-methyl-1H- benzimidazol- 2-yl)pyridin-2- yl]piperidin-4- yl}acetamide | 384.10/ 386.20 | ¹H NMR (400 MHz, DMSO-d6) δ ppm 1.26-1.48 (m, 2 H) 1.69-1.86 (m, 5 H) 2.90-3.14 (m, 2 H) 3.68 (s, 3 H) 3.77- 3.90 (m, 1 H) 4.22 (d, J = 13.4 Hz, 2 H) 7.11 (s, 1 H) 7.24- 7.32 (m, 1 H) 7.32-7.40 (m, 1 H) 7.65 (d, J = 7.8 Hz, 1 H) 7.71 (d, J = 7.8 Hz, 1 H) 7.81 (d, J = 7.6 Hz, 1 H) 8.32 (s, 1 H) |
| A-23 | 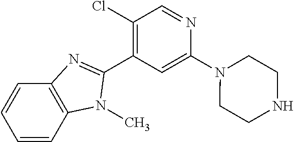 | 2-(5-chloro-2- piperazin-1- ylpyridin-4-yl)- 1-methyl-1H- benzimidazole | 328.15/ 330.10 | ¹H NMR (400 MHz, DMSO-d6) δ ppm 2.72-2.82 (m, 4 H) 3.42- 3.50 (m, 4 H) 3.68 (s, 3 H) 7.03 (s, 1 H) 7.24-7.31 (m, 1 H) 7.31-7.40 (m, 1 H) 7.64 (d, J = 7.8 Hz, 1 H) 7.70 (d, J = 7.8 Hz, 1 H) 8.31 (s, 1 H) |
| A-24 | 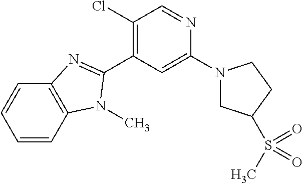 | 2-{5-chloro-2-[3- (methylsulfonyl) pyrrolidin-1- yl]pyridin-4-yl}- 1-methyl-1H- benzimidazole | 391.15/ 393.10 | ¹H NMR (400 MHz, DMSO-d6) δ ppm 2.36-2.46 (m, 2 H) 3.07 (s, 3 H) 3.52 (dt, J = 10.0, 7.0 Hz, 1 H) 3.56-3.66 (m, 1 H) 3.69 (s, 3 H) 3.76-3.91 (m, 2 H) 4.07-4.17 (m, 1 H) 6.81 (s, 1 H) 7.25-7.32 (m, 1 H) 7.32-7.39 (m, 1 H) 7.65 (d, J = 7.8 Hz, 1 H) 7.71 (d, J = 7.8 Hz, 1 H) 8.34 (s, 1 H) |
| A-25 | 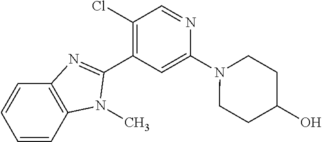 | 1-[5-chloro-4- (1-methyl-1H- benzimidazol- 2-yl)pyridin-2- yl]piperidin-4-ol | 343.15/ 345.10 | ¹H NMR (400 MHz, DMSO-d6) δ ppm 1.29-1.47 (m, 2 H) 1.71-1.84 (m, 2 H) 3.12- 3.25 (m, 2 H) 3.68 (s, 3 H) 3.73 (td, J = 8.6, 4.3 Hz, 1 H) 4.02 (ddd, 2 H) 4.72 (d, J = 4.3 Hz, 1 H) 7.08 (s, 1 H) 7.25- 7.31 (m, 1 H) 7.32-7.40 (m, 1 H) 7.65 (d, J = 7.8 Hz, 1 H) 7.71 (d, J = 7.8 Hz, 1 H) 8.30 (s, 1 H) |
| A-26 | 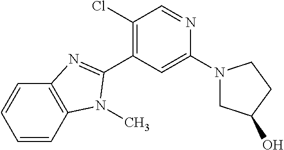 | (3R)-1-[5- chloro-4-(1- methyl-1H- benzimidazol- 2-yl)pyridin-2- yl]pyrrolidin-3-ol | 329.10/ 331.15 | ¹H NMR (400 MHz, DMSO-d6) δ ppm 1.85-1.96 (m, 1 H) 1.97-2.11 (m, 1 H) 3.17 (d, J = 5.1 Hz, 1 H) 3.42-3.57 (m, 3 H) 3.68 (s, 3 H) 4.40 (br. s., 1 H) 5.00 (d, J = 3.5 Hz, 1 H) 6.66 (s, 1 H) 7.24-7.31 (m, 1 H) 7.32-7.38 (m, 1 H) 7.65 (d, J = 7.8 Hz, 1 H) 7.71 (d, J = 7.8 Hz, 1 H) 8.28 (s, 1 H) |
| A-27 | 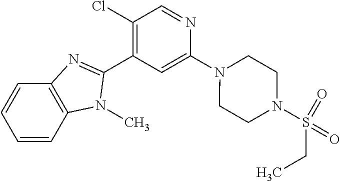 | 2-{5-chloro-2-[4- (ethylsulfonyl) piperazin-1- yl]pyridin-4-yl}- 1-methyl-1H- benzimidazole | 420.10/ 422.05 | ¹H NMR (400 MHz, DMSO-d6) δ ppm 1.22 (t, J = 7.3 Hz, 3 H) 3.10 (q, J = 7.2 Hz, 2 H) 3.21- 3.30 (m, 4 H) 3.68 (s, 7 H) 7.16 (s, 1 H) 7.31 (s, 1 H) 7.35 (s, 1 H) 7.66 (d, J = 7.8 Hz, 1 H) 7.71 (d, J = 8.3 Hz, 1 H) 8.37 (s, 1 H) |
| A-28 | 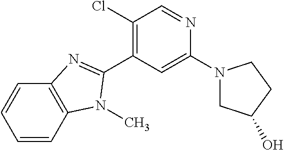 | (3S)-1-[5- chloro-4-(1- methyl-1H- benzimidazol- 2-yl)pyridin-2- yl]pyrrolidin-3-ol | 329.10/ 331.15 | ¹H NMR (400 MHz, DMSO-d6) δ ppm 1.85-1.96 (m, 1 H) 1.97-2.11 (m, 1 H) 3.26-3.39 (m, 2 H) 3.42-3.56 (m, 2 H) 3.62-3.73 (m, 3 H) 4.40 (br. s., 1 H) 5.00 (d, J = 3.5 Hz, 1 H) 6.66 (s, 1 H) 7.25-7.32 (m, 1 H) 7.32-7.41 (m, 1 H) 7.65 (d, J = 8.1 Hz, 1 H) 7.71 (d, J = 7.8 Hz, 1 H) 8.28 (s, 1 H) |
| A-29 | 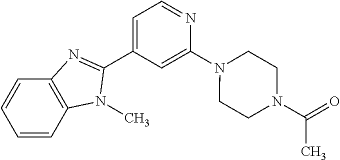 | 2-[2-(4-acetyl- piperazin-1-yl) pyridin-4-yl]-1- methyl-1H- benzimidazole | 336.20 | ¹H NMR (400 MHz, DMSO-d6) δ ppm 2.07 (s, 3 H) 3.56-3.61 (m, 6 H) 3.67 (d, J = 5.1 Hz, 2 H) 3.93 (s, 3 H) 7.11 (d, J = 5.3 Hz, 1 H) 7.23 (s, 1 H) 7.25- 7.31 (m, 1 H) 7.31-7.39 (m, 1 H) 7.67 (d, J = 7.8 Hz, 1 H) 7.72 (d, J = 7.8 Hz, 1 H) 8.32 (d, J = 5.3 Hz, 1 H) |
| A-30 | 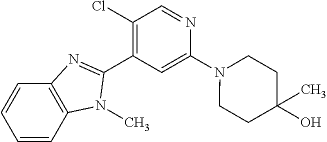 | 1-[5-chloro-4- (1-methyl-1H- benzimidazol- 2-yl)pyridin-2- yl]-4-methyl- piperidin-4-ol | 357.10/ 359.20 | ¹H NMR (400 MHz, DMSO-d6) δ ppm 8.29 (s, 1 H) 7.70 (d, J = 7.8 Hz, 1 H) 7.64 (d, J = 8.1 Hz, 1 H) 7.31-7.41 (m, 1 H) 7.20-7.31 (m, 1 H) 7.06 (s, 1 H) 4.38 (s, 1 H) 3.79-3.95 (m, 2 H) 3.68 (s, 3 H) 3.31-3.46 (m, 2 H) 1.36-1.61 (m, 4 H) 1.15 (s, 3 H) |
| A-31 | 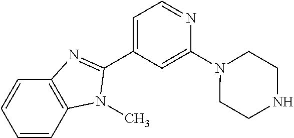 | 2,2′-(piperazine- 1,4-diyldi- pyridine-2,4- diyl)bis(1- methyl-1H- benzimidazole) | 294.20 | ¹H NMR (400 MHz, DMSO-d6) δ ppm 2.74-2.87 (m, 4 H) 3.33 (br. s., 1 H) 3.46-3.55 (m, 4 H) 3.92 (s, 3 H) 7.05 (d, J = 4.8 Hz, 1 H) 7.16 (s, 1 H) 7.25-7.31 (m, 1 H) 7.31-7.38 (m, 1 H) 7.66 (d, J = 7.6 Hz, 1 H) 7.71 (d, J = 7.8 Hz, 1 H) 8.29 (d, J = 5.1 Hz, 1 H) |
| A-32 | 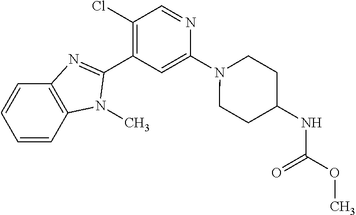 | methyl {1-[5- chloro-4-(1- methyl-1H- benzimidazol- 2-yl)pyridin-2- yl]piperidin-4- yl}carbamate | 400.15/ 402.10 | ¹H NMR (400 MHz, DMSO-d6) δ ppm 8.31 (s, 1 H) 7.71 (d, J = 7.8 Hz, 1 H) 7.65 (d, J = 8.1 Hz, 1 H) 7.32-7.41 (m, 1 H) 7.24-7.31 (m, 1 H) 7.16 (d, J = 7.8 Hz, 1 H) 7.10 (s, 1 H) 4.24 (d, J = 13.4 Hz, 2 H) 3.68 (s, 3 H) 3.58 (br. s., 1 H) 3.52 (s, 3 H) 3.03 (t, J = 12.0 Hz, 2 H) 1.70-1.86 (m, 2 H) 1.28- 1.45 (m, 2 H) |
| A-33 | 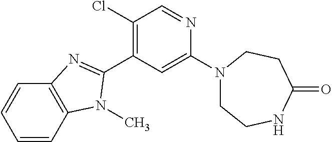 | 1-[5-chloro-4- (1-methyl-1H- benzimidazol- 2-yl)pyridin-2- yl]-1,4- diazepan-5-one | 356.20/ 358/15 | ¹H NMR (400 MHz, DMSO-d6) δ ppm 8.34 (s, 1 H) 7.71 (d, J = 7.8 Hz, 1 H) 7.57-7.68 (m, 2 H) 7.32-7.42 (m, 1 H) 7.22- 7.32 (m, 1 H) 7.09 (s, 1 H) 3.74-3.93 (m, 4 H) 3.69 (s, 3 H) 3.21 (t, J = 5.8 Hz, 2 H) 2.49-2.56 (m, 2 H) |
| A-34 | 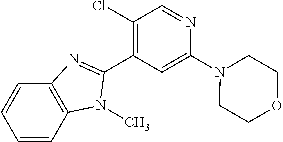 | 2-(5-chloro-2- morpholin-4- ylpyridin-4-yl)- 1-methyl-1H- benzimidazole | 330.10/ 332.05 | ¹H NMR (400 MHz, DMSO-d6) δ ppm 8.69 (s, 1 H) 7.76 (d, J = 7.8 Hz, 1 H) 7.68 (d, J = 8.1 Hz, 1 H) 7.35-7.47 (m, 1 H) 7.23-7.35 (m, 1 H) 3.90 (s, 3 H) 3.71-3.81 (m, 4 H) 3.64- 3.71 (m, 4 H) |
| A-35 | 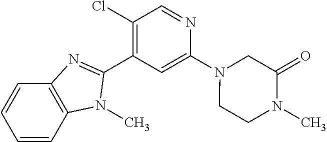 | 4-[5-chloro-4- (1-methyl-1H- benzimidazol- 2-yl)pyridin-2- yl]-1-methyl- piperazin-2-one | 356.05/ 358/15 | ¹H NMR (400 MHz, DMSO-d6) δ ppm 8.37 (s, 1 H) 7.71 (d, J = 7.8 Hz, 1 H) 7.66 (d, J = 8.1 Hz, 1 H) 7.32-7.41 (m, 1 H) 7.25-7.32 (m, 1 H) 7.14 (s, 1 H) 4.12 (s, 2 H) 3.86 (t, 2 H) 3.68 (s, 3 H) 3.43 (t, J = 5.3 Hz, 2 H) 2.90 (s, 3 H) |
| A-36 | 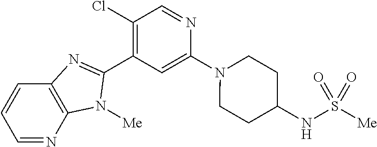 | N-{1-[5-chloro- 4-(3-methyl-3H- imidazo[4,5- b]pyridin-2- yl)pyridin-2- yl]piperidin-4- yl}methane- sulfonamide | 421.10 | ¹H NMR (400 MHz, DMSO-d6) δ ppm 1.27-1.40 (m, 2 H) 1.76-1.85 (m, 2 H) 2.86 (s, 3 H) 2.92-3.02 (m, 2 H) 3.31- 3.42 (m, 1 H) 3.62 (s, 3 H) 4.10-4.19 (m, 2 H) 7.04 (d, J = 7.33 Hz, 1 H) 7.08 (s, 1 H) 7.29 (dd, J = 8.08, 4.80 Hz, 1 H) 8.07 (dd, J = 7.96, 1.39 Hz, 1 H) 8.25 (s, 1 H) 8.37 (dd, J = 4.80, 1.26 Hz, 1 H) |
| A-37 | 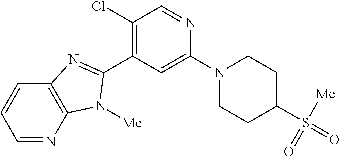 | 2-{5-chloro-2-[4- (methylsulfonyl) piperidin-1-yl] pyridin-4-yl}- 3-methyl-3H- imidazo[4,5- b]pyridine | 406.20 | ¹H NMR (400 MHz, DMSO-d6) δ ppm 1.43-1.57 (m, 2 H) 1.94-2.04 (m, 2 H) 2.83-2.93 (m, 5 H) 3.27-3.39 (m, 1 H) 3.62 (s, 3 H) 4.42 (d, J = 13.39 Hz, 1 H) 7.14 (s, 1 H) 7.29 (dd, J = 8.08, 4.80 Hz, 1 H) 8.08 (dd, J = 8.08, 1.52 Hz, 1 H) 8.28 (s, 1 H) 8.38 (dd, J = 4.67, 1.39 Hz, 1 H) |
| A-38 | 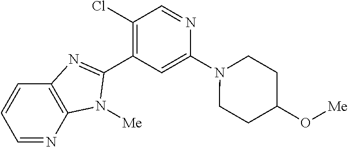 | 2-[5-chloro-2- (4-methoxy- piperidin-1-yl) pyridin-4-yl]-3- methyl-3H- imidazo[4,5- b]pyridine | 358.20 | ¹H NMR (400 MHz, DMSO-d6) δ ppm 1.39-1.51 (m, 2 H) 1.84-1.96 (m, 2 H) 3.22-3.36 (m, 5 H) 3.41-3.49 (m, 1 H) 3.71 (s, 3 H) 3.98 (ddd, J = 13.33, 4.86, 4.55 Hz, 2 H) 7.17 (s, 1 H) 7.39 (dd, J = 8.08, 4.80 Hz, 1 H) 8.17 (dd, J = 7.96, 1.14 Hz, 1 H) 8.34 (s, 1 H) 8.47 (dd, J = 4.55, 1.26 Hz, 1 H) |
| A-39 | 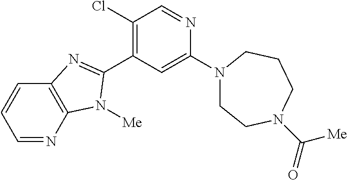 | 2-[2-(4-acetyl- 1,4-diazepan- 1-yl)-5- chloropyridin-4- yl]-3-methyl-3H- imidazo[4,5- b]pyridine | 385.20 | ¹H NMR (400 MHz, DMSO-d6) δ ppm 1.76 (t, J = 5.31 Hz, 1 H) 1.82-1.90 (m, 1 H) 1.92 (d, J = 1.26 Hz, 3 H) 2.00 (s, 3 H) 3.42 (dt, J = 18.95, 5.81 Hz, 2 H) 3.58-3.79 (m, 8 H) 3.86 (t, J = 5.56 Hz, 1 H) 7.05 (d, J = 9.85 Hz, 1 H) 7.39 (dd, J = 8.08, 4.80 Hz, 1 H) 8.18 (dd, J = 8.08, 1.01 Hz, 1 H) 8.33 (d, J = 5.05 Hz, 1 H) 8.44- 8.50 (m, 1 H) |
| A-40 | 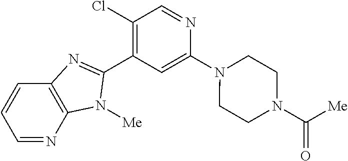 | 2-[2-(4-acetyl- piperazin-1-yl)-5- chloropyridin-4- yl]-3-methyl-3H- imidazo[4,5- b]pyridine | 371.20 | ¹H NMR (400 MHz, DMSO-d6) δ ppm 1.96 (s, 3 H) 3.43-3.51 (m, 6 H) 3.52-3.59 (m, 3 H) 3.62 (s, 3 H) 7.09 (s, 1 H) 7.30 (dd, J = 7.96, 4.67 Hz, 1 H) 8.08 (dd, J = 8.08, 1.26 Hz, 1 H) 8.29 (s, 1 H) 8.38 (dd, J = 4.55, 1.26 Hz, 1 H) |
| A-41 | 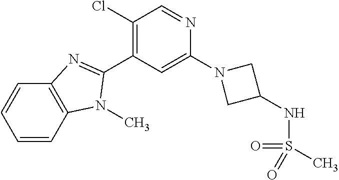 | N-{1-[5-chloro- 4-(1-methyl-1H- benzimidazol- 2-yl)pyridin-2- yl]azetidin-3- yl}methane- sulfonamide | 392.3 | ¹H NMR (400 MHz, CDCl3) δ ppm 8.25 (s, 1 H), 7.84-7.82 (d, 1 H), 7.44-7.32 (m, 3 H) 6.53 (s, 1 H), 5.40-5.38 (d, 1 H), 4.48-4.39 (m, 3 H), 3.94-3.91 (m, 2 H), 3.71 (s, 3 H), 2.98 (s, 3 H) |
| A-42 | 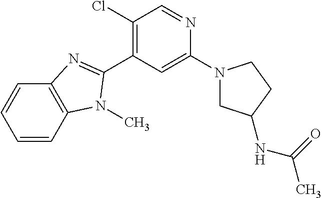 | N-{1-[5-chloro- 4-(1-methyl-1H- benzimidazol- 2-yl)pyridin-2- yl]pyrrolidin-3- yl}acetamide | 370.2 | ¹H NMR (400 MHz, CDCl3) δ ppm 8.19 (s, 1 H), 7.77-7.76 (d, 1 H), 7.38-7.36 (d, 1 H), 7.33-7.26 (m, 2 H), 6.51 (s, 1 H), 5.76 (m, 1 H), 4.56-4.55 (m, 1 H), 3.83-3.60 (m, 4 H), 3.54-3.46 (m, 2 H), 3.33-3.30 (m, 1 H), 2.29-2.21 (m, 1 H), 2.04-1.84 (m, 4 H) |
| A-43 | 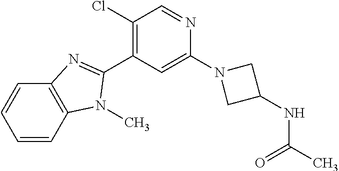 | N-{1-[5-chloro- 4-(1-methyl-1H- benzimidazol- 2-yl)pyridin-2- yl]azetidin-3- yl}acetamide | 356.4 | ¹H NMR (400 MHz, CDCl3) δ ppm 8.25 (s, 1 H), 7.84-7.82 (d, 1 H), 7.51-7.38 (m, 3 H), 6.50 (s, 1 H), 6.07-6.05 (d, 1 H), 4.90-4.84 (m, 1 H), 4.40- 4.36 (t, 2 H), 3.86-3.82 (m, 2 H), 3.76 (s, 3 H), 2.02 (s, 3 H) |
| A-44 | 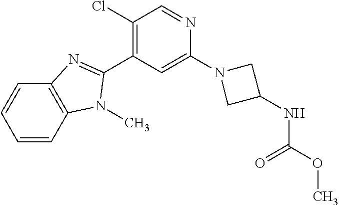 | methyl {1-[5- chloro-4-(1- methyl-1H- benzimidazol- 2-yl)pyridin-2- yl]azetidin-4- yl}carbamate | 372.3 | ¹H NMR (400 MHz, CDCl3) δ ppm 8.25 (s, 1 H), 7.84-7.83 (d, 1 H), 7.51-7.32 (m, 3 H), 6.51 (s, 1 H), 5.14 (s, 1 H), 4.68-4.64 (s, 1 H), 4.40-4.36 (t, 2 H), 3.88-3.85 (m, 2 H), 3.71-3.70 (m, 6 H) |
| A-45 | 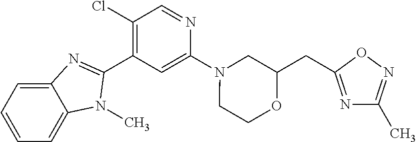 | 2-(5-chloro-2- {2-[(3-methyl- 1,2,4- oxadiazol-5- yl)methyl] morpholin-4- yl}pyridin-4-yl)- 1-methyl-1H- benzimidazole | 425 | ND |
| A-46 | 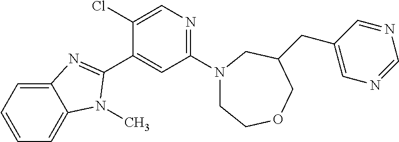 | 2-{5-chloro-2- [6-(pyrimidin-5- ylmethyl)-1,4- oxazepan-4- yl]pyridin-4-yl}- 1-methyl-1H- benzimidazole | 435 | ND |
| A-47 | 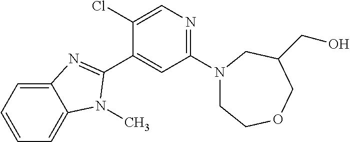 | {4-[5-chloro-4- (1-methyl-1H- benzimidazol- 2-yl)pyridin-2- yl]-1,4- oxazepan-6- yl}methanol | 373 | ND |
| A-48 | 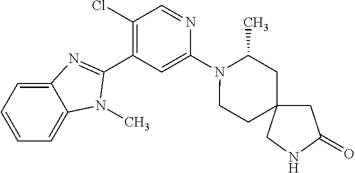 | (5R,7S)-8-[5- chloro-4-(1- methyl-1H- benzimidazol- 2-yl)pyridin-2- yl]-7-methyl-2,8- diazaspiro[4.5] decan-3-one | 410 | ND |
| A-49 | 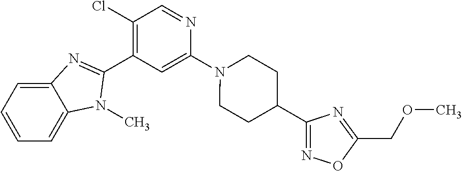 | 2-(5-chloro-2- {4-[5-(methoxy- methyl)-1,2,4- oxadiazol-3- yl]piperidin-1- yl}pyridin-4-yl)- 1-methyl-1H- benzimidazole | 439 | ND |
| A-50 | 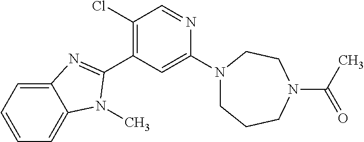 | 2-[2-(4-acetyl- 1,4-diazepan- 1-yl)-5-chloro- pyridin-4-yl]-1- methyl-1H- benzimidazole | 384 | ND |
| A-51 | 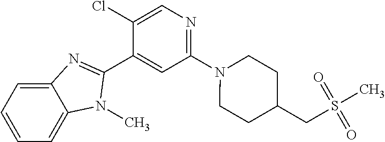 | 2-(5-chloro-2-{4- [(methylsulfonyl) methyl]piperidin- 1-yl}pyridin-4- yl)-1-methyl-1H- benzimidazole | 419 | ND |
| A-52 | 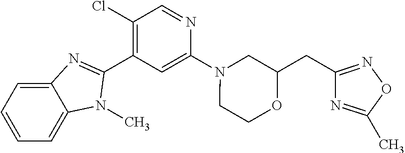 | 2-(5-chloro-2- {2-[(5-methyl- 1,2,4- oxadiazol-3- yl)methyl]mor- pholin-4-yl} pyridin-4-yl)- 1-methyl-1H- benzimidazole | 425 | ND |
| A-53 | 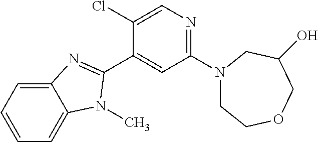 | 4-[5-chloro-4- (1-methyl-1H- benzimidazol- 2-yl)pyridin-2- yl]-1,4- oxazepan-6-yl | 359 | ND |
| A-54 | 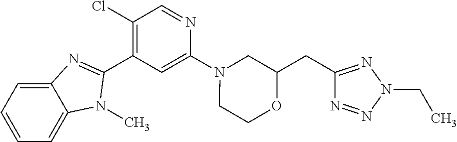 | 2-{5-chloro-2- [2-(2-ethyl-2H- tetrazol-5- yl)morpholin-4- yl]pyridin-4-yl}- 1-methyl-1H- benzimidazole | 425 | ND |
| A-55 | 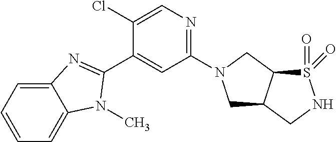 | 2-{5-chloro-2- [(3aS,6aR)-1,1- dioxidohexa- hydro-5H- pyrrolo[3,4- d]isothiazol-5- yl]pyridin-4-yl}- 1-methyl-1H- benzimidazole | 404 | ND |
| A-56 | 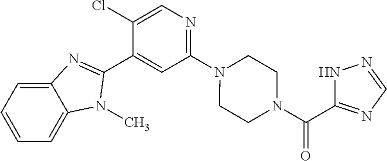 | 2-{5-chloro-2- [4-(1H-1,2,4- triazol-3-yl- carbonyl) piperazin-1- yl]pyridin-4-yl}- 1-methyl-1H- benzimidazole | 423 | ND |
| A-57 | 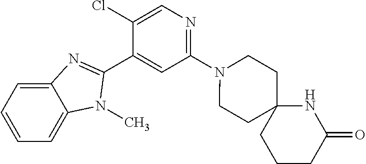 | 9-[5-chloro-4- (1-methyl-1H- benzimidazol- 2-yl)pyridin-2- yl]-1,9- diazaspiro[5.5] undecan-2-one | 410 | ND |
| A-58 | 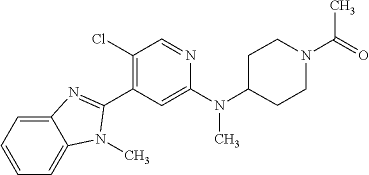 | N-(1-acetyl- piperidin-4- yl)-5-chloro- N-methyl-4-(1- methyl-1H- benzimidazol- 2-yl)pyridin-2- amine | 398 | ND |
| A-59 | 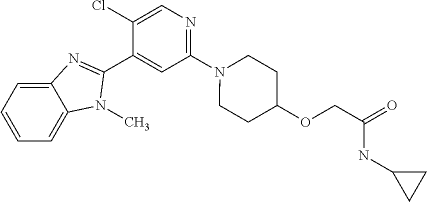 | 2-({1-[5-chloro- 4-(1-methyl-1H- benzimidazol- 2-yl)pyridin-2- yl]piperidin-4- yl}oxy)-N- cyclopropyl- acetamide | 440 | ND |
| A-60 | 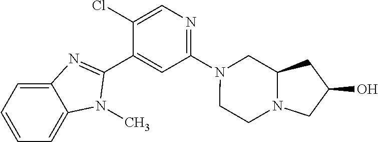 | (7R,8aS)-2-[5- chloro-4-(1- methyl-1H- benzimidazol- 2-yl)pyridin-2- yl]octahydro- pyrrolo[1,2- a]pyrazin-7-ol | 384 | ND |
| A-61 | 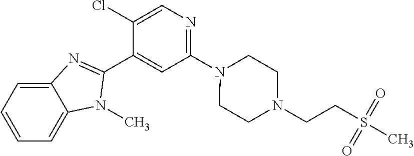 | 2-(5-chloro-2- {4-[2- (methylsulfonyl) ethyl]piperazin- 1-yl}pyridin-4- yl)-1-methyl-1H- benzimidazole | 434 | ND |
| A-62 | 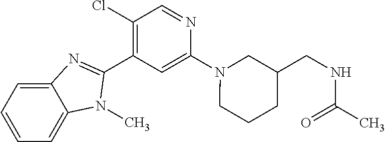 | N-({1-[5-chloro- 4-(1-methyl-1H- benzimidazol- 2-yl)pyridin-2- yl]piperidin-3- yl}methyl) acetamide | 398 | ND |
| A-63 | 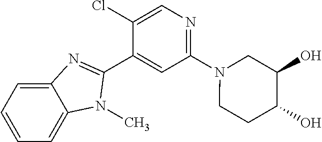 | (3R,4R)-1-[5- chloro-4-(1- methyl-1H- benzimidazol- 2-yl)pyridin-2- yl]piperidine- 3,4-diol | 359 | ND |
| A-64 | 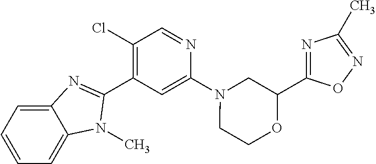 | 2-{5-chloro-2- [2-(3-methyl- 1,2,4-oxadiazol- 5-yl)morpholin- 4-yl]pyridin-4- yl}-1-methyl-1H- benzimidazole | 411 | ND |
| A-65 | 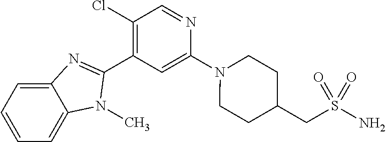 | 1-{1-[5-chloro- 4-(1-methyl-1H- benzimidazol- 2-yl)pyridin-2- yl]piperidin-4- yl}methane- sulfonamide | 420 | ND |
| A-66 | 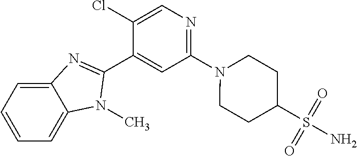 | 1-[5-chloro-4- (1-methyl-1H- benzimidazol- 2-yl)pyridin-2- yl]piperidine-4- sulfonamide | 406 | ND |
| A-67 | 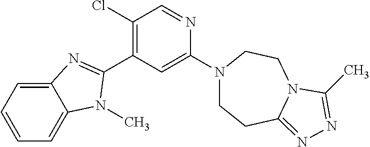 | 7-[5-chloro-4- (1-methyl-1H- benzimidazol- 2-yl)pyridin-2- yl]-3-methyl- 6,7,8,9-tetra- hydro-5H-[1,2,4] triazolo[4,3-d] [1,4]diazepine | 394 | ND |
| A-68 | 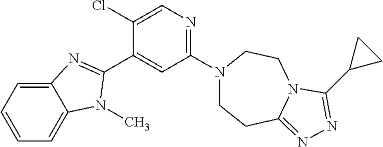 | 7-[5-chloro-4- (1-methyl-1H- benzimidazol- 2-yl)pyridin-2- yl]-3-cyclopropyl- 6,7,8,9-tetra- hydro-5H-[1,2,4] triazolo[4,3-d] [1,4]diazepine | 420 | ND |
| A-69 | 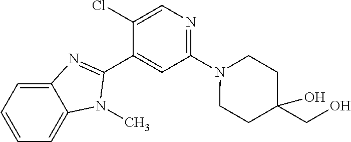 | 1-[5-chloro-4- (1-methyl-1H- benzimidazol- 2-yl)pyridin-2- yl]-4- (hydroxymethyl) piperidin-4-ol | 373 | ND |
| A-70 | 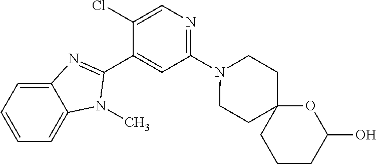 | 9-[5-chloro-4- (1-methyl-1H- benzimidazol- 2-yl)pyridin-2- yl]-1-oxa-9- azaspiro[5.5] undecan-4-ol | 413 | ND |
| A-71 | 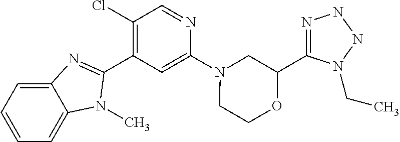 | 2-{5-chloro-2- [2-(1-ethyl-1H- tetrazol-5- yl)morpholin-4- yl]pyridin-4-yl}- 1-methyl-1H- benzimidazole | 425 | ND |
| A-72 | 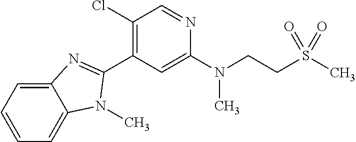 | 5-chloro-N- methyl-4-(1- methyl-1H- benzimidazol- 2-yl)-N-[2- (methylsulfonyl) ethyl]pyridin- 2-amine | 379 | ND |
| A-73 | 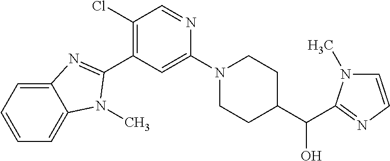 | {1-[5-chloro-4- (1-methyl-1H- benzimidazol- 2-yl)pyridin-2- yl]piperidin-4- yl}(1-methyl- 1H-imidazol-2- yl)methanol | 437 | ND |
| A-74 | 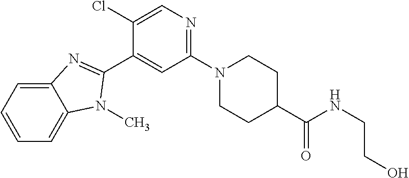 | 1-[5-chloro-4- (1-methyl-1H- benzimidazol- 2-yl)pyridin-2- yl]-N-(2- hydroxyethyl) piperidine-4- carboxamide | 4145 | ND |
| A-75 | 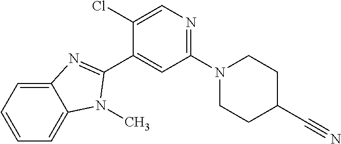 | 1-[5-chloro-4- (1-methyl-1H- benzimidazol- 2-yl)pyridin-2- yl]piperidine-4- carbonitrile | 352 | ND |
| A-76 | 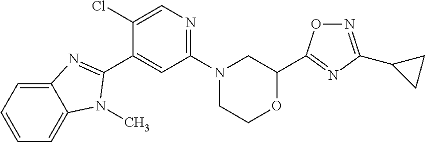 | 2-{5-chloro-2- [2-(3-cyclo- propyl-1,2,4- oxadiazol-5- yl)morpholin-4- yl]pyridin-4-yl}- 1-methyl-1H- benzimidazole | 437 | ND |
| A-77 | 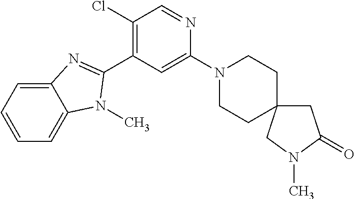 | 8-[5-chloro-4- (1-methyl-1H- benzimidazol- 2-yl)pyridin-2- yl]-2-methyl-2,8- diazaspiro[4.5] decan-3-one | 410 | ND |
| A-78 | 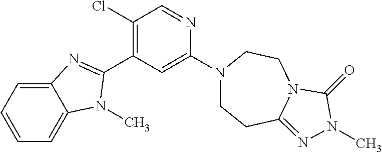 | 7-[5-chloro-4- (1-methyl-1H- benzimidazol- 2-yl)pyridin-2- yl]-2-methyl- 2,5,6,7,8,9- hexahydro-3H- [1,2,4]triazolo [4,3-d][1,4] diazepin-3-one | 410 | ND |
| A-79 | 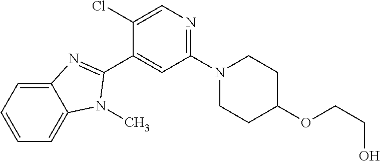 | 2-({1-[5-chloro- 4-(1-methyl-1H- benzimidazol- 2-yl)pyridin-2- yl]piperidin-4- yl}oxy)ethanol | 387 | ND |
| A-80 | 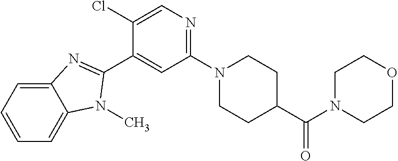 | 2-{5-chloro-2- [4-(morpholin-4- ylcarbonyl) piperidin-1- yl]pyridin-4-yl}- 1-methyl-1H- benzimidazole | 440 | ND |
| A-81 | 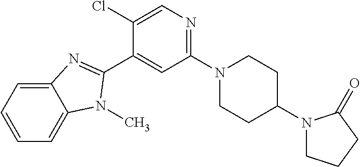 | 1-{1-[5-chloro- 4-(1-methyl-1H- benzimidazol- 2-yl)pyridin-2- yl]piperidin-4- yl}pyrrolidin-2- one | 410 | ND |
| A-82 | 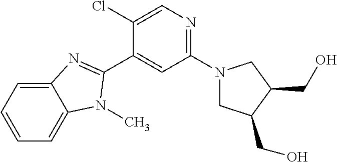 | {(3R,4S)-1-[5- chloro-4-(1- methyl-1H- benzimidazol- 2-yl)pyridin-2-yl] pyrrolidine-3,4- diyl}dimethanol | 373 | ND |
| A-83 | 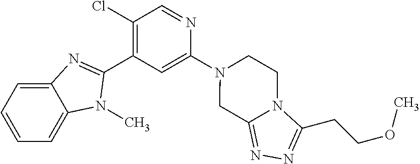 | 7-[5-chloro-4- (1-methyl-1H- benzimidazol- 2-yl)pyridin-2- yl]-3-(2-methoxy- ethyl)-5,6,7,8- tetrahydro[1,2,4] triazolo[4,3-a] pyrazine | 424 | ND |
| A-84 | 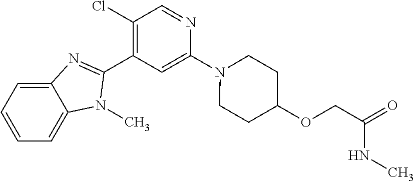 | 2-({1-[5-chloro- 4-(1-methyl-1H- benzimidazol- 2-yl)pyridin-2- yl]piperidin-4- yl}oxy)-N- methylacetamide | 414 | ND |
| A-85 | 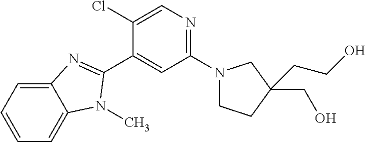 | 2-{1-[5-chloro- 4-(1-methyl-1H- benzimidazol- 2-yl)pyridin-2- yl]-3-(hydroxy- methyl) pyrrolidin-3- yl}ethanol | 387 | ND |
| A-86 | 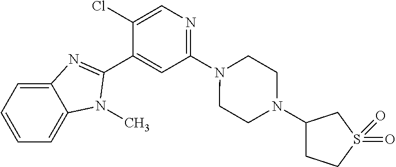 | 2-{5-chloro-2- [4-(1,1-dioxido- tetrahydro-3- thienyl)piperazin- 1-yl]pyridin-4- yl}-1-methyl-1H- benzimidazole | 446 | ND |
| A-87 | 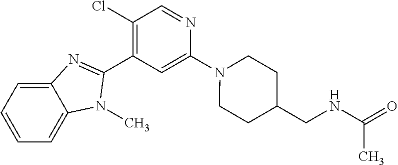 | N-({1-[5-chloro- 4-(1-methyl-1H- benzimidazol- 2-yl)pyridin-2- yl]piperidin-4- yl}methyl) acetamide | 398 | ND |
| A-88 | 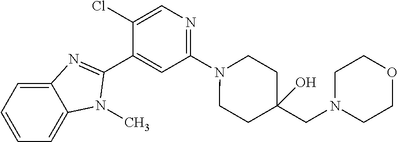 | 1-[5-chloro-4- (1-methyl-1H- benzimidazol- 2-yl)pyridin-2- yl]-4- (morpholin-4- ylmethyl) piperidin-4-ol | 442 | ND |
| A-89 | 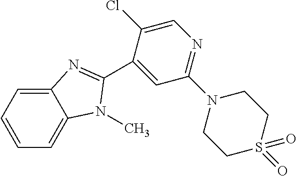 | 2-[5-chloro-2- (1,1-dioxidothio- morpholin-4- yl)pyridin-4-yl]- 1-methyl-1H- benzimidazole | 377 | ND |
| A-90 | 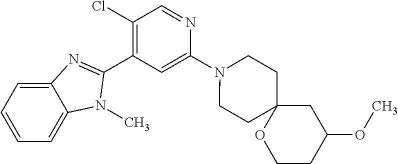 | 9-[5-chloro-4- (1-methyl-1H- benzimidazol- 2-yl)pyridin-2- yl]-4-methoxy- 1-oxa-9- azaspiro[5.5] undecane | 427 | ND |
| A-91 | 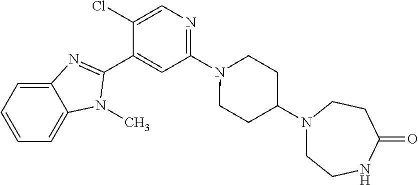 | 1-{1-[5-chloro- 4-(1-methyl-1H- benzimidazol- 2-yl)pyridin-2- yl]piperidin-4- yl}-1,4-diazepan- 5-one | 439 | ND |
| A-92 | 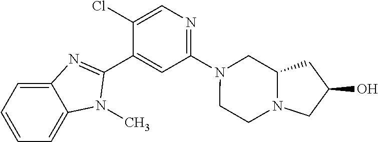 | (7R,8aR)-2-[5- chloro-4-(1- methyl-1H- benzimidazol- 2-yl)pyridin-2- yl]octahydro- pyrrolo[1,2- a]pyrazin-7-ol | 384 | ND |
| A-93 | 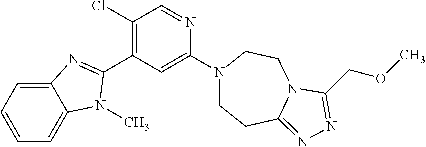 | 7-[5-chloro-4- (1-methyl-1H- benzimidazol- 2-yl)pyridin-2- yl]-3-(methoxy- methyl)-6,7,8,9- tetrahydro-5H- [1,2,4]triazolo [4,3-d][1,4] diazepine | 424 | ND |
| A-94 | 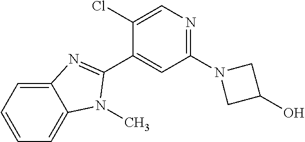 | 1-[5-chloro-4- (1-methyl-1H- benzimidazol- 2-yl)pyridin-2- yl]azetidin-3-ol | 315 | ND |
| A-95 | 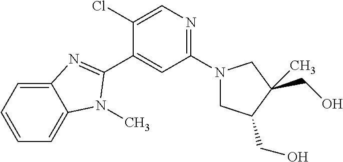 | {(3R,4S)-1-[5- chloro-4-(1- methyl-1H- benzimidazol- 2-yl)pyridin-2- yl]-3-methyl- pyrrolidine-3,4- diyl}dimethanol | 387 | ND |
| A-96 | 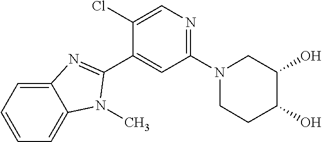 | (3S,4R)-1-[5- chloro-4-(1- methyl-1H- benzimidazol- 2-yl)pyridin-2- yl]piperidine- 3,4-diol | 359 | ND |
| A-97 | 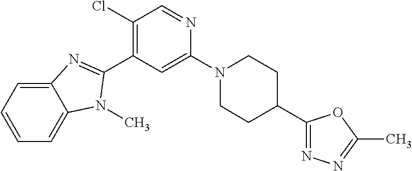 | 2-{5-chloro-2- [4-(5-methyl- 1,3,4- oxadiazol-2- yl)piperidin-1- yl]pyridin-4-yl}- 1-methyl-1H- benzimidazole | 409 | ND |
| A-98 | 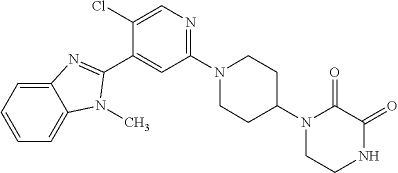 | 1-{1-[5-chloro- 4-(1-methyl-1H- benzimidazol- 2-yl)pyridin-2- yl]piperidin-4- yl}piperazine- 2,3-dione | 439 | ND |
| A-99 | 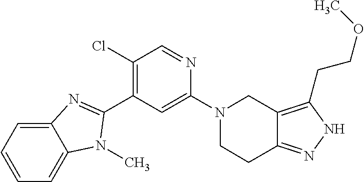 | 5-[5-chloro-4- (1-methyl-1H- benzimidazol- 2-yl)pyridin-2- yl]-3-(2-methoxy- ethyl)-4,5,6,7- tetrahydro-1H- pyrazolo[4,3- c]pyridine | 423 | ND |
| A-100 | 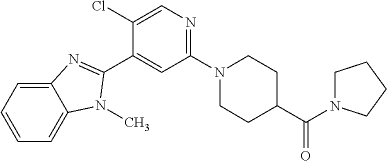 | 2-{5-chloro-2- [4-(pyrrolidin-1- ylcarbonyl) piperidin-1- yl]pyridin-4-yl}- 1-methyl-1H- benzimidazole | 424 | ND |
| A-101 | 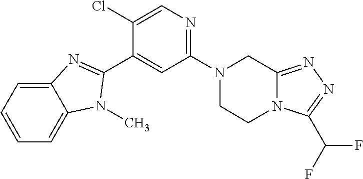 | 7-[5-chloro-4- (1-methyl-1H- benzimidazol- 2-yl)pyridin-2- yl]-3-(difluoro- methyl)-5,6,7,8- tetrahydro[1,2,4] triazolo[4,3- a]pyrazine | 416 | ND |
| A-102 | 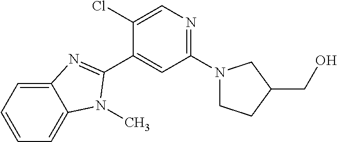 | {1-[5-chloro-4- (1-methyl-1H- benzimidazol- 2-yl)pyridin-2- yl]pyrrolidin-3- yl}methanol | 343 | ND |
| A-103 | 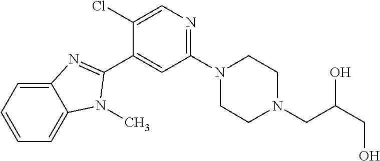 | 3-{4-[5-chloro- 4-(1-methyl-1H- benzimidazol 2-yl)pyridin-2- yl]piperazin-1- yl}propane-1,2- diol | 402 | ND |
| A-104 | 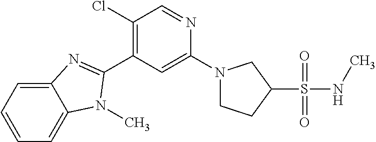 | 1-[5-chloro-4- (1-methyl-1H- benzimidazol- 2-yl)pyridin-2- yl]-N-methyl- pyrrolidine-3- sulfonamide | 406 | ND |
| A-105 | 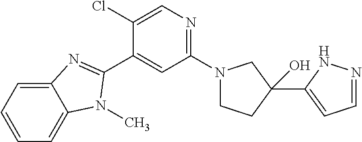 | 1-[5-chloro-4- (1-methyl-1H- benzimidazol- 2-yl)pyridin-2- yl]-3-(1H- pyrazol-5-yl) pyrrolidin-3-ol | 395 | ND |
| A-106 | 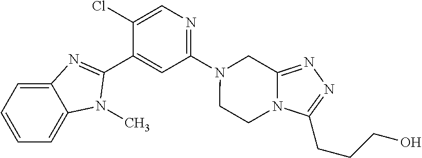 | 3-{7-[5-chloro- 4-(1-methyl-1H- benzimidazol- 2-yl)pyridin-2- yl]-5,6,7,8-tetra- hydro[1,2,4] triazolo[4,3- a]pyrazin-3- yl}propan-1-ol | 424 | ND |
| A-107 | 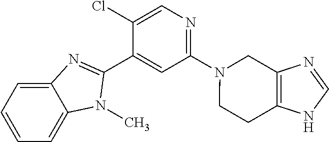 | 5-[5-chloro-4- (1-methyl-1H- benzimidazol- 2-yl)pyridin-2- yl]-4,5,6,7- tetrahydro-1H- imidazo[4,5- c]pyridine | 365 | ND |
| A-108 | 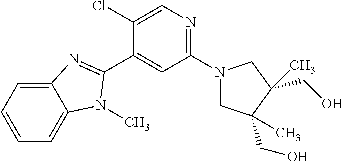 | {(3R,4S)-1-[5- chloro-4-(1- methyl-1H- benzimidazol- 2-yl)pyridin-2- yl]-3,4-dimethyl- pyrrolidine-3,4- diyl}dimethanol | 401 | ND |
| A-109 | 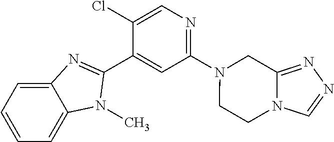 | 7-[5-chloro-4- (1-methyl-1H- benzimidazol- 2-yl)pyridin-2- yl]-5,6,7,8- tetrahydro[1,2,4] triazolo[4,3- a]pyrazine | 366 | ND |
| A-110 | 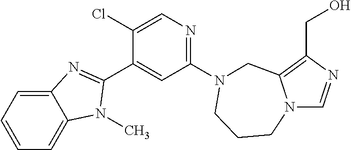 | {8-[5-chloro-4- (1-methyl-1H- benzimidazol- 2-yl)pyridin-2- yl]-6,7,8,9- tetrahydro-5H- imidazo[1,5- a][1,4]diazepin- 1-yl}methanol | 409 | ND |
| A-111 | 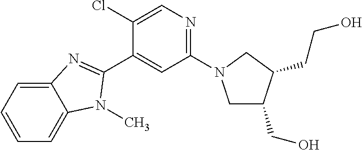 | 2-[(3R,4S)-1- [5-chloro-4-(1- methyl-1H- benzimidazol- 2-yl)pyridin-2- yl]-4-(hydroxy- methyl)pyrroli- din-3-yl]ethanol | 387 | ND |
| A-112 | 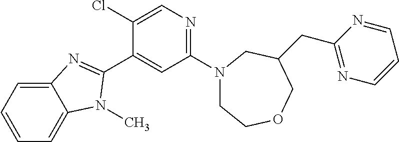 | 2-{5-chloro-2- [6-(pyrimidin-2- ylmethyl)-1,4- oxazepan-4- yl]pyridin-4-yl}- 1-methyl-1H- benzimidazole | 435 | ND |
| A-113 | 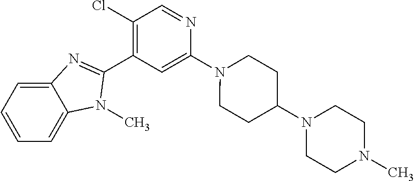 | 2-{5-chloro-2- [4-(4-methyl- piperazin-1- yl)piperidin-1- yl]pyridin-4-yl}- 1-methyl-1H- benzimidazole | 425 | ND |
| A-114 | 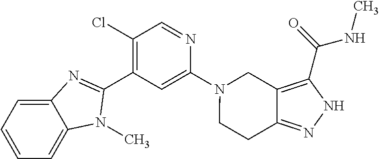 | 5-[5-chloro-4- (1-methyl-1H- benzimidazol- 2-yl)pyridin-2- yl]-N-methyl- 4,5,6,7- tetrahydro-1H- pyrazolo[4,3- c]pyridine-3- carboxamide | 422 | ND |
| A-115 | 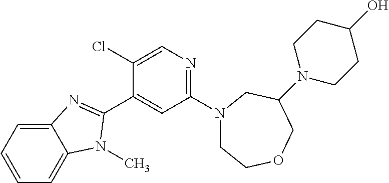 | 1-{4-[5-chloro- 4-(1-methyl-1H- benzimidazol- 2-yl)pyridin-2- yl]-1,4- oxazepan-6- yl}piperidin-4-ol | 442 | ND |
| A-116 | 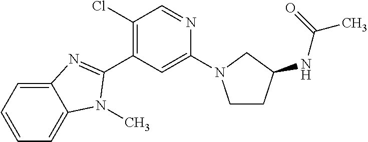 | N-{(3R)-1-[5- chloro-4-(1- methyl-1H- benzimidazol- 2-yl)pyridin-2- yl]pyrrolidin-3- yl}acetamide | 370 | ND |
| A-117 | 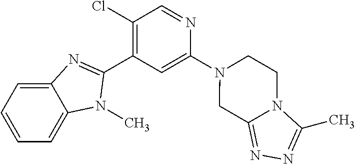 | 7-[5-chloro-4- (1-methyl-1H- benzimidazol- 2-yl)pyridin-2- yl]-3-methyl- 5,6,7,8- tetrahydro[1,2,4] triazolo[4,3- a]pyrazine | 380 | ND |
| A-118 | 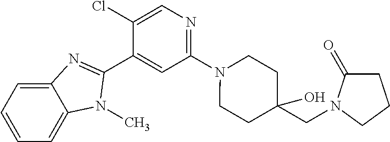 | 1-({1-[5-chloro- 4-(1-methyl-1H- benzimidazol- 2-yl)pyridin-2- yl]-4-hydroxy- piperidin-4- yl}methyl) pyrrolidin-2-one | 440 | ND |
| A-119 | 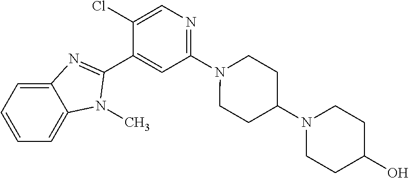 | 1′-[5-chloro-4- (1-methyl-1H- benzimidazol- 2-yl)pyridin-2- yl]-1,4′- bipiperidin-4-ol | 426 | ND |
| A-120 | 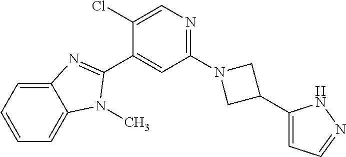 | 2-{5-chloro-2- [3-(1H-pyrazol- 5-yl)azetidin-1- yl]pyridin-4-yl}- 1-methyl-1H- benzimidazole | 365 | ND |
| A-121 | 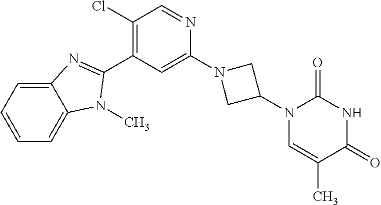 | 1-{1-[5-chloro- 4-(1-methyl-1H- benzimidazol- 2-yl)pyridin-2- yl]azetidin-3- yl}-5-methyl- pyrimidine- 2,4(1H,3H)-dione | 423 | ND |
| A-122 | 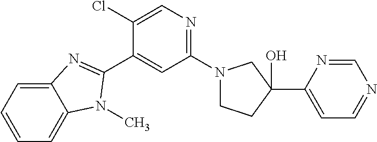 | 1-[5-chloro-4- (1-methyl-1H- benzimidazol- 2-yl)pyridin-2- yl]-3-pyrimidin- 4-ylpyrrolidin- 3-ol | 407 | ND |
| A-123 | 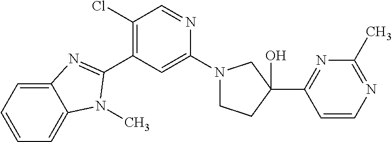 | 1-[5-chloro-4- (1-methyl-1H- benzimidazol- 2-yl)pyridin-2- yl]-3-(2-methyl- pyrimidin-4- yl)pyrrolidin-3-ol | 421 | ND |
| A-124 | 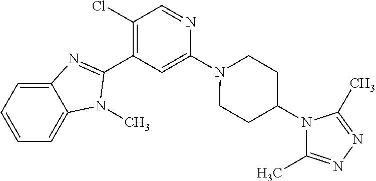 | 2-{5-chloro-2- [4-(3,5- dimethyl-4H- 1,2,4-triazol- 4-yl)piperidin-1- yl]pyridin-4-yl}- 1-methyl-1H- benzimidazole | 422 | ND |
| A-125 | 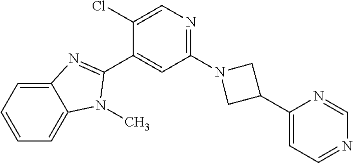 | 2-[5-chloro-2- (3-pyrimidin-4- ylazetidin-1- yl)pyridin-4-yl]- 1-methyl-1H- benzimidazole | 377 | ND |
| A-126 | 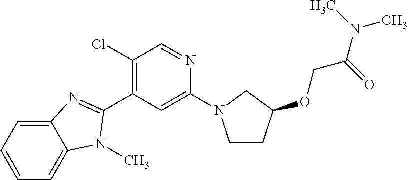 | 2-({(3R)-1-[5- chloro-4-(1- methyl-1H- benzimidazol- 2-yl)pyridin-2- yl]pyrrolidin-3- yl}oxy)-N,N- dimethyl- acetamide | 414 | ND |
| A-127 | 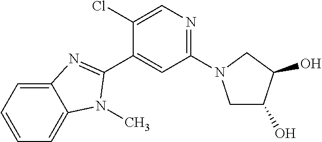 | (3R,4R)-1-[5- chloro-4-(1- methyl-1H- benzimidazol- 2-yl)pyridin-2- yl]pyrrolidine- 3,4-diol | 345 | ND |
| A-128 | 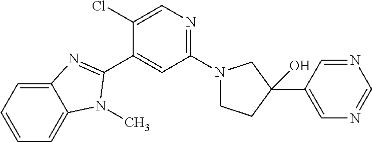 | 1-[5-chloro-4- (1-methyl-1H- benzimidazol-2- yl)pyridin-2-yl]- 3-pyrimidin-5- ylpyrrolidin-3-ol | 407 | ND |
| A-129 | 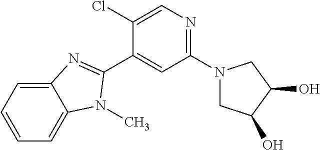 | (3R,4S)-1-[5- chloro-4-(1- methyl-1H- benzimidazol- 2-yl)pyridin-2- yl]pyrrolidine- 3,4-diol | 345 | ND |
| A-130 | 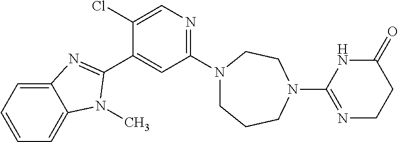 | 2-{4-[5-chloro- 4-(1-methyl-1H- benzimidazol- 2-yl)pyridin-2- yl]-1,4- diazepan-1-yl}- 5,6-dihydro- pyrimidin-4(3H)- one | 438 | ND |
| A-131 | 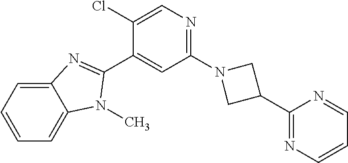 | 2-[5-chloro-2- (3-pyrimidin-2- ylazetidin-1- yl)pyridin-4-yl]- 1-methyl-1H- benzimidazole | 377 | ND |
| A-132 | 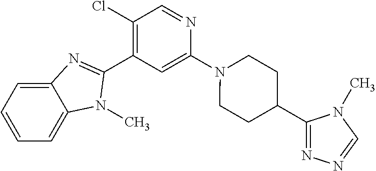 | 2-{5-chloro-2- [4-(4-methyl- 4H-1,2,4- triazol-3- yl)piperidin-1- yl]pyridin-4-yl}- 1-methyl-1H- benzimidazole | 408 | ND |
| A-133 | 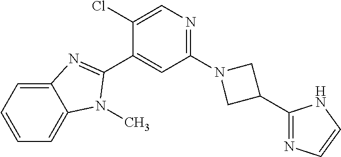 | 2-{5-chloro-2- [3-(1H- imidazol-2- yl)azetidin-1- yl]pyridin-4-yl}- 1-methyl-1H- benzimidazole | 365 | ND |
| A-134 | 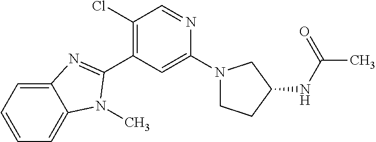 | N-{(3S)-1-[5- chloro-4-(1- methyl-1H- benzimidazol- 2-yl)pyridin-2- yl]pyrrolidin-3- yl}acetamide | 370 | ND |
| A-135 | 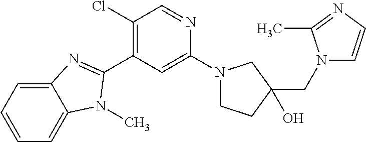 | 1-[5-chloro-4- (1-methyl-1H- benzimidazol- 2-yl)pyridin-2- yl]-3-[(2- methyl-1H- imidazol-1- yl)methyl] pyrrolidin-3-ol | 423 | ND |
| A-136 | 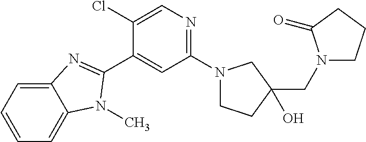 | 1-({1-[5-chloro- 4-(1-methyl-1H- benzimidazol- 2-yl)pyridin-2- yl]-3-hydroxy- pyrrolidin-3- yl}methyl) pyrrolidin-2-one | 426 | ND |
| A-137 | 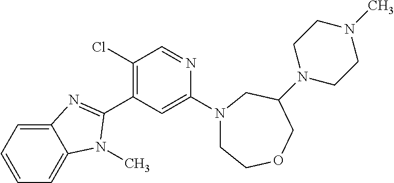 | 2-{5-chloro-2- [6-(4-methyl- piperazin-1-yl)- 1,4-oxazepan- 4-yl]pyridin-4- yl}-1-methyl-1H- benzimidazole | 441 | ND |
| A-138 | 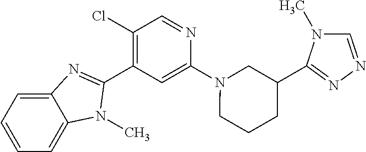 | 2-{5-chloro-2- [3-(4-methyl- 4H-1,2,4- triazol-3- yl)piperidin-1- yl]pyridin-4-yl}- 1-methyl-1H- benzimidazole | 408 | ND |
| A-139 | 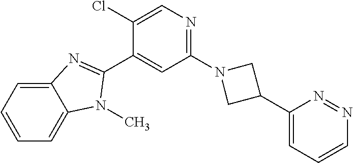 | 2-[5-chloro-2- (3-pyridazin-3- ylazetidin-1- yl)pyridin-4-yl]- 1-methyl-1H- benzimidazole | 377 | ND |
| A-140 | 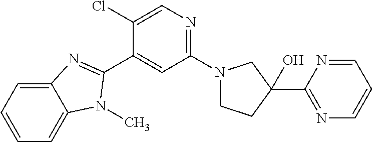 | 1-[5-chloro-4- (1-methyl-1H- benzimidazol- 2-yl)pyridin-2- yl]-3-pyrimidin- 2-ylpyrrolidin- 3-ol | 407 | ND |
| A-141 | 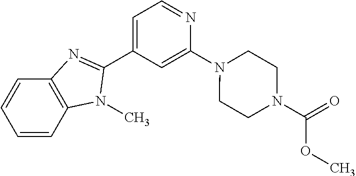 | methyl 4-[4-(1- methyl-1H- benzimidazol- 2-yl)pyridin-2- yl]piperazine-1- carboxylate | 352.20 | ¹H NMR (400 MHz, DMSO-d6) δ ppm 3.52 (d, J = 5.3 Hz, 4 H) 3.58-3.69 (m, 7 H) 3.92 (s, 3 H) 7.11 (d, J = 5.1 Hz, 1 H) 7.23 (s, 1 H) 7.25-7.31 (m, 1 H) 7.31-7.39 (m, 1 H) 7.67 (d, J = 7.8 Hz, 1 H) 7.72 (d, J = 7.8 Hz, 1 H) 8.32 (d, J = 5.1 Hz, 1 H) |
| A-142 | 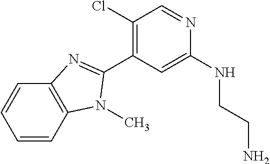 | N-[5-chloro-4- (1-methyl-1H- benzimidazol- 2-yl)pyridin-2- yl]ethane-1,2- diamine | 301.78/ 302.0 | ¹H NMR (400 MHz, DMSO-d₆) δ ppm 8.19 (s, 1 H) 7.70 (d, J = 8.08 Hz, 1 H) 7.64 (d, J = 7.83 Hz, 1 H) 7.25-7.39 (m, 2 H) 7.28 (t, J = 6.19 Hz, 1 H) 3.68 (s, 3 H) 2.81 (t, J = 6.19 Hz, 2 H) 2.54-2.58 (m, 2 H) |
| A-143 | 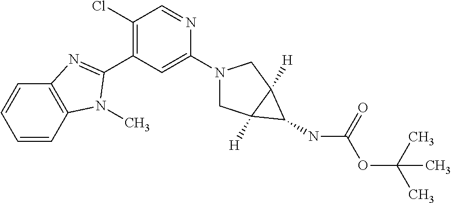 | tert-butyl {(1R,5S,6s)-3- [5-chloro-4-(1- methyl-1H- benzimidazol- 2-yl)pyridin-2- yl]-3-azabicyclo [3.1.0]hex-6-yl} carbamate | 439.95/ 440.20 | ¹H NMR (400 MHz, CHLOROFORM-d) δ ppm 8.23 (s, 1 H) 7.84 (d, J = 7.07 Hz, 1 H) 7.42-7.49 (m, 1 H) 7.29-7.42 (m, 2 H) 6.56 (s, 1 H) 4.77 (br. s., 1 H) 3.88 (s, 1 H) 3.74 (s, 3 H) 3.55-3.64 (m, 1 H) 3.26 (d, J = 9.60 Hz, 1 H) 3.11 (s, 2 H) 2.63 (s, 9 H) 1.85-1.98 (m 2 H) |
| A-144 | 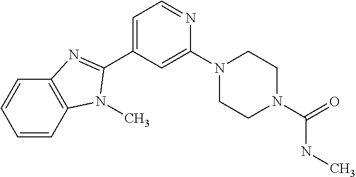 | N-methyl-4-[4- (1-methyl-1H- benzimidazol- 2-yl)pyridin-2- yl]piperazine-1- carboxamide | 351.20 | ¹H NMR (400 MHz, DMSO-d6) δ ppm 2.61 (d, J = 4.0 Hz, 3 H) 3.44 (d, J = 4.8 Hz, 4 H) 3.58 (d, J = 5.3 Hz, 4 H) 3.92 (s, 3 H) 6.54 (d, J = 4.0 Hz, 1 H) 7.09 (d, J = 4.8 Hz, 1 H) 7.22 (s, 1 H) 7.25-7.31 (m, 1 H) 7.31-7.39 (m, 1 H) 7.66 (d, J = 7.8 Hz, 1 H) 7.72 (d, J = 7.8 Hz, 1 H) 8.31 (d, J = 5.1 Hz, 1 H) |
| A-145 | 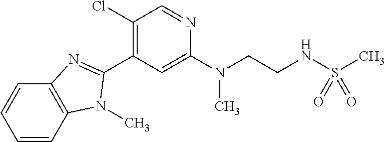 | N-(2-{[5-chloro- 4-(1-methyl-1H- benzimidazol- 2-yl)pyridin-2- yl](methyl) amino}ethyl) methane- sulfonamide | 393.89/ 394.20 | ¹H NMR (400 MHz, CHLOROFORM-d) d ppm 8.25 (s, 1 H) 7.86 (d, J = 8.40 Hz, 1 H) 7.46 (d, J = 7.46 Hz, 1 H) 7.40 (d, J = 1.77 Hz, 2 H) 6.78 (s, 1 H) 3.84-3.93 (m, 2 H) 3.77 (s, 3 H) 3.45 (d, J = 5.31 Hz, 2 H) 2.97 (s, 3 H) 2.64 (s, 3 H) |
| A-146 | 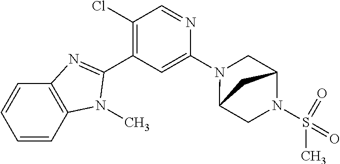 | 2-{5-chloro-2- [(1R,4R)-5- (methylsulfonyl)- 2,5-diaza- bicyclo[2.2.1] hept-2-yl] pyridin-4-yl}-1- methyl-1H- benzimidazole | 417.92/ 418.20 | ¹H NMR (400 MHz, DMSO-d₆) δ ppm 8.29 (s, 1 H) 7.70 (d, J = 8.08 Hz, 1 H) 7.65 (d, J = 7.83 Hz, 1 H) 7.49 (d, J = 2.02 Hz, 1 H) 7.25-7.38 (m, 2 H) 6.70 (s, 1 H) 3.72 (d, J = 10.86 Hz, 2 H) 3.67 (s, 3 H) 3.47 (d, J = 10.11 Hz, 2 H) 3.38 (q, J = 6.99 Hz, 2 H) 2.97 (s, 3 H) 2.33 (d, J = 1.77 Hz, 1 H) |
| A-147 | 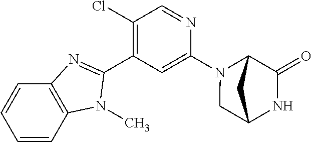 | (1S,4S)-5-[5- chloro-4-(1- methyl-1H- benzimidazol- 2-yl)pyridin-2- yl]-2,5-diaza- bicyclo[2.2.1] heptan-3-one | 353.81/ 354.20 | ¹H NMR (400 MHz, CHLOROFORM-d) δ ppm 8.36 (s, 1 H) 8.02 (t, J = 5.05 Hz, 1 H) 7.53-7.65 (m, 3 H) 7.07 (s, 1 H) 4.92 (s, 1 H) 4.29 (s., 1 H) 3.88 (s, 3 H) 3.75 (d, J = 9.35 Hz, 1 H) 3.34 (d, J = 9.85 Hz, 1 H) 3.04 (d, J = 28.55 Hz, 1 H) 2.10 (d, J = 10.77 Hz, 1 H) |
| A-148 | 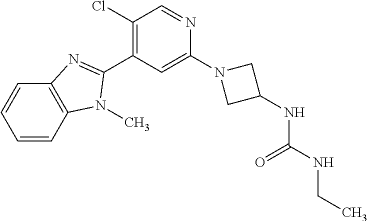 | 1-{1-[5-chloro- 4-(1-methyl-1H- benzimidazol- 2-yl)pyridin-2- yl]azetidin-3- yl}-3-ethylurea | 385.3 | ¹H NMR (400 MHz, CHLOROFORM-d) δ ppm 8.16 (s, 1 H), 7.76-7.74 (d, 1 H), 7.37-7.35 (d, 1 H), 7.33- 7.25 (m, 2 H), 6.41 (s, 1 H), 5.11-5.09 (d, 1 H), 4.74-4.65 (m, 1 H), 4.45-4.42 (t, 1 H), 4.31-4.27 (t, 2 H), 3.89-3.84 (m, 2 H), 3.70 (s, 3 H), 3.17- 3.10 (m, 2 H), 1.06-1.02 (t, 3 H) |
| A-149 | 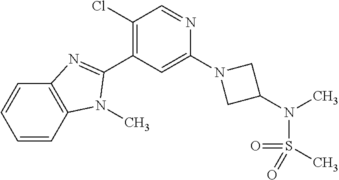 | N-{1-[5-chloro- 4-(1-methyl-1H- benzimidazol-2- yl)pyridin-2-yl] azetidin-3-yl}-N- methylmethane sulfonamide | 406.5 | ¹H NMR (400 MHz, CHLOROFORM-d) δ ppm 8.21 (s, 1 H), 7.78-7.76 (d, 1 H), 7.38-7.27 (m, 3 H), 6.50 (s, 1 H), 4.81-4.74 (m, 1 H), 4.25-4.21 (m, 2 H), 4.10- 4.07 (m, 2 H), 3.66 (s, 3 H), 2.93-2.91 (d, 3 H), 3.76 (s, 3 H) |
| A-150 | 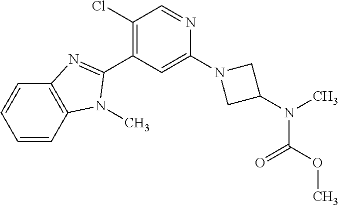 | methyl {1-[5- chloro-4-(1- methyl-1H- benzimidazol- 2-yl)pyridin-2- yl]azetidin-3- yl}methyl- carbamate | 386.3 | ¹H NMR (400 MHz, CHLOROFORM-d) δ ppm 8.19 (s, 1 H), 7.78-7.76 (d, 1 H), 7.38-7.26 (m, 3 H), 6.47 (s, 1 H), 5.13 (s, 1 H), 4.23-4.19 (m, 2 H), 4.03- 4.00 (m, 2 H), 3.71-3.66 (m, 6 H), 2.94 (s, 3 H) |
| A-151 | 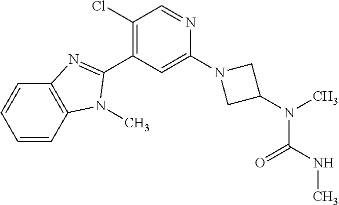 | 1-{1-[5-chloro- 4-(1-methyl-1H- benzimidazol- 2-yl)pyridin-2- yl]azetidin-3- yl}-1,3- dimethylurea | 385.3 | ¹H NMR (400 MHz, CHLOROFORM-d) δ ppm 8.19 (s, 1 H), 7.78-7.76 (d, 1 H), 7.37-7.26 (m, 3 H), 6.46 (s, 1 H), 5.37-5.31 (m, 1 H), 4.44-4.43 (m, 1 H), 4.25-4.21 (m, 2 H), 3.976- 3.93 (m, 2 H), 3.66 (s, 3 H), 2.91 (s, 3 H), 2.77-2.74 (t, 3 H) |
| A-152 | 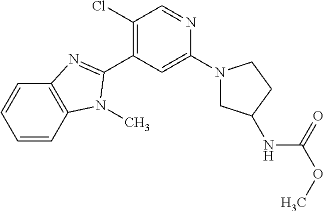 | methyl {1-[5- chloro-4-(1- methyl-1H- benzimidazol- 2-yl)pyridin-2- yl]pyrrolidin-3- yl}carbamate | 386.2 | ¹H NMR (400 MHz, CHLOROFORM-d) δ ppm 8.24 (s, 1 H), 7.84-7.82 (d, 1 H), 7.43-7.41 (d, 1 H), 7.40- 7.32 (m, 2 H), 6.56 (s, 1 H), 4.85 (m, 1 H), 4.40 (m, 1 H), 3.76-3.60 (m, 7 H), 3.59- 3.49 (m, 2 H), 3.40-3.36 (m, 1 H), 2.34-2.26 (m, 1 H), 2.01-1.99 (m, 1 H) |
| A-153 | 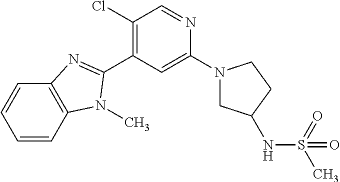 | N-{1-[5-chloro- 4-(1-methyl-1H- benzimidazol- 2-yl)pyridin-2- yl]pyrrolidin-3- yl}methane- sulfonamide | 406.5 | ¹H NMR (400 MHz, CHLOROFORM-d) δ ppm 8.25 (s, 1 H), 7.86-7.84 (d, 1 H), 7.44-7.42 (d, 1 H), 7.39-7.32 (m, 2 H), 6.58 (s, 1 H), 4.98-4.96 (m, 1 H), 4.23-4.4.17 (m, 1 H), 3.85- 3.79 (m, 1 H), 3.71 (s, 3 H), 3.60-3.42 (m, 3 H), 3.00 (s, 3 H), 2.41-2.33 (m, 1 H), 2.09-4.01 (m, 1 H) |
| A-154 | 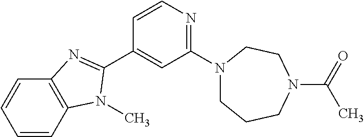 | 2-[2-(4-acetyl- 1,4-diazepan- 1-yl)pyridin-4- yl]-1-methyl-1H- benzimidazole | 350.20 | ¹H NMR (400 MHz, DMSO-d6) δ ppm 8.25 (1 H, t, J = 4.93 Hz) 7.71 (1 H, d, J = 7.83 Hz) 7.65 (1 H, d, J = 8.08 Hz) 7.33 (1 H, t, J = 7.45 Hz) 7.27 (1 H, t, J = 7.45 Hz) 7.04 (1 H, d, J = 6.82 Hz) 6.97 (1 H, t, J = 4.29 Hz) 3.91 (3 H, d, J = 5.05 Hz) 3.87-3.92 (1 H, m) 3.76 (2 H, d, J = 3.54 Hz) 3.70 (1 H, t, J = 5.68 Hz) 3.64 (2 H, t, J = 5.05 Hz) 3.42 (1 H, t, J = 5.94 Hz) 3.38 (1 H, t, J = 5.43 Hz) 1.90 (3 H, s) 1.86-1.89 (1 H, m) 1.75-1.83 (1 H, m) |
| A-155 | 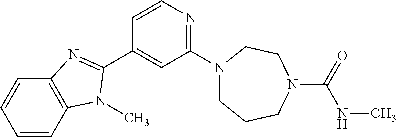 | N-methyl-4-[4- (1-methyl-1H- benzimidazol- 2-yl)pyridin-2- yl]-1,4- diazepane-1- carboxamide | 365.20 | ¹H NMR (400 MHz, DMSO-d6) δ ppm 8.24 (1 H, d, J = 5.05 Hz) 7.71 (1 H, d, J = 7.83 Hz) 7.65 (1 H, d, J = 7.83 Hz) 7.30-7.36 (1 H, m) 7.23-7.30 (1 H, m) 7.01 (1 H, s) 6.96 (1 H, dd, J = 5.05, 1.01 Hz) 6.24 (1 H, q, J = 4.21 Hz) 3.91 (3 H, s) 3.77 (2 H, t, J = 5.31 Hz) 3.68 (2 H, t, J = 5.68 Hz) 3.51 (2 H, t, J = 5.31 Hz) 3.29 (2 H, t, J = 5.94 Hz) 2.54 (3 H, d, J = 4.29 Hz) 1.78-1.89 (2 H, m) |
| A-156 | 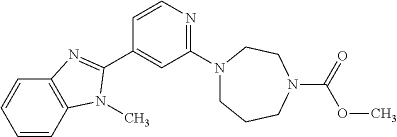 | methyl 4-[4-(1- methyl-1H- benzimidazol- 2-yl)pyridin-2- yl]-1,4- diazepane-1- carboxylate | 366.25 | ¹H NMR (400 MHz, DMSO-d6) δ ppm 8.21 (d, J = 4.88 Hz, 1 H) 7.67 (d, J = 8.78 Hz, 1 H) 7.60 (d, J = 7.81 Hz, 1 H) 7.29 (t, J = 7.32 Hz, 1 H) 7.23 (t, J = 7.32 Hz, 1 H) 6.99 (s, 1 H) 6.93 (d, J = 4.88 Hz, 1 H) 3.87 (s, 3 H) 3.78 (t, J = 5.37 Hz, 2 H) 3.70 (t, J = 5.86 Hz, 2 H) 3.43-3.57 (m, 5 H) 3.23- 3.25 (m, 1 H) 3.14 (d, J = 4.88 Hz, 1 H) 1.80 (br. s., 2 H) |
| A-157 | 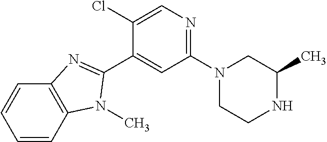 | 2-{5-chloro-2- [(3R)-3-methyl- piperazin-1-yl] pyridin-4-yl}-1- methyl-1H- benzimidazole | 342.10 | ¹H NMR (400 MHz, DMSO-d6) δ ppm 8.30 (s, 1 H) 7.70 (d, J = 7.83 Hz, 1 H) 7.65 (d, J = 7.83 Hz, 1 H) 7.35 (t, J = 7.20 Hz, 1 H) 7.29 (t, J = 7.20 Hz, 1 H) 7.05 (s, 1 H) 4.14 (d, J = 11.87 Hz, 2 H) 3.67 (s, 3 H) 2.92 (d, J = 11.37 Hz, 1 H) 2.71-2.80 (m, 1 H) 2.61- 2.71 (m, 2 H) 2.35-2.44 (m, 1 H) 2.28 (br. s., 1 H) 1.00 (d, J = 6.32 Hz, 3 H) |
| A-158 | 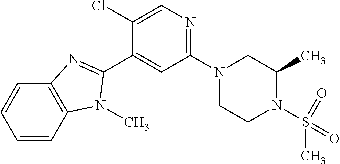 | 2-{5-chloro-2- [(3R)-3-methyl- 4-(methyl- sulfonyl) piperazin-1- yl]pyridin-4-yl}- 1-methyl-1H- benzimidazole | 420.10 | ¹H NMR (400 MHz, DMSO-d6) δ ppm 8.33 (s, 1 H) 7.71 (d, J = 7.83 Hz, 1 H) 7.66 (d, J = 7.83 Hz, 1 H) 7.32-7.39 (m, 1 H) 7.27-7.32 (m, 1 H) 7.13 (s, 1 H) 4.24 (d, J = 12.88 Hz, 1 H) 4.16 (d, J = 13.14 Hz, 1 H) 3.98-4.08 (m, 1 H) 3.68 (s, 3 H) 3.50-3.58 (m, 1 H) 3.22-3.30 (m, 2 H) 3.02-3.13 (m, 1 H) 2.99 (s, 3 H) 1.20 (d, J = 6.57 Hz, 3 H) |
| A-159 | 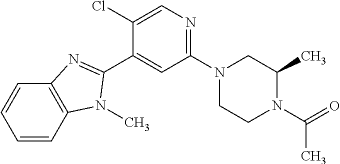 | 2-{2-[(3R)-4- acetyl-3-methyl- piperazin-1-yl]- 5-chloropyridin- 4-yl}-1- methyl-1H- benzimidazole | 384.10 | ¹H NMR (300 MHz, DMSO-d6) δ ppm 8.31 (s, 1 H) 7.69-7.74 (m, 1 H) 7.60-7.64 (m, 1 H) 7.32-7.39 (m, 1 H) 7.26-7.32 (m, 1 H) 7.05 (s, 1 H) 4.41 (br. s., 1 H) 4.05-4.21 (m, 2 H) 3.98 (br. s., 1 H) 3.69 (s, 3 H) 3.26 (dd, J = 13.28, 4.05 Hz, 2 H) 3.03 (br. s., 1 H) 2.04 (s, 3 H) 1.16 (d, J = 6.78 Hz, 3 H) |
| A-160 | 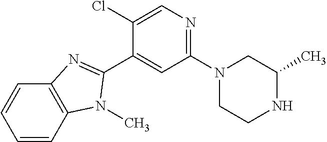 | 2-{5-chloro-2- [(3S)-3-methyl- piperazin-1-yl] pyridin-4-yl}- 1-methyl-1H- benzimidazole | 342.10 | ¹H NMR (400 MHz, DMSO-d6) δ ppm 8.30 (s, 1 H) 7.70 (d, J = 7.83 Hz, 1 H) 7.65 (d, J = 7.83 Hz, 1 H) 7.34 (t, J = 7.45 Hz, 1 H) 7.28 (t, J = 7.45 Hz, 1 H) 7.05 (s, 1 H) 4.14 (d, J = 12.13 Hz, 2 H) 3.67 (s, 3 H) 2.92 (d, J = 11.37 Hz, 1 H) 2.71-2.81 (m, 1 H) 2.61- 2.71 (m, 2 H) 2.39 (t, J = 11.37 Hz, 1 H) 2.27 (br. s., 1 H) 1.00 (d, J = 6.06 Hz, 3 H) |
| A-161 | 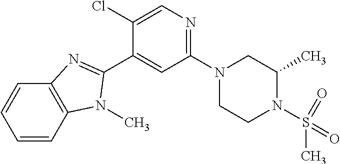 | 2-{5-chloro-2- [(3S)-3-methyl-4- (methylsulfonyl) piperazin-1- yl]pyridin-4-yl}- 1-methyl-1H- benzimidazole | 420.10 | ¹H NMR (400 MHz, DMSO-d6) δ ppm 8.33 (s, 1 H) 7.71 (d, J = 7.83 Hz, 1 H) 7.66 (d, J = 7.83 Hz, 1 H) 7.35 (t, J = 7.45 Hz, 1 H) 7.29 (t, J = 7.45 Hz, 1 H) 7.13 (s, 1 H) 4.24 (d, J = 13.39 Hz, 1 H) 4.16 (d, J = 12.38 Hz, 1 H) 3.99-4.06 (m, 1 H) 3.68 (s, 3 H) 3.54 (d, J = 13.39 Hz, 1 H) 3.21- 3.30 (m, 2 H) 3.02-3.12 (m, 1 H) 2.99 (s, 3 H) 1.20 (d, J = 6.82 Hz, 3 H) |
| A-162 | 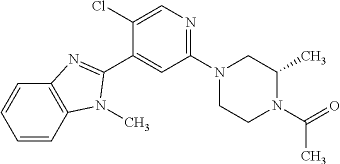 | 2-{2-[(3S)-4- acetyl-3-methyl- piperazin-1- yl]-5-chloro- pyridin-4-yl}- 1-methyl-1H- benzimidazole | 384.10 | ¹H NMR (300 MHz, DMSO-d6) δ ppm 8.31 (s, 1 H) 7.68-7.73 (m, 1 H) 7.60-7.65 (m, 1 H) 7.32-7.38 (m, 1 H) 7.25-7.32 (m, 1 H) 7.05 (s, 1 H) 4.42 (br. s., 1 H) 4.06-4.20 (m, 2 H) 3.97 (br. s., 1 H) 3.69 (s, 3 H) 3.29 (d, J = 3.96 Hz, 1 H) 3.24 (d, J = 4.14 Hz, 1 H) 2.98- 3.04 (m, 1 H) 2.04 (s, 3 H) 1.16 (d, J = 6.78 Hz, 3 H) |
| A-163 | 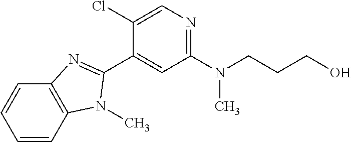 | 3-{[5-chloro-4- (1-methyl-1H- benzimidazol- 2-yl)pyridin-2- yl](methyl) amino}propan- 1-ol | 331.15 | ¹H NMR (400 MHz, DMSO-d6) δ ppm 8.28 (s, 1 H) 7.71 (d, J = 7.58 Hz, 1 H) 7.65 (d, J = 7.83 Hz, 1 H) 7.34 (t, J = 7.33 Hz, 1 H) 7.28 (t, J = 7.58 Hz, 1 H) 6.84 (s, 1 H) 4.50 (br. s., 1 H) 3.68 (s, 3 H) 3.57 (t, J = 7.07 Hz, 2 H) 3.40-3.48 (m, 2 H) 3.04 (s, 3 H) 1.64-1.77 (m, 2 H) |
| A-164 | 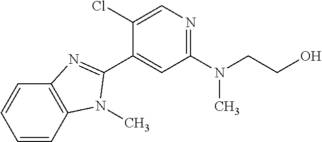 | 2-{[5-chloro-4- (1-methyl-1H- benzimidazol- 2-yl)pyridin-2- yl](methyl) amino}ethanol | 317.05 | ¹H NMR (400 MHz, DMSO-d6) δ ppm 8.27 (s, 1 H) 7.71 (d, J = 7.58 Hz, 1 H) 7.65 (d, J = 7.83 Hz, 1 H) 7.34 (td, J = 7.52, 1.14 Hz, 1 H) 7.28 (td, 1 H) 6.86 (s, 1 H) 4.69- 4.75 (m, 1 H) 3.68 (s, 3 H) 3.54-3.63 (m, 4 H) 3.08 (s, 3 H) |
| A-165 | 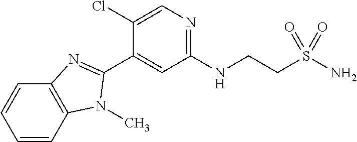 | 2-{[5-chloro-4- (1-methyl-1H- benzimidazol- 2-yl)pyridin-2- yl]amino}ethane- sulfonamide | 366.10 | ¹H NMR (400 MHz, DMSO-d6) δ ppm 8.24 (s, 1 H) 7.70 (d, J = 8.08 Hz, 1 H) 7.64 (d, J = 7.83 Hz, 1 H) 7.35 (t, J = 7.45 Hz, 1 H) 7.28 (t, J = 7.45 Hz, 1 H) 7.22 (t, J = 5.68 Hz, 1 H) 6.92 (s, 2 H) 6.72 (s, 1 H) 3.68-3.75 (m, 2 H) 3.68 (s, 3 H) 3.27 (t, J = 7.07 Hz, 2 H) |
| A-166 | 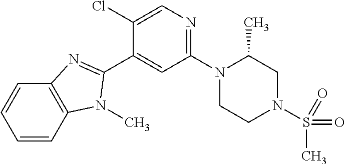 | 2-{5-chloro-2- [(2R)-2-methyl-4- (methylsulfonyl) piperazin-1- yl]pyridin-4-yl}- 1-methyl-1H- benzimidazole | 420.10 | ¹H NMR (400 MHz, DMSO-d6) δ ppm 8.37 (s, 1 H) 7.71 (d, J = 7.83 Hz, 1 H) 7.66 (d, J = 8.08 Hz, 1 H) 7.35 (t, J = 7.07 Hz, 1 H) 7.29 (t, J = 7.07 Hz, 1 H) 7.11 (s, 1 H) 4.68- 4.78 (m, 1 H) 4.24 (d, J = 13.14 Hz, 1 H) 3.69 (s, 3 H) 3.60 (d, J = 11.12 Hz, 1 H) 3.42 (d, J = 11.62 Hz, 1 H) 3.13 (td, J = 12.82, 3.16 Hz, 1 H) 2.96-3.02 (m, 1 H) 2.91 (s, 3 H) 2.83 (td, J = 11.75, 3.28 Hz, 1 H) 1.17 (d, J = 6.57 Hz, 3 H) |
| A-167 | 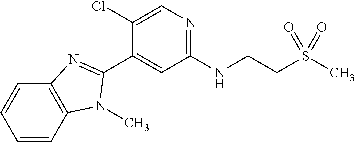 | 5-chloro-4-(1- methyl-1H- benzimidazol- 2-yl)-N-[2- (methylsulfonyl) ethyl]pyridin-2- amine | 365.05 | ¹H NMR (400 MHz, DMSO-d6) δ ppm 8.26 (s, 1 H) 7.70 (d, J = 8.08 Hz, 1 H) 7.64 (d, J = 7.83 Hz, 1 H) 7.33 (s, 1 H) 7.35 (t, J = 7.07 Hz, 1 H) 7.28 (t, J = 7.58 Hz, 1 H) 6.76 (s, 1 H) 3.73 (q, J = 6.48 Hz, 2 H) 3.68 (s, 3 H) 3.40 (t, J = 6.69 Hz, 2 H) 3.03 (s, 3 H) |
| A-168 | 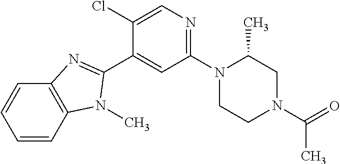 | 2-{2-[(2R)-4- acetyl-2-methyl- piperazin-1-yl]- 5-chloropyridin- 4-yl}-1- methyl-1H benzimidazole | 384.10 | ¹H NMR (400 MHz, DMSO-d6) δ ppm 8.36 (s, 1 H) 7.71 (d, J = 7.83 Hz, 1 H) 7.65 (d, J = 7.83 Hz, 1 H) 7.35 (t, J = 7.45 Hz, 1 H) 7.29 (t, J = 7.45 Hz, 1 H) 7.05 (s, 1 H) 4.56 (br. s., 1 H) 4.31 (d, J = 13.64 Hz, 0.5 H) 4.21 (d, J = 13.39 Hz, 0.5 H) 4.02-4.12 (m, 1 H) 3.87 (d, J = 10.86 Hz, 0.5 H) 3.73 (d, J = 12.88 Hz, 0.5 H) 3.68 (s, 3 H) 3.43 (dd, J = 13.39, 3.54 Hz, 0.5 H) 3.16- 3.28 (m, 1 H) 2.99-3.09 (m, 0.5 H) 2.95 (dd, J = 13.39, 3.28 Hz, 0.5 H) 2.75-2.87 (m, 0.5 H) 2.08 (s, 1.5 H) 2.03 (s, 1.5 H) 1.10 (d, J = 6.32 Hz, 1.5 H) 1.03 (d, J = 6.32 Hz, 1.5 H) |
| A-169 | 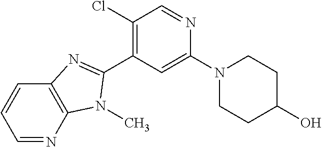 | 1-[5-chloro-4- (3-methyl-3H- imidazo[4,5- b]pyridin-2- yl)pyridin-2- yl]piperidin-4-ol | 344.20 | ¹H NMR (400 MHz, DMSO-d6) δ ppm 1.32-1.45 (m, 2 H) 1.74-1.84 (m, 2 H) 3.15-3.25 (m, 2 H) 3.71 (s, 3 H) 3.73- 3.79 (m, 1 H) 3.99-4.08 (m, 1 H) 4.75 (d, J = 4.29 Hz, 1 H) 7.15 (s, 1 H) 7.39 (dd, J = 7.83, 4.80 Hz, 1 H) 8.17 (dd, J = 7.96, 1.39 Hz, 1 H) 8.33 (s, 1 H) 8.47 (dd, J = 4.80, 1.26 Hz, 1 H) |
| A-170 | 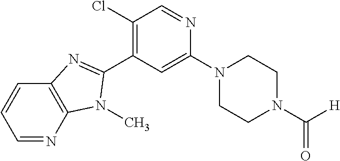 | 4-[5-chloro-4- (3-methyl-3H- imidazo[4,5- b]pyridin-2- yl)pyridin-2- yl]piperazine-1- carbaldehyde | 357.20 | ¹H NMR (400 MHz, DMSO-d6) δ ppm 1.92 (s, 3 H) 3.45- 3.53 (m, 4 H) 3.57-3.69 (m, 4 H) 3.72 (s, 3 H) 7.23 (s, 1 H) 7.39 (dd, J = 7.83, 4.80 Hz, 1 H) 8.11 (s, 1 H) 8.15-8.21 (m, 1 H) 8.40 (s, 1 H) 8.48 (d, J = 3.54 Hz, 1 H) |
| A-171 | 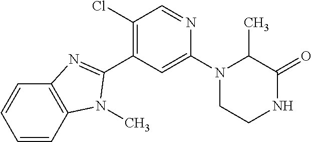 | 4-[5-chloro-4- (1-methyl-1H- benzimidazol- 2-yl)pyridin-2- yl]-3-methyl- piperazin-2-one | 355.12/ 356.20 | 1H NMR (400 MHz, DMSO-d₆) δ ppm 8.35 (s, 1 H) 8.05 (br. s., 1 H) 7.71 (d, J = 7.83 Hz, 1 H) 7.65 (d, J = 7.83 Hz, 1 H) 7.30 (d, J = 7.07 Hz, 1 H) 7.35 (t, J = 7.07 Hz, 1 H) 7.08 (s, 1 H) 4.71 (d, J = 6.82 Hz, 1 H) 4.12-4.27 (m, 1 H) 3.69 (s, 3 H) 3.23-3.29 (m, 1 H) 2.54 (s, 2 H) 1.36 (d, J = 6.82 Hz, 3 H) |
| A-172 | 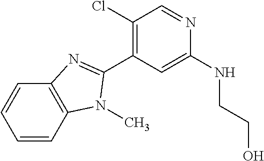 | 2-{[5-chloro-4- (1-methyl-1H- benzimidazol- 2-yl)pyridin-2- yl]amino}ethanol | 303.10/ 305.05 | 1H NMR (400 MHz, DMSO-d₆) δ ppm 3.36 (q, J = 5.98 Hz, 2 H) 3.55 (q, J = 5.64 Hz, 2 H) 3.67 (s, 3 H) 4.73 (t, J = 5.31 Hz, 1 H) 6.73 (s, 1 H) 7.07 (t, J = 5.43 Hz, 1 H) 7.26-7.37 (m, 2 H) 7.64 (d, J = 7.83 Hz, 1 H) 7.70 (d, J = 7.83 Hz, 1 H) 8.18 (s, 1 H) |
| A-173 | 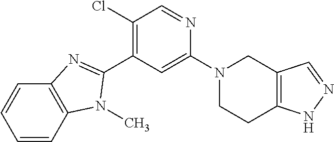 | 5-[5-chloro-4- (1-methyl-1H- benzimidazol- 2-yl)pyridin-2- yl]-4,5,6,7- tetrahydro-1H- pyrazolo[4,3- c]pyridine | 365.05/ 367.15 | 1H NMR (400 MHz, DMSO-d₆) δ ppm 2.70 (t, J = 4.67 Hz, 2 H) 3.64 (s, 3 H) 3.91 (t, J = 5.31 Hz, 2 H) 4.58 (br. S, 2 H) 7.12 (s, 1 H) 7.21-7.35 (m, 3 H) 7.61 (d, J = 7.83 Hz, 1 H) 7.67 (d, J = 7.83 Hz, 1 H) 8.30 (s, 1 H) 12.45 (br. S, 1 H) |
| A-174 | 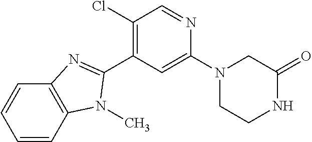 | 4-[5-chloro-4- (1-methyl-1H- benzimidazol- 2-yl)pyridin-2- yl]piperazin-2- one | 342.10/ 344.05 | ¹H NMR (400 MHz, DMSO-d6) δ ppm 8.37 (s, 1 H) 8.13 (br. s., 1 H) 7.71 (d, J = 7.83 Hz, 1 H) 7.66 (d, J = 8.08 Hz, 1 H) 7.26-7.39 (m, 2 H) 7.09 (s, 1 H) 4.06 (s, 2 H) 3.77 (t, J = 5.18 Hz, 2 H) 3.68 (s, 3 H), 3.32 (CH₂ obscured by H₂O peak) |
| A-175 | 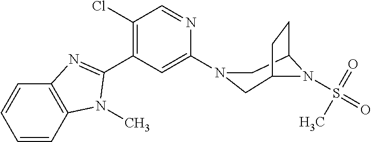 | 2-{5-chloro-2- [(1R,5S)-8- (methylsulfonyl)- 3,8-diaza- bicyclo[3.2.1]oct- 3-yl]pyridin-4- yl}-1-methyl-1H- benzimidazole | 431.95/ 432.20 | ¹H NMR (400 MHz, CHLOROFORM-d) δ ppm 8.29 (s, 1 H) 7.80-7.93 (m, 1 H) 7.38 (quin, J = 7.20 Hz, 1 H) 7.33-7.50 (m, 2 H) 6.80 (s, 1 H) 4.37 (d, J = 2.02 Hz, 1 H) 4.06 (dd, J = 12.13, 2.27 Hz, 2 H) 3.74 (s, 3 H) 3.19 (dd, J = 12.00, 1.64 Hz, 2 H) 2.99 (s, 3 H) 2.03-2.11 (m, 2 H) 1.87 (d, J = 7.58 Hz, 2 H) |
| A-176 | 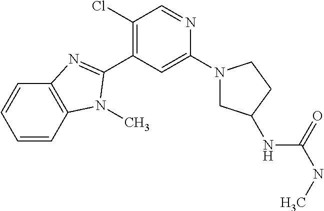 | 1-{1-[5-chloro- 4-(1-methyl-1H- benzimidazol- 2-yl)pyridin-2- yl]pyrrolidin-3- yl}-3-methylurea | 385.3 | ¹H NMR (400 MHz, CHLOROFORM-d) δ ppm 8.17 (s, 1 H), 7.75-7.73 (d, 1 H), 7.38-7.36 (d, 1 H), 7.32- 7.25 (m, 2 H), 6.47 (s, 1 H), 4.91 (m, 1 H), 4.54 (m, 1 H), 4.41 (M, 1 H), 3.65 (m, 4 H), 3.45 (m, 2 H), 3.38-3.26 (m, 1 H), 2.67-2..66 (d, 3 H), 2.21-2.19 (m, 1 H), 1.89-1.87 (m, 1 H) |
| A-177 | 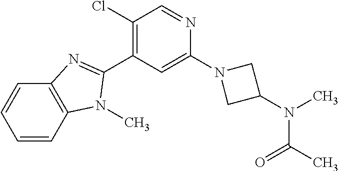 | N-{1-[5-chloro- 4-(1-methyl-1H- benzimidazol- 2-yl)pyridin-2- yl]azetidin-3- yl}-N-methyl- acetamide | 370.3 | ¹H NMR (400 MHz, DMSO-d6) δ 8.20 (s, 1 H), 7.62-7.60 (d, 1 H), 7.53-7.51 (d, 1 H), 7.27- 7.14 (m, 2 H), 6.56 (s, 1 H), 5.11 (s, 1 H), 4.15 (s, 2 H), 3.97 (s, 2 H), 3.59 (s, 3 H), 2.95-2.92 (d, 3 H), 1.94 (s, 3 H) |
| A-178 | 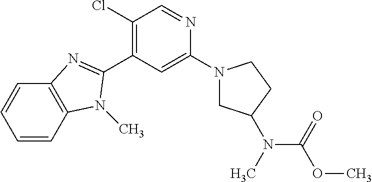 | methyl {1-[5- chloro-4-(1- methyl-1H- benzimidazol- 2-yl)pyridin-2- yl]pyrrolidin-3- yl}methyl- carbamate | 400.2 | ¹H NMR (400 MHz, CHLOROFORM-d) δ ppm 8.19 (s, 1 H), 7.78-7.77 (d, 1 H), 7.38-7.36 (d, 1 H), 7.34- 7.26 (m, 2 H), 6.52 (s, 1 H), 4.89 (m, 1 H), 3.66 (s, 6 H), 3.64-3.57 (m, 2 H), 3.43-3.32 (m, 2 H), 2.79 (s, 3 H), 2.20- 2.02 (m, 2 H) |
| A-179 | 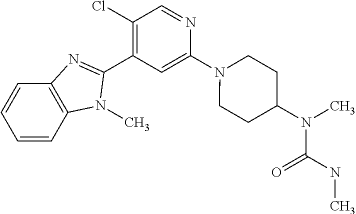 | 1-{1-[5-chloro- 4-(1-methyl-1H- benzimidazol- 2-yl)pyridin-2- yl]piperidin-4- yl}-1,3- dimethylurea | 413.3 | ¹H NMR (400 MHz, CHLOROFORM-d) δ ppm 8.25 (s, 1 H), 7.84-7.83 (d, 1 H), 7.44-7.42 (d, 1 H), 7.39- 7.25 (m, 2 H), 6.87 (s, 1 H), 4.52-4.34 (m, 4 H), 3.72 (s, 3 H), 2.99-2.93 (t, 2 H), 2.83- 2.82 (d, 3 H), 2.68 (s, 3 H), 1.70-1.64 (m, 4 H) |
| A-180 | 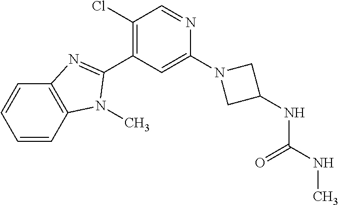 | 1-{1-[5-chloro- 4-(1-methyl-1H- benzimidazol- 2-yl)pyridin-2- yl]azetidin-3- yl}-3-methylurea | 371.2 | ¹H NMR (400 MHz, MeOD) δ ppm 8.12 (s, 1 H), 7.63- 7.61 (d, 1 H), 7.52-7.50 (d, 1 H), 7.31-7.26 (m, 2 H), 6.54 (s, 1 H), 4.55-4.51 (m, 1 H), 4.27-4.23 (m, 2 H), 3.79- 3.75 (m, 2 H), 3.65 (s, 3 H), 2.60 (s, 3 H) |
| A-181 | 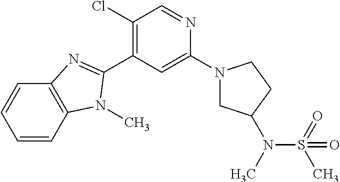 | N-{1-[5-chloro- 4-(1-methyl-1H- benzimidazol- 2-yl)pyridin-2- yl]pyrrolidin-3- yl}-N-methyl- methane sulfonamide | 420.4 | ¹H NMR (400 MHz, CHLOROFORM-d) δ ppm 8.20 (s, 1 H), 7.78-7.77 (d, 1 H), 7.38-7.36 (d, 1 H), 7.33- 7.26 (m, 2 H), 6.54 (s, 1 H), 4.68-4.60 (m, 1 H), 3.69-3.61 (m, 5 H), 3.44-3.33 (m, 2 H), 2.81 (s, 6 H), 2.27-2.09 (m, 2 H) |
| A-182 | 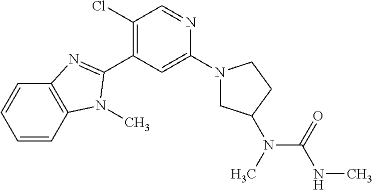 | 1-{1-[5-chloro- 4-(1-methyl-1H- benzimidazol- 2-yl)pyridin-2- yl]pyrrolidin-3- yl}-1,3- dimethylurea | 399.3 | ¹H NMR (400 MHz, CHLOROFORM-d) δ ppm 8.19 (s, 1 H), 7.78-7.76 (d, 1 H), 7.38-7.36 (d, 1 H), 7.33- 7.26 (m, 2 H), 6.51 (s, 1 H), 5.18-5.11 (m, 1 H), 4.36- 4.35 (m, 1 H), 3.66-3.54 (m, 5 H), 3.42-3.26 (m, 2 H), 2.78-2.77 (d, 3 H), 2.73 (s, 3 H), 2.17-2.13 (m, 1 H), 2.03-1.94 (m, 1 H) |
| A-183 | 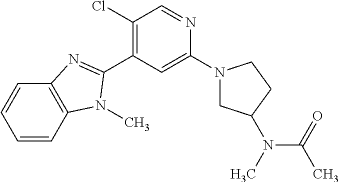 | N-{1-[5-chloro- 4-(1-methyl-1H- benzimidazol- 2-yl)pyridin-2- yl]pyrrolidin-3- yl}-N-methyl- acetamide | 384.4 | ¹H NMR (400 MHz, DMSO-d₆) δ ppm 8.33-8.32 (d, 1 H), 7.72-7.65 (dd, 2 H), 7.37- 7.27 (m, 2 H), 6.74-6.72 (d, 1 H), 5.22-5.12 (m, 0.5 H), 4.72-4.62 (m, 0.5 H), 3.69 (m, 4.4 H), 3.56 (m, 0.6 H), 3.43- 3.41 (m, 2 H), 2.89 (s, 1.7 H), 2.75 (s, 1.3 H), 2.19-2.02 (m, 5 H) |
| A-184 | 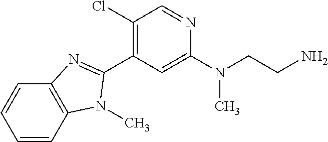 | N-[5-chloro-4- (1-methyl-1H- benzimidazol- 2-yl)pyridin-2- yl]-N-methyl- ethane-1,2- diamine | 315.81/ 316.20 | ¹H NMR (400 MHz, DMSO-d₆) δ ppm 8.27 (s, 1 H) 7.71 (d, J = 7.58 Hz, 1 H) 7.65 (d, J = 7.83 Hz, 1 H) 7.19-7.39 (m, 2 H) 6.87 (s, 1 H) 3.64-3.70 (m, 4 H) 3.06 (s, 3 H) 2.74 (t, J = 6.95 Hz, 2 H) 1.86 (s, 3 H) |
| A-185 | 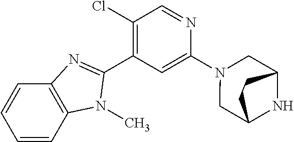 | 2-{5-chloro-2- [(1R,5S)-3,8- diaza- bicyclo[3.2.1]oct- 3-yl]pyridin-4- yl}-1-methyl-1H- benzimidazole | 353.14/ 354.20 | ¹H NMR (400 MHz, DMSO-d₆) δ ppm 8.34 (s, 1 H) 7.71 (d, J = 8.08 Hz, 1 H) 7.65 (d, J = 7.83 Hz, 1 H) 7.21-7.40 (m, 2 H) 6.99 (s, 1 H) 3.94 (d, J = 12.38 Hz, 2 H) 3.77 (s., 2 H) 3.68 (s, 3 H) 3.06 (d, J = 12.13 Hz, 2 H) 2.54 (s, 1 H) 1.91 (s, 1 H) 1.77-1.82 (m, 1 H) 1.67-1.72 (m, 1 H) |
| A-186 | 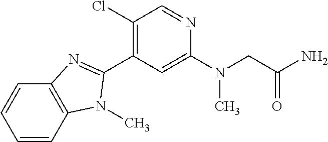 | N~2~-[5- chloro-4-(1- methyl-1H- benzimidazol- 2-yl)pyridin-2- yl]-N~2~-methyl- glycinamide | 329.10/ 330.20 | ¹H NMR (400 MHz, DMSO-d₆) δ ppm 8.28 (s, 1 H) 7.71 (d, J = 7.83 Hz, 1 H) 7.65 (d, J = 7.83 Hz, 1 H) 7.22-7.43 (m, 3 H) 7.03 (br. s., 1 H) 6.83 (s, 1 H) 4.14 (s, 2 H) 3.68 (s, 3 H) 3.07 (s, 3 H) |
| A-187 | 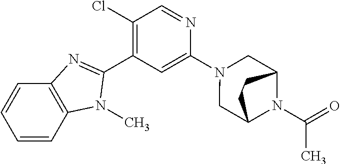 | 2-{2-[(1R,5S)- 8-acetyl-3,8- diaza- bicyclo[3.2.1]oct- 3-yl]-5- chloropyridin-4- yl}-1-methyl-1H- benzimidazole | 395.15/ 396.20 | ¹H NMR (400 MHz, CHLOROFORM-d) δ ppm 8.28 (s, 1 H) 7.84 (d, J = 7.83 Hz, 1 H) 7.42-7.49 (m, 1 H) 7.37 (qd, J = 7.20, 6.95 Hz, 2 H) 6.79 (s, 1 H) 4.85 (d, J = 6.32 Hz, 1 H) 4.26 (d, J = 6.06 Hz, 1 H) 4.13 (d, J = 12.38 Hz, 1 H) 3.90 (d, J = 11.87 Hz, 1 H) 3.73 (s, 3 H) 3.49 (s, 1 H) 3.16 (d, J = 11.87 Hz, 1 H) 3.08 (d, J = 11.87 Hz, 1 H) 2.13 (s, 3 H) 2.11 (s, 2 H) 1.81 (d, J = 10.86 Hz, 1 H) |
| A-188 | 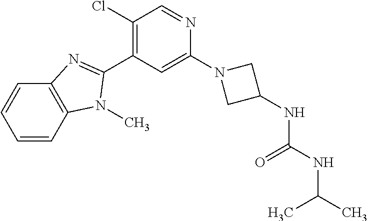 | 1-{1-[5-chloro- 4-(1-methyl-1H- benzimidazol- 2-yl)pyridin-2- yl]azetidin-3- yl}-3- isopropylurea | 399.3 | ¹H NMR (400 MHz, DMSO-d₆) δ ppm 8.23 (s, 1 H), 7.72-7.65 (q, 2 H), 7.37-7.29 (m, 2 H), 6.66 (s, 1 H), 6.47-6.45 (d, 1 H), 5.81-5.80 (d, 1 H), 4.55- 4.53 (m, 1 H), 4.25-4.21 (m, 2 H), 3.78-3.73 (m, 2 H), 3.68-3.61 (m, 4 H), 1.03-1.02 (d, 6 H) |
| A-189 | 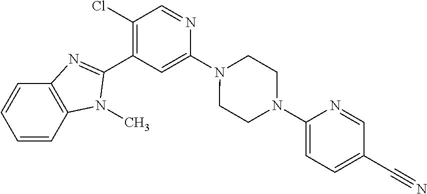 | 6-{4-[5-chloro- 4-(1-methyl-1H- benzimidazol-2- yl)pyridin-2-yl] piperazin-1-yl} nicotinonitrile | 430.15/ 432.10 | ¹H NMR (400 MHz, DMSO-d6) δ ppm 8.51 (d, J = 2.0 Hz, 1 H) 8.37 (s, 1 H) 7.88 (dd, J = 9.1, 2.3 Hz, 1 H) 7.71 (d, J = 7.6 Hz, 1 H) 7.66 (d, J = 8.1 Hz, 1 H) 7.32-7.43 (m, 1 H) 7.22- 7.32 (m, 1 H) 7.14 (s, 1 H) 6.98 (d, J = 9.1 Hz, 1 H) 3.77- 3.87 (m, 4 H) 3.70-3.76 (m, 4 H) 3.69 (s, 3 H) |
| A-190 | 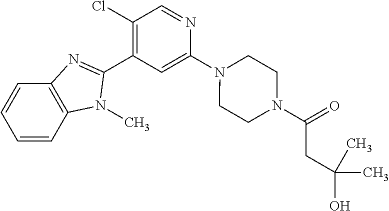 | 4-{4-[5-chloro- 4-(1-methyl-1H- benzimidazol- 2-yl)pyridin-2- yl]piperazin-1- yl}-2-methyl-4- oxobutan-2-ol | 428.20 | ¹H NMR (400 MHz, DMSO-d6) δ ppm 8.36 (s, 1 H) 7.71 (d, J = 7.83 Hz, 1 H) 7.65 (d, J = 7.83 Hz, 1 H) 7.33-7.38 (m, 1 H) 7.27-7.32 (m, 1 H) 7.12 (s, 1 H) 4.80 (s, 1 H) 3.68 (s, 3 H) 3.54-3.67 (m, 8 H) 2.50 (s, 2 H) 1.19 (s, 6 H) |
| A-191 | 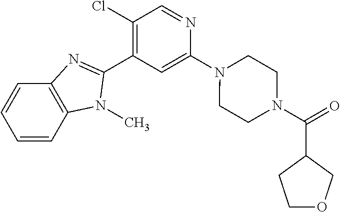 | 2-{5-chloro-2-[4- (tetrahydrofuran- 3-ylcarbonyl) piperazin-1-yl] pyridin-4-yl}- 1-methyl-1H- benzimidazole | 426.10 | ¹H NMR (400 MHz, DMSO-d6) δ ppm 8.36 (s, 1 H) 7.71 (d, J = 7.58 Hz, 1 H) 7.66 (d, J = 7.83 Hz, 1 H) 7.33-7.38 (m, 1 H) 7.27-7.32 (m, 1 H) 7.13 (s, 1 H) 3.88 (t, J = 8.21 Hz, 1 H) 3.69-3.75 (m, 2 H) 3.68 (s, 3 H) 3.67 (s, 1 H) 3.63 (s, 4 H) 3.58 (br. s., 4 H) 3.36- 3.45 (m, 1 H) 1.98-2.06 (m, 2 H) |
| A-192 | 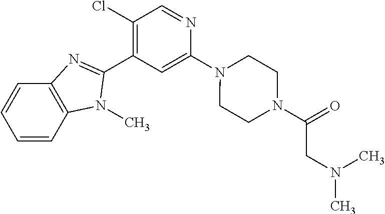 | 2-{4-[5-chloro- 4-(1-methyl-1H- benzimidazol- 2-yl)pyridin-2- yl]piperazin-1- yl}-N,N- dimethyl-2- oxoethanamine | 413.20 | ¹H NMR (400 MHz, DMSO-d6) δ ppm 8.36 (s, 1 H) 7.71 (d, J = 8.08 Hz, 1 H) 7.65 (d, J = 7.83 Hz, 1 H) 7.35 (t, J = 7.45 Hz, 1 H) 7.29 (t, J = 7.58 Hz, 1 H) 7.12 (s, 1 H) 3.68 (s, 3 H) 3.63-3.66 (m, 2 H) 3.58-3.63 (m, 2 H) 3.56 (br. s., 4 H) 3.11 (s, 2 H) 2.19 (s, 6 H) |
| A-193 | 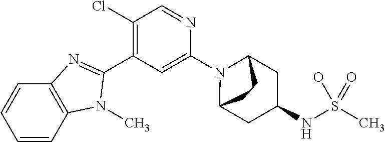 | N-{(3-endo)-8- [5-chloro-4-(1- methyl-1H- benzimidazol- 2-yl)pyridin-2- yl]-8-aza- bicyclo[3.2.1]oct- 3-yl}methane- sulfonamide | 446.20 | ¹H NMR (400 MHz, CHLOROFORM-d) δ ppm 8.28 (s, 1 H) 7.83-7.88 (m, 1 H) 7.43-7.47 (m, 1 H) 7.37 (m, 2 H) 6.75 (s, 1 H) 3.75 (s, 3 H) 2.98 (s, 3 H) 2.37 (dd, J = 8.46, 4.17 Hz, 2 H) 2.14- 2.25 (m, 2 H) 2.05-2.13 (m, 2 H) 1.81 (d, J = 14.40 Hz, 2 H) 1.27 (s, 1 H) 0.87-0.97 (m, 1 H) |
| A-194 | 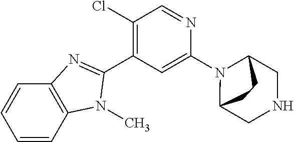 | 2-{5-chloro-2- [(1R,5S)-3,8- diaza- bicyclo[3.2.1]oct- 8-yl]pyridin-4- yl}-1-methyl-1H- benzimidazole | 354.10 | ¹H NMR (400 MHz, DMSO-d₆) δ ppm 8.46 (s, 1 H) 7.71-7.87 (m, 2 H) 7.34-7.54 (m, 2 H) 7.28 (s, 1 H) 4.64-4.74 (m., 2 H) 3.79 (s, 3 H) 3.07-3.16 (m, 4 H) 2.08-2.14 (m, 4 H) |
| A-195 | 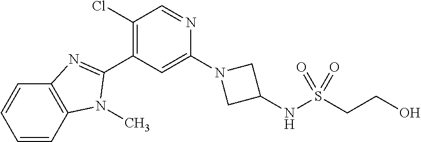 | N-{1-[5-chloro- 4-(1-methyl-1H- benzimidazol- 2-yl)pyridin-2- yl]azetidin-3-yl}- 2-methoxyethane sulfonamide | 436 | ¹H NMR (400 MHz, CDCl3) δ ppm 8.19 (s, 1 H), 7.77 (d, J = 7.2 Hz, 1 H), 7.26-7.37 (m, 3 H), 6.47 (s, 1 H), 4.99-5.01 (m, 1 H), 4.38-4.47 (m, 1 H), 4.30-4.34 (m, 2 H), 3.87-3.91 (m, 2 H), 3.74-3.75 (m, 2 H), 3.65 (s, 3 H), 3.33 (s, 3 H), 3.13-3.18 (m, 2 H) |
| A-196 | 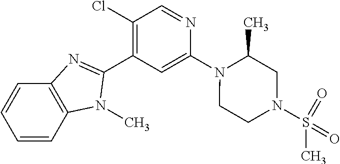 | 2-{5-chloro-2- [(2S)-2-methyl-4- (methylsulfonyl) piperazin-1- yl]pyridin-4-yl}- 1-methyl-1H- benzimidazole | 420.10 | ¹H NMR (400 MHz, DMSO-d6) δ ppm 8.37 (s, 1 H) 7.71 (d, J = 8.08 Hz, 1 H) 7.66 (d, J = 7.83 Hz, 1 H) 7.35 (t, J = 7.71 Hz, 1 H) 7.29 (t, J = 7.07 Hz, 1 H) 7.11 (s, 1 H) 4.72 (dd, J = 4.55, 3.03 Hz, 1 H) 4.24 (d, J = 13.14 Hz, 1 H) 3.69 (s, 3 H) 3.60 (d, J = 12.38 Hz, 1 H) 3.42 (d, J = 11.62 Hz, 1 H) 3.08-3.19 (m, 1 H) 2.99 (dd, J = 11.62, 2.53 Hz, 1 H) 2.91 (s, 3 H) 2.83 (td, J = 11.87, 3.54 Hz, 1 H) 1.17 (d, J = 6.57 Hz, 3 H) |
| A-197 | 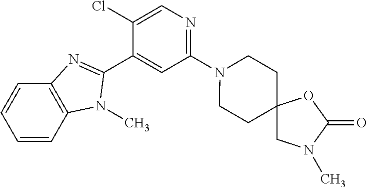 | 8-[5-chloro-4- (1-methyl-1H- benzimidazol- 2-yl)pyridin-2- yl]-3-methyl-1- oxa-3,8- diazaspiro[4.5] decan-2-one | 412.3 | ¹H NMR (400 MHz, DMSO-d6) δ ppm 8.30 (s, 1 H), 7.66-7.68 (d, 1 H), 7.61-7.63 (d, 1 H), 7.23-7.33 (m, 1 H), 7.14 (s, 1 H), 3.82-3.87 (m, 2 H) 3.65 (s, 3 H), 3.45-3.51 (m, 2 H), 3.32 (s, 2 H), 2.72 (s, 3 H), 1.77-1.78 (d, 3 H) |
| A-198 | 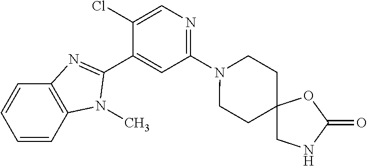 | 8-[5-chloro-4- (1-methyl-1H- benzimidazol- 2-yl)pyridin-2- yl]-1-oxa-3,8- diazaspiro[4.5] decan-2-one | 398.1 | ¹H NMR (400 MHz, DMSO-d6) δ ppm 8.30 (s, 1 H), 7.66-7.68 (d, 1 H), 7.61-7.63 (d, 2 H), 7.51 (s, 1 H), 7.23-7.33 (m, 2 H), 7.13 (s, 1 H), 3.83-3.87 (m, 2 H), 3.65 (s, 3 H), 3.44- 3.51 (m, 2 H), 3.25 (s, 2 H), 1.73-1.78 (m, 4 H) |
| A-199 | 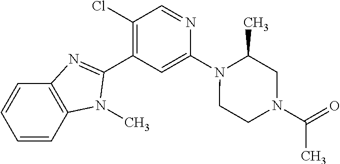 | 2-{2-[(2S)-4- acetyl-2-methyl- piperazin-1- yl]-5-chloro- pyridin-4-yl}-1- methyl-1H- benzimidazole | 384.10 | ¹H NMR (400 MHz, DMSO-d6) δ ppm 8.36 (s, 2 H) 7.71 (d, J = 7.83 Hz, 2 H) 7.65 (d, J = 7.83 Hz, 2 H) 7.32-7.38 (m, 2 H) 7.26-7.32 (m, 2 H) 7.05 (s, 2 H) 4.50-4.61 (m, 2 H) 4.31 (d, J = 13.14 Hz, 1 H) 4.21 (d, J = 12.63 Hz, 1 H) 4.04-4.12 (m, 2 H) 3.87 (d, J = 13.39 Hz, 1 H) 3.73 (d, J = 13.89 Hz, 1 H) 3.68 (s, 6 H) 3.43 (dd, J = 13.52, 3.66 Hz, 1 H) 3.14-3.30 (m, 2 H) 2.99- 3.09 (m, 1 H) 2.95 (dd, J = 13.01, 3.66 Hz, 1 H) 2.76- 2.86 (m, 1 H) 2.08 (s, 3 H) 2.03 (s, 3 H) 1.10 (d, J = 6.57 Hz, 3 H) 1.03 (d, J = 6.32 Hz, 3 H) |
| A-200 | 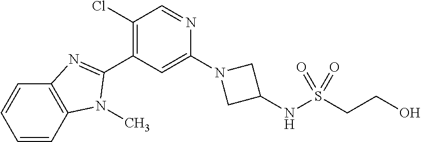 | N-{1-[5-chloro- 4-(1-methyl-1H- benzimidazol- 2-yl)pyridin-2- yl]azetidin-3- yl}-2-hydroxy- ethane sulfonamide | 422 | ¹H NMR (400 MHz, DMSO-d6) δ ppm 8.32 (s, 1 H), 7.89 (d, J = 8 Hz, 1 H), 7.65-7.72 (m, 2 H), 7.27-7.37 (m, 2 H), 6.69 (s, 1 H), 4.95-4.98 (s, 1 H), 4.28-4.36 (m, 3 H), 4.02-4.04 (m, 2 H), 3.72-3.77 (m, 2 H), 3.68 (s, 3 H), 3.16-3.20 (m, 2 H) |
| A-201 | 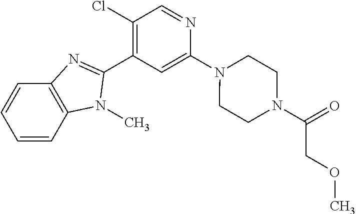 | 2-{5-chloro-2-[4- (methoxyacetyl) piperazin-1- yl]pyridin-4-yl}- 1-methyl-1H- benzimidazole | 400.15 | ¹H NMR (400 MHz, DMSO-d6) δ ppm 8.36 (s, 1 H) 7.71 (d, J = 7.83 Hz, 1 H) 7.66 (d, J = 7.83 Hz, 1 H) 7.33-7.38 (m, 1 H) 7.26-7.32 (m, 1 H) 7.13 (s, 1 H) 4.13 (s, 2 H) 3.68 (s, 3 H) 3.47-3.65 (m, 8 H) 3.30 (s, 3 H) |
| A-202 | 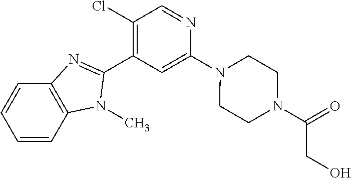 | 2-{4-[5-chloro- 4-(1-methyl-1H- benzimidazol- 2-yl)pyridin-2- yl]piperazin-1- yl}-2- oxoethanol | 386.20 | ¹H NMR (400 MHz, DMSO-d6) δ ppm 8.36 (s, 1 H) 7.71 (d, J = 7.83 Hz, 1 H) 7.66 (d, J = 7.83 Hz, 1 H) 7.32-7.38 (m, 1 H) 7.26-7.32 (m, 1 H) 7.13 (s, 1 H) 4.63 (t, J = 5.56 Hz, 1 H) 4.13 (d, J = 5.56 Hz, 2 H) 3.68 (s, 3 H) 3.55-3.65 (m, 6 H) 3.44-3.50 (m, 2 H) |
| A-203 | 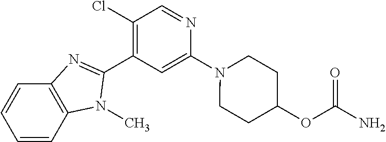 | 1-[5-chloro-4- (1-methyl-1H- benzimidazol- 2-yl)pyridin-2- yl]piperidin-4-yl carbamate | 386.3 | ¹H NMR (400 MHz, DMSO): δ 8.32 (s, 1 H), 7.71-7.63 (q, 2 H), 7.36-7.26 (m, 2 H), 7.11 (s, 1 H), 6.50 (broad, s, 2 H), 4.72-4.70 (m, 1 H), 4.02-3.98 (m, 2 H), 3.67 (s, 3 H), 1.90- 1.87 (m, 2 H), 1.54-1.49 (m, 2 H) |
| A-204 | 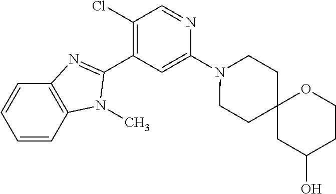 | 9-[5-chloro-4- (1-methyl-1H- benzimidazol- 2-yl)pyridin-2- yl]-1-oxa-9- azaspiro[5.5] undecan-4-ol | 413.20 | ¹H NMR (400 MHz, DMSO-d6) δ ppm 8.30 (s, 1 H) 7.70 (d, J = 7.6 Hz, 1 H) 7.64 (d, J = 8.1 Hz, 1 H) 7.32-7.44 (m, 1 H) 7.21-7.32 (m, 1 H) 7.06 (s, 1 H) 4.64 (d, J = 4.5 Hz, 1 H) 3.92 (d, J = 13.1 Hz, 2 H) 3.75-3.85 (m, 1 H) 3.63-3.75 (m, 4 H) 3.47-3.60 (m, 1 H) 3.22-3.28 (m, 1 H) 3.04- 3.19 (m, 1 H) 1.98 (br. s., 1 H) 1.74 (dd, J = 12.3, 3.7 Hz, 2 H) 1.51-1.68 (m, 2 H) 1.38- 1.49 (m, 1 H) 1.21-1.36 (m, 1 H) 1.15 (dd, J = 12.6, 10.6 Hz, 1 H) |
| A-205 | 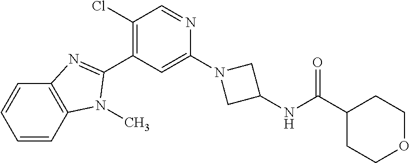 | N-{1-[5-chloro- 4-(1-methyl-1H- benzimidazol- 2-yl)pyridin-2- yl]azetidin-3- yl}tetrahydro- 2H-pyran-4- carboxamide | 426.2 | ¹H NMR (400 MHz, DMSO-d6) δ ppm 8.51-8.49 (d, 1 H), 8.31 (s, 1 H), 7.71-7.64 (dd, 2 H), 7.36-7.28 (m, 2 H), 6.68 (s, 1 H), 4.62-4.61 (m, 1 H), 4.27- 4.23 (m, 2 H) 3.86-3.79 (m, 4 H), 3.67 (s, 3 H), 3.39-3.26 (m, 2 H), 2.39-2.32 (m, 1 H), 1.59-1.51 (m, 4 H) |
| A-206 | 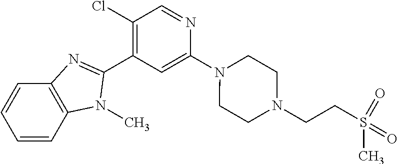 | 2-(5-chloro-2- {4-[2-(methyl- sulfonyl)ethyl] piperazin-1-yl} pyridin-4-yl)-1- methyl-1H- benzimidazole | 434.05 | ¹H NMR (400 MHz, DMSO-d6) δ ppm 8.33 (s, 1 H) 7.71 (d, J = 7.83 Hz, 1 H) 7.65 (d, J = 7.83 Hz, 1 H) 7.32-7.38 (m, 1 H) 7.26-7.32 (m, 1 H) 7.11 (s, 1 H) 3.68 (s, 3 H) 3.53-3.60 (m, 4 H) 3.33 (t, J = 6.82 Hz, 2 H) 3.04 (s, 3 H) 2.75 (t, J = 6.82 Hz, 2 H) 2.51- 2.56 (m, 4 H) |
| A-207 | 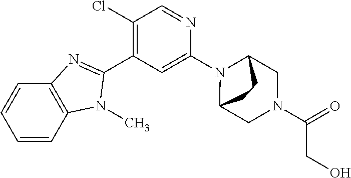 | 2-{(1R,5S)-8- [5-chloro-4-(1- methyl-1H- benzimidazol- 2-yl)pyridin-2- yl]-3,8-diaza- bicyclo[3.2.1] oct-3-yl}-2- oxoethanol | 412.15 | ¹H NMR (400 MHz, CHLOROFORM-d) δ ppm 8.31 (s, 1 H) 7.82-7.89 (m, 1 H) 7.33-7.49 (m, 3 H) 6.87 (s, 1 H) 4.53-4.69 (m, 2 H) 4.21-4.31 (m, 2 H) 4.04 (d, J = 15.16 Hz, 1 H) 3.75 (s, 3 H) 3.45-3.58 (m, 1 H) 3.16 (d, J = 11.12 Hz, 2 H) 2.63 (s, 1 H) 2.08-2.13 (m, 2 H) 1.70- 1.91 (m, 2 H) |
| A-208 | 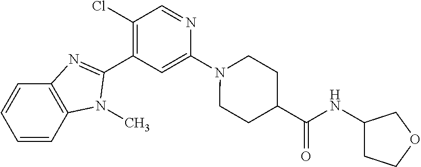 | 1-[5-chloro-4- (1-methyl-1H- benzimidazol- 2-yl)pyridin-2- yl]-N-(tetra- hydrofuran-3- yl)piperidine-4- carboxamide | 440.20/ 442.15 | ¹H NMR (400 MHz, DMSO-d6) δ ppm 8.31 (s, 1 H) 8.05 (d, J = 6.8 Hz, 1 H) 7.71 (d, J = 7.8 Hz, 1 H) 7.65 (d, J = 7.8 Hz, 1 H) 7.31-7.44 (m, 1 H) 7.23-7.31 (m, 1 H) 7.09 (s, 1 H) 4.34 (d, J = 12.9 Hz, 2 H) 4.15-4.25 (m, 1 H) 3.70-3.84 (m, 2 H) 3.68 (s, 3 H), 3.59- 3.67 (m, 1 H) 3.41 (dd, J = 8.8, 3.8 Hz, 1 H) 2.90 (t, J = 11.5 Hz, 2 H) 2.35-2.46 (m, 1 H) 1.94-2.12 (m, 1 H) 1.64- 1.79 (m, 3 H) 1.46-1.62 (m, 2 H) |
| A-209 | 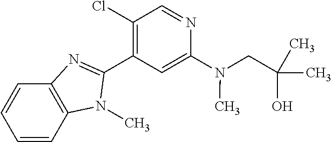 | 1-{[5-chloro-4- (1-methyl-1H- benzimidazol- 2-yl)pyridin-2-yl] (methyl)amino}- 2-methyl- propan-2-ol | 345.05/ 347.10 | ¹H NMR (400 MHz, DMSO-d6) δ ppm 8.25 (br. s., 1 H) 7.54- 7.82 (m, 2 H) 7.14-7.44 (m, 2 H) 6.94 (br. s., 1 H) 4.55 (br. s., 1 H) 3.67 (br. s., 3 H) 3.54 (br. s., 2 H) 3.11 (br. s., 3 H) 1.11 (br. s., 6 H) |
| A-210 | 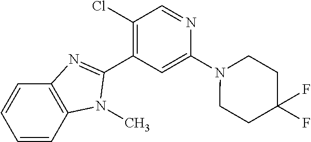 | 2-[5-chloro-2- (4,4-difluoro- piperidin-1- yl)pyridin-4-yl]- 1-methyl-1H- benzimidazole | 363.2 | ¹H NMR (400 MHz, CDCl3): δ 8.32 (s, 1 H), 7.89-7.88 (d, 1 H), 7.49-7.35 (m, 3 H), 6.96 (s, 1 H), 3.82-3.78 (m, 7 H), 2.12-2.03 (m, 4 H) |
| A-211 | 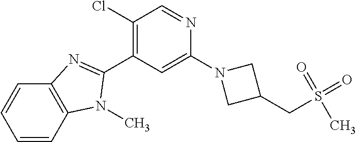 | 2-(5-chloro-2-{3- [(methylsulfonyl) methyl]azetidin- 1-yl}pyridin-4- yl)-1-methyl-1H- benzimidazole | 391.1 | ¹H NMR (400 MHz, DMSO-d6) δ ppm 8.31 (s, 1 H) 7.71 (d, J = 8.1 Hz, 1 H) 7.66 (d, J = 7.6 Hz, 1 H) 7.36 (t, J = 7.6 Hz, 1 H) 7.30 (t, 1 H) 6.67 (s, 1 H) 4.20 (t, J = 8.3 Hz, 2 H) 3.88 (dd, J = 8.1, 6.3 Hz, 2 H) 3.69 (s, 3 H) 3.58 (d, J = 7.3 Hz, 2 H) 3.21-3.31 (m, 1 H) 2.99 (s, 3 H) |
| A-212 | 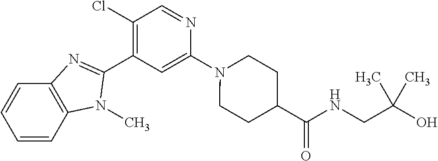 | 1-[5-chloro-4- (1-methyl-1H- benzimidazol- 2-yl)pyridin-2- yl]-N-(2- hydroxy-2- methylpropyl) piperidine-4- carboxamide | 442.15/ 444.25 | ¹H NMR (400 MHz, DMSO-d6) δ ppm 8.31 (s, 1 H) 7.58-7.76 (m, 3 H) 7.32-7.41 (m, 1 H) 7.22-7.32 (m, 1 H) 7.09 (s, 1 H) 4.41 (s, 1 H) 4.34 (d, J = 13.4 Hz, 2 H) 3.68 (s, 3 H) 3.02 (d, J = 6.1 Hz, 2 H) 2.91 (t, J = 11.6 Hz, 2 H) 2.46-2.58 (m, 1 H) 1.73 (d, J = 10.1 Hz, 2 H) 1.58 (d, J = 3.5 Hz, 2 H) 1.03 (s, 6 H) |
| A-213 | 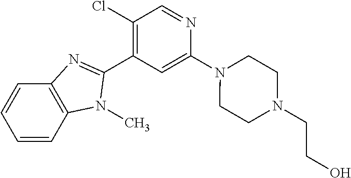 | 2-{4-[5-chloro- 4-(1-methyl-1H- benzimidazol- 2-yl)pyridin-2- yl]piperazin-1- yl}ethanol | 372.10/ 374.20 | ¹H NMR (400 MHz, DMSO-d6) δ ppm 8.32 (s, 1 H) 7.71 (d, J = 7.8 Hz, 1 H) 7.65 (d, J = 7.8 Hz, 1 H) 7.31-7.44 (m, 1 H) 7.22-7.32 (m, 1 H) 7.08 (s, 1 H) 4.43 (t, 1 H) 3.68 (s, 3 H) 3.46-3.59 (m, 6 H) 2.43 (t, J = 6.2 Hz, 2 H) |
| A-214 | 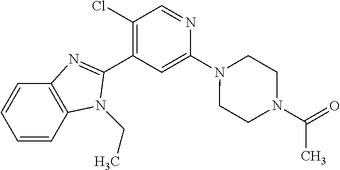 | 2-[2-(4-acetyl- piperazin-1-yl)- 5-chloropyridin- 4-yl]-1-ethyl-1H- benzimidazole | 384.10/ 386.20 | ¹H NMR (400 MHz, DMSO-d6) δ ppm 8.36 (s, 1 H) 7.70 (dd, J = 7.7, 4.2 Hz, 2 H) 7.31-7.42 (m, 1 H) 7.23-7.31 (m, 1 H) 7.14 (s, 1 H) 4.12 (q, J = 7.3 Hz, 2 H) 3.60-3.68 (m, 2 H) 3.55 (s, 6 H) 2.04 (s, 3 H) 1.23 (t, J = 7.2 Hz, 3 H) |
| A-215 | 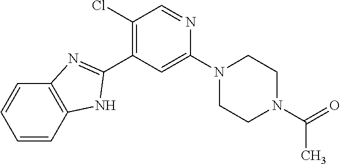 | 2-[2-(4-acetyl- piperazin-1-yl)- 5-chloropyridin- 4-yl]-1H- benzimidazole | 356.20/ 358.15 | ¹H NMR (400 MHz, DMSO-d6) δ ppm 12.85 (s, 1 H) 8.33 (s, 1 H) 7.74 (d, J = 7.6 Hz, 1 H) 7.61 (d, J = 7.6 Hz, 1 H) 7.33 (s, 1 H) 7.22-7.32 (m, 2 H) 3.61-3.69 (m, 2 H) 3.57 (s, 6 H) 2.05 (s, 3 H) |
| A-216 | 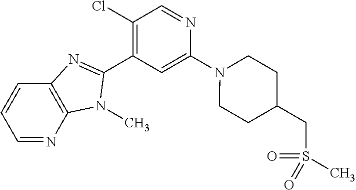 | 2-(5-chloro-2-{4- [(methylsulfonyl) methyl]piperidin- 1-yl}pyridin-4- yl)-3-methyl-3H- imidazo[4,5- b]pyridine | 420.2 | ¹H NMR (400 MHz, DMSO-d6) δ ppm 8.51 (dd, J = 4.7, 1.3 Hz, 1 H) 8.39 (s, 1 H) 8.22 (dd, J = 8.1, 1.3 Hz, 1 H) 7.43 (dd, J = 8.0, 4.8 Hz, 1 H) 7.20 (s, 1 H) 4.36 (d, J = 13.2 Hz, 2 H) 3.76 (s, 3 H) 3.20 (d, J = 6.4 Hz, 2 H) 2.92-3.12 (m, 5 H) 2.19-2.39 (m, 1 H) 1.97 (d, J = 11.1 Hz, 2 H) 1.25- 1.50 (m, 2 H) |

### Preparation of intermediate 7: 2-(3,6-dichloropyridin-2-yl)-1-methyl-1H-benzimidazole

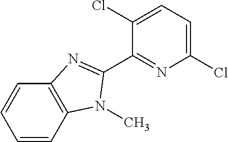

To a solution of 3,6-dichloropyridine-2-carboxylic acid (500 mg, 2.60 mmol) in pyridine (13 mL) was added triphenyl phosphite (1.21 g, 3.91 mmol) and N-methyl-1,2-phenylenediamine (318 mg, 2.60 mmol). The resulting solution was subjected to microwave irradiation at 160° C. for 30 mins. The crude reaction mixture was taken up in EtOAc and washed with 0.5 M CuSO₄ until the wash layers were no longer dark purple. The organic layer was then washed with water (3×), dried over MgSO₄, and concentrated to a dark red oil. The crude product was purified by Biotage flash chromatography (40 S column, loaded with DCM, eluted with EtOAc/heptane) to afford the title compound (270 mg, 37%) as a white foam. ¹H NMR (400 MHz, DMSO-d6) δ ppm 3.88 (s, 3H) 7.32-7.48 (m, 4H) 7.87 (d, J=4.04 Hz, 1H) 7.89 (d, J=3.54 Hz, 1H). m/z (APCI+) for C₁₃H₉Cl₂N₃ 278.05/280.00 (M+H)⁺.

### Preparation of example B1: 2-(3-chloro-6-piperazin-1-ylpyridin-2-yl)-1-methyl-1H-benzimidazole formate salt

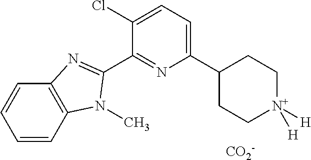

To a solution of 2-(3,6-dichloropyridin-2-yl)-1-methyl-1H-benzimidazole (260 mg, 0.935 mmol) and piperazine (805 mg, 9.35 mmol) in DMSO (4.7 mL) was added CsF (426 mg, 2.80 mmol). The mixture was heated to 100° C. for 1 h. The reaction was cooled to room temperature and diluted with water. The resulting solution was extracted with EtOAc (3×). The combined organics were washed with water (2×). LCMS indicated the presence of product in the wash layers. The wash layers were extracted with EtOAc (2×) and combined with the previous organic extracts. The organics were dried over MgSO₄ and concentrated. The crude material was purified by HPLC (formic acid/water/MeOH) to afford the title compound (255 mg, 73%) as a white solid. ¹H NMR (400 MHz, DMSO-d6) δ ppm 2.87 (d, J=4.04 Hz, 4H) 3.49-3.57 (m, 4H) 3.77 (s, 3H) 7.07 (d, J=9.09 Hz, 1H) 7.24-7.30 (m, 1H) 7.31-7.39 (m, 1H) 7.63 (d, J=8.08 Hz, 1H) 7.70 (d, J=8.08 Hz, 1H) 7.82 (d, J=9.09 Hz, 1H) 8.24 (s, 1H). m/z (APCI+) for C₁₇H₁₈ClN₅ 328.10/330.10 (M+H)⁺.

### Preparation of example B2: 2-[6-(4-acetylpiperazin-1-yl)-3-chloropyridin-2-yl]-1-methyl-1H-benzimidazole

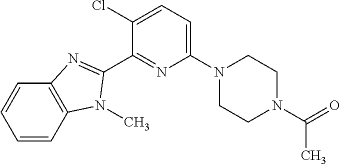

To a fine suspension of 2-(3-chloro-6-piperazin-1-ylpyridin-2-yl)-1-methyl-1H-benzimidazole formate salt (80 mg, 0.21 mmol) in DCM (2.14 mL) was added DIPEA (166 mg, 1.28 mmol) and acetyl chloride (84 mg, 1.07 mmol). The mixture was stirred at RT for 5 min. The reaction mixture was concentrated and purified by HPLC (formic acid/water/MeOH) to afford the title compound (26 mg, 29%) as a white solid. ¹H NMR (400 MHz, DMSO-d6) δ ppm 2.04 (s, 3H) 3.50-3.57 (m, 6H) 3.60 (d, J=5.81 Hz, 2H) 3.77 (s, 3H) 7.10 (d, J=9.09 Hz, 1H) 7.28 (td, J=7.58, 1.26 Hz, 1H) 7.35 (td, J=7.58, 1.26 Hz, 1H) 7.65 (s, 1H) 7.71 (d, J=7.83 Hz, 1H) 7.85 (d, J=9.09 Hz, 1H). m/z (APCI+) for C₁₉H₂₀ClN₅O 370.10/372.10 (M+H)⁺.

### Preparation of example B3: 4-[5-chloro-6-(1-methyl-1H-benzimidazol-2-yl)pyridin-2-yl]-N-methylpiperazine-1-carboxamide

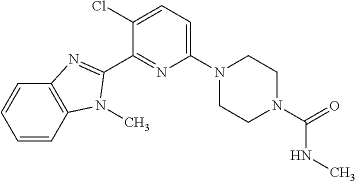

To a fine suspension of 2-(3-chloro-6-piperazin-1-ylpyridin-2-yl)-1-methyl-1H-benzimidazole formate salt (80 mg, 0.21 mmol) in DCM (2.14 mL) was added methyl isocyanate (61 mg, 1.07 mmol). After 5 min, the reaction had turned to a clear solution. The reaction mixture was concentrated and purified by SFC to afford the title compound (58 mg, 62%) as a white solid. ¹H NMR (400 MHz, DMSO-d6) δ ppm 2.58 (d, J=2.78 Hz, 3H) 3.35-3.43 (m, 4H) 3.48-3.57 (m, 4H) 3.77 (s, 3H) 6.48-6.57 (m, 1H) 7.10 (d, J=9.35 Hz, 1H) 7.28 (t, J=7.58 Hz, 1H) 7.35 (t, J=7.07 Hz, 1H) 7.64 (d, J=8.34 Hz, 1H) 7.71 (d, J=7.58 Hz, 1H) 7.83 (d, J=9.09 Hz, 1H). m/z (APCI+) for C₁₉H₂₁ClN₆O 385.10/387.10 (M+H)⁺.

### Preparation of example B4: 2-{3-chloro-6-[4-(methylsulfonyl)piperazin-1-yl]pyridin-2-yl}-1-methyl-1H-benzimidazole

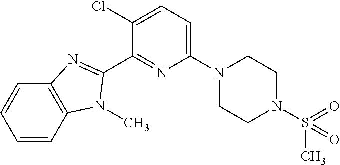

To a fine suspension of 2-(3-chloro-6-piperazin-1-ylpyridin-2-yl)-1-methyl-1H-benzimidazole formate salt (67 mg, 0.18 mmol) in DCM (2 mL) was added mesyl chloride (23.4 mg, 0.21 mmol) and DIPEA (26.4 mg, 0.21 mmol). After 15 min, TLC showed incomplete reaction. More mesyl chloride (23.4 mg, 0.21 mmol) and DIPEA (26.4 mg, 0.21 mmol) were added. After 10 min, reaction was complete. The crude material was purified by Biotage flash chromatography (25 S column, eluted with EtOAc/heptane) to afford the title compound (18 mg, 25%) as a white solid. ¹H NMR (400 MHz, DMSO-d6) δ ppm 2.90 (s, 3H) 3.21 (d, J=4.29 Hz, 4H) 3.66-3.72 (m, 4H) 3.77 (s, 3H) 7.14 (d, J=9.09 Hz, 1H) 7.28 (t, J=7.45 Hz, 1H) 7.35 (t, J=7.45 Hz, 1H) 7.64 (d, J=7.83 Hz, 1H) 7.71 (d, J=8.08 Hz, 1H) 7.87 (d, J=9.09 Hz, 1H). m/z (APCI+) for C₁₈H₂₀ClN₅O₂S 406.10/408.10 (M+H)⁺.

### Preparation of example B5: 4-[5-chloro-6-(1-methyl-1H-benzimidazol-2-yl)pyridin-2-yl]piperazine-1-carbaldehyde

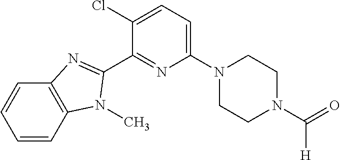

The title compound (44 mg, 69%) was isolated from the reaction described above (preparation of 2-{3-chloro-6-[4-(methylsulfonyl)piperazin-1-yl]pyridin-2-yl}-1-methyl-1H-benzimidazole) as a white solid. ¹H NMR (400 MHz, DMSO-d6) δ ppm 3.44-3.52 (m, 4 H) 3.53-3.58 (m, 2H) 3.58-3.65 (m, 2H) 3.77 (s, 3H) 7.14 (d, J=9.35 Hz, 1H) 7.28 (t, J=7.20 Hz, 1H) 7.35 (t, J=7.20 Hz, 1H) 7.64 (d, J=8.08 Hz, 1H) 7.71 (d, J=7.83 Hz, 1 H) 7.86 (d, J=9.09 Hz, 1H) 8.09 (s, 1H). m/z (APCI+) for C₁₈H₁₈ClN₅O 356.20/358.15 (M+H)⁺.

### Preparation of example B6: 1-[5-chloro-6-(1-methyl-1H-benzimidazol-2-yl)pyridin-2-yl]piperidin-4-ol

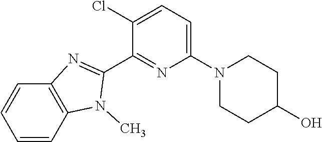

To a solution of 2-(3,6-dichloropyridin-2-yl)-1-methyl-1H-benzimidazole (100 mg, 0.36 mmol) and piperidin-4-ol (36.4 mg, 0.36 mmol) in DMSO (5.1 mL) was added CsF (328 mg, 2.16 mmol). The mixture was heated to 100° C. overnight. The reaction was cooled to room temperature and diluted with water, at which point solid product crashed out of solution. The solid was filtered off and washed thoroughly with water. The crude material was purified by Biotage flash chromatography (25 S column, eluted 1:19:80 NH₄OH/EtOH/EtOAc in heptane (10-50%) to afford the title compound (62 mg, 50%) as a white foam. ¹H NMR (400 MHz, DMSO-d6) δ ppm 7.77 (d, J=9.35 Hz, 1H) 7.70 (d, J=7.83 Hz, 1H) 7.63 (d, J=8.08 Hz, 1H) 7.34 (t, J=7.20 Hz, 1H) 7.27 (t, J=7.20 Hz, 1 H) 7.07 (d, J=9.09 Hz, 1H) 4.72 (d, J=4.04 Hz, 1H) 4.01 (t, J=3.79 Hz, 1H) 3.98 (t, J=4.17 Hz, 1H) 3.77 (s, 3H) 3.65-3.76 (m, 1H) 3.12-3.23 (m, 2H) 1.72-1.83 (m, 2 H) 1.30-1.44 (m, 2H). m/z (APCI+) for C₁₈H₁₉ClN₄O 343.15 (M+H)⁺.

### Preparation of intermediate 8: 2-(6-bromopyridin-2-yl)-1-methyl-1H-benzimidazole

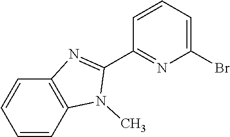

To a solution of 6-bromopyridine-2-carbaldehyde (430 mg, 2.31 mmol) in DMA (7.7 mL) was added N-methyl-1,2-phenylenediamine (0.263 mL, 2.31 mmol) and sulfur (74 mg, 2.31 mmol). The mixture was stirred at room temperature for 2 h then heated to 40° C. overnight. Water was added, at which point solid product crashed out. The solid was filtered off, washed thoroughly with water, and left to dry overnight. The title compound (457 mg, 69%) was collected as a light brown solid and was used without further purification. ¹H NMR (400 MHz, DMSO-d6) δ ppm 8.34 (1H, dd, J=7.83, 0.76 Hz) 7.96 (1H, t, J=7.96 Hz) 7.79 (1H, dd, J=7.96, 0.63 Hz) 7.74 (1H, d, J=7.83 Hz) 7.67 (1H, d, J=8.08 Hz) 7.36 (1H, td, J=7.64, 1.14 Hz) 7.27-7.33 (1H, m) 4.21 (3H, s). m/z (APCI+) for C₁₃H₁₀BrN₃ 288.00/290.00 (M+H)⁺.

### Preparation of intermediate 9: 1-methyl-2-(6-piperazin-1-ylpyridin-2-yl)-1H-benzimidazole

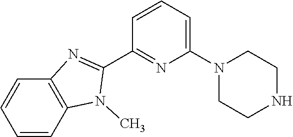

To a solution of 2-(6-bromopyridin-2-yl)-1-methyl-1H-benzimidazole (450 mg, 1.56 mmol) and piperazine (1.0 g, 11.9 mmol) in isopropanol (7.2 mL) was added DMSO to dissolve starting materials (2 mL). Then CsF (1.1 g, 7.14 mmol) was added, and the mixture was heated to reflux for three days. The reaction was cooled to RT, and the IPA was removed under reduced pressure. The residue was partitioned between water and EtOAc. The layers were separated, and the aqueous layer was extracted with EtOAc (2×). The combined organics were washed with water (2×) and brine (1×), dried over MgSO₄, and concentrated. The crude material was purified by Biotage flash chromatography (25S column, eluted 1:19:80 NH₄OH/EtOH/EtOAc in heptane (10-50%)) to afford the title compound (372 mg, 81%) as a pale yellow foam. ¹H NMR (400 MHz, DMSO-d6) δ ppm 7.83 (1H, d, J=7.33 Hz) 7.73 (1H, d, J=7.32 Hz) 7.66 (1H, t, J=7.96 Hz) 7.42 (1H, d, J=7.33 Hz) 7.28-7.37 (2H, m) 6.74 (1H, d, J=8.59 Hz) 4.26 (3H, s) 3.56-3.67 (4H, m) 3.01-3.11 (4H, m). m/z (APCI+) for C₁₇H₁₉N₅ 294.25 (M+H)⁺.

### Preparation of example B7: 1-methyl-2-{6-[4-(methylsulfonyl)piperazin-1-yl]pyridin-2-yl}-1H-benzimidazole

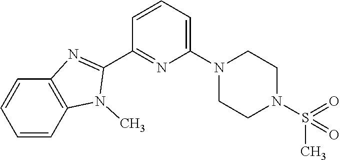

To a solution of 1-methyl-2-(6-piperazin-1-ylpyridin-2-yl)-1H-benzimidazole (65 mg, 0.22 mmol) in DCM (2.2 mL) were added DIPEA (28.7 mg, 0.22 mmol) and methanesulfonyl chloride (25.4 mg, 0.22 mmol). After 10 min, TLC showed complete reaction. The reaction mixture was loaded directly onto a pre-conditioned Biotage column for purification (25 S column, eluted with 50-100% EtOAc/heptane). The title compound (74 mg, 90%) was recovered as a white solid. ¹H NMR (400 MHz, DMSO-d6) δ ppm 7.74-7.81 (1H, m) 7.69 (1H, d, J=7.83 Hz) 7.65 (1H, d, J=8.08 Hz) 7.62 (1 H, d, J=7.58 Hz) 7.29-7.35 (1H, m) 7.22-7.29 (1H, m) 7.05 (1H, d, J=8.59 Hz) 4.20 (3H, s) 3.66-3.79 (4H, m) 3.19-3.30 (4H, m) 2.92 (3H, s). m/z (APCI+) for C₁₈H₂₁N₅O₂S 372.10 (M+H)⁺.

The following examples listed in Table 2 were prepared with appropriate substitutions in analogous ways to examples B1-B7.

TABLE 2

| | | | LRMS | |
|---|---|---|---|---|
| Example | | Compound | m/z | |
| Number | Structure | Name | (M + H) | ¹H NMR |
| B-1 | 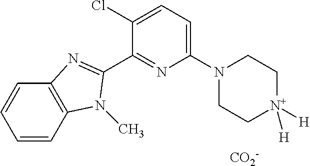 | 2-(3-chloro-6- piperazin-1- ylpyridin-2-yl)- 1-methyl-1H- benzimidazole formate salt | 328.15 | ¹H NMR (400 MHz, DMSO-d6) δ ppm 8.24 (s, 1 H) 7.82 (d, J = 9.09 Hz, 1 H) 7.70 (d, J = 8.08 Hz, 1 H) 7.63 (d, J = 8.08 Hz, 1 H) 7.31-7.39 (m, 1 H) 7.24-7.30 (m, 1 H) 7.07 (d, J = 9.09 Hz, 1 H) 3.77 (s, 3 H) 3.49-3.57 (m, 4 H) 2.87 (d, J = 4.04 Hz, 4 H) |
| B-2 | 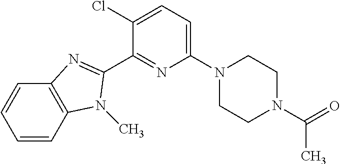 | 2-[6-(4- acetylpiperazin- 1-yl)-3- chloropyridin- 2-yl]-1-methyl- 1H- benzimidazole | 370.15 | ¹H NMR (400 MHz, DMSO-d6) δ ppm 7.85 (d, J = 9.09 Hz, 1 H) 7.71 (d, J = 7.83 Hz, 1 H) 7.65 (s, 1 H) 7.35 (td, J = 7.58, 1.26 Hz, 1 H) 7.28 (td, J = 7.58, 1.26 Hz, 1 H) 7.10 (d, J = 9.09 Hz, 1 H) 3.77 (s, 3 H) 3.60 (d, J = 5.81 Hz, 2 H) 3.50-3.57 (m, 6 H) 2.04 (s, 3 H) |
| B-3 | 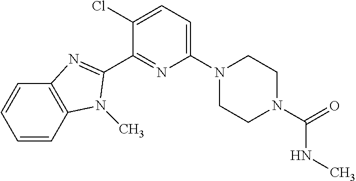 | 4-[5-chloro-6- (1-methyl-1H- benzimidazol- 2-yl)pyridin-2- yl]-N- methylpiperazine- 1- carboxamide | 385.15 | ¹H NMR (400 MHz, DMSO-d6) δ ppm 7.83 (d, J = 9.09 Hz, 1 H) 7.71 (d, J = 7.58 Hz, 1 H) 7.64 (d, J = 8.34 Hz, 1 H) 7.35 (t, J = 7.07 Hz, 1 H) 7.28 (t, J = 7.58 Hz, 1 H) 7.10 (d, J = 9.35 Hz, 1 H) 6.48-6.57 (m, 1 H) 3.77 (s, 3 H) 3.48-3.57 (m, 4 H) 3.35-3.43 (m, 4 H) 2.58 (d, J = 2.78 Hz, 3 H) |
| B-4 | 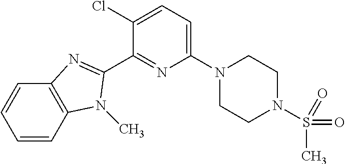 | 2-{3-chloro-6- [4- (methylsulfonyl) piperazin-1- yl]pyridin-2- yl}-1-methyl- 1H- benzimidazole | 406.15 | ¹H NMR (400 MHz, DMSO-d6) δ ppm 7.87 (d, J = 9.09 Hz, 1 H) 7.71 (d, J = 8.08 Hz, 1 H) 7.64 (d, J = 7.83 Hz, 1 H) 7.35 (t, J = 7.45 Hz, 1 H) 7.28 (t, J = 7.45 Hz, 1 H) 7.14 (d, J = 9.09 Hz, 1 H) 3.77 (s, 3 H) 3.66- 3.72 (m, 4 H) 3.21 (d, J = 4.29 Hz, 4 H) 2.90 (s, 3 H) |
| B-5 | 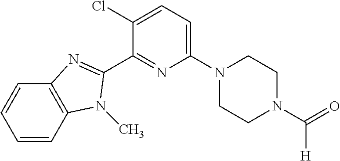 | 4-[5-chloro-6- (1-methyl-1H- benzimidazol- 2-yl)pyridin-2- yl]piperazine- 1- carbaldehyde | 356.20 | ¹H NMR (400 MHz, DMSO-d6) δ ppm 8.09 (s, 1 H) 7.86 (d, J = 9.09 Hz, 1 H) 7.71 (d, J = 7.83 Hz, 1 H) 7.64 (d, J = 8.08 Hz, 1 H) 7.35 (t, J = 7.20 Hz, 1 H) 7.28 (t, J = 7.20 Hz, 1 H) 7.14 (d, J = 9.35 Hz, 1 H) 3.77 (s, 3 H) 3.58-3.65 (m, 2 H) 3.53- 3.58 (m, 2 H) 3.44-3.52 (m, 4 H) |
| B-6 | 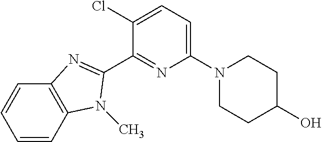 | 1-[5-chloro-6- (1-methyl-1H- benzimidazol- 2-yl)pyridin-2- yl]piperidin-4- ol | 343.15 | ¹H NMR (400 MHz, DMSO-d6) δ ppm 7.77 (d, J = 9.35 Hz, 1 H) 7.70 (d, J = 7.83 Hz, 1 H) 7.63 (d, J = 8.08 Hz, 1 H) 7.34 (t, J = 7.20 Hz, 1 H) 7.27 (t, J = 7.20 Hz, 1 H) 7.07 (d, J = 9.09 Hz, 1 H) 4.72 (d, J = 4.04 Hz, 1 H) 4.01 (t, J = 3.79 Hz, 1 H) 3.98 (t, J = 4.17 Hz, 1 H) 3.77 (s, 3 H) 3.65- 3.76 (m, 1 H) 3.12-3.23 (m, 2 H) 1.72-1.83 (m, 2 H) 1.30-1.44 (m, 2 H) |
| B-7 | 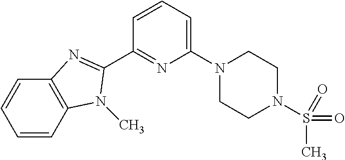 | 1-methyl-2-{6- [4- (methylsulfonyl) piperazin-1- yl]pyridin-2- yl}-1H- benzimidazole | 372.10 | ¹H NMR (400 MHz, DMSO-d6) δ ppm 7.74-7.81 (1 H, m) 7.69 (1 H, d, J = 7.83 Hz) 7.65 (1 H, d, J = 8.08 Hz) 7.62 (1 H, d, J = 7.58 Hz) 7.29- 7.35 (1 H, m) 7.22-7.29 (1 H, m) 7.05 (1 H, d, J = 8.59 Hz) 4.20 (3 H, s) 3.66-3.79 (4 H, m) 3.19-3.30 (4 H, m) 2.92 (3 H, s) |
| B-8 | 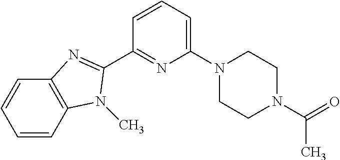 | 2-[6-(4- acetylpiperazin- 1-yl)pyridin- 2-yl]-1-methyl- 1H- benzimidazole | 336.25 | ¹H NMR (400 MHz, DMSO-d6) δ ppm 7.76 (1 H, t, J = 7.96 Hz) 7.69 (1 H, d, J = 7.83 Hz) 7.64 (1 H, d, J = 8.08 Hz) 7.61 (1 H, d, J = 7.58 Hz) 7.32 (1 H, t, J = 7.07 Hz) 7.25 (1 H, t, J = 7.20 Hz) 7.01 (1 H, d, J = 8.34 Hz) 4.21 (3 H, s) 3.54-3.69 (8 H, m) 2.06 (3 H, s) |
| B-9 | 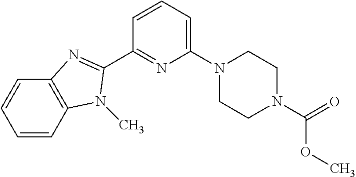 | methyl 4-[6- (1-methyl-1H- benzimidazol- 2-yl)pyridin-2- yl]piperazine- 1-carboxylate | 352.15 | ¹H NMR (400 MHz, DMSO-d6) δ ppm 7.75 (1 H, t, J = 7.83 Hz) 7.69 (1 H, d, J = 7.58 Hz) 7.64 (1 H, d, J = 7.83 Hz) 7.60 (1 H, d, J = 7.58 Hz) 7.31 (1 H, t, J = 7.58 Hz) 7.25 (1 H, t, J = 7.45 Hz) 7.00 (1 H, d, J = 8.59 Hz) 4.20 (3 H, s) 3.64 (3 H, s) 3.59- 3.63 (4 H, m) 3.51-3.57 (4 H, m) |
| B-10 | 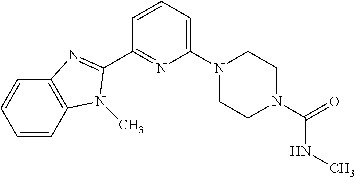 | N-methyl-4-[6- (1-methyl-1H- benzimidazol- 2-yl)pyridin-2- yl]piperazine- 1- carboxamide | 351.25 | ¹H NMR (400 MHz, DMSO-d6) δ ppm 7.72-7.77 (1 H, m) 7.69 (1 H, d, J = 7.58 Hz) 7.64 (1 H, d, J = 8.08 Hz) 7.60 (1 H, d, J = 7.33 Hz) 7.29- 7.34 (1 H, m) 7.22-7.28 (1 H, m) 7.01 (1 H, d, J = 8.59 Hz) 6.54 (1 H, q, J = 4.04 Hz) 4.21 (3 H, s) 3.54- 3.60 (4 H, m) 3.43-3.49 (4 H, m) 2.60 (3 H, d, J = 4.29 Hz) |
| B-11 | 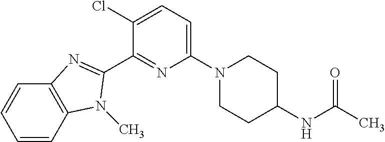 | N-{1-[5- chloro-6-(1- methyl-1H- benzimidazol- 2-yl)pyridin-2- yl]piperidin-4- yl}acetamide | 384.10 | ¹H NMR (400 MHz, DMSO-d6) δ ppm 7.81 (d, 1 H) 7.78 (d, J = 9.09 Hz, 1 H) 7.70 (d, J = 7.83 Hz, 1 H) 7.63 (d, J = 7.83 Hz, 1 H) 7.31-7.37 (m, 1 H) 7.25-7.30 (m, 1 H) 7.09 (d, J = 9.09 Hz, 1 H) 4.19 (d, J = 13.64 Hz, 2 H) 3.78-3.87 (m, 1 H) 3.77 (s, 3 H) 2.99-3.10 (m, 2 H) 1.78 (s, 3 H) 1.78 (dd, J = 15.79, 4.17 Hz, 2 H) 1.28-1.41 (m, 2 H) |
| B-12 | 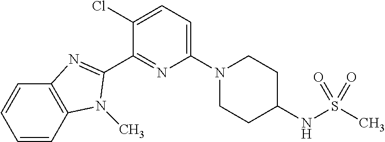 | N-{1-[5- chloro-6-(1- methyl-1H- benzimidazol- 2-yl)pyridin-2- yl]piperidin-4- yl}methane- sulfonamide | 420.10 | ¹H NMR (400 MHz, DMSO-d6) δ ppm 7.79 (d, J = 9.09 Hz, 1 H) 7.71 (d, J = 7.83 Hz, 1 H) 7.63 (d, J = 8.08 Hz, 1 H) 7.31-7.39 (m, 1 H) 7.25- 7.30 (m, 1 H) 7.12 (br. s., 1 H) 7.09 (d, J = 9.35 Hz, 1 H) 4.19 (d, J = 12.63 Hz, 2 H) 3.77 (s, 3 H) 3.39-3.51 (m, 1 H) 2.99-3.10 (m, 2 H) 2.94 (s, 3 H) 1.89 (dd, J = 12.63, 2.53 Hz, 2 H) 1.36-1.49 (m, 2 H) |
| B-13 | 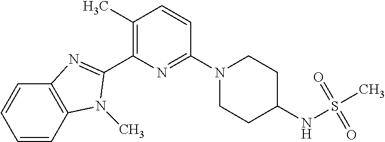 | N-{1-[5- methyl-6-(1- methyl-1H- benzimidazol- 2-yl)pyridin-2- yl]piperidin-4- yl}methane- sulfonamide | 400.5 | ¹H NMR (500 MHz, DMSO-d₆) ppm 1.33-1.48 (m, 2 H) 2.27 (s, 3 H) 2.46 (br. s., 2 H) 2.89 (s, 3 H) 2.92-3.02 (m, 2 H) 3.81 (s, 3 H) 4.14 (d, J = 13.17 Hz, 2 H) 6.93 (d, J = 8.78 Hz, 1 H) 7.01 (d, J = 7.32 Hz, 1 H) 7.22 (t, J = 7.56 Hz, 1 H) 7.28 (t, J = 7.56 Hz, 1 H) 7.58 (d, J = 8.30 Hz, 2 H) 7.65 (d, J = 8.78 Hz, 1 H) |

### Preparation of intermediate 9: 5-chloro-N-[2-(methylamino)phenyl]-2-(methylthio)pyrimidine-4-carboxamide

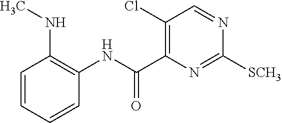

A mixture of 5-chloro-2-(methylthio)pyrimidine-4-carboxylic acid (15 g, 73.5 mmol), HATU (142 g, 110.3 mmol) and DIPEA (28.5 g, 220.6 mmol) in DMF (300 mL) was stirred at room temperature for 15 min. N-methyl-O-phenylenediamine (9 g, 73.5 mmol) was added in one portion. The resulting mixture was stirred at room temperature overnight. The solvent was evaporated and the resulting residue poured into 500 mL water. The mixture was filtered and the cake was collected and dried in vacuo to give the title compound (18 g, 80%) as a brown solid.

### Preparation of intermediate 10: 2-[5-chloro-2-(methylthio)pyrimidin-4-yl]-1-methyl-1H-benzimidazole

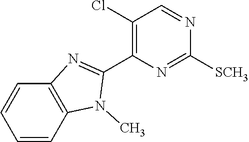

5-chloro-N-[2-(methylamino)phenyl]-2-(methylthio)pyrimidine-4-carboxamide (18 g, 58.44 mmol) in AcOH (300 mL) was stirred at 90˜100° C. for 4 hours. TLC (petroleum ether:EtOAc=4:1) show the reaction was completed. The mixture was concentrated and then adjusted pH=7-8 with NaHCO₃ and extracted with EtOAc (300 mL×3). The combined organic layers were washed with brine, dried over Na₂SO₄ and concentrated. The residue was purified by silica gel chromatography (petroleum ether:EtOAc=20:1 to 3:1) to give the title compound (12.7 g, 68%) as yellow solid.

### Preparation of intermediate 11: 2-[5-chloro-2-(methylsulfonyl)pyrimidin-4-yl]-1-methyl-1H-benzimidazole

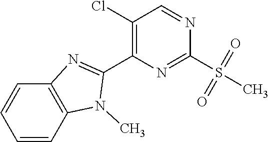

To a solution of 2-[5-chloro-2-(methylthio)pyrimidin-4-yl]-1-methyl-1H-benzimidazole (11 g, 37.93 mmol) in THF/H₂O (250 mL) was added Oxone® (46.6 g, 75.86 mmol) (Oxone®=potassium peroxomonosulfate). The resulting mixture was stirred at room temperature for 3 hours. TLC (petroleum ether:EtOAc=3:1) show the reaction was complete. The reaction mixture was extracted with dichloromethane (500 mL×4) and the combined organic layers were washed with brine, dried over sodium sulfate and concentrated to give the title compound (12 g, 100%) as yellow solid. ¹H NMR (400 MHz, CDCl₃): δ ppm 8.99 (s, 1H), 7.87-7.85 (d, 1H), 7.45-7.38 (m, 1H), 7.35-7.31 (m, 1H), 4.06-4.04 (d, 3H), 3.33 (s, 3H). m/z for C₁₃H₁₁N₄ClO₂S 323.30 (M+H)⁺.

### Preparation of intermediate 12: 2-(5-chloro-2-piperazin-1-ylpyrimidin-4-yl)-1-methyl-1H-benzimidazole

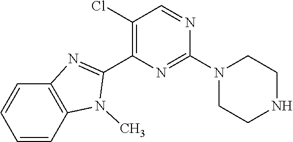

The mixture of 2-[5-chloro-2-(methylsulfonyl)pyrimidin-4-yl]-1-methyl-1H-benzimidazole (3.8 g, 11.8 mmol) and piperazine (2.03 g, 23.6 mmol) in THF (50 mL) was refluxed overnight. TLC (dichloromethane:methanol=10:1) showed the reaction was complete. The reaction mixture was concentrated and purified by silica gel chromatography (petroleum ether:EtOAc=1:10 to dichloromethane:methanol=100:1) to give the title compound (2.5 g, 65%) as yellow solid.

### Preparation of Example C1: 2-[2-(4-Acetylpiperazin-1-yl)-5-chloropyrimidin-4-yl]-1-methyl-1H-benzimidazole

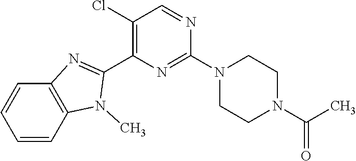

The mixture of compound 2-(5-chloro-2-piperazin-1-ylpyrimidin-4-yl)-1-methyl-1H-benzimidazole (0.4 g, 1.220 mmol) and Et₃N (370 mg, 3.68 mmol) in dichloromethane (8 mL) was stirred at room temperature under N₂ atmosphere. Acetyl chloride (143 mg, 1.829 mmol) was added in one portion and the resulting mixture was stirred at room temperature for 3 hours. TLC (dichloromethane:methanol=10:1) showed the reaction was complete. The reaction mixture was concentrated and purified by silica gel chromatography (petroleum ether:EtOAc=2:1 to 0:1) to afford the title compound (300 mg, 66%) as white solid. ¹H NMR (400 MHz, DMSO-d₆): δ ppm 8.70 (s, 1H), 7.75-7.77 (d, 1H), 7.69-7.71 (d, 1H), 7.39-7.41 (m, 1H), 7.31-7.36 (m, 1H), 3.33 (s, 3H), 3.82-3.83 (d, 2H), 3.75-3.76 (d, 2H), 3.55-3.58 (m, 4H), 2.06 (s, 3H). m/z for C₁₈H₁₉N₆C10 371.10 (M+H)⁺.

### Preparation of Example C2: 2-[2-(4-Acetylpiperazin-1-yl)pyrimidin-4-yl]-1-methyl-1H-benzimidazole

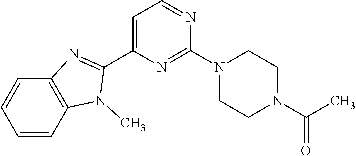

The mixture of 2-[2-(4-acetylpiperazin-1-yl)-5-chloropyrimidin-4-yl]-1-methyl-1H-benzimidazole (100 mg, 0.27 mol), NaOH (20 mg) and Pd/C (40 mg) in methanol (20 mL) was stirred at room temperature under H₂ atmosphere (45 psi) overnight. TLC (petroleum ether:EtOAc=3:1) showed the reaction was complete. The reaction mixture was filtered and purified by silica gel chromatography (dichloromethane:methanol=50:1 to 20:1) to give the title compound (94 mg, 98%) as a white solid. ¹H NMR (400 MHz, CDCl₃): δ ppm 8.43-8.44 (d, 1H), 7.77-7.79 (d, 1H), 7.53-7.55 (d, 1H), 7.38-7.4 (d, 1H), 7.25-7.34 (m, 2H), 4.21 (s, 3H), 3.82-3.90 (m, 4H), 3.70-3.71 (d, 2H), 3.53-3.54 (m, 2H), 2.11 (s, 3H). m/z for C₁₈H₂₀N₆O 337.50 (M+H)⁺.

### Preparation of Example C7: 4-[5-Chloro-4-(1-methyl-1H-benzimidazol-2-yl)pyrimidin-2-yl]-N-methylpiperazine-1-carboxamide

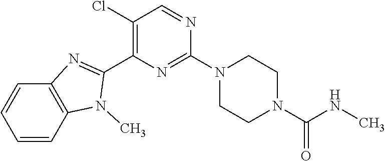

The mixture of 2-(5-chloro-2-piperazin-1-ylpyrimidin-4-yl)-1-methyl-1H-benzimidazole (650 mg, 1.98 mmol) and Et₃N (1 g, 9.9 mmol) in dichloromethane (8 mL) was stirred at −30° C., followed with adding triphosgene (196 mg, 0.66 mmol) in dichloromethane (2 mL) dropwise. The resulting mixture was stirred at −30° C. to room temperature for 2 hours. TLC (dichloromethane:methanol=10:1) indicated the reaction was complete. MeNH₂ (0.6 g, 19.98 mmol) was added. The resulting mixture was stirred at room temperature for 2 hours. LC-MS indicated the reaction was complete. The reaction mixture was concentrated and purified by silica gel chromatography (petroleum ether:EtOAc=2:1 to 0:1) to give the title compound (148 mg, 19%) as a light yellow solid. ¹H NMR (400 MHz, CDCl₃): δ ppm 8.40 (s, 1H), 7.82-7.84 (d, 1H), 7.26-7.39 (m, 3H), 4.38-4.39 (d, 1H), 3.79-3.84 (m, 7H), 3.33-3.49 (m, 4H), 2.77-2.78 (d, 3H). m/z for C₁₈H₂₀ClN₇O 386.20 (M+H)⁺.

### Preparation of Example C8: N-Methyl-4-[4-(1-methyl-1H-benzimidazol-2-yl)pyrimidin-2-yl]piperazine-1-carboxamide

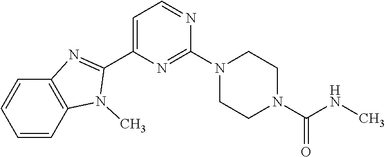

A mixture of 4-[5-chloro-4-(1-methyl-1H-benzimidazol-2-yl)pyrimidin-2-yl]-N-methylpiperazine-1-carboxamide (130 mg, 0.338 mol), NaOH (28 mg) and Pd/C (50 mg) in methanol (25 mL) was stirred at room temperature under H₂ atmosphere (45 psi) overnight. TLC (petroleum ether:EtOAc=3:1) showed the reaction was complete. The reaction mixture was filtered and purified by silica gel chromatography (dichloromethane:methanol=50:1 to 20:1) to give the title compound (84 mg, 80%) as a light yellow solid. ¹H NMR (400 MHz, CDCl₃): δ ppm 8.41-8.42 (d, 1H), 7.76-7.78 (d, 1H), 7.52-7.58 (d, 1H), 7.20-7.51 (m, 3H), 4.48-4.49 (t, 1H), 4.21 (s, 3H), 3.84-3.87 (m, 4H), 3.45-3.48 (m, 4H), 2.78-2.80 (d, 3H). m/z for C₁₈H₂₁N₇O 352.00 (M+H)⁺.

### Preparation of Example C9: N-{1-[5-chloro-4-(1-methyl-1H-benzimidazol-2-yl)pyrimidin-2-yl]piperidin-4-yl}methanesulfonamide

### Preparation of intermediate 13: Tert-butyl {1-[5-chloro-4-(1-methyl-1H-benzimidazol-2-yl)pyrimidin-2-yl]piperidin-4-yl}carbamate

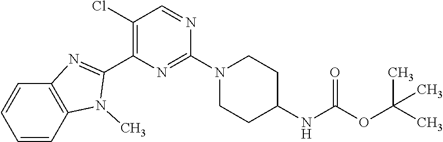

The mixture of 2-[5-chloro-2-(methylsulfonyl)pyrimidin-4-yl]-1-methyl-1H-benzimidazole (1 g, 3.1 mmol) and 4-Boc-aminopiperidine (0.745 g, 3.7 mmol) in THF (30 mL) was refluxed overnight. TLC (petroleum ether:EtOAc=1:1) showed the reaction was complete. The reaction mixture was concentrated and purified by silica gel chromatography (petroleum ether:EtOAc=10:1 to 3:1) to give the title compound (0.75 g, 54.6%) as a yellow solid.

### Preparation of intermediate 14: 1-[5-chloro-4-(1-methyl-1H-benzimidazol-2-yl)pyrimidin-2-yl]piperidin-4-amine

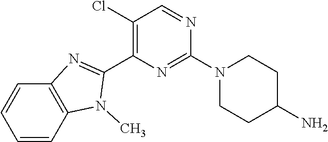

To a solution of compound 7 (750 mg, 1.7 mmol) in 1,4-dioxane (10 mL) was added HCl(g)/dioxane (10 mL, 4M). The resulting mixture was stirred at room temperature for 2 hours. TLC (petroleum ether:EtOAc=1:1) showed the reaction was complete. The reaction mixture was concentrated to give the title compound (800 mg, ˜100%) as yellow solid.

### Preparation of Example C9: N-{1-[(5-chloro-4-(1-methyl-1H-benzimidazol-2-yl)pyrimidin-2-yl]piperidin-4-yl}methanesulfonamide

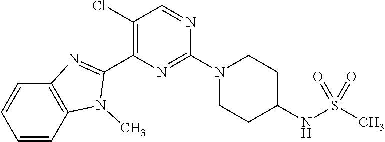

The mixture of compound 1-[5-chloro-4-(1-methyl-1H-benzimidazol-2-yl)pyrimidin-2-yl]piperidin-4-amine (0.4 g, 0.85 mmol) and Et₃N (430 mg, 4.25 mmol) in dichloromethane (10 mL) was stirred at room temperature under N₂ atmosphere. Methanesulfonyl chloride (200 mg, 1.7 mmol) was added in one portion. The resulting mixture was stirred at room temperature for 54 hours. TLC (petroleum ether:EtOAc=1:1) showed the reaction was complete. The reaction mixture was concentrated and purified by silica gel chromatography (petroleum ether:EtOAc=10:1 to 3:1) to afford the title compound (110 mg, 30%) as a light yellow solid. ¹H NMR (400 MHz, DMSO-d₆): δ ppm 8.54 (s, 1H), 7.74-7.76 (d, 1H), 7.67-7.69 (d, 1H), 7.37-7.40 (m, 1H), 7.29-7.33 (m, 1H), 7.14-7.16 (m, 1H), 4.44-4.47 (t, 2H), 3.89 (s, 3H), 3.47 (m, 1H), 3.15-3.23 (m, 2H), 2.94 (s, 3H), 1.91-1.93 (m, 2H), 1.37-1.45 (m, 2H). m/z for C₁₈H₂₁ClN₆O₂S 421.30 (M+H)⁺.

The following examples listed in Table 3 were prepared with appropriate substitutions in analogous ways to examples C1-C9 using appropriate reagents.

TABLE 3

| | | | LRMS | |
|---|---|---|---|---|
| Example | | | m/z | |
| Number | Structure | Compound Name | (M + H) | ¹H NMR |
| C-1 | 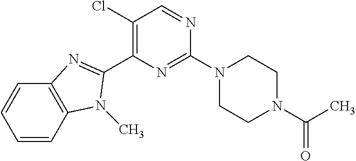 | 2-[2-(4- Acetylpiperazin- 1-yl)-5- chloropyrimidin- 4-yl]-1-methyl-1H- benzimidazole | 371.10 | ¹H NMR (400 MHz, DMSO-d6): δ ppm 8.70 (s, 1 H), 7.75- 7.77 (d, 1H), 7.69-7.71 (d, 1H), 7.39-7.41 (m, 1H), 7.31-7.36 (m, 1H), 3.33 (s, 3H), 3.82-3.83 (d, 2H), 3.75-3.76 (d, 2H), 3.55-3.58 (m, 4H), 2.06 (s, 3H) |
| C-2 | 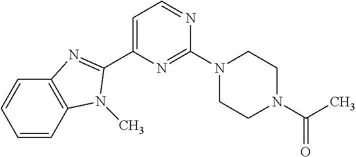 | 2-[2-(4- Acetylpiperazin- 1-yl)pyrimidin- 4-yl]-1-methyl-1H- benzimidazole | 337.50 | ¹H NMR (400 MHz, CDCl3): δ ppm 8.43- 8.44 (d, 1H), 7.77- 7.79 (d, 1H), 7.53- 7.55 (d, 1H), 7.38-7.4 (d, 1H), 7.25-7.34 (m, 2H), 4.21 (s, 3H), 3.82-3.90 (m, 4H), 3.70-3.71 (d, 2H), 3.53-3.54 (m, 2H), 2.11 (s, 3H) |
| C-3 | 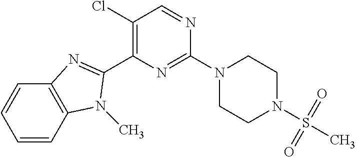 | 2-{5-Chloro-2-[4- (methylsulfonyl) piperazin-1- yl]pyrimidin-4- yl}-1-methyl-1H- benzimidazole | 371.10 | ¹H NMR (400 MHz, CDCl3): δ ppm 8.53 (s, 1H), 7.93-7.95 (d, 1H), 7.38-7.51 (m, 3H), 4.02-4.06 (m, 4H), 3.94 (s, 3H), 3.33-3.35 (t, 4H), 2.84 (s, 3H) |
| C-4 | 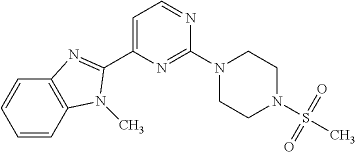 | 1-Methyl-2-{2-[4- (methylsulfonyl) piperazin-1- yl]pyrimidin-4- yl}-1H- benzimidazole | 373.20 | ¹H NMR (400 MHz, CDCl3): δ ppm 8.43- 8.44 (d, 1H), 7.77- 7.79 (d, 1H), 7.54- 7.55 (d, 1H), 7.38- 7.40 (d, 1H), 7.25- 7.34 (m, 2H), 4.20 (s, 3H), 3.97-3.99 (m, 4H), 3.27-3.29 (m, 4H), 2.75 (s, 3H) |
| C-5 | 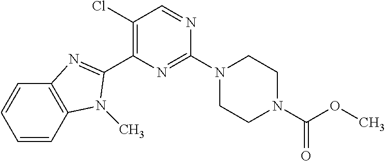 | Methyl 4-[5- chloro-4-(1- methyl-1H- benzimidazol- 2-yl)pyrimidin- 2-yl]piperazine- 1-carboxylate | 387.30 | ¹H NMR (400 MHz, CDCl3): δ ppm 8.51- 8.52 (d, 1H), 7.93- 7.95 (t, 1H), 7.30-7.50 (m, 3H), 3.40 (s, 3H), 3.88-3.91 (m, 4H), 3.79 (s, 3H), 3.59-3.62 (m, 4H) |
| C-6 | 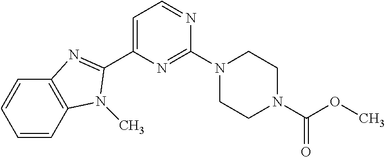 | Methyl 4-[4-(1- methyl-1H- benzimidazol- 2-yl)pyrimidin- 2-yl]piperazine- 1-carboxylate | 353.50 | ¹H NMR (400 MHz, CDCl3): δ ppm 8.42- 8.43 (d, 1H), 7.77- 7.79 (d, 1H), 7.51- 7.53 (d, 1H), 7.38- 7.40 (d, 1H), 7.25- 7.34 (m, 2H), 4.21 (s, 3H), 3.82-3.85 (m, 4H), 3.69 (s, 3H), 3.54-3.56 (m, 4H) |
| C-7 | 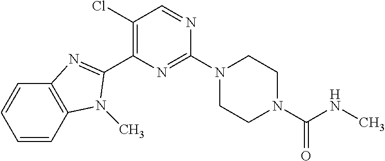 | 4-[5-Chloro-4- (1-methyl-1H- benzimidazol- 2-yl)pyrimidin- 2-yl]-N- methylpiperazine- 1-carboxamide | 386.20 | ¹H NMR (400 MHz, CDCl3): δ ppm 8.40 (s, 1H), 7.82-7.84 (d, 1H), 7.26-7.39 (m, 3H), 4.38-4.39 (d, 1H), 3.79-3.84 (m, 7H), 3.33-3.49 (m, 4H), 2.77-2.78 (d, 3H) |
| C-8 | 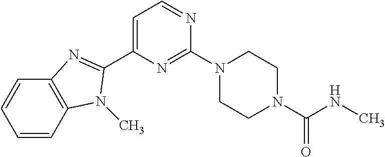 | N-Methyl-4-[4- (1-methyl-1H- benzimidazol- 2-yl)pyrimidin- 2-yl]piperazine- 1-carboxamide | 352.00 | ¹H NMR (400 MHz, CDCl3): δ ppm 8.41- 8.42 (d, 1H), 7.76- 7.78 (d, 1H), 7.52- 7.58 (d, 1H), 7.20- 7.51 (m, 3H), 4.48- 4.49 (t, 1H), 4.21 (s, 3H), 3.84-3.87 (m, 4H), 3.45-3.48 (m, 4H), 2.78-2.80 (d, 3H) |
| C-9 | 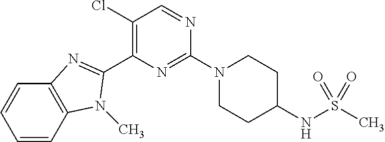 | N-{1-[5-chloro- 4-(1-methyl-1H- benzimidazol- 2-yl)pyrimidin- 2-yl]piperidin-4- yl}methane- sulfonamide | 421.30 | ¹H NMR (400 MHz, DMSO-d6): δ ppm 8.54 (s, 1H), 7.74-7.76 (d, 1H), 7.67-7.69 (d, 1H), 7.37-7.40 (m, 1H), 7.29-7.33 (m, 1H), 7.14-7.16 (m, 1H), 4.44-4.47 (t, 2H), 3.89 (s, 3H), 3.47 (m, IH), 3.15-3.23 (m, 2H), 2.94 (s, 3H), 1.91-1.93 (m, 2H), 1.37-1.45 (m, 2H) |
| C-10 | 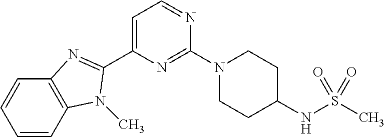 | N-{1-[4-(1- methyl-1H- benzimidazol- 2-yl)pyrimidin- 2-yl]piperidin-4- yl}methane- sulfonamide | 387.30 | ¹H NMR (400 MHz, DMSO-d6): δ ppm 8.57-8.58 (d, 1H), 7.72-7.80 (m, 2H), 7.32-7.49 (m, 3H), 7.18-7.20 (d, 1H), 4.58-4.61 (m, 2H), 4.29 (s, 3H), 3.56 (m, 1H), 3.55 (s, 3H), 3.21-3.27 (m, 2H), 3.00 (s, 3H), 1.98-2.01 (m, 2H), 1.47-1.50 (m, 2H) |
| C-11 | 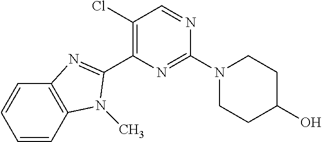 | 1-[5-chloro-4- (1-methyl-1H- benzimidazol- 2-yl)pyrimidin- 2-yl]piperidin- 4-ol | 344.05/ 346.15 | ¹H NMR (400 MHz, DMSO-d6) δ ppm 8.63 (s, 1 H) 7.75 (d, J = 8.1 Hz, 1 H) 7.68 (d, J = 7.8 Hz, 1 H) 7.35-7.45 (m, 1 H) 7.21-7.35 (m, 1 H) 4.22 (ddd, J = 13.1, 4.5, 4.3 Hz, 2 H) 3.90 (s, 3 H) 3.77 (m, J = 8.2, 8.2, 4.0, 3.8 Hz, 1 H) 3.38 (ddd, J = 13.2, 9.8, 3.0 Hz, 2 H) 1.80 (ddd, J = 8.5, 4.4, 4.3 Hz, 2 H) 1.31-1.46 (m, 2 H) |
| C-12 | 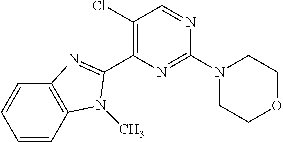 | 2-(5-chloro-2- morpholin-4- ylpyrimidin-4- yl)-1-methyl- 1H- benzimidazole | 330.10/ 332.05 | ¹H NMR (400 MHz, DMSO-d6) δ ppm 8.69 (s, 1 H) 7.76 (d, J = 7.8 Hz, 1 H) 7.68 (d, J = 8.1 Hz, 1 H) 7.35-7.47 (m, 1 H) 7.23-7.35 (m, 1 H) 3.90 (s, 3 H) 3.71- 3.81 (m, 4 H) 3.64- 3.71 (m, 4 H) |
| C-13 | 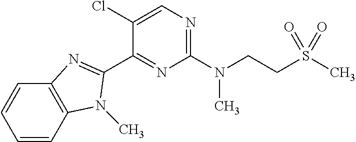 | 5-chloro-N- methyl-4-(1- methyl-1H- benzimidazol- 2-yl)-N-[2- (methylsulfonyl) ethyl]pyrimidin- 2-amine | 380.05 | ¹H NMR (400 MHz, DMSO-d6) δ ppm 8.70 (s, 1 H) 7.76 (d, J = 7.83 Hz, 1 H) 7.69 (d, J = 8.08 Hz, 1 H) 7.37-7.42 (m, 1 H) 7.29-7.34 (m, 1 H) 4.05 (t, J = 7.07 Hz, 2 H) 3.94 (s, 3 H) 3.49 (t, J = 7.07 Hz, 2 H) 3.19 (s, 3 H) 3.02 (s, 3 H) |
| C-14 | 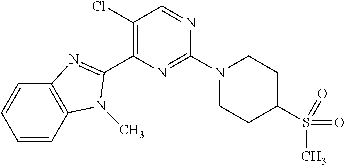 | 2-{5-chloro-2-[4- (methylsulfonyl) piperidin-1- yl]pyrimidin-4- yl}-1-methyl-1H- benzimidazole | 406.15/ 408.10 | ¹H NMR (400 MHz, DMSO-d6) δ ppm 8.68 (s, 1 H) 7.76 (d, J = 7.8 Hz, 1 H) 7.69 (d, J = 8.1 Hz, 1 H) 7.36-7.48 (m, 1 H) 7.19-7.35 (m, 1 H) 4.75 (br. s., 2 H) 3.91 (s, 3 H) 3.44 (br. s., 1 H) 3.05 (t, J = 11.9 Hz, 2 H) 2.95 (s, 3 H) 2.12 (d, J = 11.1 Hz, 2 H) 1.51-1.70 (m, 2 H) |
| C-15 | 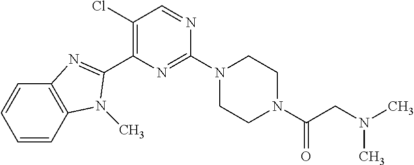 | 2-{4-[5-chloro- 4-(1-methyl-1H- benzimidazol- 2-yl)pyrimidin- 2-yl]piperazin- 1-yl}-N,N- dimethyl-2- oxoethanamine | 413.91 | ¹H NMR (400 MHz, D2O) δ ppm 8.48 (s, 1H), 7.76-7.74 (d, 1H), 7.63-7.61 (d, 1H), 7.46-7.40 (m, 2H), 3.83 (s, 3H), 3.76-3.73 (m, 4H), 3.64-3.58 (m, 4H), 3.51 (m, 2H), 2.39 (s, 6H) |
| C-16 | 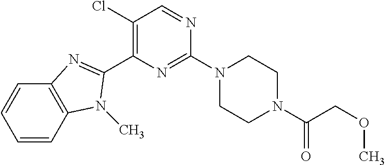 | 2-{5-chloro-2-[4- (methoxyacetyl) piperazin-1- yl]pyrimidin-4- yl}-1-methyl-1H- benzimidazole | 400.87 | ¹H NMR (400 MHz, CDCl3) δ ppm 8.9 (s, 1H), 7.92-7.90 (d, 1H), 7.45-7.36 (m, 3H), 4.16 (s, 2H), 3.91-3.89 (m, 7H), 3.72 (m, 2H), 3.60 (m, 2H), 3.45 (m, 3H) |
| C-17 | 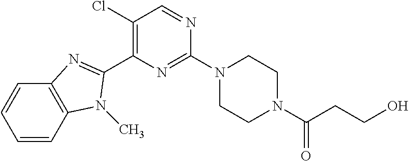 | 3-{4-[5-chloro- 4-(1-methyl-1H- benzimidazol- 2-yl)pyrimidin- 2-yl]piperazin- 1-yl}-3- oxopropan-1-ol | 400.87 | ¹H NMR (400 MHz, CDCl3) δ ppm 8.47 (s, 1H), 7.89-7.87 (d, 1H), 7.45-7.32 (m, 3H), 3.89-3.84 (m, 9H), 3.73-3.71 (m, 2H), 3.53 (m, 2H), 3.30 (brs, 1H), 2.59-2.57 (m, 2H) |
| C-18 | 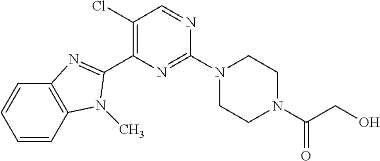 | 2-{4-[5-chloro- 4-(1-methyl-1H- benzimidazol- 2-yl)pyrimidin- 2-yl]piperazin- 1-yl}-2- oxoethanol | | ¹H NMR (400 MHz, CDCl3) δ ppm 8.53 (s, 1H), 7.95-7.93 (d, 1H), 7.50-7.38 (m, 3H), 4.26 (s, 2H), 3.94 (m, 7H), 3.81-3.80 (m, 2H), 3.61 (brs, 1H), 3.40 (m, 2H) |

### Preparation of intermediate 15: 2-(2-bromo-5-fluoropyridin-4-yl)-1-methyl-1H-benzimidazole

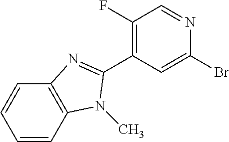

To a solution of 2-bromo-5-fluoropyridine-4-carboxylic acid (4 g, 18.2 mmol) and HATU (9.7 g, 25.5 mmol) in DMF (20 mL) was added DIPEA (4.7 g, 36.4 mmol) and N-methylbenzene-1,2-diamine (2.44 g, 20 mmol). The resulting mixture was stirred at room temperature overnight. TLC (Petroleum ether:EtOAc=4:1) showed the reaction was complete. The reaction mixture was partitioned between 100 mL of CH₂Cl₂ and 50 mL of water. The aqueous layer was extracted with CH₂Cl₂ (50 mL×2). The combined organic layers were washed with water (30 mL×4), brine (30 mL), dried and concentrated to give the crude coupled intermediate, which was purified by flash column chromatography with EtOAc/Petroleum (1/6). This amide was taken up in 150 mL of acetic acid and was heated to reflux for 2 hours. TLC (Petroleum ether:EtOAc=3:1) showed the reaction was complete. The reaction mixture was cooled to room temperature and concentrated. The residue was neutralized with aq. NaHCO₃ and extracted with EtOAc (100 mL×3). The combined organic layers were dried over anhydrous Na₂SO₄, filtered and concentrated to give the crude product, which was purified by flash column chromatography with EtOAc/petroleum (1/5) to afford the product (4.17 g, 74.7% over two steps).

### Preparation of intermediate 16: tert-butyl 4-[5-fluoro-4-(1-methyl-1H-benzimidazol-2-yl)pyridin-2-yl]piperazine-1-carboxylate

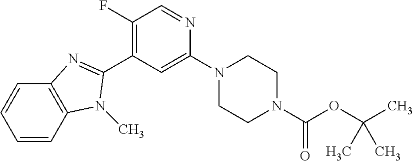

A mixture of 2-(2-bromo-5-fluoropyridin-4-yl)-1-methyl-1H-benzimidazole (800 mg, 2.62 mmol), N-BOC-piperazine (580 mg, 3.15 mmol) and CsF (800 mg, 5.24 mmol) in DMSO (20 mL) was stirred at 120° C. for 20 hours. TLC (dichloromethane:methanol=20:1) showed the reaction was complete. The solvent was evaporated. The residue was purified by Biotage F/C to give the title compound (1 g, 92%) as yellow solid.

### Preparation of intermediate 17: 2-[5-fluoro-2-(piperazin-1-yl)pyridin-4-yl]-1-methyl-1H-benzimidazole

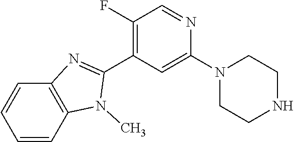

To a solution of tert-butyl 4-[5-fluoro-4-(1-methyl-1H-benzimidazol-2-yl)pyridin-2-yl]piperazine-1-carboxylate (1 g, 2.43 mmol) in dioxane (5 mL) was added HCl/Dioxane (40 mL). The mixture was stirred at room temperature overnight. TLC (dichloromethane:methanol=5:1) showed the reaction was complete. The solvent was evaporated to give the title compound (800 mg, 95%) as a yellow solid.

### Preparation of example D-6: 2-{5-fluoro-2-[4-(methylsulfonyl)piperazin-1-yl]pyridin-4-yl}-1-methyl-1H-benzimidazole

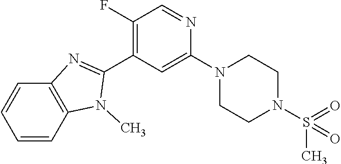

To a solution of 2-[5-fluoro-2-(piperazin-1-yl)pyridin-4-yl]-1-methyl-1H-benzimidazole (500 mg, 1.30 mmol) and Et₃N (526 mg, 5.21 mmol) in CH₂Cl₂ (10 mL) was added MsCl (225 mg, 1.95 mmol). The resulting mixture was stirred at room temperature for one hour. TLC (dichloromethane:methanol=10:1) showed the reaction was complete. CH₂Cl₂ (50 mL) was added and the mixture was washed with aq. NH₄Cl, brine, dried over Na₂SO₄, filtered and concentrated to give crude product, which was purified by Biotage F/C to give the title compound (400 mg, 79%) as a yellow solid.

### Preparation of example D-16: 1-({4-[5-fluoro-4-(1-methyl-1H-benzimidazol-2-yl)pyridin-2-yl]piperazin-1-yl}sulfonyl)-2-methylpropan-2-ol

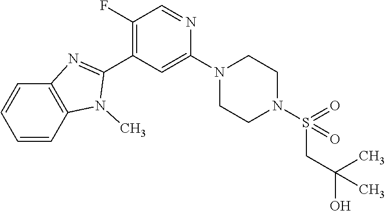

A solution of 2-{5-fluoro-2-[4-(methylsulfonyl)piperazin-1-yl]pyridin-4-yl}-1-methyl-1H-benzimidazole (400 mg, 1.03 mmol) in THF (60 mL) was cooled to −78° C. and n-BuLi (2 mL, 5.14 mmol) was added dropwise. The mixture was stirred at −78° C. for 15 min. Acetone (42 mg, 0.72 mmol) in THF (10 mL) was added dropwise. After the addition was complete, TLC (dichloromethane:methanol=10:1) showed about 60% of the starting material was consumed. The reaction was quenched with aq. NH₄Cl and mixture was diluted with water (50 mL) and extracted with EtOAc (2×30 mL). The combined organic layers were washed with brine, dried over Na₂SO₄ and concentrated in vacuo to give crude product, which was purified by silica gel chromatography (methanol:dichloromethane=6%) to give 110 mg crude product (72% in HPLC), which was re-purified by prep. HPLC to afford the title compound (55.1 mg, 12%) as light yellow solid. ¹H NMR (400 MHz, CDCl3) δ ppm 8.24 (s, 1H), 7.86-7.84 (t, 1H), 7.45-7.36 (m, 3H), 7.04-7.03 (d, 1H), 3.80 (s, 3H), 3.72-3.69 (m, 4H), 3.39-3.36 (m, 4H), 3.06 (s, 2H), 1.46 (s, 6H). m/z for C₂₁H₂₆FN₅O₃S 448.3 (M+H)⁺.

### Preparation of intermediate 18: 2-(chlorosulfonyl)ethyl acetate

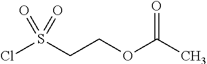

2-sulfanylethanol (20 mL, 0.29 mol) was dissolved in a 1:1 mixture (100 mL) of water and acetic acid, and the solution cooled in an ice bath. Chlorine was bubbled into this solution with vigorous stirring for 30 minutes. The yellow solution was extracted with CH₂Cl₂ (3×30 mL). The organic layers were combined and dried over Na₂SO₄ and concentrated to yield a crude product, which was then distilled under reduced pressure (bp 70-72° C., 0.1 Torr, 1 mmHg) to give the title compound as a colorless oil (7 g, 12.9%).

### Preparation of intermediate 19: 2-{2-[4-(ethenylsulfonyl)piperazin-1-yl]-5-fluoropyridin-4-yl}-1-methyl-1H-benzimidazole

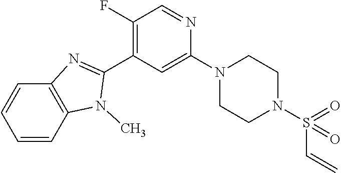

To a mixture of 2-[5-fluoro-2-(piperazin-1-yl)pyridin-4-yl]-1-methyl-1H-benzimidazole (0.65 g, 1.87 mmol) in CH₂Cl₂ (20 mL) was added DIPEA (1 mL, 5.61 mmol) at −20° C., followed by 2-(chlorosulfonyl)ethyl acetate (0.42 g, 2.2 mmol) dropwise. The mixture was stirred at room temperature for 1 hour. The reaction mixture was washed with aq. NH₄Cl and the organic layer was dried over Na₂SO₄ and concentrated to give the crude title compound (0.65 g) as a yellow syrup, which was used for the next step without further purification.

### Preparation of intermediate 20: 2-(5-fluoro-2-{4-[(2-methoxyethyl)sulfonyl]piperazin-1-yl}pyridin-4-yl)-1-methyl-1H-benzimidazole

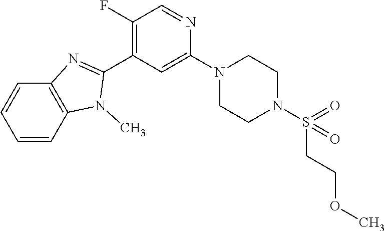

To a mixture of 2-{2-[4-(ethenylsulfonyl)piperazin-1-yl]-5-fluoropyridin-4-yl}-1-methyl-1H-benzimidazole (0.65 g, 1.87 mmol) in MeOH (20 mL) and water (3 mL) was added NaOH (0.75 g, 18.7 mmol). The reaction mixture was stirred at 50° C. for 3 hours. TLC(CH₂Cl₂/MeOH=10:1) indicated the reaction was complete. The reaction mixture was concentrated to dryness and the residue was purified by Biotage F/C eluting with EtOAc/Petroleum ether 80% to give the title compound (200 mg, 24%) as white solid. ¹H NMR (400 MHz, CDCl3) δ ppm 8.24 (s, 1H), 7.85 (d, 1H), 7.345-7.46 (m, 3H), 7.03 (d, 1H), 3.80 (d, 3H), 3.77 (t, 2H), 3.66-3.68 (m, 4H), 3.36-3.41 (m, 4H), 3.34 (s, 3H), 3.23 (t, 2H). m/z for C₂₀H₂₄FN₅O₃S 434.4 (M+H)⁺.

### Preparation of example D-15: 2-({4-[5-fluoro-4-(1-methyl-1H-benzimidazol-2-yl)pyridin-2-yl]piperazin-1-yl}sulfonyl)ethanol

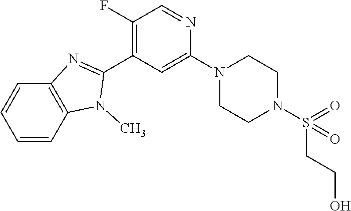

To a mixture of 2-(5-fluoro-2-{4-[(2-methoxyethyl)sulfonyl]piperazin-1-yl}pyridin-4-yl)-1-methyl-1H-benzimidazole (100 mg, 0.23 mmol) in DCM (20 mL) at 0° C. was added BBr₃ (0.3 mL) dropwise and the reaction was stirred at room temperature for one hour. The reaction mixture was then diluted with DCM (30 mL), washed with aq. NaHCO₃ and brine, dried over Na₂SO₄ and concentrated to give the title compound (90 mg, 93%) as yellow solid. ¹H NMR (400 MHz, CDCl₃) δ ppm 8.24 (s, 1H), 7.85 (d, 1H), 7.34-7.46 (m, 3H), 7.03 (d, 1H), 4.08 (m, 2H), 3.80 (d, 3H), 3.68-3.74 (m, 4H), 3.41-3.43 (m, 4H), 3.18 (t, 2H), 2.69 (br, 1H). m/z for C₁₉H₂₂FN₅O₃S 420.4 (M+H)⁺.

### Preparation of intermediate 21: tert-butyl {1-[5-fluoro-4-(1-methyl-1H-benzimidazol-2-yl)pyridin-2-yl]piperidin-4-yl}carbamate

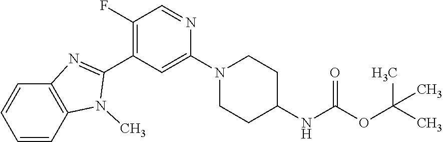

A mixture of 2-(2-bromo-5-fluoropyridin-4-yl)-1-methyl-1H-benzimidazole (3.5 g, 0.0114 mol), tert-butyl piperidin-4-ylcarbamate (2.51 g, 0.0125 mol), BINAP (1.42 g, 2.28 mmol), Pd₂(dba)₃ (1.04 g, 1.14 mmol) and K₃PO₄ (7.26 g, 0.0342 mol) in dry dioxane (40 mL) under N₂ atmosphere was heated to reflux and stirred overnight. The mixture was then cooled to room temperature and CH₂Cl₂ (50 mL) was poured into the mixture and the precipitate was removed by filtration. The filtrate was concentrated and purified by flash chromatography with CH₂Cl₂/EtOAc from 100/0 to 98/2 to give the title compound (1.3 g, 27%) as a yellow solid.

### Preparation of intermediate 22: 1-[5-fluoro-4-(1-methyl-1H-benzimidazol-2-yl)pyridin-2-yl]piperidin-4-amine

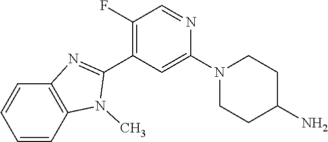

A solution of tert-butyl {1-[5-fluoro-4-(1-methyl-1H-benzimidazol-2-yl)pyridin-2-yl]piperidin-4-yl}carbamate (1.0 g, 2.35 mmol) in TFA (5 mL) and CH₂Cl₂ (5 mL) was stirred at room temperature overnight. The resulting mixture was concentrated and dried in vacuo to give the title compound (850 mg) as brown solid, which was used for next steps directly.

The following examples listed in Table 4 were prepared with appropriate substitutions with non critical method changes in analogous ways to examples in section A (using a fluorinated intermediate analogous to intermediate 5) using appropriate reagents:

TABLE 4

| Exam- | | | LRMS | |
|---|---|---|---|---|
| ple | | | m/z | |
| Num- | | Compound | (M + | |
| ber | Structure | Name | H) | ¹H NMR |
| D-1 | 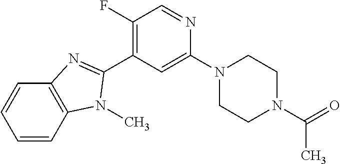 | 2-[2-(4- acetylpiperazin- 1-yl)-5- fluoropyridin- 4-yl]-1-methyl- 1H- benzimidazole | 376.3 | ¹H NMR (400 MHz, CDCl3) δ ppm 8.27 (s, 1H), 7.53-7.52 (d, 1H), 7.49-7.18 (m, 3H), 6.80- 6.79 (d, 1H), 3.88-3.86 (d, 3H), 3.81-3.75 (m, 2H), 3.66-3.58 (m, 4H), 3.56-3.51 (m, 2H), 2.15 (s, 3H) |
| D-2 | 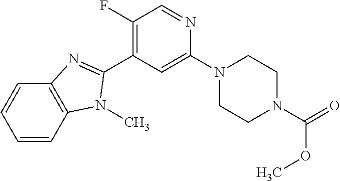 | methyl 4-[5- fluoro-4-(1- methyl-1H- benzimidazol- 2-yl)pyridin-2- yl]piperazine- 1-carboxylate | 370.3 | ¹H NMR (400 MHz, CDCl3) δ ppm 8.21 (s, 1H), 7.83 (d, 1H), 7.43 (d, 1H), 7.35 (m, 2H), 6.97 (d, 1H), 3.77 (s, 3H), 3.73 (s, 3H), 3.56 (d, 8H) |
| D-3 | 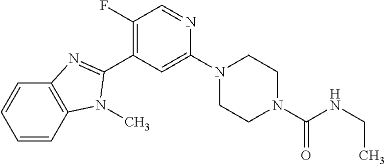 | N-ethyl-4-[5- fluoro-4-(1- methyl-1H- benzimidazol- 2-yl)pyridin-2- yl]piperazine- 1- carboxamide | 383.3 | ¹H NMR (400 MHz, CDCl3) δ ppm 8.21 (s, 1H), 7.83 (d, 1H), 7.43 (d, 1H), 7.35 (m, 2H), 6.96 (d, 1H), 4.45 (s, 1H), 3.77 (s, 3H), 3.58 (m, 4H), 3.50 (m, 4H), 3.30 (m, 2H), 1.16 (m, 3H) |
| D-4 | 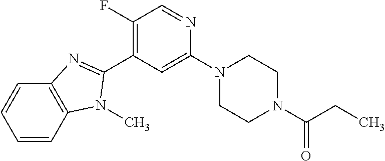 | 2-[5-fluoro-2- (4- propionyl- piperazin-1- yl)pyridin-4- yl]-1-methyl- 1H- benzimidazole | 369.3 | ¹H NMR (400 MHz, CDCl3) δ ppm 8.16 (s, 1H), 7.82-7.80 (d, 1H), 7.41-7.39 (d, 1H), 7.37- 7.29 (m, 2H), 6.95-6.93 (d, 1H), 4.40 (m, 1H), 3.75-3.74 (d, 3H), 3.56-3.54 (m, 4H), 3.46-3.44 (m, 4H), 2.78 (s, 3H) |
| D-5 | 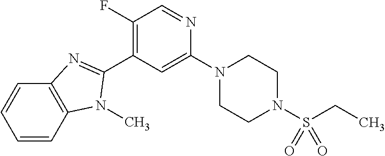 | 2-{2-[4- (ethylsulfonyl) piperazin-1- yl]-5- fluoropyridin- 4-yl}-1-methyl- 1H- benzimidazole | 404.4 | ¹H NMR (400 MHz, CDCl3) δ ppm 8.23 (s, 1H), 7.85-7.83 (d, 1H), 7.45-7.44 (d, 1H), 7.40- 7.33 (m, 2H), 7.02-7.01 (s, 1H), 3.79 (s, 3H), 3.68-3.58 (m, 4H), 3.48-3.39 (m, 4H), 3.01-2.96 (p, 2H), 1.41-1.38 (t, 3H) |
| D-6 | 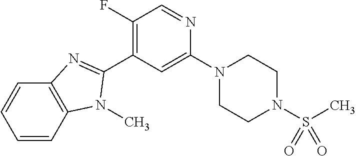 | 2-{5-fluoro-2- [4- (methylsulfonyl) piperazin-1- yl]pyridin-4- yl}-1-methyl- 1H- benzimidazole | 390.3 | ¹H NMR (400 MHz, CDCl3) δ ppm 8.23 (s, 1H), 7.85-7.83 (d, 1H), 7.46-7.44 (d, 1H), 7.41- 7.33 (m, 2H), 7.03-7.02 (s, 1H), 3.79 (s, 3H), 3.72-3.69 (m, 4H), 3.35-3.33 (m, 4H), 2.82 (s, 3H) |
| D-7 | 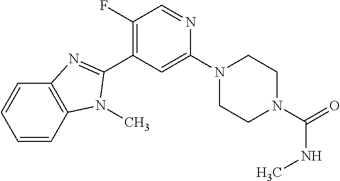 | 4-[5-fluoro-4- (1-methyl-1H- benzimidazol- 2-yl)pyridin-2- yl]-N- methylpiperazine- 1- carboxamide | 368.3 | ¹H NMR (400 MHz, CDCl3) δ ppm 8.16 (s, 1H), 7.79-7.77 (d, 1H), 7.39-7.37 (d, 1H), 7.34- 7.26 (m, 2H), 6.94-6.93 (d, 1H), 3.73-3.68 (m, 5H), 3.54-3.39 (m, 6H), 2.36-2.31 (q, 2H), 1.13- 1.10 (m, 3H) |
| D-8 | 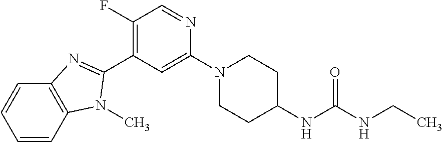 | 1-ethyl-3-{1- [5-fluoro-4-{1- methyl-1H- benzimidazol- 2-yl)pyridin-2- yl]piperidin-4- yl}urea | 419.5 | ¹H NMR (400 MHz, DMSO) δ ppm 8.32 (s, 1H), 7.74-7.72 (d, 1H), 7.68-7.66 (d, 1H), 7.36- 7.34 (m, 1H), 7.32-7.30 (m, 1H), 7.10-7.08 (m, 1H), 5.84-5.82 (d, 1H), 5.67 (m, 1H), 4.15-4.12 (d, 2H), 3.76 (s, 3H), 3.70-3.60 (brs, 1H), 3.06-2.98 (m, 4H), 1.82-1.80 (m, 2H), 1.33-1.30 (m, 2H), 0.99-0.96 (t, 3H) |
| D-9 | 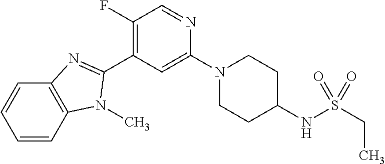 | N-{1-[5-fluoro- 4-(1-methyl- 1H- benzimidazol- 2-yl)pyridin-2- yl]piperidin-4- yl}ethanesulfon- amide | 418.4 | ¹H NMR (400 MHz, DMSO-d6) δ ppm 8.32 (s, 1H), 7.72-7.87 (d, 1H), 7.66-7.68 (d, 1H), 7.29- 7.39 (m, 2H), 7.09-7.16 (m, 2H), 4.31-4.32 (d, 2H), 3.76 (s, 3H), 3.39-3.52 (m, 1H), 2.96-3.05 (m, 4H), 1.85-1.88 (d, 2H), 1.41- 1.49 (m, 2H), 1.18-1.22 (t, 3H) |
| D-10 | 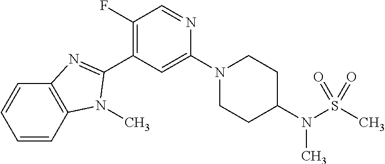 | N-{1-[5-fluoro- 4-(1-methyl- 1H- benzimidazol- 2-yl)pyridin-2- yl]piperidin-4- yl}-N- methylmethane- sulfonamlde | 418.5 | ¹H NMR (400 MHz, DMSO-d6) δ ppm 8.32 (s, 1H), 7.65-7.74 (dd, 2H), 7.28-7.38 (m, 2H), 7.11-7.12 (s, 1H), 4.39-4.42 (d, 2H), 3.82-3.87 (m, 1H), 2.87- 2.94 (m, 5H), 2.67 (s, 3H), 1.70 (brs, 4H) |
| D-11 | 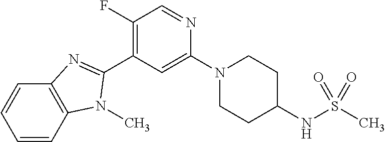 | N-{1-[5-fluoro- 4-(1-melhyl- 1H- benzimidazol- 2-yl)pyridin-2- yl]piperidin-4- yl}methanesul- fonamide | 404.4 | ¹H NMR (400 MHz, DMSO-d6) δ ppm 8.20 (s, 1H), 7.84-7.86 (d, 1H), 7.44-7.45 (d, 1H), 7.33- 7.40 (m, 2H), 7.00-7.01 (s, 1H), 4.22-4.28 (m, 3H), 3.78-3.79 (s, 3H), 3.55-3.61 (m, 1H), 3.02- 3.07 (m, 5H), 2.09-2.12 (d, 2H), 1.65-1.70 (m, 2H) |
| D-12 | 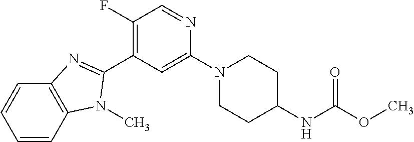 | methyl {1-[5- fluoro-4-(1- methy-1H- benzimidazol- 2-yl)pyridin-2- yl]plpetidin-4- yl}carbamate | 384.3 | ¹H NMR (400 MHz, DMSO-d6) δ ppm 8.19 (s, 1H), 7.83-7.85 (d, 1H), 7.42-7.44 (m, 1H), 7.32- 7.39 (m, 2H), 6.98-6.99 (s, 1H), 4.59 (brs, 1H), 4.19-4.22 (d, 2H), 3.67-3.78 (m, 7H), 3.01- 3.07 (t, 2H), 2.03-2.06 (m, 2H), 1.41-1.48 (m, 2H) |
| D-13 | 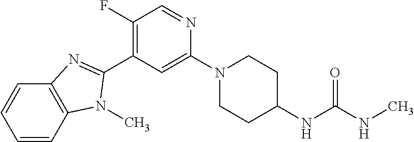 | 1-{1-[5-fluoro- 4-(1-methyl- 1H- benzimidazol- 2-yl)pyridin-2- yl]piperidin-4- yl}-3- methylurea | 383.3 | ¹H NMR (400 MHz, DMSO-d6) δ ppm 8.20 (s, 1H), 7.83-7.85 (d, 1H), 7.45-7.51 (d, 1H), 7.33- 7.40 (m, 2H), 6.97-6.99 (s, 1H), 4.34-4.39 (m, 2H), 4.11-4.22 (d, 2H), 3.89 (s, 3H), 2.97-3.07 (t, 2H), 2.76-2.89 (s, 3H), 2.02- 2.04 (d, 2H), 1.38-1.47 (m, 2H) |
| D-14 | 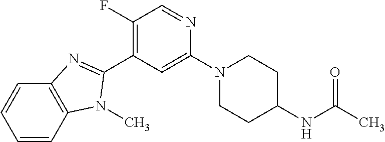 | N-{1-[5-fluoro- 4-(1-methyl- 1H- benzimidazol- 2-yl)pyridin-2- yl]piperidin-4- yl}acetamide | 368.3 | ¹H NMR (400 MHz, CDCl3) δ ppm 8.19 (s, 1H), 7.82-7.84 (d, 1H), 7.31-7.44 (m, 3H), 6.97- 6.98 (d, 1H), 5.49-5.51 (d, 1H), 4.22-4.25 (d, 2H), 3.97-4.07 (m, 1H), 3.77-3.78 (d, 3H), 2.98- 3.05 (m, 2H), 2.01-2.09 (m, 2H), 1.97 (s, 3H), 1.39-1.49 (m, 2H) |
| D-15 | 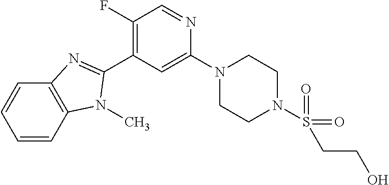 | 2-({4-[5-fluoro- 4-(1-methyl- 1H- benzimidazol- 2-yl)pyridin-2- yl]piperazin-1- yl}sulfonyl) ethanol | 420.4 | ¹H NMR (400 MHz, CDCl3) δ ppm 8.24 (s, 1H), 7.85 (d, 1H), 7.34-7.46 (m, 3H), 7.03 (d, 1H), 4.08 (m, 2H), 3.80 (d, 3H), 3.68- 3.74 (m, 4H), 3.41-3.43 (m, 4H) 3.18 (t, 2H), 2.69 (br, 1H) |
| D-16 | 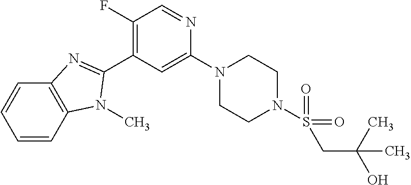 | 1-({4-[5-fluoro- 4-(1-methyl- 1H- benzimidazol- 2-yl)pyridin-2- yl]piperazin-1- yl}sulfonyl)- 2- methylpropan- 2-ol | 448.3 | ¹H NMR (400 MHz, CDCl3) δ ppm 8.24 (s, 1H), 7.86-7.84 (t, 1H), 7.45-7.36 (m, 3H), 7.04- 7.03 (d, 1H), 3.80 (s, 3H), 3.72- 3.69 (m, 4H), 3.39-3.36 (m, 4H) 3.06 (s, 2H), 1.46 (s, 6H) |
| D-17 | 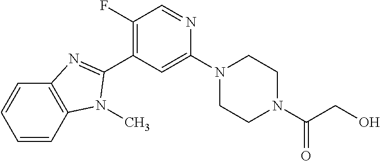 | 1-{4-[5-fluoro- 4-(1-methyl- 1H- benzimidazol- 2-yl)pyridin-2- yl]piperazin-1- yl}-2- hydroxy- ethanone | 370.3 | ¹H NMR (400 MHz, CDCl3) δ ppm 8.23 (s, 1H), 7.88-7.86 (d, 1H), 7.48-7.36 (m, 3H), 7.07- 7.06 (d, 1H), 4.22 (s, 2H), 3.81- 3.78 (m, 5H), 3.63-3.60 (m, 4H) 3.42-3.40 (m, 2H) |
| D-18 | 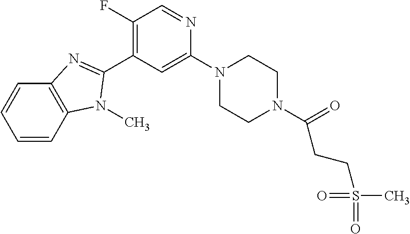 | 1-{4-[5-fluoro- 4-(1-methyl- 1H- benzimidazol- 2-yl)pyridin-2- yl]piperazin-1- yl}-3- (methylsulfonyl) propan-1- one | 446.4 | ¹H NMR (400 MHz, CDCl3) δ ppm 8.25 (s, 1H), 7.86-7.84 (d, 1H), 7.50-7.38 (m, 3H), 7.06- 7.05 (d, 1H), 3.83-3.82 (d, 3H), 3.77-3.72 (m, 2H), 3.63-3.57 (m, SH), 3.48-3.44 (m, 2H), 2.99 (s, 3H), 2.96-2.93 (m, 2H) |

### Preparation of intermediate 23: 1-methyl-2-(3-methylpyridin-2-yl)-1H-benzimidazole

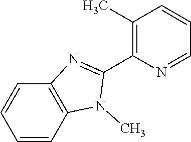

To a solution of 3-methylpyridine-2-carbaldehyde (1.00 g, 8.26 mmol) in DMSO (16 mL) was added N-methylbenzene-1,2-diamine (1.01 g, 8.26 mmol). The reaction mixture was stirred at room temperature for 5 minutes, and then sulfur was added (8.26 mmol). After being degassed with nitrogen, the reaction mixture was warmed up to 60° C. and stirred for 2 hours. The reaction mixture was cooled to RT and added to a bi-phasic stirred solution of DCM and water (80 mL ea). The resulting emulsion was extracted with DCM (2×40 mL) and the combined organics were washed with water (3×40 mL), dried over MgSO₄, filtered and stripped to a red gum. The crude product was purified by flash column chromatography (40 g silica gel, 0-6% MeOH/DCM) to provide the title compound (1.41 g, 76.5%).

### Preparation of intermediate 24: 1-methyl-2-(3-methyl-1-oxidopyridin-2-yl)-1H-benzimidazole

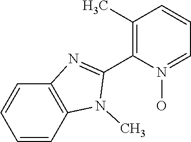

To a solution of 1-methyl-2-(3-methylpyridin-2-yl)-1H-benzimidazole (1.40 g, 6.27 mmol) in DCE (25 mL) was added mCPBA (4.77 g, 21.35 mmol). The reaction mixture was stirred at 60° C. for 18 hours. 1 M NaOH (25 ml) was added and the mixture was stirred to a dark bi-phasic solution. The organic layer was removed and the aqueous layer was extracted with DCM (3×50 mL). The organic layers were combined, dried over MgSO₄, filtered and stripped to yield an orange solid.

The crude product was purified by flash column chromatography (40 g silica gel, 1-8% MeOH/DCM) to provide the title compound (592 mg, 40%).

### Preparation of intermediate 25: 2-(6-chloro-3-methylpyridin-2-yl)-1-methyl-1H-benzimidazole

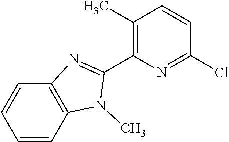

A solution of POCl₃ (880 mg, 5.74 mmol) in DCE (5 mL) was added dropwise at 10° C. to a suspension of 1-methyl-2-(3-methyl-1-oxidopyridin-2-yl)-1H-benzimidazole (572 mg, 2.39 mmol) and NEt₃ (580 mg, 5.74 mmol) in DCE (10 mL). The resulting mixture was stirred at room temperature for 10 minutes, and then heated to 45° C. for 3 hours. The reaction mixture was poured into water (25 mL) and diluted with DCM (25 mL). The mixture was neutralized by adding 3 M NaOH, and phases were separated. The organic layer was washed with brine, dried over MgSO₄, filtered and stripped to a dark gum. The crude product was purified by SFC to provide pure 2-(6-chloro-3-methylpyridin-2-yl)-1-methyl-1H-benzimidazole (245 mg, 39.8%).

Note: a regioisomeric byproduct, 2-(4-chloro-3-methylpyridin-2-yl)-1-methyl-1H-benzimidazole (81.7 mg, 13.3%) was obtained as well.

### Preparation of intermediate 26: 2-(2-fluoro-5-methylpyridin-4-yl)-1-methyl-1H-benzimidazole

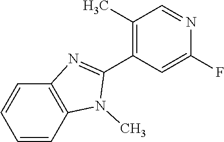

A mixture of 2-fluoro-4-iodo-5-methylpyridine (1.52 g, 6.29 mmol), 1-methyl benzimidazole (700 mg, 5.2 mmol), copper iodide (998 mg, 5.24 mmol), triphenyl phosphine (275 mg, 1.05 mmol) and sodium carbonate (1.11 g, 10.5 mmol) in DMSO (20 mL) under nitrogen was stirred at 160° C. for 17 hours. The reaction mixture was cooled to room temperature and poured into a mixture of water (100 mL) and ethylenediamine (12 mL). The combined mixture was extracted with EtOAc (2×150 mL), washed with saturated NaCl solution (150 mL), dried with sodium sulfate, filtered and concentrated. The crude product was purified by flash column chromatography (40 g silica gel, 5-50% EtOAc/Heptane) to provide the title compound (535 mg, 42%).

The following examples listed in Table 5 were prepared with appropriate substitutions with non critical method changes in analogous ways to examples in section A from intermediate 25 or 26.

TABLE 5

| Example | | Compound | LRMS m/z | |
|---|---|---|---|---|
| Number | Structure | Name | (M + H) | ¹H NMR |
| E-1 | 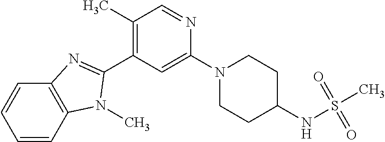 | N-{1-[5- methyl-4-(1- methyl-1H- benzimidazol- 2-yl)pyridin-2- yl]piperidin-4- yl}methanesul- fonamide | 400.2 | ¹H NMR (300 MHz, CHLOROFORM-d) δ ppm 1.50- 1.66 (m, 2 H) 2.63 (s, 4 H) 2.96- 3.10 (m, 5 H) 3.56 (br. s., 1 H) 3.66 (s, 3 H) 4.18-4.29 (m, 2 H) 4.35 (br. s., 1 H) 6.75 (s, 1 H) 7.34-7.39 (m, 2 H) 7.39-7.46 (m, 1 H) 7.81-7.89 (m, 1 H) 8.19 (s, 1 H) |
| E-2 | 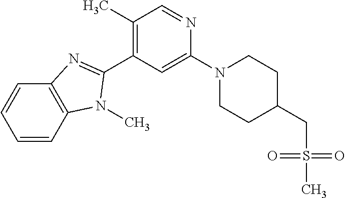 | 1-methyl-2-(5- methyl-2-{4- [(methylsul- fonyl)methyl]piper- idin-1- yl}pyridin-4-yl)- 1H- benzimidazole | 399.2 | ¹H NMR (500 MHz, DMSO-d6) δ ppm 1.27-1.43 (m, 2 H) 1.90 (d, J = 12.69 Hz, 2 H) 2.04 (s, 3 H) 2.20 (d, J = 5.86 Hz, 1 H) 2.89 (t, J = 12.20 Hz, 2 H) 2.98 (s, 3 H) 3.12 (d, J = 6.34 Hz, 2 H) 3.66 (s, 3 H) 4.25 (d, J = 12.69 Hz, 2 H) 6.90 (s, 1 H) 7.23- 7.29 (m, 1H) 7.32 (t, J = 7.56 Hz, 1 H) 7.61 (d, J = 7.81 Hz, 1 H) 7.68 (d, J = 7.81 Hz, 1 H) 8.15 (s, 1 H) |
| E-3 | 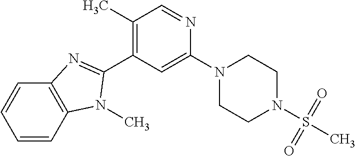 | 1-methyl-2-{5- methyl-2-[4- (methylsulfonyl)- piperazin-1- yl]pyridin-4-yl}- 1H- benzimidazole | 386.3 | ¹H NMR (500 MHz, DMSO-d6) δ ppm 2.04-2.15 (m, 3 H) 2.90 (s, 3 H) 3.17-3.24 (m, 4 H) 3.61- 3.75 (m, 7 H) 7.00 (s, 1 H) 7.24- 7.30 (m, 1 H) 7.30-7.37 (m, 1 H) 7.63 (d, J = 7.81 Hz, 1 H) 7.69 (d, J = 8.30 Hz, 1 H) 8.20 (s, 1 H) |
| E-4 | 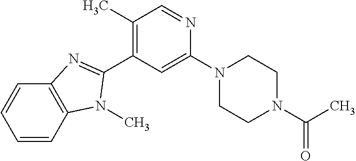 | 2-[2-(4- acetylpiperazin- 1-yl)-5- methylpyridin- 4-yl]-1-methyl- 1H- benzimidazole | 350.3 | ¹H NMR (500 MHz, DMSO-d₆) δ ppm 2.04 (s, 3 H) 2.07 (s, 3 H) 3.50 (br. s., 2 H) 3.55 (br. s., 6 H) 3.67 (s, 3 H) 6.95 (s, 1 H) 7.23- 7.30 (m, 1 H) 7.30-7.35 (m, 1 H) 7.62 (d, J = 8.30 Hz, 1 H) 7.69 (d, J = 8.30 Hz, 1 H) 8.19 (s, 1 H) |
| E-5 | 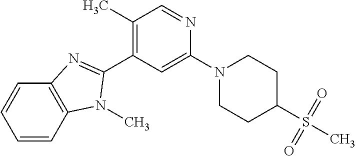 | 1-methyl-2-{5- methyl-2-[4- (methylsulfonyl)- piperidin-1- yl]pyridin-4-yl)- 1H- benzimidazole | 385.1 | ¹H NMR (500 MHz, DMSO-d₆) δ ppm 1.61 (br. s., 2 H) 2.06 (s, 5 H) 2.54 (s, 1 H) 2.87 (s, 2 H) 2.93 (s, 3 H) 3.67 (s, 3 H) 4.48 (br. s., 2 H) 6.98 (s, 1 H) 7.28 (s, 1 H) 7.32 (s, 1 H) 7.61 (s, 1 H) 7.69 (d, J = 7.81 Hz, 1 H) 8.18 (s, 1 H) |
| E-6 | 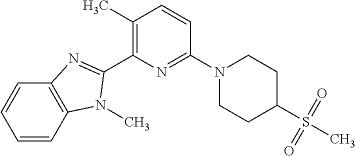 | 1-methyl-2-{3- methyl-6-[4- (methylsulfonyl)- piperidin-1- yl]pyridin-2-yl}- 1H- benzimidazole | 385.3 | ¹H NMR (500 MHz, DMSO-d₆) δ ppm 1.51-1.65 (m, 2 H) 2.02 (d, J = 5.37 Hz, 2 H) 2.28 (s, 3 H) 2.89 (s, 5 H) 3.29-3.39 (m, 1 H) 3.81 (s, 3 H) 4.41 (d, J = 13.18 Hz, 2 H) 6.97 (d, J = 9.27 Hz, 1 H) 7.18-7.25 (m, 1 H) 7.28 (t, J = 7.56 Hz, 1 H) 7.55-7.60 (m, 2 H) 7.65 (d, J = 7.81 Hz, 1 H) |

As noted above, the compounds of the invention are useful as inhibitors of SMO. Methods for determining the in vitro activity of these compounds are described below.

### Smo Radioligand Competition Binding Assay

Membranes were prepared from a stable cell line created in HEK293FlpIn-TetR cells (Invitrogen) using FIp recombinase-mediated insertion of the pSecTag-FRT/V5-His vector containing a cDNA encoding amino acids 181-787 of human Smo fused to the murine lgk leader sequence to produce a cell surface expressed Smo 181-781 protein. Hygromycin-resistant clones were obtained and stained for LacZ expression (no expression indicates a correct knock-in of my fusion cDNA). LacZ-negative cells were analyzed for binding tritiated Smo antagonist PF-03451358. For membrane preparation, the HEK293 cells expressing Smo 181-781 were grown to 90% confluence in nine to fifteen 245 mm×245 mm×22 mm dishes, washed with Dulbecco's PBS (15 ml per dish) and harvested via scraping in 10 ml of DPBS. The cells were collected and centrifuged at 1500 rpm (400×g) for 10 min at 4° C. The cell pellets were re-suspended in 40 ml of cold DPBS and washed by centrifugation at 2300 rpm (950×g max) for 10 minutes at 4° C. The supernatant was aspirated and the cell pellet was snap frozen in a methanol/dry ice bath and stored at −70° C. For membrane preparation, 15 ml of Membrane Preparation Buffer (50 mM Tris-HCl pH 7.5, 250 mM sucrose with Roche complete protease cocktail tablets) is added to the tube containing the cell pellet, then cells are rapidly thawed, and homogenized using an Ultra-Turrax T8 (IKA Labortechnik) set on “6” for 15 seconds for 5-6 times in icy water bath. This homogenate was diluted up to 50 ml using Membrane Preparation Buffer and centrifuged at 35,000 rpm in a Beckman Ti45 rotor (140,000×g) for 35 minutes at 4° C. followed by aspiration of the supernatant and re-suspension of the pellet in 5 ml of Assay Buffer (50 mM Tris-HCl pH 7.5, 100 mM NaCl, 25 mM MgCl₂, 1 mM EDTA, and 0.1% protease free bovine serum albumin). The re-suspended pellet is then homogenized in a glass tissue grinder. The re-suspended membranes are aliquoted (0.5 ml aliquots), snap frozen and stored at −70° C. Total protein in the membrane preparation is determined using the Pierce BCA protein assay (Pierce Chemical).

For the binding competition assay, 100 μl of Assay Buffer is added to all the wells of a 96 well GF/B filter plate (Millipore MultiScreen-HTS-FB cat# MSFBN6B50) for 10 minutes to pre-wet the filter prior to evacuation of the buffer (8 inches Hg for 8 seconds). To the pre-wet wells is added: 20 μl of Assay Buffer, 10 μl diluted test agent, 20 μl of ³H-PF-3451358 (15 nM stock solution), and 50 μl of membrane preparation (40 μg total protein per well). The plates are sealed and mixed at room temperature for 5 min, incubated at room temperature for 2 hours, then washed 5 times with 100 μl/each of wash buffer and vacuum dried for 8 seconds at 8 inch Hg. The plate is then dried for one hour in a 60° C. oven prior to the addition of 45 μl of Microscint 20 (Packard, #6013621) to each well and incubation at RT for 30 minutes to 1 hour. The plate is counted in a TopCount scintillation counter (Perkin Elmer).

Data analysis uses Excel for % Inhibition and Graphpad Prism for IC₅₀ calculation. Total binding (TB, in the absence of inhibitors)=average of ³H PF-03451358 3 nM+SMO membrane (40 μg/wells (approx 5000-7000 CPM). Non-specific binding (NSB)=average of ³H PF-03451358 (3 nM)+cold PF-03451358 (30 μM)+SMO membrane (approx 600-1200 CPM). Specific binding (SB)=(total binding−non-specific binding). % Inhibition=[1−(compound Specific binding/control Specific binding)]×100%. IC₅₀ is calculated by fitting the data to the four parameter sigmoidal dose-response curve (variable slope) Y=Bottom+(Top-Bottom)/(1+10̂((LogEC₅₀−X)*HillSlope)). X is the logarithm of the inhibitor concentration. Y is the response; Y starts at Bottom and goes to Top with a sigmoid shape.

### Gli-Luc/MEF Assay

The Gli-Luc/MEF cells obtained from Gli-Luc transgenic mice (Pfizer CoE, Genetically Modified Mice) contain a luciferase reporter gene under the control of the Gli response element. Luciferase activity stimulated with Sonic hedgehog ligand was inhibited by Smo inhibitors, and IC₅₀ was subsequently calculated.

Gli-Luc/MEF cells were grown in Knockout DMEM media (Invitrogen 10829-18) supplemented with 10% Heat inactive Fetal Bovine Serum (FBS, Hyclone), 2 mM L-glutamine (Invitrogen 25030-80), and 0.55 mM β-mercaptoethanol) until 90% confluence. On day one, cells were trypsinized and seeded into white 384-well plates (corning #3704) in 20 uL/well of OptiMEM media (Invitrogen 11058-021) that was supplemented with 1% Heat inactive FBS and 1 mM Sodium Pyruvate at a concentration of 7,500 cells/well. Plates were incubated at 37° C. and 5% CO₂ overnight. On day two, cells were dosed with test compounds at a final concentration ranging from 3 uM to 50 μM at a 3-time series dilution. Immediately after dosing cells with compounds, recombinant mouse sonic hedgehog (Shh, R&D Systems 464-SH) was added to a final concentration of 2 μg/mL. The cells were incubated with compounds and Shh for 48 hours at 37° C. and 5% CO₂. Luciferase assays were conducted on Day 4 using the Bright-Glo Luciferase assay system (Promega E2620) according to Promega's protocol. Briefly, Bright-Glo luciferase reagent was made up and 25 uL were added to each well of the 384-well plate containing media. Plates were kept at room temperature for 5 minutes, and then read on an Envision Luminescence plate reader (Perkin-Elmer). IC₅₀ of the inhibition was calculated by using GraphPad Prism.

The results of the Smo radioligand competition binding assay (SMO % inhibition (inh.) and SMO IC₅₀ values) and the Gli-Luc/MEF assay (Gli IC₅₀ values) for the compounds tested are listed in Table 6.

TABLE 6

| Example | SMO % inh. @ | SMO IC₅₀ | Gli IC₅₀ |
|---|---|---|---|
| Number | 0.05 μM | (nM) | (nM) |
| A-1 | 90 | 15.2 | 4 |
| A-2 | 87 | 17.2 | 3 |
| A-3 | 93 | 14.1 | 9 |
| A-4 | 91 | 29.2 | 3 |
| A-5 | 97 | 8 | 0.6 |
| A-6 | 96 | ND | 3 |
| A-7 | 39 | ND | ND |
| A-8 | 94 | 64 | 15 |
| A-9 | 74 | 189 | 23 |
| A-10 | 80 | 95 | 38 |
| A-11 | 52 | 486 | 275 |
| A-12 | 82 | 48 | 23 |
| A-13 | 66 | 423 | 89 |
| A-14 | 84 | 31 | 84 |
| A-15 | 53 | 404 | 180 |
| A-16 | 81 | 102 | 65 |
| A-17 | 99 | 13 | 6 |
| A-18 | 79 | 75 | 115 |
| A-19 | 84 | 25 | 15 |
| A-20 | 87 | 19 | 8 |
| A-21 | 78 | 152 | 90 |
| A-22 | 94 | 11 | 6 |
| A-23 | 88 | 56 | 31 |
| A-24 | 82 | 54 | 20 |
| A-25 | 91 | 8 | 7 |
| A-26 | 64 | 166 | 49 |
| A-27 | 93 | 49 | 5 |
| A-28 | 82 | 79 | 52 |
| A-29 | 78 | 61 | 110 |
| A-30 | 96 | 28 | 11 |
| A-31 | 54 | 645 | 128 |
| A-32 | 89 | 7 | 1 |
| A-33 | 98 | 9 | 8 |
| A-34 | 95 | 22 | 16 |
| A-35 | ND | ND | ND |
| A-36 | 93 | 23 | 12 |
| A-37 | 67 | 247 | ND |
| A-38 | 51 | 463 | ND |
| A-39 | 92 | 45 | 30 |
| A-40 | 66 | 158 | ND |
| A-41 | 98 | 7 | 4 |
| A-42 | 72 | 131 | 92 |
| A-43 | 86 | 190 | 92 |
| A-44 | 89 | 59 | 22 |
| A-45 | 102 | 13 | 9 |
| A-46 | 101 | 14 | 9 |
| A-47 | 101 | 9 | 7 |
| A-48 | 100 | 7 | 7 |
| A-49 | 100 | 11 | 7 |
| A-50 | 100 | 7 | 4 |
| A-51 | 99 | 6 | 7 |
| A-52 | 99 | 13 | 13 |
| A-53 | 98 | 12 | 9 |
| A-54 | 98 | 22 | 27 |
| A-55 | 98 | 26 | 10 |
| A-56 | 98 | 21 | 9 |
| A-57 | 97 | 20 | 7 |
| A-58 | 97 | 15 | 31 |
| A-59 | 96 | 12 | 3 |
| A-56 | 98 | 21 | 9 |
| A-57 | 97 | 20 | 7 |
| A-58 | 97 | 15 | 31 |
| A-59 | 96 | 12 | 3 |
| A-60 | 96 | 27 | 22 |
| A-61 | 96 | 18 | 73 |
| A-62 | 95 | 24 | 21 |
| A-63 | 95 | 25 | 18 |
| A-64 | 95 | 28 | 7 |
| A-65 | 95 | 6 | 2 |
| A-66 | 94 | 14 | 5 |
| A-67 | 94 | 33 | 26 |
| A-68 | 94 | 21 | 20 |
| A-69 | 94 | 27 | 10 |
| A-70 | 94 | 20 | 10 |
| A-71 | 94 | 18 | 10 |
| A-72 | 94 | 25 | 12 |
| A-73 | 93 | 45 | 25 |
| A-74 | 93 | 27 | 11 |
| A-75 | 92 | 23 | 11 |
| A-76 | 92 | 35 | 18 |
| A-77 | 92 | 29 | 15 |
| A-78 | 92 | 14 | 11 |
| A-79 | 92 | 23 | 7 |
| A-80 | 92 | 45 | 8 |
| A-81 | 91 | 24 | 10 |
| A-82 | 91 | 43 | 16 |
| A-83 | 91 | 49 | 23 |
| A-84 | 91 | 21 | 42 |
| A-85 | 90 | 17 | 7 |
| A-86 | 90 | 61 | 43 |
| A-87 | 89 | 58 | 9 |
| A-88 | 89 | 61 | 28 |
| A-89 | 88 | 40 | 44 |
| A-90 | 88 | 71 | 16 |
| A-91 | 88 | 55 | 16 |
| A-92 | 88 | 79 | 46 |
| A-93 | 87 | 53 | 31 |
| A-94 | 87 | 87 | 46 |
| A-95 | 86 | 70 | 38 |
| A-96 | 86 | 46 | 16 |
| A-97 | 85 | 32 | 25 |
| A-98 | 85 | 122 | 66 |
| A-99 | 84 | 84 | 28 |
| A-100 | 83 | 55 | 21 |
| A-101 | 81 | 103 | 95 |
| A-102 | 80 | 91 | 40 |
| A-103 | 80 | 153 | 27 |
| A-104 | 80 | 37 | 23 |
| A-105 | 79 | 65 | 24 |
| A-106 | 78 | 217 | 116 |
| A-107 | 77 | 123 | 37 |
| A-108 | 76 | 109 | 59 |
| A-109 | 75 | 129 | 140 |
| A-110 | 74 | 161 | 93 |
| A-111 | 73 | 116 | 25 |
| A-112 | 72 | 159 | 116 |
| A-113 | 72 | 125 | 17 |
| A-114 | 71 | 140 | 47 |
| A-115 | 69 | ND | 221 |
| A-116 | 68 | ND | 86 |
| A-117 | 68 | ND | 101 |
| A-118 | 65 | ND | 110 |
| A-119 | 64 | ND | 111 |
| A-120 | 62 | ND | 114 |
| A-121 | 62 | ND | 94 |
| A-122 | 59 | ND | 38 |
| A-123 | 59 | ND | 114 |
| A-124 | 59 | ND | 52 |
| A-125 | 57 | ND | 135 |
| A-126 | 57 | ND | 109 |
| A-127 | 57 | ND | 128 |
| A-128 | 54 | ND | 80 |
| A-129 | 53 | ND | 105 |
| A-130 | 53 | ND | 129 |
| A-131 | 52 | ND | 161 |
| A-132 | 51 | ND | 199 |
| A-133 | 48 | ND | ND |
| A-134 | 48 | ND | ND |
| A-135 | 46 | ND | ND |
| A-136 | 46 | ND | ND |
| A-137 | 45 | ND | ND |
| A-138 | 45 | ND | ND |
| A-139 | 42 | ND | ND |
| A-140 | 42 | ND | ND |
| A-141 | 96 | 34 | 27 |
| A-142 | 44 | ND | ND |
| A-143 | ND | ND | ND |
| A-144 | 78 | 90 | 159 |
| A-145 | 97 | 22 | 22 |
| A-146 | 75 | 202 | 92 |
| A-147 | ND | ND | ND |
| A-148 | 95 | 21 | 9 |
| A-149 | 95 | 14 | 8 |
| A-150 | 87 | 56 | 23 |
| A-151 | 83 | 90 | 26 |
| A-152 | 87 | 69 | 21 |
| A-153 | 93 | 23 | 15 |
| A-154 | 90 | 33 | 20 |
| A-155 | 97 | 22 | 12 |
| A-156 | 95 | 23 | 11 |
| A-157 | 95 | 35 | 24 |
| A-158 | 99 | 9 | 2 |
| A-159 | 100 | 10 | 4 |
| A-160 | 98 | 16 | 7 |
| A-161 | 98 | 9 | 3 |
| A-162 | 91 | 7 | 5 |
| A-163 | 98 | 17 | 14 |
| A-164 | 97 | 15 | 14 |
| A-165 | 62 | 374 | 280 |
| A-166 | 102 | ND | 4 |
| A-167 | 64 | 287 | ND |
| A-168 | 97 | 8 | 7 |
| A-169 | 76 | 219 | 138 |
| A-170 | 81 | 140 | 118 |
| A-171 | 97 | 31 | 28 |
| A-172 | 80 | 107 | 52 |
| A-173 | 83 | 108 | 24 |
| A-174 | 86 | 39 | 39 |
| A-175 | 97 | 17 | 9 |
| A-176 | 82 | 84 | 22 |
| A-177 | 73 | 174 | 38 |
| A-178 | 92 | 43 | 17 |
| A-179 | 97 | 10 | 7 |
| A-180 | 89 | 60 | 43 |
| A-181 | 98 | 11 | 9 |
| A-182 | 85 | 50 | 31 |
| A-183 | 89 | 81 | 65 |
| A-184 | 50 | 342 | ND |
| A-185 | 91 | 20 | 14 |
| A-186 | 78 | 97 | 65 |
| A-187 | 90 | 27 | 10 |
| A-188 | 97 | 15 | 10 |
| A-189 | 99 | 11 | 0.79 |
| A-190 | 98 | 7 | 1.4 |
| A-191 | 101 | 6 | 1.7 |
| A-192 | 87 | 15 | 2.4 |
| A-193 | 103 | 13 | 2.5 |
| A-194 | 111 | 13 | 3.7 |
| A-195 | 101 | 9.5 | 4.9 |
| A-196 | 99 | 8 | 6.7 |
| A-197 | 96 | 10 | 6.9 |
| A-198 | 87 | 9 | 7 |
| A-199 | 100 | 9 | 7.2 |
| A-200 | 98 | 15 | 7.6 |
| A-201 | 96 | 14 | 8.8 |
| A-202 | 97 | 11 | 9.0 |
| A-203 | 94 | 11 | 12.5 |
| A-204 | 96 | 19 | 12.6 |
| A-205 | 93 | ND | 14.2 |
| A-206 | 99 | 17 | 17.1 |
| A-207 | 94 | 10 | 17.3 |
| A-208 | 102 | 23 | 18.1 |
| A-209 | 97 | 18 | 19.6 |
| A-210 | 87 | 57 | 22.0 |
| A-211 | 104 | 36 | 28.4 |
| A-212 | 99 | 12 | 29.1 |
| A-213 | 83 | 78 | 56.8 |
| A-214 | 63 | 432 | 124 |
| A-215 | 79 | 189 | 63 |
| A-216 | 98 | 10 | 7 |
| B-1 | 59 | ND | ND |
| B-2 | 95 | 18 | 32 |
| B-3 | 88 | 49 | 20 |
| B-4 | ND | ND | ND |
| B-5 | 92 | 27 | 27 |
| B-6 | 95 | 10 | 12 |
| B-7 | 78 | 97 | 189 |
| B-8 | 90 | 32 | 46 |
| B-9 | 82 | 122 | 143 |
| B-10 | 72 | 82 | 89 |
| B-11 | 98 | 16 | 7 |
| B-12 | 100 | 10 | 1 |
| B-13 | 98 | 11 | 8.2 |
| C-1 | 96 | 12 | 7 |
| C-2 | 65 | 447 | ND |
| C-3 | 96 | 9 | 19 |
| C-4 | 45 | ND | ND |
| C-5 | 96 | 14 | 9 |
| C-6 | 27 | ND | ND |
| C-7 | 95 | 14 | 18 |
| C-8 | 54 | 538 | 408 |
| C-9 | 100 | 6 | 2 |
| C-10 | 97 | 44 | 57 |
| C-11 | 98 | 16 | 24 |
| C-12 | 93 | 47 | 60 |
| C-13 | ND | ND | ND |
| C-14 | 90 | 43 | 37 |
| C-15 | 95 | 64 | 8.9 |
| C-16 | 99 | 14 | 12.7 |
| C-17 | 98 | ND | 17.2 |
| C-18 | 98 | 15 | 36.0 |
| D-1 | 97 | 22 | 27 |
| D-2 | 95 | 13 | 13 |
| D-3 | 98 | 8 | 10 |
| D-4 | 98 | 19 | 20 |
| D-5 | 97 | 18 | 17 |
| D-6 | 97 | 12 | 22 |
| D-7 | 88 | 47 | 54 |
| D-8 | 95 | 6 | 6 |
| D-9 | 99 | 5 | 3.9 |
| D-10 | 102 | 7 | 4.0 |
| D-11 | 98 | 6 | 4.2 |
| D-12 | 96 | 8 | 7.7 |
| D-13 | 97 | 23 | 13.4 |
| D-14 | 97 | 19 | 17.2 |
| D-15 | 101 | 19 | 11 |
| D-16 | 94 | 9 | 3 |
| D-17 | 96 | 15 | 13 |
| D-18 | 100 | 11 | 3 |
| E-1 | 98 | 19 | 19.8 |
| E-2 | 99 | 18 | 20.9 |
| E-3 | 97 | 33 | 38.1 |
| E-4 | 92 | 61 | 52.5 |
| E-5 | 89 | 59 | 58.9 |
| E-6 | 89 | 51 | 61.6 |

While the invention has been illustrated by reference to specific embodiments, those skilled in the art will recognize that variations and modifications may be made through routine experimentation and practice of the invention. Thus, the invention is not intended to be limited by the foregoing description, but to be defined by the appended claims and their equivalents. The foregoing detailed description and examples have been provided for clarity of understanding only.

What is claimed is:

1. A compound of formula (I): 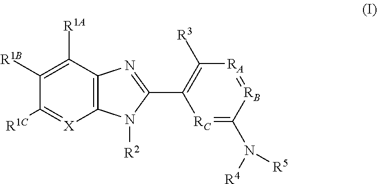
wherein:
X is selected from N and CR⁶;
R_{A}, R_{B}, and R_{C} are each independently selected from CH and N, provided that at least one of R_{A}, R_{B}, and Rc is N;
R^{1A}, R^{1B}, R^{1C} and R² are each independently selected from H, halo, —CN, C₁₋₁₀ alkyl, C₂₋₆ alkenyl, C₂₋₆ alkynyl, —NR⁶R⁷, —OR⁶, —C(O)R⁶, —C(O)OR⁶, —C(O)NR⁶R⁷, C₃₋₁₀ cycloalkyl, 3-12 membered heterocyclyl, C₆₋₁₀ aryl and 5-12 membered heteroaryl;
R³ is selected from H, halo, —CN, C₁₋₁₀ alkyl, C₂₋₆ alkenyl, O₂₋₆ alkynyl, —NR⁶R⁷, —OR⁶, —C(O)R⁶, —C(O)OR⁶, C₃₋₁₀ cycloalkyl, 3-12 membered heterocyclyl, C₆₋₁₀ aryl and 5-12 membered heteroaryl, wherein each of said C₃₋₁₀ cycloalkyl, 3-12 membered heterocyclyl, C₆₋₁₀ aryl and 5-12 membered heteroaryl of said R³ moiety is optionally substituted with at least one R⁶ group;
R⁴ and R⁵ are each independently selected from H, —(CR¹³R¹⁴)ₘCN, —(CR¹³R¹⁴)ₘ C₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘS(O)₂(R⁷), —(CR¹³R¹⁴)ₘNR⁶R⁷, —(CR¹³R¹⁴)ₘNR⁶OR⁷, —(CR¹³R¹⁴)ₘNR⁶C(O)R⁷, —(CR¹³R¹⁴)ₘNR⁶C(O)OR⁷, —(CR¹³R¹⁴)ₘNR⁶S(O)₂R⁷, —NR⁶(CR¹³R¹⁴)ₘS(O)₂NR⁶R⁷, —(CR¹³R¹⁴)ₘNR¹³(CR¹³R¹⁴)ₘOR⁷, —(CR¹³R¹⁴)ₘS(O)₂NR⁶R⁷, —(CR¹³R¹⁴)ₘOR⁶, —(CR¹³R¹⁴)ₘC(O)R⁶, —(CR¹³R¹⁴)ₘC(O)OR⁶, —(CR¹³R¹⁴)ₘC(O)NR⁶R⁷, —(CR¹³R¹⁴)ₘ(O)C(O)NR⁶R⁷, —(CR¹³R¹⁴)ₘ(O)(CR¹³R¹⁴)ₘOR⁶, —(CR¹³R¹⁴)ₘ(O)(CR¹³R¹⁴)ₘNR⁶R⁷, —(CR¹³R¹⁴)ₘC₃₋₁₀ cycloalkyl, —(CR¹³R¹⁴)ₘ(3-12 membered heterocyclyl), —(CR¹³R¹⁴)ₘ(C₆₋₁₀ aryl) and —(CR¹³R¹⁴)ₘ(5-12 membered heteroaryl), wherein each of said R⁴ and R⁵ moieties is optionally substituted with at least one R¹⁰ group;
or R⁴ and R⁵, together with the nitrogen atom to which they are attached, form a 3-12 membered heterocyclyl optionally substituted with at least one R⁶ group;
each R⁶ and R⁷ is independently selected from H, —(CR¹³R¹⁴)ₘhalo, —(CR¹³R¹⁴)ₘOH, —(CR¹³R¹⁴)ₘCN, —(CR¹³R¹⁴)ₘC₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘNR⁸R⁹, —(CR¹³R¹⁴)ₘNR⁸C(O)R⁹, —(CR¹³R¹⁴)ₘNR⁸C(O)OR⁹, —(CR¹³R¹⁴)ₘN(R⁸)S(O)₂R⁹, —(CR¹³R¹⁴)ₘN(R⁸)(CR¹³R¹⁴)ₘNR⁸R⁹, —(CR¹³R¹⁴)ₘN(R⁸)(CR¹³R¹⁴)ₘN(R⁸)S(O)₂R⁹, —(CR¹³R¹⁴)ₘN(R⁸)(CR¹³R¹⁴)ₘS(O)₂NR⁸R⁹, —(CR¹³R¹⁴)ₘ(O)(CR¹³R¹⁴)ₘNR⁸R⁹, —(CR¹³R¹⁴)ₘ(O)(CR¹³R¹⁴)ₘC(O)NR⁸R⁹, —(CR¹³R¹⁴)ₘS(O)₂R⁸, —(CR¹³R¹⁴)ₘS(O)₂NR⁸R⁹, —(CR¹³R¹⁴)ₘC(O)R⁸, —(CR¹³R¹⁴)ₘC(O)OR⁸, —(CR¹³R¹⁴)ₘC(O)NR⁸R⁹, —(CR¹³R¹⁴)ₘ(O)C(O)R⁸, —(CR¹³R¹⁴)ₘOC(O)NR⁸R⁹, —(CR¹³R¹⁴)ₘOR⁸, —(CR¹³R¹⁴)ₘ(O)(CR¹³R¹⁴)ₘOR⁸, —(CR¹³R¹⁴)ₘ(C₃₋₁₀ cycloalkyl), —(CR¹³R¹⁴)ₘ(3-12 membered heterocyclyl), —(CR¹³R¹⁴)ₘC₆₋₁₀ aryl and —(CR¹³R¹⁴)ₘ(5-12 membered heteroaryl), wherein each of said R⁶ and R⁷ moieties is optionally substituted with at least one R¹⁰ group;
each R⁸, R⁹ and R¹⁰ is independently selected from H, —(CR¹³R¹⁴)ₘhalo, —(CR¹³R¹⁴)ₘCN, —(CR¹³R¹⁴)ₘC₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘC₃₋₁₀ cycloalkyl, —(CR¹³R¹⁴)ₘC(O)R¹¹, —(CR¹³R¹⁴)ₘC(O)OR¹¹, —(CR¹³R¹⁴)ₘC(O)NR¹¹R¹², —(CR¹³R¹⁴)ₘ NR¹¹R¹², —(CR¹³R¹⁴)ₘS(O)₂R¹¹, —(CR¹³R¹⁴)ₘN(R¹¹)C(O)R¹², —(CR¹³R¹⁴)ₘ(O)(CR¹³R¹⁴)ₘC(O)NR¹¹R¹², —(CR¹³R¹⁴)ₘOR¹¹, —(CR¹³R¹⁴)ₘ(3-12 membered heterocyclyl), —(CR¹³R¹⁴)ₘ(C₆₋₁₀ aryl) and —(CR¹³R¹⁴)ₘ(5-12 membered heteroaryl);
each R¹¹ and R¹² is independently selected from H, halo, —(CR¹³R¹⁴)ₘOH, —(CR¹³R¹⁴)ₘCN, —(CR¹³R¹⁴)ₘ(C₁₋₁₀ alkyl), —(CR¹³R¹⁴)ₘ(C₂₋₆ alkenyl), —(CR¹³R¹⁴)ₘ(C₂₋₆ alkynyl), —(CR¹³R¹⁴)ₘ(C₃₋₁₀ cycloalkyl), —(CR¹³R¹⁴)ₘ(3-12 membered heterocyclyl), —(CR¹³R¹⁴)ₘ(C₆₋₁₀ aryl) and —(CR¹³R¹⁴)ₘ(5-12 membered heteroaryl);
each R¹³ and R¹⁴ is independently selected from H, C₁₋₁₀ alkyl, —OH and halo; and
each m is independently selected from 0, 1, 2, 3, 4, 5 and 6;
or a pharmaceutically acceptable salt thereof.

2. The compound of claim 1, or a pharmaceutically acceptable salt thereof, wherein R² is selected from H, halo, —CN, C₁₋₁₀ alkyl, C₂₋₆ alkenyl, C₂₋₆ alkynyl, —NR⁶R⁷, —OR⁶, —C(O)R⁶, —C(O)OR⁶ and —C(O)NR⁶R⁷.

3. The compound of claim 1, or a pharmaceutically acceptable salt thereof, wherein:
X is CH;
R^{1A}; R^{1B} and R^{1C} are H;
R² is H, halo, C₁₋₁₀ alkyl or C₃₋₁₀ cycloalkyl; and
R³ is halo or C₁₋₁₀ alkyl.

4. The compound of claim 1, or a pharmaceutically acceptable salt thereof, wherein:
X is N;
R^{1A}; R^{1B} and R^{1C} are H;
R² is H, halo, C₁₋₁₀ alkyl or C₃₋₁₀ cycloalkyl; and
R³ is halo or C₁₋₁₀ alkyl.

5. The compound of claim 1, or a pharmaceutically acceptable salt thereof, wherein:
X is CH;
R_{B} is N;
R^{1A}, R^{1B} and R_{1C} are H;
R² is H, halo, C₁₋₁₀ alkyl or C₃₋₁₀ cycloalkyl; and
R³ is halo or C₁₋₁₀ alkyl.

6. The compound of claim 1, or a pharmaceutically acceptable salt thereof, wherein:
X is N;
R_{B} is N;
R^{1A}, R^{1B} and R^{1C} are H;
R² is H, halo, C₁₋₁₀ alkyl or C₃₋₁₀ cycloalkyl; and
R³ is halo or C₁₋₁₀ alkyl.

7. The compound of claim 1, or a pharmaceutically acceptable salt thereof, wherein:
X is CH or N;
R_{C} is N;
R^{1A}, R^{1B} and R^{1C} are H;
R² is H, halo, C₁₋₁₀ alkyl or C₃₋₁₀ cycloalkyl; and
R³ is halo or C₁₋₁₀ alkyl.

8. The compound of claim 1, or a pharmaceutically acceptable salt thereof, wherein:
X is CH or N;
R_{B} and R_{C} are N;
R^{1A}, R^{1B} and R^{1C} are H;
R² is H, halo, C₁₋₁₀ alkyl or C₃₋₁₀ cycloalkyl; and
R³ is halo or C₁₋₁₀ alkyl.

9. The compound of claim 1, or a pharmaceutically acceptable salt thereof, wherein R³ is selected from H, halo, —CN, C₁₋₁₀ alkyl, C₂₋₆ alkenyl, C₂₋₆ alkynyl, —NR⁶R⁷, —OR⁶, —C(O)R⁶, —C(O)OR⁶, C₃₋₁₀ cycloalkyl, 3-12 membered heterocyclyl, C₆₋₁₀ aryl and 5-12 membered heteroaryl.

10. The compound of claim 1, or a pharmaceutically acceptable salt thereof, wherein:
X is CH;
R^{1A}, R^{1B} and R^{1C} are H;
R² is H, halo, C₁₋₁₀ alkyl or C₃₋₁₀ cycloalkyl;
R³ is halo or C₁₋₁₀ alkyl; and
R⁴ and R⁵ are independently selected from H, —(CR¹³R¹⁴)ₘCN, —(CR¹³R¹⁴)ₘC₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘS(O)₂(R⁷), —(CR¹³R¹⁴)ₘNR⁶R⁷, —(CR¹³R¹⁴)ₘNR⁶OR⁷, —(CR¹³R¹⁴)ₘNR⁶C(O)R⁷, —(CR¹³R¹⁴)ₘNR⁶C(O)OR⁷, —(CR¹³R¹⁴)ₘNR⁶S(O)₂R⁷, —NR⁶(CR¹³R¹⁴)ₘS(O)₂NR⁶R⁷, —(CR¹³R¹⁴)ₘNR¹³(CR¹³R¹⁴)ₘOR⁷, —(CR¹³R¹⁴)ₘS(O)₂NR⁶R⁷, —(CR¹³R¹⁴)ₘOR⁶, —(CR¹³R¹⁴)ₘC(O)R⁶, —(CR¹³R¹⁴)ₘC(O)OR⁶, —(CR¹³R¹⁴)ₘC(O)NR⁶R⁷, —(CR¹³R¹⁴)ₘ(O)C(O)NR⁶R⁷, —(CR¹³R¹⁴)ₘ(O)(CR¹³R¹⁴)ₘOR⁶, and —(CR¹³R¹⁴)ₘ(O)(CR¹³R¹⁴)ₘNR⁶R⁷, wherein each of said R⁴ and R⁵ moieties is optionally substituted with at least one R¹⁰ group.

11. The compound of claim 1, or a pharmaceutically acceptable salt thereof, wherein:
X is CH;
R_{B} is N, R_{C} is N, or R_{B} and R_{C} are N;
R^{1A}; R^{1B} and R^{1C} are H;
R² is H, halo, C₁₋₁₀ alkyl or C₃₋₁₀ cycloalkyl;
R³ is halo or C₁₋₁₀ alkyl; and
R⁴ and R⁵ are independently selected from H, —(CR¹³R¹⁴)ₘCN, —(CR¹³R¹⁴)ₘC₁₋₁₀ alkyl, —(CR¹³R¹⁴)ₘC₂₋₆ alkenyl, —(CR¹³R¹⁴)ₘC₂₋₆ alkynyl, —(CR¹³R¹⁴)ₘS(O)₂(R⁷), —(CR¹³R¹⁴)ₘNR⁶R⁷, —(CR¹³R¹⁴)ₘNR⁶OR⁷, —(CR¹³R¹⁴)ₘNR⁶C(O)R⁷, —(CR¹³R¹⁴)ₘNR⁶C(O)OR⁷, —(CR¹³R¹⁴)ₘNR⁶S(O)₂R⁷, —NR⁶(CR¹³R¹⁴)ₘS(O)₂NR⁶R⁷, —(CR¹³R¹⁴)ₘNR¹³(CR¹³R¹⁴)ₘOR⁷, —(CR¹³R¹⁴)ₘS(O)₂NR⁶R⁷, —(CR¹³R¹⁴)ₘOR⁶, —(CR¹³R¹⁴)ₘC(O)R⁶, —(CR¹³R¹⁴)ₘC(O)OR⁶, —(CR¹³R¹⁴)ₘC(O)NR⁶R⁷, —(CR¹³R¹⁴)ₘ(O)C(O)NR⁶R⁷, —(CR¹³R¹⁴)ₘ(O)(CR¹³R¹⁴)ₘOR⁶, and —(CR¹³R¹⁴)ₘ(O)(CR¹³R¹⁴)ₘNR⁶R⁷, wherein each of said R⁴ and R⁵ moieties is optionally substituted with at least one R¹⁰ group.

12. The compound of claim 1, or a pharmaceutically acceptable salt thereof, wherein:
X is CH;
R^{1A}, R^{1B} and R^{1C} are H;
R² is H, halo, C₁₋₁₀ alkyl or C₃₋₁₀ cycloalkyl;
R³ is halo or C₁₋₁₀ alkyl; and
R⁴ and R⁵, together with the nitrogen atom to which they are attached, form a 3-12 membered heterocyclyl optionally substituted with at least one R¹⁰ group.

13. The compound of claim 1, or a pharmaceutically acceptable salt thereof, wherein:
X is CH;
R_{B} is N, R_{C} is N, or R_{B} and R_{C} are N;
R^{1A}, R^{1B} and R^{1C} are H;
R² is H, halo, C₁₋₁₀ alkyl or C₃₋₁₀ cycloalkyl;
R³ is halo or C₁₋₁₀ alkyl; and
R⁴ and R⁵, together with the nitrogen atom to which they are attached, form a 3-12 membered heterocyclyl optionally substituted with at least one R¹⁰ group.

14. The compound of claim 1, or a pharmaceutically acceptable salt thereof, wherein:
X is N;
R^{1A}, R^{1B} and R^{1C} are H;
R² is H, halo, C₁₋₁₀ alkyl or C₃₋₁₀ cycloalkyl;
R³ is halo or C₁₋₁₀ alkyl; and
R⁴ and R⁵, together with the nitrogen atom to which they are attached, form a 3-12 membered heterocyclyl optionally substituted with at least one R¹⁰ group.

15. The compound of claim 1, or a pharmaceutically acceptable salt thereof, wherein:
X is N;
R_{B} is N, R_{C} is N, or R_{B} and R_{C} are N;
R^{1A}, R^{1B} and R^{1C} are H;
R² is H, halo, C₁₋₁₀ alkyl or C₃₋₁₀ cycloalkyl;
R³ is halo or C₁₋₁₀ alkyl; and
R⁴ and R⁵, together with the nitrogen atom to which they are attached, form a 3-12 membered heterocyclyl optionally substituted with at least one R¹⁰ group.

16. The compound of claim 1, selected from the group consisting of: 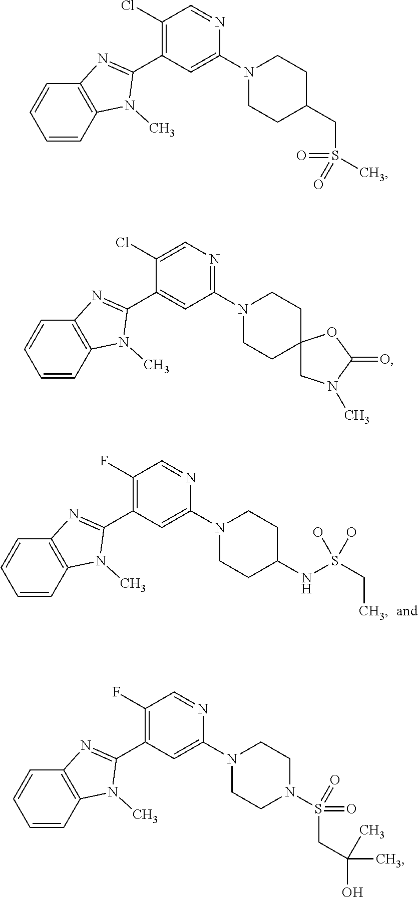
or a pharmaceutically acceptable salt thereof.

17. A method for the treatment of abnormal cell growth in a mammal, comprising administering to said mammal an amount of a compound of claim 1, or a pharmaceutically acceptable salt thereof, that is effective in treating abnormal cell growth.

18. A pharmaceutical composition, comprising a compound of claim 1, or a pharmaceutically acceptable salt thereof, and a pharmaceutically acceptable carrier or diluent.

19. The pharmaceutical composition of claim 18, further comprising at least one substance selected from an anti-angiogenesis agent, a signal transduction inhibitor, and an antiproliferative agent.

20. (canceled)

21. A pharmaceutical composition, comprising a compound of claim 16, or a pharmaceutically acceptable salt thereof, and a pharmaceutically acceptable carrier or diluent.
